(19) United States
(12) Patent Application Publication
(10) Pub. No.: US 2011/0287423 A1
(43) Pub. Date: Nov. 24, 2011

(54) DIAGNOSIS OF VIRAL INFECTIONS BY DETECTION OF GENOMIC AND INFECTIOUS VIRAL DNA BY MOLECULAR COMBING

(75) Inventors: CHARLOTTE MAHIET, PARIS (FR); FABRICE SALVAIRE, BENZONS (FR); EMMANUEL CONSEILLER, PARIS (FR); SEBASTIEN BARRADEAU, PARIS (FR)

(73) Assignee: GENOMIC VISION, PARIS (FR)

(21) Appl. No.: 13/092,210

(22) Filed: Apr. 22, 2011

Related U.S. Application Data
(60) Provisional application No. 61/327,397, filed on Apr. 23, 2010.

(51) Int. Cl. C12Q 1/68; C12N 7/00; G01N 33/53

(52) U.S. Cl. 435/6.11; 436/501; 435/235.1

(57) ABSTRACT

A method for detecting in vitro the presence of a genome of a DNA virus or a viral derived DNA in an infected eukaryotic cell, tissue or biological fluid using Molecular Combing or other nucleic acid stretching methods together with probes, especially nucleic acid probes, having a special design. A method for monitoring in vitro the effects of anti-viral treatment by following the presence of genomic viral or viral derived DNA polynucleotides in a virus-infected cell, tissue or biological fluid. Detection of an infectious form of a virus using Molecular Combing and DNA hybridization. A kit comprising probes used to carry out these methods and a composition comprising the probes.

### CROSS-REFERENCE TO RELATED APPLICATIONS

This application claims priority under 35 U.S.C. 119(e) to U.S. Provisional Application 61/327,397 filed Apr. 23, 2010 which is incorporated by reference in its entirety.

### STATEMENT REGARDING FEDERALLY SPONSORED RESEARCH OR DEVELOPMENT

(None)

### REFERENCE TO MATERIAL ON COMPACT DISK

(None)

### BACKGROUND OF THE INVENTION

1. Field of the Invention

A method for easily, rapidly and accurately detecting the presence of infectious viral DNA or other infectious or pathogenic DNA sequences in a viral host cell, tissue or biological fluid obtained from a subject or patient using Molecular Combing and/or DNA stretching in combination with specially designed probes for the infectious viral or pathogenic DNA sequence. A method for monitoring in vitro the effects of anti-viral treatment by following the presence of genomic viral polynucleotides in a virus-infected cell, tissue or a fluid of a patient to be tested as infected by a virus. Detection of infectious virus based on DNA hybridization. A kit comprising the probes used to carry out a method of the invention and a composition comprising said probes.

2. Description of the Related Art

Molecular Combing

Molecular Combing technology has been disclosed in various patents and scientific publications, for example in U.S. Pat. No. 6,303,296, WO 9818959, WO 0073503, U.S. 2006/257910, U.S. 2004/033510, U.S. Pat. No. 6,130,044, U.S. Pat. No. 6,225,055, U.S. 6,054,327, WO 2008/028931, WO 2010/035140, and Michalet, Ekong et al. 1997, Herrick, Michalet et al. 2000, Herrick, Stanislawski et al. 2000, Gad, Aurias et al. 2001, Gad, Caux-Moncoutier et al. 2002, Gad, Klinger et al. 2002, Herrick, Jun et al. 2002, Pasero, Bensimon et al. 2002, Gad, Bieche et al. 2003, Lebofsky and Bensimon 2003, Herrick, Conti et al. 2005, Lebofsky and Bensimon 2005, Lebofsky, Heilig et al. 2006, Patel, Arcangioli et al. 2006, Rao, Conti et al. 2007 and Schurra and Bensimon 2009 (see citations below). The techniques, supplies and equipment described in these references, specifically those pertaining or relating to Molecular Combing, are hereby incorporated by reference to the publications cited above.

Bensimon, et al., U.S. Pat. No. 6,303,296, discloses DNA stretching procedures, Lebofsky, et al., WO 2008/028931 discloses detections of genomic sequences, and Lebofsky, et al., WO 2010/035140 A1, discloses a method for analysis of D4Z4 tandem repeat arrays on human chromosomes 4 and 10 based on stretching of nucleic acid and on Molecular Combing.

Molecular Combing is a technique enabling the direct visualization of individual nucleic acid molecules and has been successfully used for the location of myc and certain Human Papillomavirus (HPV) sequences in cervical cancer cell lines as well as for the study of replication kinetics on previously characterized MYC/papillomavirus (HPV18) amplicon and particularly on its origin of replication (Herrick, et al., Cancer Res. 65(4): 1174-1179 2005). Nucleic acid rearrangements and amplifications in tumor cells were correlated with cancer outcome using this technique. Herrick, et al. probed combed nucleic acids from cultured tumor cell lines IC1, IC2, IC4 and IC5 with probes specific to N-myc and c-myc genes and probes to detect HPV nucleic acids containing full-length HPV-18 and HPV-44 (˜7.8 kb). Conti, et al., Genes, Chromosomes & Cancer 46:724-734 (2007) studied origin activation in an HPV/MYC amplicon containing certain HPV 18 nucleic acid sequences and analyzed origin activity and fork movement on DNA stretched by Molecular Combing from the IC1 tumor cell line. Conti identifies gene rearrangements and amplifications as hallmarks of cancer cells focusing on the mechanisms involving both HPV and MYC nucleic acid sequences. Both these studies used cultured tumor cells in which HPV nucleic acid sequences act as oncogenes but not as infectious genomic viral nucleic acids.

Episomal and integrated Epstein-Barr virus DNA obtained from cultured Burkitt's lymphoma cell lines was characterized by FISH on combed DNA, (Reisinger, et al., Int. J. Cancer 118: 1603-1608 (2006). Reisinger's objective was to study the genomic organization of viral genomes and understand and attempt to discriminate between the episomal and integrated forms of EBV in infected eukaryotic cells. DNA from cultured Burkitt's lymphoma cell lines was stretched by Molecular Combing and probed with EBV-specific DNA probes that cover genomic sequences but not the region between the terminal repeat (TR) and the internal repeat 1 (IR1) sequences.

Reisinger et al. and Herrick et al. also do not describe methods for detecting rearrangements of viral genomes using probe sets tagged with different haptens that can generate a different color-fluorescence array that will allows the identification of rearrangements within this specific region of the viral DNA. These two references describe the detection of viral DNA in cultured cells but not in tissue or in biological fluids.

In contrast, in the present invention, the standard extraction procedure is modified to allow the extraction of viral DNA from viral particles in order to analyze by Molecular Combing the structure of the viral DNA in the infectious particles. Moreover, the invention develops a methodology of extraction of high molecular weight DNA from solid tissues such as cornea that is compatible with the analysis by Molecular Combing and that allows the detection of the presence of the infectious forms of HSV DNA in an infected cornea. These two methodologies have never been described before this invention.

Stretching nucleic acid, in particular viral or genomic DNA provides immobilized nucleic acids in linear and parallel strands and is preferably performed with a controlled stretching factor on an appropriate surface (e.g. surface-treated glass slides). After stretching, it is possible to hybridize sequence-specific probes detectable for example by fluorescence microscopy (Lebofsky, Heilig et al. 2006). Thus, a particular sequence may be directly visualized on a single molecule level. The length of the fluorescent signals and/or their number, and their spacing on the slide provides a direct reading of the size and relative spacing of the probes. However, up to now, it was not shown that this technology can be used to detect the infectious forms of viruses in an infected eukaryotic cell or viral sequences or genome of a derived mutated viral sequence infecting a cell or tissue or present in a fluid of mammals.

Virus Structure and Genome

The viruses that cause human diseases are placed into seven groups based upon the existence of an envelope, the type of the capsid, the nature of the genome (DNA or RNA, double-stranded or single-stranded), size, and target cells (International Committee on Taxonomy of Viruses, http://_www.ictvonline.org; last accessed Apr. 7, 2011). The enveloped DNA viruses include the poxviruses, herpesviruses, and hepadnaviruses. The nonenveloped group includes the adenoviruses, papovaviruses, and parvoviruses.

An enormous variety of genomic structures can be seen among viral species. Indeed, there are millions of different types of viruses (Breitbart and Rohwer 2005), though only about 5,000 of them have been described in detail (Dimmock, Easton et al. 2007). A viral genome is either single-stranded or double-stranded. Single-stranded genomes consist of an unpaired nucleic acid, analogous to one-half of a ladder split down the middle. All RNA viruses are single-stranded except for the double-stranded reoviruses. Double-stranded genomes consist of two complementary paired nucleic acids, analogous to a ladder. All DNA viruses are double-stranded except for the parvoviruses, which have single-stranded DNA. Some viruses, such as those belonging to the Hepadnaviridae, contain a genome that is partially double-stranded and partially single-stranded (Collier and Oxford 2006)

Viral genomes are circular, as in the polyomaviruses, or linear, as in the adenoviruses (Fields, Knipe et al. 2007). The type of nucleic acid is irrelevant to the shape of the genome. Among RNA viruses, the genome is often divided up into separate parts within the virion and is called segmented. Each segment often codes for one protein and they are usually found together in one capsid. Every segment is not required to be in the same virion for the overall virus to be infectious, as demonstrated by the brome mosaic virus (Collier and Oxford 2006).

For viruses with RNA or single-stranded DNA, the strands are said to be either positive-sense (called the plus-strand) or negative-sense (called the minus-strand), depending on whether it is complementary to the viral messenger RNA (mRNA) (Fields, Knipe et al. 2007). Positive-sense viral RNA is identical to viral mRNA and thus can be immediately translated by the host cell. Negative-sense viral RNA is complementary to mRNA and thus must be converted to positive-sense RNA by an RNA polymerase before translation. DNA nomenclature is similar to RNA nomenclature, in that the coding strand for the viral mRNA is complementary to it (−), and the non-coding strand is a copy of it (+). Some single-stranded RNA viruses called retrovirus (which includes HIV) replicate in the host cell via the enzyme reverse transcriptase to produce DNA from its RNA genome. The DNA is then incorporated into the host's genome by an integrase enzyme. The virus genome, or provirus, remains in the genome of the infected cell and thereafter replicates as part of the host cell's DNA.

Genome size varies greatly amongst different kinds or species of viruses ranging from 1.7 kb for the hepatitis delta virus (HDV) to 309 kb for the Poxvirus. RNA viruses generally have smaller genome sizes than DNA viruses because of a higher error-rate when replicating, and have a maximum upper size limit. Beyond this limit, errors in the genome when replicating render the virus useless or uncompetitive. To compensate for this, RNA viruses often have segmented genomes where the genome is split into smaller molecules, thus reducing the chance of error. In contrast, DNA viruses generally have larger genomes because of the high fidelity of their replication enzymes (Pressing and Reanney 1984).

Viruses can undergo genetic changes that occur through different mechanisms. The first process is called genetic drift where individual bases in the DNA or RNA mutate to other bases. Most of these point mutations are “silent” meaning that they do not change the protein that the gene encodes but others can confer evolutionary advantages such as resistance to antiviral drugs. Antigenic shift occurs when there is a major change in the genome of the virus (Pan, Li et al. 2007). This can be a result of recombination or reassortment. When this happens with influenza viruses (Hampson and Mackenzie 2006), pandemics might result. RNA viruses often exist as quasispecies or swarms of viruses of the same species but with slightly different genome nucleoside sequences. Such quasispecies are a prime target for natural selection (Metzner 2006). Secondly, segmented genomes confer also evolutionary advantages; different strains of a virus with a segmented genome can shuffle and combine genes and produce progeny viruses that have unique and new characteristics. This is called reassortment or viral sex (Goudsmit 1998). Finally, genetic recombination which is the process by which a strand of DNA is broken and then joined to the end of a different DNA molecule can arise. This can occur when viruses infect cells simultaneously and studies of viral evolution have shown that recombination has been rampant in the species studied (Worobey and Holmes 1999). Recombination is common to both RNA and DNA viruses (Umene 1999; Lukashev 2005).

The HSV are among the larger viruses with a diameter ranging from about 150 nm to 200 nm. They are enclosed within a loosely fitting envelope that contains glycoprotein spikes (Wildy, Russell et al. 1960). Like other enveloped viruses, herpes viruses are prone to deactivation by organic solvents or detergents and are unstable outside the host's body. The icosahedral capsid houses a core of double-stranded DNA that winds around a proteinaceous spindle in some viruses. The genome sequence of HSV-1 strain 17, first published in 1988, contains 152,261 residues in each strand (Genbank accession number: NC_{—}001806.1; last accessed Apr. 7, 2011) (McGeoch, Dalrymple et al. 1988). The HSV genome is regarded as being composed of two covalently joined segments, the long (L) and short (S) regions (Roizman 1979). The L region consists of a unique sequence (U_{L}) flanked by a pair of oppositely oriented repeat elements (termed R_{L}, with the terminal and internal copies specifically referred to as TR_{L} and IR_{L}) (Wadsworth, Jacob et al. 1975). The S region similarly consists of IR_{S}, U_{S}, and TR_{S}. The sequences of the R_{L} and R_{S} elements are distinct, except that there is a 400 base pairs (bp) direct repeat at the genome termini, termed the a sequence, and at least one further copy of this is found at the junction of L and S regions, in the orientation opposite to the terminal copies (Roizman 1979; Davison and Wilkie 1981). Each terminus possesses one overhanging residue, with the 3′-hydroxyl group free (Mocarski and Roizman 1982). Preparations of HSV DNA contain equimolar amounts of four sequence-orientation isomers, in which U_{L} and U_{S} each lie independently in one of two possible orientations with respect to the joint between L and S sequences (Hayward, Jacob et al. 1975; Bataille and Epstein 1997). One isomer is defined as the prototype (Roizman 1979). HSV-1 DNA has a base composition of 68.3% G+C (McGeoch, Dalrymple et al. 1988). The G+C content is not constant throughout the genome; the 6.6-kbp R_{S} elements deviate most notably from the mean, with a G+C content of 79.5% (McGeoch, Dolan et al. 1986). The genome of HSV-2 has not been completely sequenced, but it closely resembles that of HSV-1, albeit with a slightly higher G+C content.

The genomic polynucleotide sequences of numerous viruses are well-known to those of skill in the art and are incorporated by reference to Fields, B. N., D. M. Knipe, et al. (2007), Fields' Virology. Philadelphia, Wolters kluwer/Lippincott Williams & Wilkins; and to the NCBI Virus Genomes database accessible at http://www._ncbi.nlm.nih.gov/genomes/genlist.cgi?taxid=10239&type=5&name=Viruses (last accessed Apr. 7, 2011).

Viral Pathogenesis

Many types of viral pathogens are recognized. For a recent review on viral infection, one can refer to Fields, et al. (Fields, Knipe et al. 2007) and references therein which are incorporated by reference. A viral infection occurs when a virus enters the body through such processes as breathing air contaminated with a virus, eating contaminated food, or by having sexual contact with a person who is infected with a virus. A viral infection may also be caused by an insect bite. In a viral infection, the virus invades the inside of the body's cells in order to reproduce. A virus then spreads to other cells and repeats the process. This process of viral infection results in a variety of symptoms that vary in character and severity depending on the type of viral infection and individual factors. Common symptoms of a viral infection include fatigue, flu-like symptoms and fever.

Many types of viral infections, such as a common cold, are self limiting in generally healthy people. This means that the viral infection causes illness for period of time, then it resolves and symptoms disappear. However, some people are at risk for developing serious complications of viral infection. In addition, certain types of viral infections, such as HIV/AIDS, are not self limiting and cause serious complications and/or can eventually be fatal. As an example of serious damage, some forms of measles virus infection lead to brain's inflammation, and consequently permanent brain injury.

There are many types of viruses that cause a wide variety of viral infections or viral diseases. For example, there are over 200 different viruses that can cause a cold or an upper respiratory infection. Other common viruses include the influenza virus which causes influenza or the flu. The Epstein-Barr virus and the cytomegalovirus cause infectious mononucleosis, the varicella zoster virus (VZV) causes shingles and chickenpox, and HIV causes AIDS.

Viruses have different mechanisms by which they produce disease in an organism, which largely depends on the viral species. Mechanisms at the cellular level primarily include cell lysis, the breaking open and subsequent death of the cell. In multicellular organisms, if enough cells die the whole organism will start to suffer the effects. Although viruses cause disruption of healthy homeostasis, resulting in disease, they can also exist relatively harmlessly within an organism. An example would include the ability of the herpes simplex virus (HSV), which causes cold sores, to remain in a dormant state within the human body. This is called latency (Margolis, Elfman et al. 2007) and is a characteristic of the all herpes viruses including the EBV, which causes glandular fever, and the VZV. Most people have been infected with at least one of these types of HSV (Whitley and Roizman 2001). However, these latent viruses might sometimes be beneficial, as the presence of the virus can increase immunity against bacterial pathogens, such as Yersinia pestis (Barton, White et al. 2007). On the other hand, latent chickenpox infections return in later life as the disease called shingles.

Some viruses can cause life-long or chronic infections, where the viruses continue to replicate in the body despite the host's defense mechanisms (Bertoletti and Gehring 2007). This is common in hepatitis B and C virus infections. People chronically infected are known as carriers, as they serve as reservoirs of infectious virus. In populations with a high proportion of carriers, the disease is said to be endemic (Rodrigues, Deshmukh et al. 2001). In contrast to acute lytic viral infections this persistence implies compatible interactions with the host organism.

HSV-1 infection is one example of a viral infection. HSV was named for the tendency of some herpes infections to produce a creeping rash (Taylor, Brockman et al. 2002). It is the common name for a large family whose members include herpes simplex 1 and 2, the cause of fever blisters and genital infections; VZV, the cause of chickenpox and shingles; cytomegalovirus (CMV), which affects the salivary glands and other viscera; EBV, associated with infection of the lymphoid tissue; and some recently identified viruses (HSV-6, -7, and -8). Prominent features of the family are its tendency toward viral latency and recurrent infections.

HSV infections, often colloquially called herpes, usually target the mucous membranes. The virus enters cracks or cuts in the membrane surface and then multiplies in basal and epithelial cells in the immediate vicinity. This results in inflammation, edema, cell lysis, and formation of a characteristic thin-walled vesicle. The main diseases of HSV are facial herpes (oral, optic, and pharyngeal), genital herpes, neonatal herpes, and disseminated disease. Herpes labialis otherwise known as fever blisters or cold sores is the most common recurrent HSV-1 infection. Vesicles usually crop up on the muco-cutaneous junction of the lips or on adjacent skin. Herpetic keratitis (also called ocular herpes) is an infective inflammation of the eye in which a latent virus travels into the ophthalmic rather than the mandibular branch of the trigeminal nerve. Preliminary symptoms are a gritty feeling in the eye, conjunctivitis, sharp pain, and sensitivity to light. Some patients develop characteristic branched or opaque corneal lesions as well. In 25% to 50% of cases, keratitis is recurrent and chronic and can interfere with vision.

The primary infection, asymptomatic in more than 90% of cases (Liesegang 1989) takes place in the oral mucosae, as a consequence of contact with infected particles of saliva. Soon after infection, the linear viral genome circularizes and DNA replication initiates at an origin (for review: (Boehmer and Lehman 1997). DNA replication initially proceeds by a theta mechanism and subsequently switches to a sigma or rolling-circle mode to yield long head-to-tail concatemers. Multiple DNA replication forks that arise from homologous recombination, a sequence-mediated genome isomerization, and other events, lead to the formation of an extensive network of branched DNA intermediates. Finally, these structures are resolved into unit-length genomes and packaged into preassembled capsids. Nevertheless, DNA molecules shorter than the full-length standard HSV-1 viral DNA can become encapsidated within nuclear capsids provided they contain the cleavage/packaging signal (Vlazny, Kwong et al. 1982). However, capsids containing HSV-1 genome significantly shorter than standard viral genome are not infective.

After replication in epithelial tissues, viruses propagate in neurons before becoming latent. Type 1 HSV enters primarily the trigeminal, or fifth cranial, nerve, which has extensive innervations in the oral region while type 2 HSV usually becomes latent in the ganglion of the lumbo-sacral spinal nerve trunk. Following various triggering factors such as fever, UV radiation, stress, or mechanical injury, the virus migrates to the body surface and produces a local skin or membrane lesion, often in the same site as a previous infection. Since the principal location of latent HSV-1 is the trigeminal ganglion, responsible for sensory innervation of the face, most recurrences are located in the eyes or the lips. The seroprevalence of HSV-1 in the general population ranges from 24.5% to 67%, with 30 to 70% of positive subjects experiencing recurrent herpes labialis (Liesegang 2001). Indeed, almost everyone may potentially exhibit an HSV-1 infection event since recent studies using Polymerase Chain Reaction (PCR) on post-mortem tissues showed that nearly 100% of people at least 60 years of age have several HSV-1 genome copies present in the trigeminal ganglion (Wang, Lau et al. 2005). The eye, and particularly the cornea, is the second most frequent location of HSV-1 infection. The prevalence of herpes keratitis is 149/100,000 (Liesegang, Melton et al. 1989), with more than 30 new events per 100,000 inhabitants annually (Labetoulle, Auquier et al. 2005). Once the eye has been clinically affected by an herpetic infection, the rate of relapse is 23% within 2 years and 40% within 5 years of follow-up (Wilhelmus, Coster et al. 1981; Liesegang 1989). The visual prognosis is poor, especially in deep corneal infections (stromal keratitis), with up to 60% of affected eyes reaching a visual acuity of less than 20/40 after 5 years of follow-up (Kabra, Lalitha et al. 2006). As a consequence, HSV-1 is a leading cause of blindness throughout the world. In less developed countries, HSV-1 ocular infection accounts for about 10% of patients attending corneal clinics (Pramod, Rajendran et al. 1999), and despite the use of topical or oral antiviral agents in the last three decades, herpes simplex keratitis also remains the most frequent cause of infectious corneal opacities in the most developed areas of the world (group 1998), accounting for about 10% of patients undergoing corneal transplantation (Leger, Larroque et al. 2001). Moreover, the natural risk of HSV-1 reactivation in recently grafted cornea is about 25% in the first year following surgery (Lomholt, Baggesen et al. 1995), and about 33% of primary graft failure (no clearing of the graft) have shown to be associated with the presence of the HSV-1 genome in the cornea (Cockerham, Bijwaard et al. 2000). These data explain why HSV-1 remains a major cause of corneal graft failure, accounting for about 22% of all cases of re-grafting.

The clinical complications of latency and recurrent infections become more severe with advancing age, cancer chemotherapy, or other conditions that compromise the immune defenses. The HSV are among the most common and serious opportunists among AIDS patients (Ramaswamy and Geretti 2007). Nearly 95% of this group will experience recurrent bouts of skin, mucous membrane, intestinal, and eye disease from these viruses.

Human immunodeficiency virus (HIV) is a lentivirus (a member of the retrovirus family) that causes acquired immunodeficiency syndrome (AIDS) (Weiss 1993; Douek, Roederer et al. 2009), a condition in humans in which the immune system begins to fail, leading to life-threatening opportunistic infections. Infection with HIV occurs by the transfer of blood, semen, vaginal fluid, pre-ejaculate, or breast milk. Within these bodily fluids, HIV is present as both free virus particles and virus within infected immune cells. The four major routes of transmission are unsafe sex, contaminated needles, breast milk, and transmission from an infected mother to her baby at birth (vertical transmission). Screening of blood products for HIV has largely eliminated transmission through blood transfusions or infected blood products in the developed world.

HIV infection in humans is considered pandemic by the World Health Organization (WHO). From its discovery in 1981 to 2006, AIDS killed more than 25 million people. HIV infects about 0.6% of the world's population. In 2005 alone AIDS claimed an estimated 2.4-3.3 million lives of which more than 570,000 were children. Antiretroviral treatment reduces both the mortality and the morbidity of HIV infection.

HIV infects primarily vital cells in the human immune system such as helper T cells (to be specific, CD4⁺ T cells), macrophages, and dendritic cells. HIV infection leads to low levels of CD4⁺ T cells through three main mechanisms: first, direct viral killing of infected cells; second, increased rates of apoptosis in infected cells; and third, killing of infected CD4⁺ T cells by CD8 cytotoxic lymphocytes that recognize infected cells. When CD4⁺ T cell numbers decline below a critical level, cell-mediated immunity is lost, and the body becomes progressively more susceptible to opportunistic infections.

Most people infected with HIV-1 left untreated eventually develop AIDS and eventually die from opportunistic infections or malignancies associated with the progressive failure of the immune system (Lawn 2004). HIV progresses to AIDS at a variable rate affected by viral, host, and environmental factors; most will progress to AIDS within 10 years of HIV infection: some will have progressed much sooner, and some will take much longer (Buchbinder, Katz et al. 1994; 2000). Treatment with anti-retroviral drugs increases the life expectancy of people infected with HIV and even after HIV has progressed to diagnosable AIDS, the average survival time with antiretroviral therapy was estimated to be more than 5 years as of 2005 (Schneider, Gange et al. 2005). Without antiretroviral therapy, someone who has AIDS typically dies within a year (Morgan, Mahe et al. 2002).

Diagnosis of and Treatment of Viral Infections

Viral diseases are diagnosed by a variety of clinical and laboratory methods. These include a clinical assessment of symptoms, viral isolation in cell or animal culture, and serological testing for antibodies to a virus. Making a diagnosis of a viral infection begins with taking a thorough personal and family medical history, including symptoms, and completing a physical examination. Diagnosing some viral infections, such as seasonal influenza, may be made based on a history and physical. Blood tests, such as a complete blood count may be done. A complete blood count measures the numbers of different types of blood cells, including white blood cells (WBCs). Different types of WBCs increase in number in characteristic ways during an infectious process, such as viral infection. A culture test may also be performed. This test requires taking a small sample from the body area that is suspected to be infected with a virus and grows the sample in a lab to determine the type of microorganism causing illness. Common samples tested with a culture include those from the throat, blood, and sputum from the lungs. Diagnostic tests may also include a lumbar puncture, also called a spinal tap, which involves withdrawing a small sample of cerebrospinal fluid (CSF) from the spine with a needle. The sample of CSF is tested for white blood cells and other indications of viral infection that may be in the spine or brain, such as viral meningitis. X-rays may be performed to assist in the diagnosis of some viral infections. This may include taking a chest X-ray for suspected cases of viral pneumonia. Additional tests may be performed in order to rule out or confirm other diseases that may accompany viral infection or cause similar symptoms, such as a secondary bacterial infection.

HSV infections are diagnosed by a variety of clinical and laboratory methods, including a clinical assessment of symptoms, isolation in cell or animal culture, and serological testing for antibodies to HSV. Small, painful, vesiculating lesions on the mucous membranes of the mouth or genitalia, lymphadenopathy, and exudate are typical diagnostic symptoms of herpes simplex. Further diagnostic support is available by examining scrapings from the base of such lesions stained with Giemsa, Wright (also called Tzanck preparation), or Papanicolaou (Pap) methods. The presence of multinucleate cells, giant cells, and intranuclear eosinophilic inclusion bodies can help establish herpes infection. This method, however, will not distinguish among HSV-1, HSV-2, or other herpesviruses, which require more specific subtyping. Moreover, laboratory culture and specific tests are essential for diagnosing immunosuppressed and neonatal patients with severe, disseminated herpes infection. A specimen of tissue or fluid is introduced into a primary cell line such as monkey kidney or human embryonic kidney tissue cultures and is then observed for cytopathic effects within 24 to 48 hours. Direct tests on specimens or cell cultures using fluorescent antibodies or detection of DNA using specific probes or amplification by Polymerase Chain Reaction (PCR) can differentiate among HSV-1, HSV-2, and closely related HSV, but are not useful tools to confirm that the genomes of said viruses found in a sample are intact or infectious. While serological analysis is useful for primary infection, it is inconclusive for recurrent illness because the antibody titer to HSV upon recurrence of the infection usually does not increase.

Several agents are available for treatment of HSV infections. Acyclovir is the most effective therapy developed to date that is nontoxic and highly specific to HSV. Famciclovir and valacyclovir are alternate drugs. Topical medications applied to genital and oral lesions cut the length of infection and reduce viral shedding. Systemic therapy is available for more serious complications such as herpes keratitis and disseminated herpes. New evidence indicates that a daily dose of oral acyclovir taken for a period of 6 months to one year can be effective in preventing recurrent genital herpes. Over-the-counter cold sore medications containing menthol, camphor, and local anesthetics lessen pain and may protect against secondary bacterial infections, but they probably do not affect the progress of the viral infection. Some protection in suppressing cold sores can be obtained from the amino acid lysine, taken orally in the earliest phases of recurrence. However, current treatments do not reduce the load of the DNA matrix, and thus are unable to reduce the risk of further viral reactivation. Ideally, an ultimate weapon against HSV-1 infection should be durably present in the cells, avoid questions of sensitivity and, if possible, reduce the load of viral genomes. New antiviral treatments with the aim to eliminate the virus DNA by the action of endonucleases are in development.

Virus DNA detection of HSV in cornea have been performed from tear fluid (Fukuda, Deai et al. 2008), corneal scrapings (El-Aal, El Sayed et al. 2006) and corneal explants button from healthy and diseased patients (Crouse, Pflugfelder et al. 1990) using PCR-based methods (Llorente, Hidalgo et al. 1998; Gonzalez-Villasenor 1999; Kessler, Muhlbauer et al. 2000; O'Neill, Wyatt et al. 2003; Kimura, Ihira et al. 2005; Namvar, Olofsson et al. 2005; Strick and Wald 2006; Susloparov, Susloparov et al. 2006; Engelmann, Petzold et al. 2008; Sugita, Shimizu et al. 2008; Tanaka, Kogawa et al. 2009; Wada, Mizoguchi et al. 2009; Yu, Shi et al. 2009) or DNA/DNA hybridization on infected cells (Nago, Hayashi et al. 1988; Kotronias and Kapranos 1998). However, none of these methods allowed for both the detection and the nature of an infectious HSV genome in infected cells in a single analysis.

HSV is one type of viral disease for which a large spectrum of technologies for the diagnosis of HSV infection has been disclosed in various patents and publications as for example in EP0139416, EP0263025, WO0202131, WO2004036185, and in Matsumoto, Yamada et al. 1992, Kotronias and Kapranos 1998, Kessler, Muhlbauer et al. 2000, Namvar, Olofsson et al. 2005, Susloparov et al. 2006, Sugita, Shimizu et al. 2008, and Yu, Shi et al. 2009.

The most reliable method of diagnosis of the HSV infectious disease is to isolate the virus for determination, but this method required culture cells and requires several days for the determination. There are also immunological methods but they are mostly unreliable and difficult to perform, especially during the latency phase. A large number of PCR-based methods have been develop for enhanced sensitivity and faster time to result than is possible by conventional means but these methods do not allow analysis of the whole HSV genome and cannot predict the infectivity of the sample containing nucleotide sequences of the virus because the complete genome of the said virus is not tested. Direct methods of detection of the HSV genome in cells in situ hybridization (Nago, Hayashi et al. 1988; Kotronias and Kapranos 1998) or dot blot DNA-DNA hybridization (Matsumoto, Yamada et al. 1992) have been reported but these methods are not able to determine the type of HSV. There are instances in which rapid, sensitive, and specific diagnosis of HSV disease is imperative. There is therefore, a clinical need to develop a rapid and sensitive tool to aid in the diagnosis of HSV. There also remains a need for a tool for the typing of the HSV infection. Rapid identification of the specific etiological agent involved in a viral infection provides information which can be used to determine appropriate therapy within a short period of time.

Analysis of the configuration of HSV-1 genome following lytic or latent infection has been performed by the gel electrophoresis system of Gardella et al. (Gardella, Medveczky et al. 1984). In these gels circular molecules characteristically exhibit lower mobilities than the corresponding linear forms do and this has allowed the detection of episomal forms of latent herpes virus genomes. However, this technique does not discriminate between the isomer of HSV genome in infected cells or tissues. Other techniques like pulse-field gel electrophoresis (PFGE) have been used to provide insights into the mechanism of HSV DNA replication (Severini, Morgan et al. 1994). This family of tests is highly time-consuming and in all cases requires southern blotting, implying manipulation of radioactivity, long migration and/or exposure times.

Another virus for which numerous detection and analytic methods have been reported is Human Immunodeficiency Virus, or HIV, including HIV-1 and HIV-2. HIV-1 load in blood plasma, as measured by the number of copies of HIV-1 RNA, is a major laboratory marker widely used in clinical practice. Higher virus loads are directly linked to more rapid progression to AIDS in HIV-1-infected individuals. The effectiveness of highly active antiretroviral therapy (HAART) is also assessed by measuring the HIV-1 load in plasma. A patient is considered to be successfully treated by HAART when HIV-1 load in plasma stays below the detection limit of commercial assays which is currently 50 copies of HIV-1 RNA per ml of plasma. However, in spite of its clinical success, HAART cannot eradicate the virus, mainly due to the persistence of various viral reservoirs including latently infected resting CD4⁺ cells (Hermankova, Siliciano et al. 2003; Siliciano, Kajdas et al. 2003). Recent studies demonstrated that both virus replication and evolution do continue in some patients even when HIV-1 RNA in plasma is undetectable and therapy is otherwise considered to be successful (Frenkel, Wang et al. 2003; Havlir, Strain et al. 2003; Ramratnam, Ribeiro et al. 2004; Chun, Nickle et al. 2005; Tobin, Learn et al. 2005). HAART failure as a result of development of drug-resistant HIV-1 strains is a common problem (del Rio 2006). Thus, special attention should be given to characterizing HIV-1 residual replication by studying its molecular markers in peripheral blood mononuclear cells (PBMC). In particular, the amounts of cell-associated HIV-1 RNA and proviral DNA should be quantified. Of these, the amounts of proviral DNA may reflect the size of the pool of latently infected cells. However, systematic studies of the relationships between the cellular HIV-1 RNA/DNA levels and therapy outcome are hindered by the extremely low copy numbers of HIV-1 RNA/DNA in PBMC under HAART. Therefore, development of highly sensitive methods for quantification of cellular forms of HIV-1 RNA/DNA is essential.

Real-time reverse transcription-PCR (RT-PCR) is currently the preferred method for quantification of HIV-1 RNA/DNA in cells (Douek, Brenchley et al. 2002; Fischer, Joos et al. 2004). However, despite their accuracy and specificity, single-step real-time RT-PCR methods using the TaqMan detection chemistry are unable to reliably quantify <100 copies of HIV-1 RNA/DNA target per reaction in the context of total cellular RNA/DNA (Espy, Uhl et al. 2006). This evokes the possibility of yielding false-negative results when PBMC material from patients under HAART is studied, especially when limited amounts of clinical material are available for analysis. Methods that use SYBR green-based detection chemistry to detect HIV-1 RNA/DNA may be more sensitive (Espy, Uhl et al. 2006) but are prone to false-positive results because DNA binding dyes do not bind in a sequence-specific manner. With a theoretical detection limit of one molecule per reaction, nested PCR is considered a more sensitive method than real-time PCR. However, only semi-quantitative data can be produced with this method. In addition, it requires labor-intensive and time-consuming experimental procedures. In contrast as described below the inventors have discovered that Molecular Combing allows the detection of unique events and can be used as a highly sensitive method for the detection and quantification of the HIV proviral DNA in the infected cells of patients.

To diagnose viral infection, the use of antigen detection assays is on the increase, especially those based on monoclonal antibodies. Rapid nucleic acid detection using specific probes directed against DNA or RNA or amplification methods are other options for several viruses. These have the advantage of being so sensitive that they could conceivably detect the viral nucleic acid in a single infected cell. For example, PCR technique which allows the enzymatic amplification of minute quantities of DNA often undetectable by other methods, has been widely used for detection of part of genome of several viral agents such as HSV (Rowley, Whitley et al. 1990), human immunodeficiency virus (HIV) (Ou, Kwok et al. 1988) and human papilloma viruses (HPV) (Shibata, Arnheim et al. 1988).

A significant limitation of PCR is that it does not allow one to confirm the integrity of the complete genome detected and consequently cannot be a standard of characterization of complete infectious viral genome found in a tested sample. PCR thus also lacks the specificity required for testing the efficiency of an antiviral treatment against infectious virus particles because its results do not indicate whether the viral polynucleotides detected are infectious. Serological methods do not directly detect infectious viral polynucleotides, such as chromosomally-integrated viral genomic DNA, and require that a subject mount an immune response to a virus prior to detection or that a sufficient amount of viral antigen be present in a sample.

Detection of Viral Oncogenes and Activation of Proto-Oncogenes by Virus

Many cancers originate from a viral infection; this is especially true in animals such as birds, but also in humans. Worldwide, the WHO International Agency for Research on Cancer estimated that in the year 2002 20% of human cancers were caused by infection of which 10-15% are caused by one of seven different viruses (Carrillo-Infante, Abbadessa et al. 2007). However, only a minority of persons or animals will go on to develop cancers after infection. Tumor viruses come in a variety of forms: viruses with a DNA genome, such as HPV (cervical cancer), Hepatitis B virus (liver cancer), and EBV (a type of lymphoma), and viruses with an RNA genome, like the Hepatitis C virus (HCV) can cause cancers, as can retroviruses having both DNA and RNA genomes (Human T-lymphotropic virus and hepatitis B virus, which normally replicates as a mixed double and single-stranded DNA virus but also has a retroviral replication component).

A direct oncogenic viral mechanism involves either insertion of additional viral oncogenic genes into the host cell (acutely-transforming virus) or to enhance already existing oncogenic genes (proto-oncogenes) in the genome (slowly-transforming virus) (for review, (Parsonnet 1999)). In acutely-transforming viruses the viral particles carry a gene that encodes for an overactive oncogene called viral-oncogene (v-onc) and the infected cell is transformed as soon as v-onc is expressed. In contrast, in slowly-transforming viruses, the virus genome is inserted, especially as viral genome insertion is obligatory part of retroviruses, near a proto-oncogene in the host genome. The viral promoter or other transcription regulation elements, in turn, cause over-expression of that proto-oncogene, which, in turn, induces uncontrolled cellular proliferation. Because viral genome insertion is not specific to proto-oncogenes and the chance of insertion near that proto-oncogene is low, slowly-transforming viruses have very long tumor latency compared to acutely-transforming virus, which already carries the viral-oncogene.

Some viruses are tumorigenic when they infect a cell and persist as circular episomes or plasmids, replicating separately from host cell DNA (Epstein-Barr virus and Kaposi's sarcoma-associated herpesvirus). Indirect viral oncogenicity also exists and involves chronic nonspecific inflammation occurring over decades of infection, as is the case for HCV-induced liver cancer. These two mechanisms differ in their biology and epidemiology: direct tumor viruses must have at least one virus copy in every tumor cell expressing at least one protein or RNA that is causing the cell to become cancerous. Foreign virus antigens are expressed in these tumors, consequently persons who are immunosuppressed such as AIDS or transplant patients are at higher risk for these types of cancers.

Gene or Cellular Therapy Based on the Use Viral Vectors

Gene therapy involves the insertion of genes into an individual's cells and tissues to treat diseases, such as hereditary diseases where deleterious mutant alleles of a gene are replaced with functional ones. Although the technology is still in its infancy, it has been used with some success. Viral vectors are a tool commonly used by scientific to deliver such genetic material into tissues (gene therapy) or cells (cellular therapy). These viral vectors are mainly derived from lentiviruses, retroviruses, adenoviruses or herpes viruses (for review, (Thomas, Ehrhardt et al. 2003)).

The genetic material in lentiviruses or retroviruses is in the form of RNA molecules, while the genetic material of their hosts is in the form of DNA. The genome of these classes of viruses can be modified by inserting a gene sequence of interest to be transferred in the infected tissue or cells. As the wild type virus, the recombinant viruses will introduce its RNA together with some enzymes, namely reverse transcriptase and integrase, into the cell. This RNA molecule from the recombinant virus must produce a DNA copy from its RNA molecule before it can be integrated into the genetic material of the host cell. After this DNA copy is produced and is free in the nucleus of the host cell, it must be incorporated into the genome of the host cell. That is, it must be inserted into the chromosomes in the cell by another enzyme carried in the virus called integrase. If this host cell divides later, its descendants will all contain the new genes.

One of the problems of gene therapy that can result from the use of lentiviruses or retroviruses is that the integrase enzyme can insert the genetic material of the virus into any arbitrary position in the genome of the host; it randomly shoves the genetic material into a chromosome. If genetic material happens to be inserted in the middle of one of the original genes of the host cell, this gene will be disrupted (insertional mutagenesis). If the gene happens to be one regulating cell division, uncontrolled cell division (i.e., cancer) can occur. This problem has recently begun to be addressed by utilizing single-chain homing endonucleases (Grizot, Smith et al. 2009) or by including certain sequences such as the locus control region to direct the site of integration to specific chromosomal sites (Zhou, Zhao et al. 2007).

Adenoviruses are viruses that carry their genetic material in the form of double-stranded DNA. They cause respiratory, intestinal, and eye infections in humans (especially the common cold). When these viruses infect a host cell they introduce their DNA molecule into the host. The genetic material of the adenoviruses is not incorporated into the host cell's genetic material. The DNA molecule is left free in the nucleus of the host cell and the instructions in this extra DNA molecule are transcribed just like any other gene. The only difference is that these extra genes are not replicated when the cell is about to undergo cell division so the descendants of that cell will not have the extra gene. Adeno-associated viruses (AAV) from the parvovirus family are small viruses with a genome of single stranded DNA. The wild type AAV can insert genetic material at a specific site on chromosome 19 with near 100% certainty (Huser and Heilbronn 2003). But the recombinant AAV, which does not contain any viral genes and only the therapeutic gene, does not integrate into the genome. As a result, treatment with either adenoviral vector or AAV will require readministration in a growing cell population although the absence of integration into the host cell's genome should prevent the development of cancer (Douglas 2007).

Herpes viruses are currently used as gene transfer vectors due to their specific advantages over other viral vectors. Among the unique features of HSV derived vectors are the very high transgenic capacity of the virus particle allowing to carry long sequences of foreign DNA, the genetic complexity of the virus genome, allowing to generate many different types of attenuated vectors possessing oncolytic activity, and the ability of HSV vectors to invade and establish lifelong non-toxic latent infections in neurons from sensory ganglia from where transgenes can be strongly and long-term expressed. Three different classes of vectors can be derived from HSV: replication-competent attenuated vectors, replication-incompetent recombinant vectors and defective helper-dependent vectors known as amplicons. Replication-defective HSV vectors are made by the deletion of one or more immediate-early genes, e.g. ICP4, which is then provided in trans by a complementing cell line. Oncolytic HSV vectors are promising therapeutic agents for cancer. Such HSV based vectors have been tested in glioma, melanoma and ovarian cancer patients.

The inventors have surprisingly discovered that Molecular Combing and stretching techniques in combination with specially designed probes or sets of probes can be used to diagnose, detect or monitor subjects carrying infectious viral DNA and other pathogenic genes by means of Molecular Combing and/or DNA stretching techniques. These diagnostic applications are unknown in the prior art which generally performed Molecular Combing on DNA samples easily obtained from known cultured cell lines. While Molecular Combing has been previously applied for chromosomal analysis, it was not previously recognized that this methodology or adaptations of this methodology could be usefully applied to diagnosing viral infection or detecting other pathogenic polynucleotides in a sample. On the other hand, the inventors have surprisingly found that DNA obtained directly from biological samples from a virus-infected subject can be evaluated by Molecular Combing procedures and active, infectious viral DNA detected in these samples. These methods overcome many of the problems associated with existing diagnostic methods for virus infection and provide a convenient, rapid and accurate method for detection and monitoring of infectious viral polynucleotides in a virus-infected subject or patient.

### BRIEF SUMMARY OF THE INVENTION

The invention provides a reliable, simple, fast, and inexpensive way to detect infectious or pathogenic polynucleotide sequences, including viral genomic sequences, in a sample from a subject or patient using a Molecular Combing and/or DNA stretching techniques in combination with specific probes which are able to bind with at least 5%, 10%, 25%, 50%, 75%, especially sets of probes that bind to 80-100% of the infectious or pathogenic polynucleotide sequence.

The inventors have discovered that Molecular Combing is a powerful technique (i) to detect and quantify viral DNA or viral origin-DNA in infected mammalian cells, tissues or biological fluids, such as infectious herpes simplex virus DNA in infected cornea, (ii) follow viral replication and viral genomic rearrangements that occur in the infected mammalian cells tissues or biological fluids, and (iii) identify infectious virus polynucleotides or infectious virus polynucleotides present in infected cells, tissues or biological fluids.

Unlike prior art techniques for detecting virus in infected cells or tissues, the Molecular Combing techniques of the invention can be performed simultaneously and do not require multiple time consuming and expensive procedures. The methods of the invention are easily applicable as diagnostic tools for detecting viral contamination or infection in cells or tissues, useful for determining the efficacy of antiviral treatments by quantifying or otherwise evaluating the effects or efficacies of such treatments on the quantity of infectious virus or infectious viral polynucleotides or viral replication in infected mammalian cells or tissues. The inventors have found that Molecular Combing overcomes many of the problems associated with past methods of detecting or diagnosing a viral infection, including PCR-based methods, and offers an easy and rapid way to do so. Specific embodiments of the invention include the following.

A method for detecting a viral genome or infectious viral polynucleotide in a biological sample comprising isolating, extracting or otherwise obtaining a polynucleotide from said sample; Molecular Combing said polynucleotide to form a stretched polynucleotide; contacting the stretched polynucleotide with one or more probes that recognize the infectious viral or genomic viral polynucleotide sequence; detecting hybridization of the probes to the combed sample. The polynucleotide may be infectious genomic viral DNA or infectious non-genomic viral DNA, an oncogene or a protooncogene and may be integrated into a chromosome of a subject or may be episomal or transgenic DNA. Detection of an entire viral genome or entire infectious viral DNA is importantly correlated with infection. For example, regarding the HSV causative agents of herpetic keratitis, it is essential to be able to show the presence of complete infectious genomes in the cells which can then be partially or totally generated in the infected cells.

The biological sample is generally obtained directly from a subject or patient and may constitute tissue, a cell sample, or blood, CSF, or synovial fluid sample or other fluid biological sample. A DNA molecule or fiber for stretching or for Molecular Combing may be extracted from the biological sample. A sample may be obtained from a living or non-living subject. Subjects include animals susceptible to viral diseases including humans, non-human mammals, such as cattle, bovines, sheep, goats, horses, pigs, dogs, cats and non-human primates; birds, such as chicken, turkey, duck, goose, ostrich, emu, or other birds; reptiles, amphibians and other animals.

A stretched or combed DNA molecular is contacted with one or more probes suitable for identifying the infectious or biologically active genomic, infectious viral, oncogene or protooncogene DNA. For detection of a DNA virus one or more probes, or preferably a set of probes covering 80-100% or any intermediate subrange or value of the viral genome are employed. The DNA virus may be one that is single or double stranded. Representative DNA viruses include herpes virus, such as Herpes Simplex Virus (HSV) types 1 and 2, papillomaviruses, and hepatitis viruses, such as hepatitis B virus. Probes that bind to retroviruses, such as HIV, preferably a retrovirus integrated as a provirus into a host chromosome are also selected in a similar manner and preferably a set of probes covering 80-100% of the proviral genome are employed.

The probes may correspond to at least 5% and preferably 80-100% of the genome of a virus strain already known to be infectious or may be designed to encompass the combination of genes known to be essential for replication, reproduction, or pathogenicity of a particular kind of virus.

Said probes can correspond to HSV-1 (NC_{—}001806.1)(SEQ ID NO: 15), HSV-2 (NC_{—}001798.1)(SEQ ID NO: 16), HIV1 (NC_{—}001802.1)(SEQ ID NO: 17), HIV2 (NC_{—}001722.1)(SEQ ID NO: 18), HBV (NC_{—}003977.1)(SEQ ID NO: 19), HPV16 (NC_{—}001526.2)(SEQ ID NO: 20), HPV18 (X05015.1)(SEQ ID NO: 21), HPV31 (J04353.1)(SEQ ID NO: 22), HPV33 (M12732.1)(SEQ ID NO: 23) and HPV45 (X74479.1)(SEQ ID NO: 24) which are also incorporated by reference to their accession numbers.

The one or more probes or probe set may comprise two, three or more different subsets of probes tagged with different labels. Preferably, such probes are used to determine the configurations of a genomic or infectious viral DNA or to identify different portions or segments of a viral genome or infectious viral DNA molecule or another pathogenic DNA molecule such as an oncogene or protooncogene.

In another embodiment the invention is directed to a method for detecting or identifying an infectious or genomic viral polynucleotide sequence in a mammalian cell, tissue or biological fluid comprising using Molecular Combing to detect the presence or the quantity of infectious viral polynucleotide or genomic viral polynucleotide in a cell, tissue or biological fluid. Still another embodiment constitutes a method for quantifying an infectious or genomic viral polynucleotide sequence comprising using Molecular Combing to detect quantity of infectious viral polynucleotide or genomic viral polynucleotide in a cell, tissue or biological fluid compared to an uninfected control sample or an otherwise similar sample obtained at a different point in time.

Methods for detecting the presence of a virus or detecting replication of a virus in a biological sample are also contemplated, as well as methods for longitudinally following virus replication or the rearrangement of a viral genome or infectious viral DNA especially in mammalian cells, tissue or biological fluid. Such methods may be performed by Molecular Combing DNA obtained from appropriate samples or samples taken over a specific time period for the presence of latent or replicating viral DNA or rearranged viral DNA in a cell or tissue.

The invention also pertains to method for evaluating the efficacy of anti-viral treatment using Molecular Combing to detect the presence, arrangement or quantity of infectious or genomic viral DNA in a sample obtained from a subject, treating the subject with an anti-viral agent or performing other anti-viral therapy, and monitoring or re-evaluating the presence, arrangement or quantity of infectious or genomic viral DNA after treatment using Molecular Combing techniques of described herein.

A kit for performing molecule combing containing a Molecular Combing apparatus and/or reagents, one or more probes that bind to an infectious polynucleotide, and optionally one or more cell, tissue or biological fluid sample(s) as well as other conventional ingredients for performing Molecular Combing, DNA stretching or probe hybridization. In some embodiments the kits will contain a set of probes for detecting or identifying genomic viral DNA in a combed DNA molecule covering at least 5% of a viral genome, infectious viral nucleic acids, oncogene, or proto-oncogene, especially 80, 85, 90, 95, 99 or 100%. The minimal length of the probe or set probe is 1 kb. In some embodiments, the kit will further contain software for detecting or classifying the infectious sequences and in other embodiments the kit may additionally comprise instructions for detecting a viral genome or infectious viral nucleic acids according to the methods of the invention.

### BRIEF DESCRIPTION OF THE DRAWINGS

FIG. 1. Example of HSV-1 specific-probes that can be used according to the invention. The dot plot represents the comparison between the HSV-1 genome sequence (horizontal axe) and the HSV-1 specific probes (vertical axe) to map the different probes to each other. Some of the HSV-1 fragments (HSV-S54, -B52 and -S58) hybridize with the inverted repeated sequences and consequently are present in two copies. The HSV-1 fragments listed in Table A can be associated and revealed in different colours to detect the HSV-1 genome. In this example, the association of 41 probes (on the 63 probes available, Table A) as follow allows covering 99% of the HSV-1 genome. The HSV-S54, -P4, -P5, -S4 and -B4 fragments correspond to the apparent H1 21 kb probe. The apparent H2 56 kb probe consists of 19 overlapping probes (HSV-B7, -B8, -S14, -B10, -S16, -B13, -S18, -B15, -S21, -B19, -S23, -B21, -B22, -S30, -S31, -S32, -B26, -P8, and -B28 fragments). The 45 kb H3 probe is composed of the HSV-B30, -S44, -S45, -B38, -B39, -B40, -S49, -B44, -B45, -B46, -B48 and -S54 fragments. Due to the presence of the inverted repeated sequences, both H4 (13 kb) and H6 (6.5 kb) probes are composed of HSV-B52 and -S58 fragments; the HSV-S59 fragment is also present in the H4 probe. Finally, 3 overlapping fragments (HSV-S60, -B58 and -B60) formed the H5 7.5 kb probe. The localization probes which form a signature specific for a specific region of the HSV-1 genome is in red (digoxygenin labelling, black boxes) for H1, H3 and H5 probes and in green (biotin labelling, grey boxes) for the H2, H4 and H6 probes. The sizes (rounded to the nearest kb) of the localization probes are indicated respectively below each boxes.

FIG. 2. Comparison of two methods of HSV-1 DNA extraction from viral particles. A) Example of combed HSV-1 DNA on silanized surfaces. After combing of the HSV-1 DNA solution extracted with either the standard phenol chloroform (left picture) or the modified extraction protocol from agarose plug-embedded HSV-1 (right picture), the fibers are revealed with the intercalating agent YOYO-1 and observed on a epifluorescence microscope equipped with a camera. The figure shows two pictures that are characteristic of each extraction method. Scale is indicated as a bar. B) Histogram showing the cumulative frequency of the number DNA fiber sizes within a given length interval which were contained on genomic DNA isolated from HSV-1 particles. Interval width is 10 μm. Thus, for example, the third point represents the number of measures in the [30-40 μm] interval. Left panel shows the length of every HSV-1 DNA fibers that was recorded (654 measures) in the DNA solution extracted with the standard phenol:chloroform extraction. Median size of the fibers is 18 μm that is, 36 kb. The right panel show the distribution of the length of DNA fibers (2322 measures) obtained with the alternative method. In that case, the median size of the distribution is 42 μm that is, 84 kb.

FIG. 3. Detection of isomers of the HSV-1 genome in DNA solution extracted from viral particles and infected cells. A) Examples of FISH on combed HSV-1 DNA. Schematic representation of the different possible organization of the hybridization patterns corresponding to the different isomers of the HSV-1 genome are indicated (digoxygenin labelled-H1, H3 and H5 probes are represented in black boxes; biotin-labelled-H2, H4 and H6 probes are depicted in grey boxes). The minimal requirement hybridization patterns as defined in the “Analysis of HSV-1 detected signals” section are also indicated just above the complete signal. Four representative linear hybridization chains showing each complete isomer of HSV-1 genome (White: Texas Red/Alexa 594-fluorescence: H1, H3 and H5; Black: green Alexa 488-fluorescence: H2, H4 and H6). Schematic representations of each HSV-1 genome isomer are shown above the corresponding pictures. (B) Histogram of the distribution genome isomers of the HSV-1 KOS strain in viral particles produced in Vero, COS7 and Neuro2A cell lines. Hybridization signals were selected and analyzed as described in the “Examples” section. In this example, a total of 405 hybridization signals for each experiment were identified and classified. Each bar represents the number of each isomer of the HSV-1 genome. In this example, the distribution of the HSV-1 KOS strain isomers are equivalently distributed in viral particles from COS7 cells whereas the P and IS isomers are the more frequent isomer in the viral particles produced from the Neuro2A and Vero cell lines. This latter distribution is statistically different from an equimolar distribution (Chi-2 test). (C) Histograms of the distribution of the genome isomers of HSV-1 strains Sc16 and KOS in different infected cells (BSR, COS-7, Neuro 2A and Vero cells). The hybridization signals were selected and analyzed as described in the “Examples” section. In these examples, 405 signals from each production were selected and classified. The distribution of the HSV-1 strain Sc16 produced in BSR, COS-7, Neuro 2A and Vero cells and of the HSV-1 strain KOS produced in COS-7 is statistically equivalent to an equimolar distribution (Chi-2 test). In Neuro 2A and Vero infected cells, the P and IS isomers are more frequent that the IL and ILS isomers.

FIG. 4. Method of extraction of genomic DNA from mouse and rabbit cornea. A) Example of combed genomic DNA on silanized surfaces. After extraction from cornea with the protocol described in Examples section, the genomic DNA solution is combed and revealed with the intercalating agent YOYO-1 before observation on an epifluorescence microscope equipped with a camera. Left pictures show a representative picture of combed DNA extracted from a healthy mouse cornea and the right picture from a healthy rabbit cornea. Molecular Combing is performed at low density to allow measurement of the length of the genomic DNA fiber. Scale is indicated as bar. B) Histogram showing the cumulative frequency of the number DNA fiber sizes within a given length interval which were contained on genomic DNA isolated from HSV particles. Interval width is 25 kb. Thus, for example, the fifth bar represents the number of measures in the [225-250 kb] interval. Left panel shows the length of every DNA fibers that was recorded (10069 measures) in the DNA solution extracted from the mouse cornea. Median size of the fibers is 204 kb. In this sample, around 31% of the genomic DNA fiber exhibited a size above 200 kb. The right panel show an example of the distribution of the length of DNA fibers (8336 measures) obtained from the rabbit cornea. In that case, the median size of the distribution is 196 kb with a proportion of fibers above 200 kb in length of 27%.

FIG. 5. Histogram of the distribution of isomers of HSV-1 genome in infected mouse cornea. Total DNA from HSV-1 infected mouse cornea was extracted, combed and hybridized with the HSV-1-specific probes. In this example, a total of 18 hybridization signals were identified and classified. Each bar represents the number of each isomer of the HSV-1 genome.

FIG. 6. Detection of replication intermediate of the HSV-1 in HSV-1 infected mouse cornea. The figure shows two examples of complex HSV-1 genome DNA corresponding to replication concatemers (White signal: Texas Red/Alexa 594-fluorescence: H1, H3 and H5; Black signal: green Alexa 488-fluorescence: H2, H4 and H6).The upper picture depicted a signal compose of at least two hypothetical overlapping HSV-1 genomes composed of an IS and a P isomer while the lower picture show a HSV-1 concatemer consisting of at least hypothetical ILS and IL signals.

FIG. 7. Detection of non canonical HSV-1 genomes in HSV-1 infected mouse cornea. A) Examples of non canonical HSV-1 from infected mouse cornea. The figure shows representative examples of hybridization patterns that do not correspond to one of the canonical isomers of the HSV-1 genome (White signal: Texas Red/Alexa 594-fluorescence: H1, H3 and H5; Black signal: green Alexa 488-fluorescence: H2, H4 and H6). Scale bar is indicated Canonical hybridization pattern corresponding to the ILS isomer (biotin-labelled H1, H2B and H3 probes are represented in grey boxes and signal; digoxygenin-labelled H2A and H5 are depicted in white boxes and signals, and Alexa488-labelled H4 and H6 probes are depicted in black boxes and signals) obtained on combed DNA extracts of HSV-1 strain Sc16-infected Vero cells. This example shows HSV-1 specific probes H1 to H6 labelled to evaluate the proportion of non-canonical structures in the H4/H6 region. C) Non canonical H4/H6 on HSV-1 strain Sc16 in Vero cell extracts. The first hybridization pattern shows alternation of Alexa 488 fluorescence signal (black signals) of various sizes corresponding to the H4/H6 probes and Alexa 594 fluorescence (grey signal) corresponding to fragments H1, H2B or H3 probes or AMCA/Alexa 350 fluorescence signal (white signal) corresponding to part of H2A or H5 probes of a maximum of 10kb. The second example exhibit an alternation between Alexa 488 fluorescence signal (black signals) of various sizes and AMCA/Alexa 350 fluorescence signal (white signal) that is surrounded by Alexa 594 fluorescence signal (grey signal). The third example shows a unique repetition of an Alexa 488 fluorescence signal (black signals) surrounded by Alexa 594 fluorescence signal (grey signal). Scale bar is indicated. D) Histograms of the distribution between canonical and non canonical structure in the H4/H6 regions of HSV-1. 367 hybridization signals were selected and analysed as described in the “Examples” section. In this example, 80% of the H4/H6 probes correspond to the theoretical structure.

FIG. 8. Detection of proviral HIV-1 DNA in ACH-2 cells culture. All histograms are showing number of signals within a given length interval (0.644 kb/class) in function of one FISH signal length or gap size between two FISH signals, in kilobases. Thus, for example, the second bar of the first histogram represents the number of measures in the [7.86-8.50 kb] interval. A) Examples of proviral HIV-1 DNA located at chromosome 7p15 using the G248P87988G9 and G248P86255A8 fosmids. Schematic representation of organization of the hybridization pattern corresponding to the integrated proviral HIV-1 DNA is indicated (digoxygenin labelled-HIV-1 probes are represented in a black box and signal; biotin-labelled-fosmids are depicted in white boxes and signals). Histograms on the left and right showing the distribution of the gap size between fosmid G248P87988G9 green Alexa 488-fluorescence signal and HIV-1 Texas Red/Alexa 594-fluorescence signal, and between HIV-1 Texas Red/Alexa 594-fluorescence signal and fosmid G248P86255A8 green Alexa 488-fluorescence signal, respectively. Middle histogram shows the distribution of the HIV-1 Texas Red/Alexa 594-fluorescence signal size. B) Examples of normal allele of the 7p15 locus. Schematic representation of organization of the hybridization pattern corresponding to the normal allele is indicated (biotin-labelled-fosmids are depicted in grey boxes and signals). Histogram showing the distribution of the gap size between fosmid G248P87988G9 green Alexa 488-fluorescence signal and G248P86255A8 green Alexa 488-fluorescence signal. C) Isolated proviral HIV-1 DNA. Schematic representation of organization of the hybridization pattern corresponding to the isolated form of HIV-1 is indicated (digoxygenin labelled-HIV-1 probes are represented in a black box and signal). Histogram shows the distribution of HIV-1 Texas Red/Alexa 594-fluorescence signal size. D) Examples of proviral HIV-1 DNA located at chromosome 7p15 using the G248P84833H9 fosmids. Schematic representations of organization of the hybridization patterns corresponding to the integrated proviral HIV-1 DNA (digoxygenin labelled-HIV-1 probes are represented in a black box and signal; and the wild type locus (biotin-labelled-G248P84833H9 fosmids are depicted in white boxes and signals) are indicated. The histogram shows the distribution of the HIV-1 Texas Red/Alexa 594-fluorescence signal size.

### DETAILED DESCRIPTION OF THE INVENTION

The invention enables a rapid, specific and sensitive detection of infectious viral polynucleotides or infectious viral origin DNA in a sample that avoids the significant constraints imposed by amplification methods like PCR or serological tests such as ELISA.

In contrast to prior art methods, the Molecular Combing techniques of the invention permit the successful detection of complete viral genomes or infectious or virulent portions of viral polynucleotide sequences leading to improved diagnosis of acute or latent viral infections.

The method of the invention enables to detect the type of HSV and the structure of its genome reliably, in a time- and cost-effective fashion, and with none of the constraints of manipulating radioactivity. Moreover, the method of the invention enables to follow the presence of the viral infection after antiviral treatment, whatever the type of treatment considered.

The present invention relates to a method for detecting in vitro the presence of a genome of DNA viruses in infected cells of eukaryotic cells, in particular the detection of the HSV genome. Said method comprises a hybridization step of nucleic acid representative of given virus with at least a probe or a set of probes which is (are) specific for HSV DNA.

Molecular Combing techniques which may be used in accordance with the invention are disclosed by Bensimon, et al., U.S. Pat. No. 6,303,296 and by Lebofsky, et al., WO 2008/028931 the disclosure of which are hereby incorporated by reference. The inventors recognized that no technique can detect the presence of a complete or non-rearranged genome of infectious form of a virus except by traditional culture techniques and thus sought to apply and adapt these techniques to detection of infectious viral DNA. The combing method was never tested as an efficient tool to detect such viral DNA forms but only for the location of long sequences of DNA inserted in a cellular genome in view of understanding the genetic influence of such sequence in a specific genetic environment. Non-limiting examples of detection of infectious viral (HSV) DNA by the modified methods developed by the inventors are shown below.

The inventors modified the standard extraction protocol to isolate viral genomic DNA from viral particles. Generally, 0.1% SDS is used for the lysis of viral particles in the low-melting agarose plugs instead of 0.1% sarkosyl in the standard protocol. A methodology was also developed to extract genomic DNA from cornea to investigate the presence of HSV DNA in infected cornea for purposes of diagnosis.

The present invention relates to a method for detecting viral DNA, in particular HSV DNA and HIV DNA, contained on nucleic acid of infected cells, tissue or biological fluid. Said method comprises a hybridization step of nucleic acid representative of said viral DNA with specific probes or set of probes that cover the entire said viral DNA and that permit to identify rearrangements within the nucleic acid representative of said viral DNA.

The term “nucleic acid” and in particular “nucleic acid representative of viral DNA” as used herein designates one or several molecules of any type of nucleic acid capable of being attached to and stretched on a support as defined herein, and more particularly stretched by using Molecular Combing technology; nucleic acid molecules include DNA (in particular genomic DNA, especially viral DNA, or cDNA) and RNA (in particular mRNA). A nucleic acid molecule can be single-stranded or double-stranded.

“Nucleic acid representative of said viral DNA” means that said nucleic acid contains the totality of the genetic information or the essential information with respect to the purpose of the invention, which is present on said viral DNA. This term includes genomic viral DNA, such as that integrated into a host chromosome and which can produce infectious virus, infectious viral DNA which may lack certain genomic elements but can be infectious when expressed in particular host cells, and viral genes which exert a pathogenic effect on host cells, such as viral oncogenes.

A proto-oncogene includes those normal genes, which when altered by mutation can convert into oncogenes that causes a cell to grow or divide in an unregulated manner. Proto-oncogenes have diverse cellular functions, some provide signals for cell division, and others may play roles in apoptosis. Functional and structural characteristics of oncogenes, including their nucleic acid sequences, are well-known in the art and are incorporated by reference to Human Cancer Viruses: Principles of Transformation and Pathogenesis by J. Nicolas, et al., Karger Publishers (2010), ISBN3805585764, 9783805585767 (244 pages) which is incorporated by reference.

An oncogene encompasses a defective version of a proto-oncogene. A single copy of an oncogene can cause uncontrolled cell growth. Representative oncogenes include ras, myc, src, Her-2/neu, hTERT, and Bcl-2. Functional and structural characteristics of oncogenes, including their nucleic acid sequences, are well-known in the art and are incorporated by reference to Oncogene: Gene, Mutation, Tumor, Apoptosis, Gene Expression, Protein, Cell Growth, Cellular Differentiation, by L. M. Surhone, et al., Betascript Publishers (2010), ISBN6130361599, 9786130361594 (172 pages). Further description and identification of oncogenes is incorporated by reference to http://_www.cancerquest.org/index.cfm?page=780 (last accessed Apr. 11, 2011) and to Cooper G. Oncogenes. Jones and Bartlett Publishers, 1995 and Vogelstein B, Kinzler K W; The Genetic Basis of Human Cancer. McGraw-Hill: 1998, both of which are incorporated by reference. The process of activation of proto-oncogenes to oncogenes can include viral transduction or viral integration, point mutations, insertion mutations, gene amplification, chromosomal translocation and/or protein-protein interactions. Viruses that can induce activation of proto-oncogenes include HBV and HCV (hepatocellular carcinoma), HTLV (leukaemia), HPV (cervical, anal and penile cancer), HSV-8 (Kaposi's sarcoma), Merkel cell polyomavirus (Merkel cell carcinoma) and, EBV (Burkitt's lymphoma, Hodgkin's lymphoma, post-transplantation lymphoproliferative disease and Nasopharyngeal carcinoma)

In a particular embodiment, the nucleic acid sample used for stretching is genomic DNA, in particular total genomic DNA or more preferably chromosomal genomic DNA (nuclear genomic DNA) of infected cells or tissues, and/or fragments thereof. The term “nucleic acid” is in particular used herein to designate a nucleic acid representative of one or several chromosome(s) and/or of one or several fragment(s) of chromosomes. Said fragments can be of any size, the longest molecules reaching several megabases. Said fragment are generally comprised between 10 and 2000 kb, more preferably between 20 and 500 kb and are in average of about 300 kb.

The nucleic acid sample used in the method of the invention can be obtained from a biological fluid or from a tissue of biological origin, said sample or tissue being isolated for example from a human, a non human mammal or a bird.

As defined herein, a probe is a polynucleotide, a nucleic acid/polypeptide hybrid or a polypeptide, which has the capacity to hybridize to nucleic acid representative of virus DNA as defined herein, in particular to RNA and DNA. This term encompasses RNA (in particular mRNA) and DNA (in particular viral cDNA or viral genomic DNA) molecules, peptide nuclear acid (PNA), and protein domains. Said polynucleotide or nucleic acid hybrid generally comprises or consists of at least 100, 300, 500 nucleotides, preferably at least 700, 800 or 900 nucleotides, and more preferably at least 1, 2, 3, 4 or 5 kb. For example, probes of 1, 2, 3, 4, 5, 6, 7, 8, 9, 10, 11, 12, 13, 14, 15 kb or more than 15 kb, in particular 30, 50, 100 or 150 kb can be used. Such a probe or a set of probes may correspond or cover 5, 10, 15, 20, 25, 30, 35, 40, 45, 50, 55, 60, 65, 70,75, 80, 85, 90, 95, 98, 99 or 100% of a viral genome, especially that of an infectious virus. A nucleic acid probe may be a single or double-stranded polynucleotide or a modified polynucleotide. A set of continuous set of probes will cover a particular section of the genome or the entire genome or overlap each other with respect to this section. A suitable probe may be based on a viral polynucleotide sequence, such as those disclosed herein, and may be identified or synthesized by any appropriate method. Probes may be labelled or tagged radioactivity, fluorescently or by other means known in the art.

A polypeptide probe generally specifically binds to a sequence of at least 6 nucleotides, and more preferably at least 10, 15, or 20 nucleotides. As used herein, the sequence of a probe, when the probe is a polypeptide, should be understood as the sequence to which said polypeptide specifically binds.

By “a portion of” a particular region, it is meant herein consecutive nucleotides of the sequence of said particular region. A portion according to the invention can comprise or consist of at least 15 or 20 consecutive nucleotides, preferably at least 100, 200, 300, 500 or 700 consecutive nucleotides, and more preferably at least 1, 2, 3, 4 or 5 consecutive kb of said particular region. For example, a portion can comprise or consist of 1, 2, 3, 4, 5, 6, 7, 8, 9, 10, 11, 12, 13, 14, 15 consecutive kb of said particular region.

In a particular embodiment, the probe used or at least one of the probes used is a nucleotide variant of the probe showing a complementary sequence of 100% to a portion of one strand of the target nucleic acid. The sequence of said variant can have at least 70, 80, 85, 90 or 95% complementarity to the sequence of a portion of one strand of the target nucleic acid. Said variant can in particular differ from the probe which is 100% identical or complementary by 1 to 20, preferably by 1 to 10, nucleotide deletion(s), insertion(s) and/or more preferably substitution(s), in particular by, 1, 2, 3, 4, 5, 6, 7, 8, 9 or 10 nucleotide deletion(s), insertion(s) and/or more preferably substitution(s) in the original nucleotide sequence. In a particular embodiment, the variant keeps the capacity to hybridize, in particular to specifically hybridize, to the sequence of the nucleic acid target, similarly to the probe that is 100% identical or 100% complementary to a sequence of the nucleic acid target (in particular in the hybridization conditions defined herein).

The term “complementary sequences” in the context of the invention means “complementary” and “reverse” or “inverse” sequences, i.e. the sequence of a DNA strand that would bind by Watson-Crick interaction to a DNA strand with the said sequence.

In a particular embodiment of the invention, the probes or one or several probes used to carry out the invention are labelled with one or several hapten(s) (for example biotin and digoxygenin) and revealed with specific antibodies directed against these haptens. Use of different haptens for a given probe or set of probe will allow to detect rearrangements within a given viral DNA. Said probes can be labelled as defined herein and as described in patent application WO 2008/028931, which is incorporated by reference.

A set of probes as used herein comprises of at least two probes. For example, said set of probes can consist of 2 to 20 probes (2, 3, 4, 5, 6, 7, 8, 9, 10, 11, 12, 13, 14, 15, 16, 17, 18, 19 or 20 probes). The number of probes in a set does usually not exceed 10, 20, 30, 40, 50, 60, 70, 80, 90 or 100 probes depending on the sensitivity that is required and the length of the probe; a set of probes preferably consists of 2, 3, 4, 5, 6, 7, 8, 9 or 10 probes at the most.

The probes or the set of probes of the invention not only allow the detection of the entire whole genomic viral DNA but also to identify rearrangements that can occur within the genomic viral DNA. For example, the probe sets used in the Example 1 for HSV detection allow the detection of the different HSV genome isomers that are generated by homologous recombination between the inversed repeated sequences that surround the unique regions U_{L} and U_{S}. Moreover, each probes set (H1 to H6) for the detection of HSV are composed of several probes (3 to 19 different fragments) that are labeled with different two different haptens (digoxygenin for the fragments consisting of H1, H3 and H5 probes; biotin for the fragments consisting of H2, H4 and H6 probes). Changing the haptens of one or of several fragments by other haptens (whatever the nature of this hapten) within a given probe set permits the generation of a different color-fluorescence array that allows the identification of rearrangements within this specific region of the viral DNA.

Molecular Combing can also be a useful tool for an early diagnosis of patients susceptible of developing a cancer caused by a viral infection, for example, those at risk of conversion of a proto-oncogene into an oncogene. Indeed, Molecular Combing can discriminate between integrated and episomal viral genome. In the case where the viral DNA is integrated, by using a specific set of probes Molecular Combing will allow to determine whether the integrated viral DNA contains a complete viral oncogene or whether it is integrated in a close proximity of a cellular proto-oncogene.

The invention may also be used in conjunction with gene therapy. By using specific sets of probes for each transgene and/or viral vector, Molecular Combing provides a powerful tool for an evaluation of efficacy and safety of the viral vector-based gene therapy. Indeed, Molecular Combing can discriminate between integrated and episomal transgene. In the case where the transgene is integrated (lentiviruses and retrovirus based vectors), by using a specific set of probes Molecular Combing will allow to determine whether the integrated viral DNA is integrated in a gene (insertional mutagenesis) or in a close proximity of a cellular proto-oncogene. In the case the transgene is episomal (Adenoviral vector, AAV or HSV vector); Molecular Combing will be useful for the quantification of the transgene in the growing cell population and may help to define the right time for readministration of the transgene.

Specific embodiments of the invention include the following methods and products. A method for detecting an infectious viral polynucleotide in a biological sample comprising: separating, extracting or otherwise obtaining a polynucleotide from said sample, Molecular Combing said polynucleotide to form a stretched polynucleotide, contacting said stretched polynucleotide with one or more probes that recognize the infectious polynucleotide sequence, detecting hybridization of the probes to the combed sample. This method may be performed using a biological sample that is a tissue or cell sample obtained from a subject, for example, a blood, plasma, serum, CSF, synovial fluid sample or some other kind of biological fluid from the subject. A polynucleotide used in this method may be extracted from the tissue or cell sample or from components of a biological fluid obtained from the subject. Generally, the sample will be obtained from a living subject, but samples may also be obtained from deceased subjects. Samples can be obtained from humans or other mammals such as cattle, bovines, sheep, goats, horses, pigs, dogs, cats and non-human primates, or from other animals such as avian species such as a chicken, turkey, duck, goose, ostrich, emu, or other bird. The method can be practiced with polynucleotides which are DNAs which may also contain or comprise infectious or non-infectious genomic viral DNA or non-infectious non-genomic viral polynucleotides such as DNA or RNA. The polynucleotide detected or analyzed by this method may be integrated into the DNA of the subject or can be in episomal form. The polynucleotide to be detected can be contacted with one or more probes that bind to a DNA virus, including both single-stranded and double-stranded DNA viruses. For example, the polynucleotide may be contacted with one or more probes that bind to a herpes virus, such as herpes simplex virus (HSV). Other probes may be used which bind to other viruses like papilloma virus, hepatitis B virus, or retroviruses like HIV. In some embodiments the method will use a set of probes that bind to at least 80%, 90%, 95%, or 99% of the genomic or infectious DNA of a virus. In other embodiments the one or more probes used in the method will comprise at least two sets of probes that are tagged with different labels, for example, to permit the identification of different portions or segments of a viral genome to which the different probes bind or to permit the identification of rearrangements in a viral genome.

Other specific embodiments of the invention include:

A method for detecting, identifying or visualizing an infectious or genomic viral polynucleotide sequence in a mammalian cell, tissue or biological fluid comprising using Molecular Combing to detect the presence or the quantity of infectious viral polynucleotide or genomic viral polynucleotide in a cell, tissue or biological fluid.

A method for quantifying an infectious or genomic viral polynucleotide sequence comprising using Molecular Combing to detect quantity of infectious viral polynucleotide or genomic viral polynucleotide in a cell, tissue or biological fluid compared to an uninfected control sample or an otherwise similar sample obtained at a different point in time or from a different source or clinical sample.

A method for detecting or following viral presence or replication or viral genomic rearrangements in a mammalian cell comprising Molecular Combing for the presence of latent or replicating viral DNA or rearranged viral DNA in a cell, tissue or biological fluid.

A method for evaluating the efficacy of anti-viral treatment comprising detecting using Molecular Combing the presence, arrangement or quantity of infectious or genomic viral DNA in a sample obtained from a subject, treating said subject with an anti-viral agent, and re-evaluating using Molecular Combing the presence, arrangement or quantity of infectious viral or genomic viral DNA in said subject.

A set of probes covering 80-100% of the HSV, HIV, HBV or HPV genome.

A kit for performing molecule combing comprising a Molecular Combing apparatus and/or reagents, one or more probes that bind to an infectious polynucleotide, and optionally one or more cell, tissue or biological fluid sample(s). The kit which comprises a set of probes for detecting or identifying genomic viral DNA in a combed DNA molecule. The kit further comprising software for detecting or classifying the infectious sequences.

The following biological materials which have been deposited under the terms of the Budapest Treaty at the CNCM, Institut Pasteur 25 Rue du Docteur Roux, F-75724 Paris Cedex 15: HSV-B4 (CNCM I-4298), HSV-B19 (CNCM I-4299), HSV-Sc54 (CNCM I-4300), and HSV-P4 (CNCM I-4301).

### EXAMPLES

### Example 1

### Herpes Simplex Virus Detection

Preparation of Embedded DNA Plugs from Viral Particles

HSV-1 DNA was extracted from viral particles by standard phenol:chloroform extraction (Ben-Zeev, Weinberg et al. 1974) or by a modified procedure described in Lebofsky et al. (Lebofsky, Heilig et al. 2006) which are both incorporated by reference. Briefly, HSV-1 particles were resuspended in 1× PBS at a concentration of 5·10⁶ viral particles/mL, and mixed thoroughly at a 1:1 ratio with a 1.2% w/v solution of low-melting point agarose (Nusieve GTG, ref. 50081, Cambrex) prepared in PBS, at 50° C. 90 μL of the viral particles/agarose mix was poured in a plug-forming well (BioRad, ref. 170-3713) and left to cool at least 30 min at 4° C. Embedded viral particles were lysed in 0.1% SDS-0.5M EDTA (pH 8.0) solution at 50° C. for 30 minutes. After three washing steps in 0.5M EDTA (pH 8.0) buffer of 10 minutes at room temperature, plugs were digested by overnight incubation at 50° C. with 2 mg/mL Proteinase K (Eurobio code GEXPRK01, France) in 250 μL digestion buffer (0.5M EDTA, pH 8.0). The use of 0.1% SDS instead of Sarkosyl was very productive and allows a very high quality of extracted viral DAN to be collected.

Preparation of Embedded DNA Plugs from Infected Cells

The extraction of HSV-1 DNA from infected cells culture (BSR, COS-7, Neuro 2A and Vero) was performed as previously described (Schurra and Bensimon 2009). Briefly, infected cells were pelleted by centrifugation at 5000 g for 5 minutes, resuspended at a concentration of 2·10⁶cells/mL in 1× PBS buffer and mixed thoroughly at a 1:1 ratio with a 1.2% w/v solution of low-melting point agarose (Nusieve GTG, ref. 50081, Cambrex) prepared in 1× PBS at 50° C. 90 μL of the cell/agarose mix was poured in a plug-forming well (BioRad, ref. 170-3713) and left to cool down at least 30 min at 4° C.

Lysis of cells in the blocks was performed as previously described (Schurra and Bensimon 2009). Briefly, Agarose plugs were incubated overnight at 50° C. in 250 μL of a 0.5M EDTA (pH 8), 1% Sarkosyl, 250 μg/mL proteinase K (Eurobio, code : GEXPRK01, France) solution, then washed twice in a Tris 10 mM, EDTA 1 mM solution for 30 in at room temperature.

Preparation of Embedded DNA Plugs from Infected Cornea

HSV-1 strain Sc16 infected mouse cornea was collected at a final stage of infection, and kept in Corneamax® (Eurobio code EYEMAX00, France) medium. After rinsing three times during 15 minutes at room temperature with 1× PBS solution, the entire cornea was cut into small pieces. Tissue lysis was carried out for up to 16 h at 37° C. in 0.3 mg/mL Collagenase type A (Roche, code 10 103 578 001), and 0.8 mg/mL GIBCO™ Dispase (Invitrogen, France, code 17105-041), both prepared in GIBCO™ 1× Hanks' Balanced Salt Solution HBSS buffer (Invitrogen, France, code 14060040). Lysates were pelleted by centrifugation at 5000g for 10 minutes, resuspended at a concentration of 1·10⁶ to 2·10⁶cells/mL in 1× PBS buffer and mixed thoroughly at a 1:1 ratio with a 1.2% w/v solution of low-melting point agarose (Nusieve GTG, ref 50081, Cambrex) prepared in 1× PBS at 50° C. 90 μL of the cell/agarose mix was poured in a plug-forming well (BioRad, ref 170-3713) and left to cool down at least 30 min at 4° C.

Lysis of cells in the blocks was performed as previously described (Schurra and Bensimon 2009). Briefly, Agarose plugs were incubated overnight at 50° C. in 250 μL of a 0.5M EDTA (pH 8), 1% Sarkosyl, 250 μg/mL proteinase K (Eurobio, code: GEXPRK01, France) solution, then washed twice in a Tris 10 mM, EDTA 1 mM solution for 30 in at room temperature.

Final Extraction of DNA and Molecular Combing

Plugs of embedded DNA from viral particles or corneas were treated for combing DNA as previously described (Schurra and Bensimon 2009). Briefly, plugs were melted at 68° C. in a MES 0.5 M (pH 5.5) solution for 20 min, and 1.5 units of beta-agarase (New England Biolabs, ref. M0392S, MA, USA) was added and left to incubate for up to 16 h at 42° C. The DNA solution was then poured in a Teflon reservoir and Molecular Combing was performed using the Molecular Combing System (Genomic Vision S.A., Paris, France) and Molecular Combing coverslips (20 mm×20 mm, Genomic Vision S.A., Paris, France). The combed surfaces were dried for 4 hours at 60° C.

Synthesis and Labelling of HSV-1 Probes

The coordinates of all the probes relative to the Genbank sequence NC_{—}001806.1 are listed in Table A. Probe size ranges from 1110 to 9325 bp in this example.

The HSV-1 specific probes were produced by either SacI or BspEI (New England Biolabs Inc., Beverly, Mass., USA code R0156L and R0156L, respectively) enzymatic digestion of the HSV-1 sc16 strain obtained from the CNRS (Prof. Marc Labetoulle, laboratoire de virology moléculaire et structurale, UMR CNRS 2472-INRA 1157, Gif-sur-Yvette, France) or by long-range PCR using LR Taq DNA polymerase (Roche, kit code: 11681842001) using the primers listed in table B and the DNA from HSV-1 sc16 as template DNA. SacI and BspEI HSV-1 fragments were ligated in SacI and XmaI-digested pNEB193 plasmid (New England Biolabs Inc., Beverly, Mass., USA, code N3051S), respectively. PCR products were ligated in the pCR®2.1 vector using the TOPO® TA cloning Kit (Invitrogen, France, code K455040). The two extremities of each probe were sequenced for verification purpose. The apparent H1 (21 kb), H2 (56 kb), H3 (45 kb), H4 (13 kb), H5 (7.5 kb) and H6 (6.5 kb) probes are mixes of several adjacent or overlapping probes listed in table A. For the visualisation of the non canonical structure implicated the H4 and H6 probes, the apparent H2 probe was split in two probes H2A (31 kb) and H2B (25 kb).

The labelling of the probes was performed using conventional random priming protocols. For biotin-11-dCTP labelling, the BioPrime® DNA kit (Invitrogen, code: 18094-011, CA, USA) was used according to the manufacturer's instruction, except the labelling reaction was allowed to proceed overnight. For 11-digoxygenin-dUTP and Alexa488-7-OBEA-dCTP, the dNTP mix from the kit was replaced by the mix specified in table C. 200 ng of each plasmid was labelled in separate reactions. For isomer classification, H1, H3, H5 probes were labelled with 11-digoxygenin-dUTP while H2, H4 and H6 probes were labelled with biotin-11-dCTP. For the visualisation of the non canonical structure implicated the H4 and H6 probes, H1, H2B and H3 probes were labelled with biotin-11-dCTP, H2A and H5 probes with 11-digoxygenin-dUTP and H4, H6 with Alexa 488-7-OBEA-dCTP. The reaction products were visualized on an agarose gel to verify the synthesis of DNA.

Hybridization of HSV-1 Probes on Combed Viral DNA and Detection

Subsequent steps were also performed essentially as previously described in Schurra and Bensimon, 2009 (Schurra and Bensimon 2009) which is incorporated by reference. Briefly, a mix of labelled probes (250 ng of each probe, see below for details regarding probe synthesis and labelling) were ethanol-precipitated together with 10 μg herring sperm DNA and 2.5 μg Human Cot-1 DNA (Invitrogen, ref. 15279-011, CA, USA), resuspended in 20 μL of hybridization buffer (50% formamide, 2×SSC, 0.5% SDS, 0.5% Sarkosyl, 10 mM NaCl, 30% Block-aid (Invitrogen, ref. B-10710, CA,USA). The probe solution and probes were heat-denatured together on the Hybridizer (Dako, ref. S2451) at 90° C. for 5 min and hybridization was left to proceed on the Hybridizer overnight at 37° C. Slides were washed 3 times in 50% formamide, 2×SSC and 3 times in 2×SSC solutions, for 5 min at room temperature. Detection antibody layers and their respective dilution in Block-Aid are described in table D and E. For each layer, 20 μL of the antibody solution was added on the slide and covered with a combed coverslip and the slide was incubated in humid atmosphere at 37° C. for 20 min. The slides were washed 3 times in a 2×SSC, 1% Tween20 solution for 3 min at room temperature between each layer and after the last layer. For isomer classification, detection was carried out using a Texas Red coupled mouse anti digoxygenin (Jackson Immunoresearch, France) antibody in a 1:25 dilution for H1, H3, and H5 probes, and an Alexa488-coupled streptavidin antibody (Invitrogen, France) in a 1:25 dilution for H2, H4 and H6 probes as primary antibodies. As second layer, an Alexa594-coupled goat anti mouse (Invitrogen, France) diluted at 1:25 and a biotinylated goat antistreptavidin (Vector Laboratories, UK) diluted at 1:50 were used. To amplify the Alexa488-fluorescence signal of H2, H4 and H6 probes, an additional detection layer was realized by using the same Alexa488 coupled-streptavidin used for the first layer at a 1:25 dilution. For the visualisation of the non-canonical structure implicated the H4 and H6 probes, detection was carried out using an AMCA-coupled mouse anti-digoxygenin (Jackson Immunoresearch, France) antibody in a 1:25 dilution for the H2A and H5 probes, an Alexa 594-coupled streptavidin antibody (Invitrogen, France) in a 1:25 dilution for the H1, H2B and H3 and a rabbit anti Alexa 488 in a 1:25 dilution as primary antibodies. As second layer, an Alexa 594-coupled goat anti mouse (Invitrogen, France) diluted at 1:25, a biotinylated goat anti-streptavidin (Vector Laboratories, UK) diluted at 1:50 and an Alexa 488-coupled goat anti-mouse diluted at 1:25 were used. To amplify the Alexa 594-fluorescence signal of the H1, H2B and H3 probes and the AMCA/Alexa 350 signal of the H2A and H5 probes, an additional detection layer was realized by using the same Alexa 594 coupled-streptavidin used for the first layer at a 1:25 dilution and an Alexa350 coupled goat anti rat diluted at 1:25, respectively. After the last washing steps, all glass cover slips were dehydrated in ethanol and air dried.

Analysis of HSV-1 Detected Signals

For direct visualisation of combed HSV-1 fibers, cover slips were mounted with 20 μL of a Prolong (Invitrogen, France ref P36930)-YOYO-1 iodide (Molecular Probes, code Y3601) mixture (1/1000 v/v) and scanned with inverted automated epifluorescence microscope, equipped with a 40× objective (ImageXpress Micro, Molecular Devices, USA). Length of the YOYO-1-stained DNA fibers were measured and converted to kb using an extension factor of 2 kb/μm (Schurra and Bensimon 2009), with an internal software GVlab 04.2.1 (Genomic Vision S.A., Paris, France).

For isomers classification, hybridized-combed DNA from viral particles or cornea preparation were scanned without any mounting medium using an inverted automated epifluorescence microscope, equipped with a 40× objective (ImageXpress Micro, Molecular Devices, USA) and the signals can be detected visually or automatically by an in house software (Gvlab 0.4.2). Both FISH signals composed of a continuous signal of Texas Red/Alexa 594-fluorescence for H1, H3, H5, and Alexa 488-flurorescence for H2, H4 and H6, and signals composed of a continuous signal corresponding to one of the pattern described below were considered:

(a) A minimum of 28 kb long of Texas Red/Alexa 594-fluorescence signal, directly followed by an Alexa488-flurorescence signal corresponding to H4 and another Texas Red/Alexa594-fluorescence signal of a length minimal of 3 kb.

(b) A minimum of 28 kb long of Texas Red/Alexa 594-fluorescence signal, directly followed by an Alexa 488-flurorescence signal corresponding to H6 and another Texas Red/Alexa 594-fluorescence signal of a length minimal of 3 kb.

(c) A minimum of 3 kb an Alexa 488 fluorescence signal directly followed by a Texas Red/Alexa 594 fluorescence signal corresponding to H1, next to an Alexa 488-fluorescence signal corresponding to H4 and another Texas Red/Alexa 594-fluorescence signal of a length minimal of 3 kb.

(d) A minimum of 3 kb an Alexa 488-flurorescence signal directly followed by a Texas Red/Alexa 594-fluorescence signal corresponding to H1, next to an Alexa 488-fluorescence signal corresponding to H6 and another Texas Red/Alexa 594-fluorescence signal of a length minimal of 3 kb.

FISH signals selected were then classified depending of the pattern of the continuous FISH signals analysed:

The probe array composed of H1/H2/H3/H4/H5/H6 probes or the pattern (a) is classified as a Prototype (P) form of HSV-1 (Hayward, Jacob et al. 1975)

The pattern H1/H2/H3/H6/H5/H4 or the pattern (b) is classified as a Inverted Short (IS) genomic region form of HSV-1 (Hayward, Jacob et al. 1975)

The pattern H3/H2/H1/H4/H5/H6 or the pattern (c) is classified as a Inversed Long (IL) genomic region of HSV-1 (Hayward, Jacob et al. 1975)

The pattern H3/H2/H1/H6/H5/H4 or the pattern (d) is classified as a Inversed Long and Short (ILS) genomic region form of HSV-1 (Hayward, Jacob et al. 1975)

The hypothesis that the observed distribution differ significantly to the expected distribution (an equivalent number of events for the four isomers, (Bataille and Epstein 1997) was tested by a chi-square test, and accepted when the p-value observed was below 0.05.

For the visualisation of the non canonical structure implicated the H4 and H6 probes, hybridized-combed DNA from infected cells preparation were scanned without any mounting medium using an inverted automated epifluorescence microscope, equipped with a 40× objective (ImageXpress Micro, Molecular Devices, USA) and the signals can be detected visually on an in house software (Gvlab 0.4.2). All signals composed of a continuous signal of Alexa 488-fluorescence for H4 and H6 were selected. Signals corresponding to one of the pattern below were classified as canonical structure:
(e) A continuous signal composed of a minimum of 3kb of an Alexa 594-fluorescence signal, followed of 13 kb of an Alexa 488-flurorescence signal corresponding to the H4 probe, 7.5 kb of an AMCA/Alexa 350-fluorescence signal corresponding to the H5 probe and 7 kb of an Alexa 488-flurorescence corresponding to the H6 probe.
(f) A continuous signal composed of a minimum of 3 kb of an Alexa 594-fluorescence signal, followed of 7 kb of an Alexa 488-flurorescence signal corresponding to the H6 probe, 7.5 kb of an AMCA/Alexa 350-fluorescence signal corresponding to the H5 probe and 13 kb of an Alexa 488-flurorescence corresponding to the H4 probe.

All other signals were classified as non canonical structure. The proportions of the canonical and non canonical structure were compared.

Extraction of HSV-1 DNA from Viral Particles

During sample preparation many DNA molecules are sheared at random location due to uncontrolled manipulation forces resulting in high variability in the size of DNA prepared. It has been showed that high molecular weight DNA can be stretched on by Molecular Combing using a glass coverslip when it is deproteinised in a molten agarose plug (Lebofsky and Bensimon 2003). Thus, the analyzed DNA molecules are of variable length, with an average of about 300 kb, the longest molecules reaching several megabases.

Since HSV-1 DNA has never been used for Molecular Combing, the inventors first evaluated the quality of the DNA fibers in terms of length extracted by two different methods: the standard phenol:chloroform extraction and the method described by (Lebofsky, Heilig et al. 2006) that have been slightly modified as described in the “Example” section.

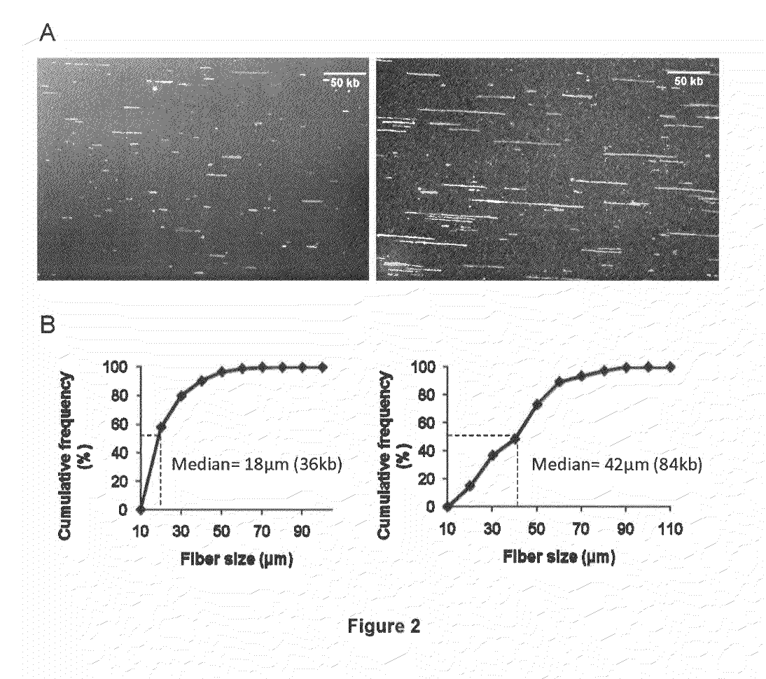

As shown in FIG. 2, no fiber of size above 80 μm was detected. This is in concordance with the maximum expected size for the HSV-1 genome (152 kb) considering a constant elongation factor of 2 kb. With both methods, we do not detect only 152 kb long DNA fibers because there is still random DNA shearing due to the mechanical manipulation that cannot be avoided and because DNA molecules shorter than the full-length standard HSV-1 viral genome can become encapsidated within nuclear capsids (Vlazny, Kwong et al. 1982).

However, the median size of DNA fibers is 36 kb with 1.2% of fiber longer than 140 kb when extraction has been performed with the standard phenol:chloroform method. In contrast, the median size of HSV-1 DNA fiber is 84 kb with 2.5% of fiber longer than 140 kb when the extraction of DNA from agarose plug-embedded viral particles using our alternative protocol has been realized. Although the proportion of long molecules is low, there are a lot of long combed DNA molecules available for analysis since there are several ten thousand fibers combed on a glass coverslip.

These results indicate that the alternative method developed by the inventors improved the quality of combed DNA extracted from viral particles compared to standard method allowing analysis by Molecular Combing.

Structure of the HSV-1 Genome in Viral Particles and its Distribution

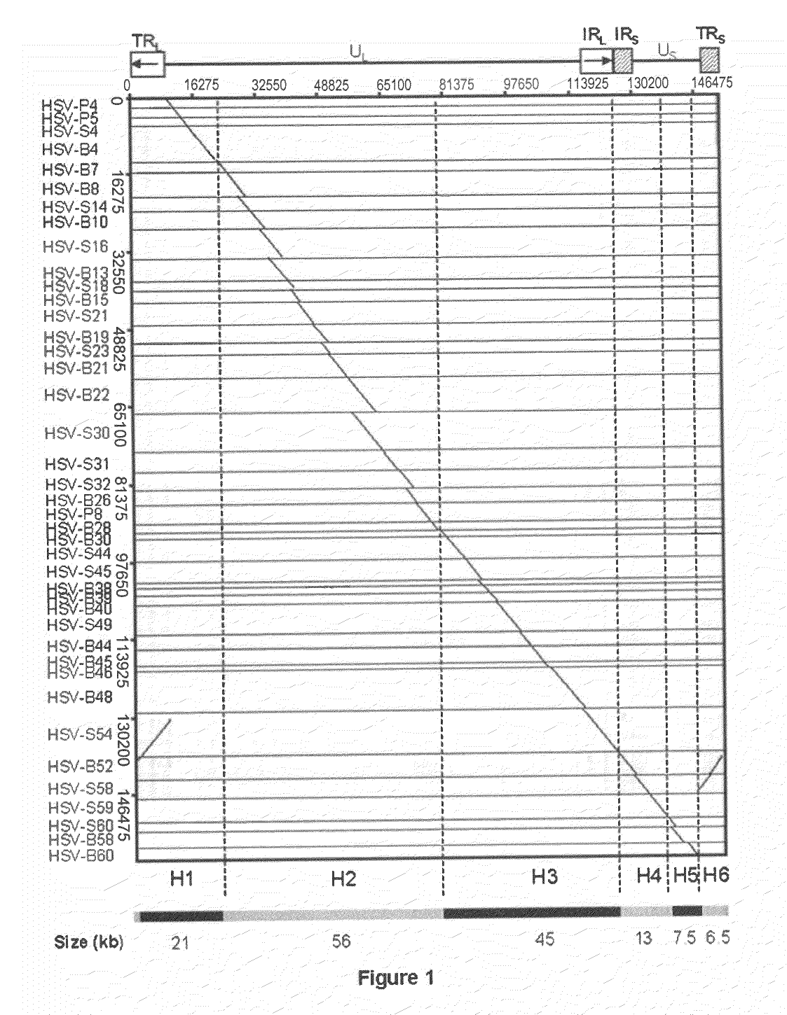

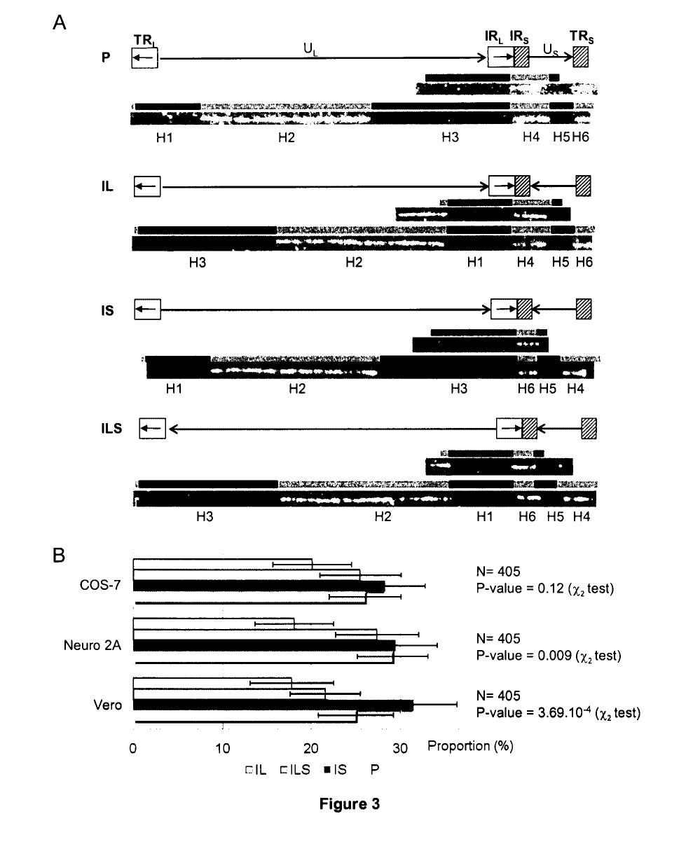

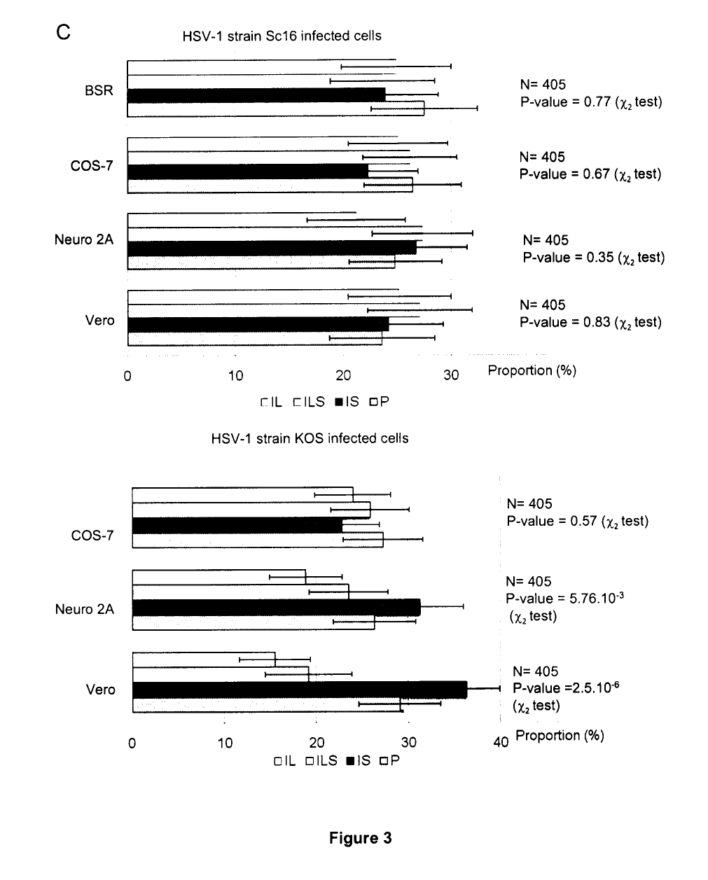

The inventors applied Molecular Combing to uniformly stretch the HSV-1 DNA extracted from viral particles and infected cells and hybridized the resulting combed HSV-1 DNA with labeled adjacent and overlapping HSV-1-specific DNA probes (FIG. 1; H1, H3, H5: red Texas Red/Alexa 594-fluorescence; H2, H4, H6: green Alexa 488-fluorescence) to determine the structure of the HSV-1 genome (FIG. 3A). Immunofluorescence microscopy (FIG. 3B.) exhibited 405 multicolor linear patterns for each production of HSV-1 KOS strain viral particles produced from COS7, Vero and Neuro2 A cell lines that fulfilled the criteria for evaluation (see “Examples” section). Classification of the signal showed that the distribution of the HSV-1 KOS strain isomers are equivalently distributed in viral particles from COS7 cells whereas the P and IS isomers are the more frequent isomer in the viral particles produced from the Neuro2A and Vero cell lines.

On hybridized combed HSV-1 infected cells, the distribution of the four isomers was compared between Sc16 and KOS HSV-1 strains produced in different cell lines (BSR, COS7, Neuro2A and Vero). 405 multicolor linear patterns corresponding to each production and that fulfilled the criteria for evaluations (see “Experimental procedures” section) were classified. The distribution between the four isomers was statistically equivalent (Chi2 test) in all production of HSV-1 Sc16 (in BSR, Vero, Neuro 2a and COS-7 cells). In the same way, the distribution was found equivalent for HSV-1 strain KOS produced in COS-7 cells. Strikingly, for the first time, the inventors have found that the IS and P isomers are the predominant forms of the HSV-1 DNA strain KOS preparation from Vero and Neuro2A cells while IL isomers is the less present isomers.

The inventors have found that the Molecular Combing techniques as described herein are powerful methods for analysis of the structure of the HSV-I genome DNA at the level of the unique molecule and to quantify its distribution in a biological sample.

Extraction of Genomic DNA from Mouse and Rabbit Cornea

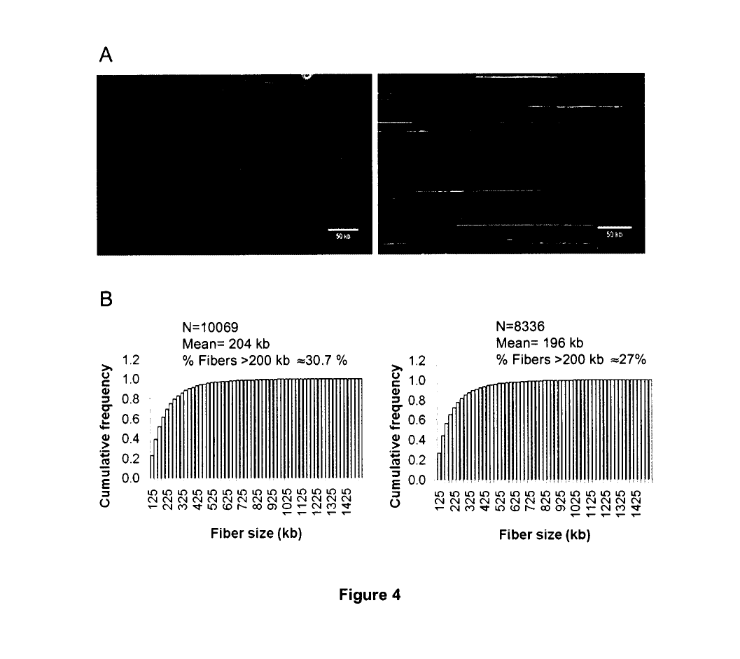

Molecular Combing has been successfully performed with DNA solution from isolated cells including cultured cells (i.e., established cell strains, immortalized primary cells) or biological fluids (i.e., peripheral blood lymphocytes, amniotic cells) (Gad, Klinger et al. 2002; Caburet, Conti et al. 2005). However, the human cornea is a solid tissue with a complex structure composed of 5 layers: the corneal epithelium, the collagen-rich Bowman's membrane, the corneal stroma which consisting of regularly-arranged collagen fibers along with sparsely distributed interconnected keratocytes), the acellular Descemet's membrane and, the corneal endothelium. In order to extract genomic DNA from cornea, the inventors developed a specific method to isolate corneal cells before proceeding with the standard procedure. Different methods including mechanical disruption and enzymatic digestion of cornea were tested. The latter was given the best results and was optimized using different types of proteases (i.e., trypsin, collagenase A, dispase . . . ) at different concentration. FIG. 4A and B shows an example of results obtained with both mouse and rabbit cornea that were digested with 0.3 mg/ml collagenase A and 0.8 mg/ml Dispase for 16 h. As for standard extraction for Molecular Combing genomic DNA is broken at random locations. Thus, the analyzed genomic DNA molecules are of variable length, with an average of about 200 kb, the longest molecules above 1 Mb (megabases) for both type of cornea. The size of DNA fiber extracted from cornea is slightly inferior to the typical size that can be obtained from isolated cells (an average of about 300 kb with the longest molecules reaching several megabases).

Detection of HSV-1 Infection in Mouse Cornea

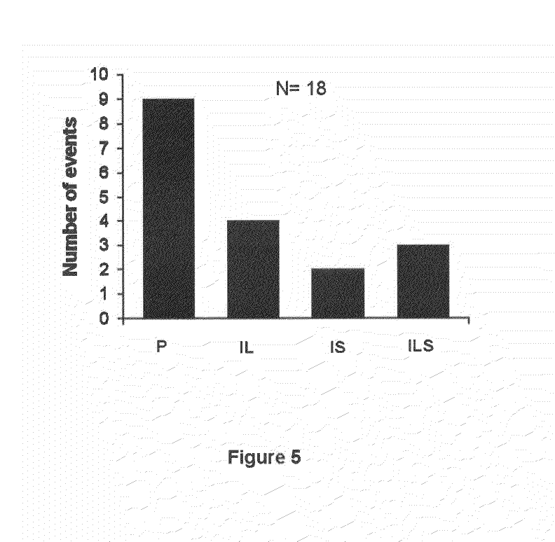

The inventors therefore adapted and applied Molecular Combing on DNA extracted from HSV-1 infected mouse cornea and hybridized with the HSV-1 specific probes as described above. As shown in FIG. 5, this enabled detection all the types of isomers of HSV-1 genome in mouse infected cornea.

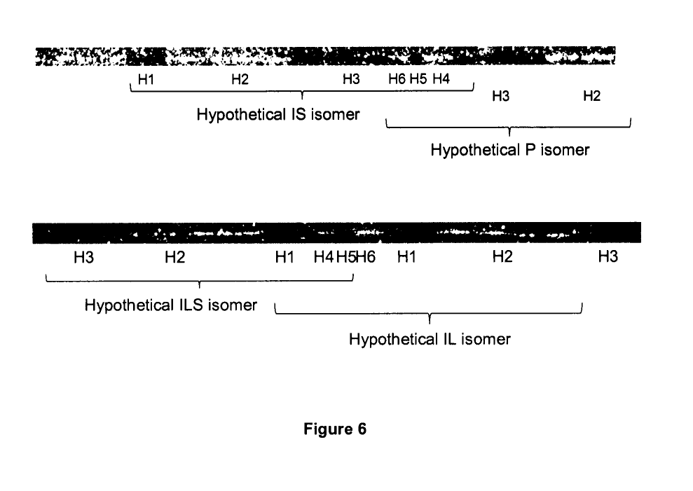

In addition to the detection of mature HSV-1 genome, the Molecular Combing procedures of the invention allow the detection of concatemers (FIG. 6) indicating that the virus is actively replicating in the cornea of the analyzed sample.

Detection of Non-Canonical Forms in Infected Cells and Mouse Cornea

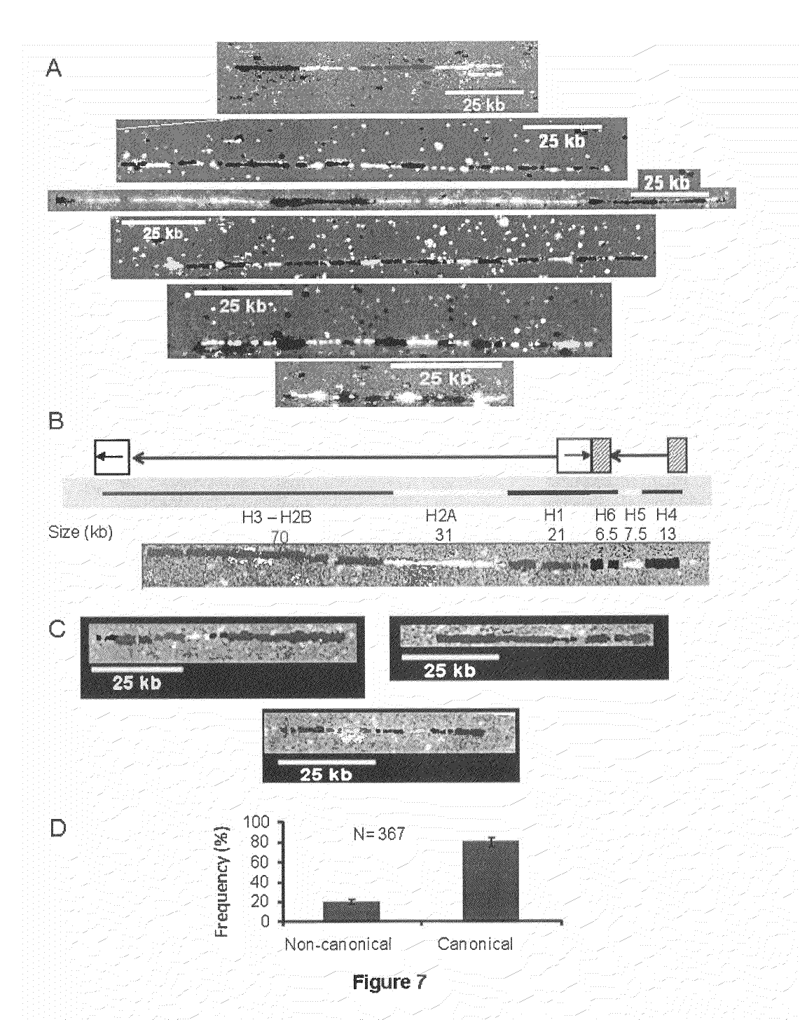

The inventors detected non-canonical structure of the HSV-1 genome (FIG. 7A) that probably arises from recombination during the replication of the virus in infected mouse cornea extract and in infected cells extracts.

The labelled adjacent and overlapping HSV-1 specific probes were hybridized on combed DNA extracts from HSV-1 strain Sc16 infected Vero cells (FIG. 7B; H1, H2A, H3: red Alexa 594-fluorescence that appears in grey; H2B, H5: blue AMCA/Alexa 350-fluorescence that appears in white; H4, H6: green Alexa 488-fluorescence that appears in black) to evaluate the proportion of the non-canonical structures in the H4/H6 region. A total of 367 multicolour linear patterns were classified, 20% (73) are found to have non-canonical H4/H6 structure.

Molecular Combing enables the visualisation of non canonical structure and by their infinity of combination of barcode possible is a powerful method to analyse them.

TABLE A

| Name | Start | End | Size (bp) |
|---|---|---|---|
| HSV-B1 | 1 | 1323 | 1323 |
| HSV-B2 | 1324 | 7259 | 5936 |
| HSV-P4 | 9237 | 11276 | 2004 |
| HSV-P5 | 11090 | 13245 | 2156 |
| HSV-Sc4 | 13088 | 14971 | 1884 |
| HSV-P6 | 14554 | 17565 | 3065 |
| HSV-B4 | 14827 | 22438 | 7595 |
| HSV-Sc7 | 17853 | 20762 | 2910 |
| HSV-B7 | 22953 | 25152 | 2200 |
| HSV-Sc11 | 23400 | 25447 | 2048 |
| HSV-B8 | 25153 | 29997 | 4845 |
| HSV-Sc14 | 27944 | 31215 | 3272 |
| HSV-B10 | 30903 | 34697 | 3795 |
| HSV-Sc16 | 33044 | 39471 | 6428 |
| HSV-B13 | 35891 | 40272 | 4382 |
| HSV-Sc18 | 40315 | 42288 | 1974 |
| HSV-B15 | 41017 | 43898 | 2882 |
| HSV-Sc21 | 42621 | 47358 | 4738 |
| HSV-P8 | 44192 | 46987 | 2795 |
| HSV-B18 | 44682 | 47174 | 2493 |
| HSV-B19 | 47175 | 50931 | 3757 |
| HSV-Sc23 | 49040 | 51392 | 2353 |
| HSV-B21 | 50959 | 56138 | 5180 |
| HSV-Sc24 | 51393 | 53348 | 1956 |
| HSV-Sc25 | 53349 | 56049 | 2701 |
| HSV-B22 | 56139 | 63370 | 7232 |
| HSV-Sc30 | 56775 | 64599 | 7825 |
| HSV-Sc31 | 64600 | 69017 | 4418 |
| HSV-B24/25 | 65757 | 66423 | 4740 |
| HSV-Sc32 | 69018 | 72802 | 3785 |
| HSV-B26 | 70497 | 73717 | 3221 |
| HSV-P8 | 73229 | 77332 | 4103 |
| HSV-B27 | 73718 | 77164 | 3447 |
| HSV-B28 | 77165 | 79105 | 1941 |
| HSV-B30 | 79937 | 81056 | 1120 |
| HSV-Sc44 | 80991 | 85801 | 4811 |
| HSV-B31 | 81507 | 83780 | 2724 |
| HSV-B33 | 84298 | 85576 | 1279 |
| HSV-Sc45 | 85802 | 90164 | 4363 |
| HSV-B35 | 86304 | 87747 | 1444 |
| HSV-B38 | 89249 | 90453 | 1205 |
| HSV-B39 | 90453 | 92176 | 1723 |
| HSV-B40 | 92177 | 94195 | 2019 |
| HSV-Sc47 | 91545 | 93723 | 2179 |
| HSV-Sc49 | 94122 | 100285 | 6164 |
| HSV-B41 | 94196 | 96148 | 1953 |
| HSV-B42 | 96149 | 99454 | 3306 |
| HSV-B44 | 99492 | 102440 | 2949 |
| HSV-B45 | 102441 | 106065 | 3625 |
| HSV-B46 | 106066 | 107175 | 1110 |
| HSV-B48 | 108009 | 116579 | 8571 |
| HSV-P8c | 109960 | 113148 | 3188 |
| HSV-Sc54 | 115744 | 125068 | 9325 |
| HSV-B52 | 125044 | 129901 | 4858 |
| HSV-Sc56 | 125079 | 128601 | 3523 |
| HSV-Sc58 | 129089 | 133046 | 3958 |
| HSV-B55 | 130841 | 135542 | 4702 |
| HSV-Sc59 | 133047 | 137945 | 4899 |
| HSV-B56 | 135543 | 137690 | 2148 |
| HSV-Sc60 | 137946 | 140155 | 2210 |
| HSV-P8d | 138148 | 139821 | 1673 |
| HSV-B58 | 138757 | 141926 | 3170 |
| HSV-B60 | 142840 | 145515 | 2276 |
| HSV-Sc64 | 144918 | 149148 | 4231 |

TABLE B

| Probes | Forward Primer | Reverse Primer |
|---|---|---|
| HSV-P4 | TGG TTG TGT TAC | TCG ATC GAC GAC |
| | TGG GCA AA | ACC ATA AA |
| | (SEQ ID NO: 1) | (SEQ ID NO: 2) |
| HSV-P5 | CAG ATA CGA CTC | CGA CGA CCT CGA |
| | CCG CAG AT | CGT TAT TT |
| | (SEQ ID NO: 3) | (SEQ ID NO: 4) |
| HSV-P6 | CGT GAG GTC CAA | GAC AGG CAA GCT |
| | AAT CAC CT | CAA AGT CC |
| | (SEQ ID NO : 5) | (SEQ ID NO : 6) |
| HSV-P8 | AGA TGT CCA CGA | CCT GAC TTT GTG |
| | GCA CCA G | GGG CTA AA |
| | (SEQ ID NO: 7) | (SEQ ID NO: 8) |

Primers sequences used for the synthesis of probes by long-range PCR. An extract of DNA from HSV-1 strain Sc16 is used as template.

TABLE C

| | Non-labelled dNTPs | Labelled dNTP |
|---|---|---|
| | (Invitrogen, ref. | (Roche, ref. 11 558 |
| Labelling | 10297-018, CA, USA) | 706 910, France) |
| Dig-dUTP | dATP, dCTP, dGTP 40 μM | Dig-11-dUTP 20 μM |
| | each | |
| | dTTP 20 μM | |
| Alexa488-7- | dATP, dTTP, dGTP 40 μM | Alexa488-7-OBEA-dCTP |
| OBEA-dCTP | each | 20 μM |
| | dCTP 20 μM | |

Mixes used in replacement of the dNTP mix of the random priming kit for labelling with dig-dUTP and Alexa488-7-OBEA-dCTP. The concentrations indicated are the final concentration in the labelling reaction. The non-labelled dNTPs and the labelled dNTP were added together in replacement of the provided dNTP mix intended for labelling with biotin-11-dCTP-.

TABLE D

| Description | Abbreviation | Supplier |
|---|---|---|
| Streptavidin, coupled to | Strep/A488 | Invitrogen (France, |
| Alexa Fluor 488 | | S11223) |
| Goat anti-streptavidin, | G anti-strep/biotin | Vector Laboratories |
| coupled to biotin | | (France; BA-0500) |
| Mouse anti-dig, coupled to | M anti-DIG/TR | Jackson Immuno |
| Texas Red | | Research (France; |
| | | 200-072-156) |
| Goat anti-mouse, coupled to | G anti-M/A594 | Invitrogen (France; |
| A594 | | A11005) |
| Streptavidin, coupled to | Strep/A594 | Invitrogen (France, |
| Alexa Fluor 594 | | S11227) |
| Mouse anti-Dig, AMCA | M anti-DIG/AMCA | Jackson Immuno |
| coupled | | Research (France; |
| | | 200-152-156) |
| Rat anti-mouse, AMCA | R anti-M/AMCA | Jackson Immuno |
| coupled | | Research (France; |
| | | 415-155-166) |
| Goat anti-rat, Alexa 350 | G anti-R/A350 | Invitrogen (France, |
| coupled | | A21093) |
| Rabbit anti Alexa 488 | R anti-A488 | Invitrogen (France, |
| | | A11094) |
| Goat anti mouse, coupled | G anti-M/A488 | Invitrogen (France, |
| Alexa 488 | | A11001) |

List of antibodies and other hapten-binding molecules used for the detection of probes.

TABLE E

| | 1^{st} layer | 2^{nd} layer | 3^{rd} layer |
|---|---|---|---|
| 1-color scheme | | | |
| Biotin/green | strep/A488 | Goat anti-strep/ | strep/A488 |
| | (1/25) | biotin | (1/25) |
| | | (1/50) | |
| 2-color scheme | | | |
| Biotin/green | strep/A488 | Goat anti-strep/ | strep/A488 |
| | (1/25) | biotin | (1/25) |
| | | (1/50) | |
| Dig/red | Mouse anti- | Goat anti-M/ | — |
| | DIG/TR | A594 | |
| | (1/25) | (1/25) | |
| 3-color scheme | | | |
| Biotin/red | Strep/A594 | G anti-strep/ | Strep/A594 |
| | (1/25) | biotin | (1/25) |
| | | (1/50) | |
| Dig/blue | M anti- | R anti-M/AMCA | G anti-R/A350 |
| | DIG/AMCA | (1/25) | (1/25) |
| | (1/25) | | |
| A488/green | R anti-A488 | G anti-M/A488 | — |
| | (1/25) | (1/25) | |

Composition of the 2 or 3 layers for the detection of probes by fluorescence. The dilution for each detection agent is indicated in brackets. The abbreviations refer to table D.

### Example 2

### Human Immunodeficiency Virus Detection

Preparation of Embedded DNA Plugs from ACH-2 Cells Culture

ACH-2 cell lines (Clouse, Powell et al. 1989) were cultivated according to the authors' instructions. DNA was extracted as described in (Schurra and Bensimon 2009). Briefly, cells were resuspended in 1× PBS at a concentration of 10⁷ cells/mL mixed thoroughly at a 1:1 ratio with a 1.2% w/v solution of low-melting point agarose (Nusieve GTG, ref. 50081, Cambrex) prepared in 1× PBS at 50° C. 90 μL of the cell/agarose mix was poured in a plug-forming well (BioRad, ref. 170-3713) and left to cool down at least 30 min at 4° C. Agarose plugs were incubated overnight at 50° C. in 250 μL of a 0.5M EDTA (pH 8), 1% Sarkosyl, 250 μg/mL proteinase K (Eurobio, code: GEXPRK01, France) solution, then washed twice in a Tris 10 mM, EDTA 1 mM solution for 30 in at room temperature.

Final Extraction of DNA and Molecular Combing

Plugs of embedded DNA from ACH-2 cells were treated for combing DNA as previously described (Schurra and Bensimon 2009). Briefly, plugs were melted at 68° C. in a MES 0.5 M (pH 5.5) solution for 20 min, and 1.5 units of beta-agarase (New England Biolabs, ref. M0392S, MA, USA) was added and left to incubate for up to 16 h at 42° C. The DNA solution was then poured in a Teflon reservoir and Molecular Combing was performed using the Molecular Combing System (Genomic Vision S.A., Paris, France) and Molecular Combing coverslips (20 mm×20 mm, Genomic Vision S.A., Paris, France). The combed surfaces were dried for 4 hours at 60° C.

Synthesis and Labelling of HIV-1 Probes

The coordinates of the three probes relative to the Genbank sequence M19921.1 are listed in table F. Probe size ranges from 2927 to 3749 bp in this example.

The HIV specific probes were produced by long-range PCR using LR Taq DNA polymerase (Roche, kit code: 11681842001) using the primers listed in table G and the DNA from HIV pNL4-3 as template DNA. PCR products were ligated in the pCR®2.1 vector using the TOPO® TA cloning Kit (Invitrogen, France, code K455040). The two extremities of each probe were sequenced for verification purpose.

Two fosmids G248P87988G9 and G248P86255A8 flanking the insertion site of HIV-1 or one fosmid G248P84833H9 encompassing the HIV-1 provirus insertion site in ACH-2 cells (Ishida, Hamano et al. 2006), according to Human March 2006 Assembly (NCBI Build 36.1), and the HIV-1 probes were labeled using conventional random priming protocols. For biotin-11-dCTP labelling, the BioPrime® DNA kit (Invitrogen, code: 18094-011, CA, USA) was used according to the manufacturer's instruction, except the labelling reaction was allowed to proceed overnight. For digoxygenin-11-dUTP, the dNTP mix from the kit was replaced by the mix specified in table C. 200 ng of each plasmid/fosmid was labelled in separate reactions. For entire HIV-1 detection, HIV-1 was labelled with digoxygenin-11-dUTP while fosmids were labelled with biotin-11-dCTP. The reaction products were visualized on an agarose gel to verify the synthesis of DNA.

Hybridization of HIV-1 Probes on Combed Viral DNA and Detection

Subsequent steps were also performed essentially as previously described in Schurra and Bensimon, 2009 (Schurra and Bensimon 2009). Briefly, a mix of labelled probes (250 ng of each probe) were ethanol-precipitated together with 10 μg herring sperm DNA and 2.5 μg Human Cot-1 DNA (Invitrogen, ref. 15279-011, CA, USA), resuspended in 20 μL of hybridization buffer (50% formamide, 2×SSC, 0.5% SDS, 0.5% Sarkosyl, 10 mM NaCl, 30% Block-aid (Invitrogen, ref. B-10710, CA,USA). The probe solution and probes were heat-denatured together on the Hybridizer (Dako, ref. S2451) at 90° C. for 5 min and hybridization was left to proceed on the Hybridizer overnight at 37° C. Slides were washed 3 times in 50% formamide, 2×SSC and 3 times in 2×SSC solutions, for 5 min at room temperature. Detection antibody layers and their respective dilution in Block-Aid are described in table D and E. For each layer, 20 μL of the antibody solution was added on the slide and covered with a combed coverslip and the slide was incubated in humid atmosphere at 37° C. for 20 min. The slides were washed 3 times in a 2×SSC, 1% Tween20 solution for 3 min at room temperature between each layer and after the last layer.

Detection of entire HIV-1 was carried out using a Texas Red coupled mouse anti-digoxygenin (Jackson Immunoresearch, France) antibody in a 1:25 dilution for HIV-1 probes, and an Alexa488-coupled streptavidin antibody (Invitrogen, France) in a 1:25 dilution for fosmids as primary antibodies. As second layer, an Alexa594-coupled goat anti mouse (Invitrogen, France) diluted at 1:25 and a biotinylated goat antistreptavidin (Vector Laboratories, UK) diluted at 1:50 were used. To amplify the Alexa488-fluorescence signal of fosmids, an additional detection layer was realized by using the same Alexa488 coupled-streptavidin used for the first layer at a 1:25 dilution. After the last washing steps, all glass cover slips were dehydrated in ethanol and air dried.

Analysis of HIV-1 Detected Signals

Hybridized-combed DNA from ACH-2 cells preparation were scanned without any mounting medium using an inverted automated epifluorescence microscope, equipped with a 40× objective (ImageXpress Micro, Molecular Devices, USA) and the signals can be detected visually or automatically by an in house software (Gvlab 0.4.2):
Using the two fosmids G248P87988G9 and G248P86255A8 flanking the insertion site of HIV-1, FISH signals corresponding to one of the pattern as follow were considered and measured, using an extension factor of 2 kb/μm (Schurra and Bensimon 2009):
   FISH signals composed of a continuous signal of Texas Red/Alexa 594-fluorescence for HIV-1. The entire signal that correspond of isolated HIV proviral DNA is measured
   FISH signal array composed of signal chain of Texas Red/Alexa 594-fluorescence for HIV-1, flanked by two gaps, and two continuous signals of Alexa 488-flurorescence corresponding to the fosmid sequences. The entire Texas Red/Alexa 594 fluorescence signal and gaps length flanking this signal were measured and corresponds to HIV-1 proviral DNA integrated in the chromosome 7 at 7p15 (Ishida, Hamano et al. 2006).
   FISH signal array with two Alexa 488-flurorescence signals separated by a gap. Measurement of the gap length that corresponds to the 7p15 locus without integration of HIV-1 proviral DNA was performed.
Using the fosmid G248P84833H9 encompassing the HIV-1 provirus insertion site, FISH signals corresponding to one of the pattern as follow were considered and measured, using an extension factor of 2 kb/μm (Schurra and Bensimon 2009):
   FISH signals composed of a continuous signal of Texas Red/Alexa 594-fluorescence for HIV-1. The entire signal that correspond of isolated HIV proviral DNA is measured
   FISH signal array composed of a continuous signal chain of Texas Red/Alexa 594-fluorescence for HIV-1, flanked by two signals of Alexa 488-flurorescence corresponding to the fosmid sequences. The entire Texas Red/Alexa 594 fluorescence signal was measured and corresponds to HIV-1 proviral DNA integrated in the chromosome 7 at 7p15 (Ishida, Hamano et al. 2006).
   FISH signal with one long Alexa 488-flurorescence signals corresponds to the 7p15 locus without integration of HIV-1 proviral DNA.

Detection of HIV-1 in ACH-2 Cells Culture

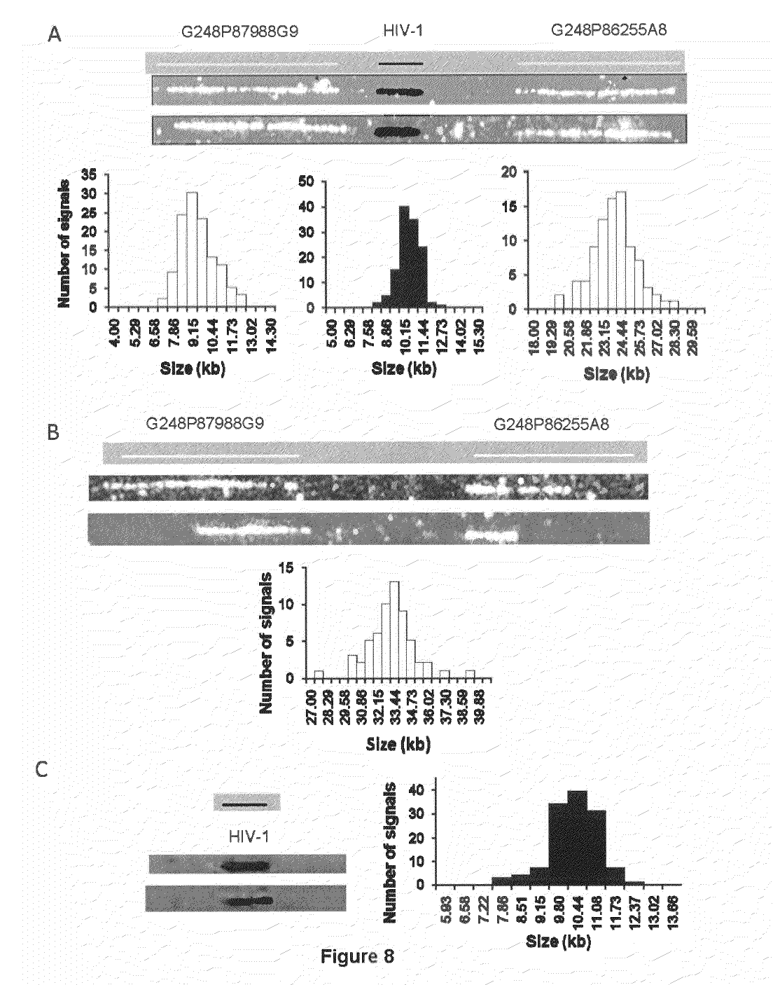

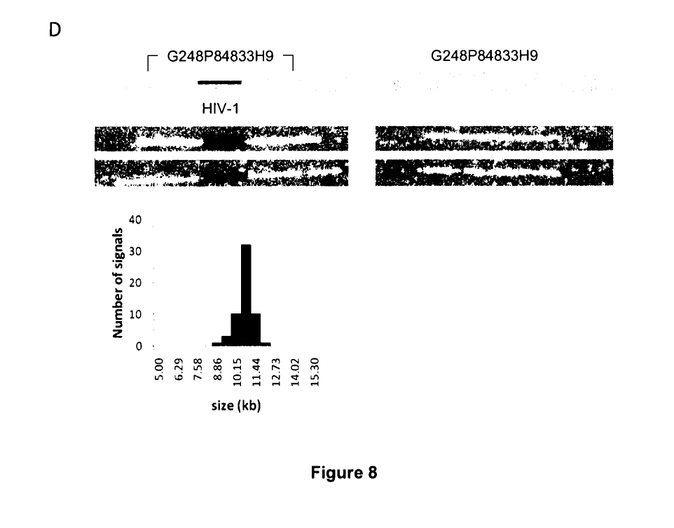

The inventors have applied Molecular Combing to detect complete HIV-1 integrated provirus in ACH-2 cell lines (Clouse, Powell et al. 1989), which contain a unique integrated form of HIV-1 in its genome in NT5C3 (Cytosolic 5′-nucleotidase III) gene on 7p14.3 (Ishida, Hamano et al. 2006). Labeled fosmids flanking the insertion site (G248P87988G9 and G248P86255A8) were hybridized on combed ACH-2 DNA simultaneously than labelled HIV-1 probes. A mean size of 10.2 kb+/−0.8 kb was obtained from measurement of 124 HIV-1 FISH signals flanking by one or both fosmids, corresponding to the expected size of HIV-1 (9.7 kb) (FIG. 8A, HIV probes: red Texas Red/Alexa 594-fluorescence, Fosmids: green Alexa 488-fluorescence). Normal alleles of NT5C3 gene are detected and measurement of the gap length between fosmids G248P87988G9 and G248P86255A8 FISH signal leads to a mean size of 32.8±1.8 kb, lightly superior to the expected size of 31 kb, according to Human March 2006 Assembly (NCBI Build 36.1). This result shows that Molecular Combing by its resolution may have bring some further information about this locus (FIG. 8B). Furthermore, 133 isolated HIV-1 FISH signals were measured in this combed ACH-2 DNA preparation, with a mean size of 10.05 kb+/−0.79 kb (FIG. 8C). This contrasts with the expected unique HIV-1 site of insertion described previously (Ishida, Hamano et al. 2006) and suggests that it exists in ACH-2 genome another or others insertion site(s) of HIV-1, or that a non integrated form of HIV-1 is persistent in ACH-2 nucleus. Similar observations are performed when the labeled fosmide encompassing the insertion site (G248P84833H9) was hybridized on combed ACH-2 DNA simultaneously than labelled HIV-1 probes (FIG. 8D). A mean size of 10.4 kb+/−0.5 kb was obtained from measurement of 57 HIV-1 FISH signals within the fosmide signal, corresponding to the expected size of HIV-1 provirus (9.7 kb) (HIV probes: red Texas Red/Alexa 594-fluorescence, fosmid: green Alexa 488-fluorescence). Furthermore, 35 isolated HIV-1 FISH signals were also detected and measured in this combed ACH-2 DNA preparation, with a mean size of 10.03 kb+/−0.82 kb (not shown).

These results indicate that Molecular Combing is a powerful method to analyze the structure of the HIV genome DNA and to quantify its integration in genomic DNA at the level of the unique molecule and in any biological sample.

TABLE F

| Name | Start | End | Size (bp) |
|---|---|---|---|
| HIV-S1 | 1 | 3026 | 3026 |
| HIV-S2 | 3018 | 5944 | 2927 |
| HIV-S3 | 5961 | 9709 | 3749 |

Coordinates of the three probes used in this example, relative to the Genbank sequence M19921.1

TABLE G

| Probes | Forward Primer | Reverse Primer |
|---|---|---|
| HIV-S1 | TGGAAGGGCTAATTT | TATTGCTGGTGATCCT |
| | GGTC | TTCC |
| | (SEQ ID NO : 9) | (SEQ ID NO : 10) |
| HIV-S2 | CCAGCAATATTCCAGT | TGAAACAAACTTGGCA |
| | GTAGC | ATGA |
| | (SEQ ID NO : 11) | (SEQ ID NO : 12) |
| HIV-S3 | CATCTCCTATGGCAGG | TGCTAGAGATTTTCCA |
| | AAGA | CACTGA |
| | (SEQ ID NO : 13) | (SEQ ID NO : 14) |

Primers sequences used for the synthesis of probes by long-range PCR. An extract of DNA from HIV-1 pNL-4 is used as template.

### Example 3

### Detecting an Oncogene by Molecular Combing

In a manner to analogous to the detection of HSV genomic DNA in Example 1, probes are designed to detect the presence of a viral oncogene. Probes are designed to complement 80-100% of the active viral oncogene of interest and Molecular Combing is performed. The results indicate the presence of an active oncogene in a subject, leading to diagnosis and therapeutic intervention.

### Example 4

### Detecting Rearrangements of Infectious Viral DNA Using Molecular Combing

In a manner to analogous to the detection of HSV genomic DNA in Example 1, probes are designed to detect the presence of different arrangements of infectious viral DNA. Different sets of probes tagged with different haptens recognized by different colored fluorescent probes are designed to complement 80-100% of the active viral oncogene of interest and Molecular Combing is performed. Variations in the arrangement of viral genes in a subject's cells, tissue or biological fluid are used to diagnose or prognose the risks of progression of the viral disease or disease associated with rearrangement of the viral genome, such as the risk of or induction of a tumorigenic properties by conversion of proto-oncogenes into oncogenes.

### Example 5

### Monitoring Genetic Therapy by Molecular Combing

In a manner to analogous to the detection of proviral forms of HIV-1 in Example 2, probes are specially designed to complement 80-100% of an integrated therapeutic adenovirus vector or the transgenetic sequence(s) it carries. Molecular Combing is performed using the specially designed probes to detect the presence of transgenic material integrated into a host chromosome and whether it is arranged in form that can actively express the transgene(s). The quantity of transgene(s) in the subject is followed longitudinally and a determination is made when and whether to re-administer the therapeutic adenovirus vector.

### REFERENCES

Patents

U.S. Pat. No. 6,130,044: Surfaces for biological reactions, process for preparing them and process for their use Bensimon D., Bensimon A., Heslot F. Oct. 10, 2000

U.S. Pat. No. 6,303,296, US2006257910, U.S. Pat. No. 6,054,327: Process for aligning macromolecules by passage of a meniscus and applications, Bensimon D., Bensimon A., Heslot F., Oct. 16, 2001.

U.S. Pat. No. 6,225,055: Apparatus for the parallel alignment of macromolecules, and use thereof, Bensimon A., Bensimon D, May 22, 2002.

U.S. 2004033510: Method for the diagnosis of genetic diseases by molecular combing and diagnostic kit, Bensimon A., Bensimon D., Michalet X., Feb. 19, 2004

EP0263025: Fractions containing antigens specific for herpes virus type 1 (HSV-1) and type 2 (HSV-2), method for isolating these fractions and diagnostic method specific for HSV-1 and/or HSV-2 using these fractions, Markoulatos P., Jun. 30, 1993.

EP0139416: Molecularly cloned diagnostic product and method of use, Berman, P. W., Lasky L. A, Nov. 5, 2003.

WO9818959: Procédé de diagnostic de maladies génétiques par Peignage Moléculaire et coffret de diagnostic, Bensimon A., Bensimon D., Michalet X., 1998 May 7.

WO0073503: Utilisation du peignage dans l'identification des origines de réplication d'ADN, Bensimon A., Herrick J., Hyrien O., 2000 Dec. 7.

WO0202131 : Compositions et méthodes de diagnostic et de traitement de l'infection du virus de l'herpès. Hosken N. A, Day C. H., Dillon D. C., McGowan P., Sleath P. R., 2002 Jan. 10.

WO2004036185 Diagnostic et traitement des maladies du virus de l'herpes simplex (HSV), Kriesel J. D.; Leppert M. F.; Spruance S. L., Otterud B. E M, Peiffer A. P., 2004 Apr. 29.

WO2008028931: Genomic Morse Code, Lebofsky R., Bensimon A., Walrafen P., 2008 Mar. 13.

### Scientific Publications

Barton, E. S., D. W. White, et al. (2007). “Herpesvirus latency confers symbiotic protection from bacterial infection.” Nature 447(7142): 326-329.

Bataille, D. and A. L. Epstein (1997). “Equimolar generation of the four possible arrangements of adjacent L components in herpes simplex virus type 1 replicative intermediates.” J Virol 71(10): 7736-7743.

Ben-Zeev, A., E. Weinberg, et al. (1974). “Isolation of herpes simplex virus DNA from virus particles and infected cells by electrophoresis in polyacrylamide gels.” J Gen Virol 25(1): 63-73.

Bertoletti, A. and A. Gehring (2007). “Immune response and tolerance during chronic hepatitis B virus infection.” Hepatol Res 37 Suppl 3: S331-338.

Boehmer, P. E. and I. R. Lehman (1997). “Herpes simplex virus DNA replication.” Annu Rev Biochem 66: 347-384.

Breitbart, M. and F. Rohwer (2005). “Here a virus, there a virus, everywhere the same virus?” Trends Microbiol 13(6): 278-284.

Caburet, S., C. Conti, et al. (2005). “Human ribosomal RNA gene arrays display a broad range of palindromic structures.” Genome Res 15(8): 1079-1085.

Cockerham, G. C., K. Bijwaard, et al. (2000). “Primary graft failure : a clinicopathologic and molecular analysis.” Ophthalmology 107(11): 2083-2090; discussion 2090-2081.

Collier, L. H. and J. S. Oxford (2006). Human virology : a text for students of medicine, dentistry, and microbiology. Oxford ; New York, Oxford University Press.

Crouse, C. A., S. C. Pflugfelder, et al. (1990). “Detection of herpes viral genomes in normal and diseased corneal epithelium.” Curr Eye Res 9(6): 569-581.

Davison, A. J. and N. M. Wilkie (1981). “Nucleotide sequences of the joint between the L and S segments of herpes simplex virus types 1 and 2.” J Gen Virol 55(Pt 2): 315-331.

Dimmock, N. J., A. J. Easton, et al. (2007). Introduction to modern virology. Malden, MA, Blackwell Pub.

El-Aal, A. M., M. El Sayed, et al. (2006). “Evaluation of herpes simplex detection in corneal scrapings by three molecular methods.” Curr Microbiol 52(5): 379-382.

Fields, B. N., D. M. Knipe, et al. (2007). Fields' virology. Philadelphia, Wolters kluwer/Lippincott Williams & Wilkins.

Fukuda, M., T. Deai, et al. (2008). “Presence of a large amount of herpes simplex virus genome in tear fluid of herpetic stromal keratitis and persistent epithelial defect patients.” Semin Ophthalmol 23(4): 217-220.

Gad, S., A. Aurias, et al. (2001). “Color bar coding the BRCA1 gene on combed DNA: a useful strategy for detecting large gene rearrangements.” Genes Chromosomes Cancer 31(1): 75-84.

Gad, S., I. Bieche, et al. (2003). “Characterisation of a 161 kb deletion extending from the NBR1 to the BRCA1 genes in a French breast-ovarian cancer family.” Hum Mutat 21(6): 654.

Gad, S., V. Caux-Moncoutier, et al. (2002). “Significant contribution of large BRCA1 gene rearrangements in 120 French breast and ovarian cancer families.” Oncogene 21(44): 6841-6847.

Gad, S., M. Klinger, et al. (2002). “Bar code screening on combed DNA for large rearrangements of the BRCA1 and BRCA2 genes in French breast cancer families.” J Med Genet 39(11): 817-821.

Gardella, T., P. Medveczky, et al. (1984). “Detection of circular and linear herpesvirus DNA molecules in mammalian cells by gel electrophoresis.” J Virol 50(1): 248-254.

Goudsmit, J. (1998). Viral sex : the nature of AIDS. New York ; Oxford, Oxford University Press.

group, T. H. E. D. S. (1998). “Acyclovir for the prevention of recurrent herpes simplex virus eye disease. Herpetic Eye Disease Study Group.” N Engl J Med 339(5): 300-306.

Hampson, A. W. and J. S. Mackenzie (2006). “The influenza viruses.” Med J Aust 185(10 Suppl): S39-43.

Hayward, G. S., R. J. Jacob, et al. (1975). “Anatomy of herpes simplex virus DNA: evidence for four populations of molecules that differ in the relative orientations of their long and short components.” Proc Natl Acad Sci USA 72(11): 4243-4247.

Herrick, J., C. Conti, et al. (2005). “Genomic organization of amplified MYC genes suggests distinct mechanisms of amplification in tumorigenesis.” Cancer Res 65(4): 1174-1179.

Herrick, J., S. Jun, et al. (2002). “Kinetic model of DNA replication in eukaryotic organisms.” J Mol Biol 320(4): 741-750.

Herrick, J., X. Michalet, et al. (2000). “Quantifying single gene copy number by measuring fluorescent probe lengths on combed genomic DNA.” Proc Natl Acad Sci USA 97(1): 222-227.

Herrick, J., P. Stanislawski, et al. (2000). “Replication fork density increases during DNA synthesis in X. laevis egg extracts.” J Mol Biol 300(5): 1133-1142.

Kabra, A., P. Lalitha, et al. (2006). “Herpes simplex keratitis and visual impairment: a case series.” Indian J Ophthalmol 54(1): 23-27.

Kessler, H. H., G. Muhlbauer, et al. (2000). “Detection of Herpes simplex virus DNA by real-time PCR.” J Clin Microbiol 38(7): 2638-2642.

Kotronias, D. and N. Kapranos (1998). “Detection of herpes simplex virus DNA in human spermatozoa by in situ hybridization technique.” In Vivo 12(4): 391-394.

Labetoulle, M., P. Auquier, et al. (2005). “Incidence of herpes simplex virus keratitis in France.” Ophthalmology 112(5): 888-895.

Lebofsky, R. and A. Bensimon (2003). “Single DNA molecule analysis: applications of molecular combing.” Brief Funct Genomic Proteomic 1(4): 385-396.

Lebofsky, R. and A. Bensimon (2005). “DNA replication origin plasticity and perturbed fork progression in human inverted repeats.” Mol Cell Biol 25(15): 6789-6797.

Lebofsky, R., R. Heilig, et al. (2006). “DNA replication origin interference increases the spacing between initiation events in human cells.” Mol Biol Cell 17(12): 5337-5345.

Leger, J., B. Larroque, et al. (2001). “Influence of severity of congenital hypothyroidism and adequacy of treatment on school achievement in young adolescents: a population-based cohort study.” Acta Paediatr 90(11): 1249-1256.

Liesegang, T. J. (1989). “Epidemiology of ocular herpes simplex. Natural history in Rochester, Minn., 1950 through 1982.” Arch Ophthalmol 107(8): 1160-1165.

Liesegang, T. J. (2001). “Herpes simplex virus epidemiology and ocular importance.” Cornea 20(1): 1-13.

Liesegang, T. J., L. J. Melton, 3rd, et al. (1989). “Epidemiology of ocular herpes simplex. Incidence in Rochester, Minn., 1950 through 1982.” Arch Ophthalmol 107(8): 1155-1159.

Lomholt, J. A., K. Baggesen, et al. (1995). “Recurrence and rejection rates following corneal transplantation for herpes simplex keratitis.” Acta Ophthalmol Scand 73(1): 29-32.

Lukashev, A. N. (2005). “Role of recombination in evolution of enteroviruses.” Rev Med Virol 15(3): 157-167.

Margolis, T. P., F. L. Elfman, et al. (2007). “Spontaneous reactivation of herpes simplex virus type 1 in latently infected murine sensory ganglia.” J Virol 81(20): 11069-11074.

Matsumoto, T., O. Yamada, et al. (1992). “Rapid DNA diagnosis of herpes simplex virus serotypes.” J Virol Methods 40(1): 119-125.

McGeoch, D. J., M. A. Dalrymple, et al. (1988). “The complete DNA sequence of the long unique region in the genome of herpes simplex virus type 1.” J Gen Virol 69 (Pt 7): 1531-1574.

McGeoch, D. J., A. Dolan, et al. (1986). “Complete DNA sequence of the short repeat region in the genome of herpes simplex virus type 1.” Nucleic Acids Res 14(4): 1727-1745.

Metzner, K. J. (2006). “Detection and significance of minority quasispecies of drug-resistant HIV-1.” J HIV Ther 11(4): 74-81.

Michalet, X., R. Ekong, et al. (1997). “Dynamic molecular combing: stretching the whole human genome for high-resolution studies.” Science 277(5331): 1518-1523.

Mocarski, E. S. and B. Roizman (1982). “Structure and role of the herpes simplex virus DNA termini in inversion, circularization and generation of virion DNA.” Cell 31(1): 89-97.

Namvar, L., S. Olofsson, et al. (2005). “Detection and typing of Herpes Simplex virus (HSV) in mucocutaneous samples by TaqMan PCR targeting a gB segment homologous for HSV types 1 and 2.” J Clin Microbiol 43(5): 2058-2064.

Ou, C. Y., S. Kwok, et al. (1988). “DNA amplification for direct detection of HIV-1 in DNA of peripheral blood mononuclear cells.” Science 239(4837): 295-297.

Pan, X. P., L. J. Li, et al. (2007). “Differences of YMDD mutational patterns, precore/core promoter mutations, serum HBV DNA levels in lamivudine-resistant hepatitis B genotypes B and C.” J Viral Hepat 14(11): 767-774.

Pasero, P., A. Bensimon, et al. (2002). “Single-molecule analysis reveals clustering and epigenetic regulation of replication origins at the yeast rDNA locus.” Genes Dev 16(19): 2479-2484.

Patel, P. K., B. Arcangioli, et al. (2006). “DNA replication origins fire stochastically in fission yeast.” Mol Biol Cell 17(1): 308-316.

Pramod, N. P., P. Rajendran, et al. (1999). “Herpes simplex keratitis in South India: clinico-virological correlation.” Jpn J Ophthalmol 43(4): 303-307.

Pressing, J. and D. C. Reanney (1984). “Divided genomes and intrinsic noise.” J Mol Evol 20(2): 135-146.

Ramaswamy, M. and A. M. Geretti (2007). “Interactions and management issues in HSV and HIV coinfection.” Expert Rev Anti Infect Ther 5(2): 231-243.

Rao, V. A., C. Conti, et al. (2007). “Endogenous gamma-H2AX-ATM-Chk2 checkpoint activation in Bloom's syndrome helicase deficient cells is related to DNA replication arrested forks.” Mol Cancer Res 5(7): 713-724.

Reisinger, J., S. Rumpler, et al. (2006). “Visualization of episomal and integrated Epstein-Barr virus DNA by fiber fluorescence in situ hybridization.” Int J Cancer 118(7): 1603-1608.

Rodrigues, C., M. Deshmukh, et al. (2001). “Significance of HBV DNA by PCR over serological markers of HBV in acute and chronic patients.” Indian J Med Microbiol 19(3): 141-144.

Roizman, B. (1979). “The structure and isomerization of herpes simplex virus genomes.” Cell 16(3): 481-494.

Rowley, A. H., R. J. Whitley, et al. (1990). “Rapid detection of herpes-simplex-virus DNA in cerebrospinal fluid of patients with herpes simplex encephalitis.” Lancet 335(8687): 440-441.

Schurra, C. and A. Bensimon (2009). “Combing genomic DNA for structural and functional studies.” Methods Mol Biol 464: 71-90.

Severini, A., A. R. Morgan, et al. (1994). “Study of the structure of replicative intermediates of HSV-1 DNA by pulsed-field gel electrophoresis.” Virology 200(2): 428-435.

Shibata, D. K., N. Arnheim, et al. (1988). “Detection of human papilloma virus in paraffin-embedded tissue using the polymerase chain reaction.” J Exp Med 167(1): 225-230.

Sugita, S., N. Shimizu, et al. (2008). “Use of multiplex PCR and real-time PCR to detect human herpes virus genome in ocular fluids of patients with uveitis.” Br J Ophthalmol 92(7): 928-932.

Susloparov, M. A., I. M. Susloparov, et al. (2006). “[Herpes simplex virus type 1 and 2 (HSV-1,2) DNA detection by PCR during genital herpes].” Mol Gen Mikrobiol Virusol(1): 38-41.

Taylor, T. J., M. A. Brockman, et al. (2002). “Herpes simplex virus.” Front Biosci 7: d752-764.

Umene, K. (1999). “Mechanism and application of genetic recombination in herpesviruses.” Rev Med Virol 9(3): 171-182.

Vlazny, D. A., A. Kwong, et al. (1982). “Site-specific cleavage/packaging of herpes simplex virus DNA and the selective maturation of nucleocapsids containing full-length viral DNA.” Proc Natl Acad Sci USA 79(5): 1423-1427.

Wadsworth, S., R. J. Jacob, et al. (1975). “Anatomy of herpes simplex virus DNA. II. Size, composition, and arrangement of inverted terminal repetitions.” J Virol 15(6): 1487-1497.

Wang, K., T. Y. Lau, et al. (2005). “Laser-capture microdissection: refining estimates of the quantity and distribution of latent herpes simplex virus 1 and varicella-zoster virus DNA in human trigeminal Ganglia at the single-cell level.” J Virol 79(22): 14079-14087.

Whitley, R. J. and B. Roizman (2001). “Herpes simplex virus infections.” Lancet 357(9267): 1513-1518.

Wildy, P., W. C. Russell, et al. (1960). “The morphology of herpes virus.” Virology 12: 204-222.

Wilhelmus, K. R., D. J. Coster, et al. (1981). “Prognosis indicators of herpetic keratitis. Analysis of a five-year observation period after corneal ulceration.” Arch Ophthalmol 99(9): 1578-1582.

Worobey, M. and E. C. Holmes (1999). “Evolutionary aspects of recombination in RNA viruses.” J Gen Virol 80 (Pt 10): 2535-2543.

Yu, X., B. Shi, et al. (2009). “A random PCR screening system for the identification of type 1 human herpes simplex virus.” J Virol Methods 161(1): 91-97.

HIV-Related References

(2000). “Time from HIV-1 seroconversion to AIDS and death before widespread use of highly-active antiretroviral therapy: a collaborative re-analysis. Collaborative Group on AIDS Incubation and HIV Survival including the CASCADE EU Concerted Action. Concerted Action on SeroConversion to AIDS and Death in Europe.” Lancet 355(9210): 1131-1137.

Buchbinder, S. P., M. H. Katz, et al. (1994). “Long-term HIV-1 infection without immunologic progression.” AIDS 8(8): 1123-1128.

Carrillo-Infante, C., G. Abbadessa, et al. (2007). “Viral infections as a cause of cancer (review).” Int J Oncol 30(6): 1521-1528.

Chun, T. W., D. C. Nickle, et al. (2005). “HIV-infected individuals receiving effective antiviral therapy for extended periods of time continually replenish their viral reservoir.” J Clin Invest 115(11): 3250-3255.

Clouse, K. A., D. Powell, et al. (1989). “Monokine regulation of human immunodeficiency virus-1 expression in a chronically infected human T cell clone.” J Immunol 142(2): 431-438.

del Rio, C. (2006). “Current concepts in antiretroviral therapy failure.” Top HIV Med 14(3): 102-106.

Douek, D. C., J. M. Brenchley, et al. (2002). “HIV preferentially infects HIV-specific CD4+ T cells.” Nature 417(6884): 95-98.

Douek, D. C., M. Roederer, et al. (2009). “Emerging concepts in the immunopathogenesis of AIDS.” Annu Rev Med 60: 471-484.

Douglas, J. T. (2007). “Adenoviral vectors for gene therapy.” Mol Biotechnol 36(1): 71-80.

Espy, M. J., J. R. Uhl, et al. (2006). “Real-time PCR in clinical microbiology: applications for routine laboratory testing.” Clin Microbiol Rev 19(1): 165-256.

Fischer, M., B. Joos, et al. (2004). “Cellular viral rebound after cessation of potent antiretroviral therapy predicted by levels of multiply spliced HIV-1 RNA encoding nef.” J Infect Dis 190(11): 1979-1988.

Frenkel, L. M., Y. Wang, et al. (2003). “Multiple viral genetic analyses detect low-level human immunodeficiency virus type 1 replication during effective highly active antiretroviral therapy.” J Virol 77(10): 5721-5730.

Grizot, S., J. Smith, et al. (2009). “Efficient targeting of a SCID gene by an engineered single-chain homing endonuclease.” Nucleic Acids Res 37(16): 5405-5419.

Havlir, D. V., M. C. Strain, et al. (2003). “Productive infection maintains a dynamic steady state of residual viremia in human immunodeficiency virus type 1-infected persons treated with suppressive antiretroviral therapy for five years.” J Virol 77(20): 11212-11219.

Hermankova, M., J. D. Siliciano, et al. (2003). “Analysis of human immunodeficiency virus type 1 gene expression in latently infected resting CD4+ T lymphocytes in vivo.” J Virol 77(13): 7383-7392.

Herrick, J., C. Conti, et al. (2005). “Genomic organization of amplified MYC genes suggests distinct mechanisms of amplification in tumorigenesis.” Cancer Res 65(4): 1174-1179.

Huser, D. and R. Heilbronn (2003). “Adeno-associated virus integrates site-specifically into human chromosome 19 in either orientation and with equal kinetics and frequency.” J Gen Virol 84(Pt 1): 133-137.

Ishida, T., A. Hamano, et al. (2006). “5′ long terminal repeat (LTR)-selective methylation of latently infected HIV-1 provirus that is demethylated by reactivation signals.” Retrovirology 3: 69.

Lawn, S. D. (2004). “AIDS in Africa: the impact of coinfections on the pathogenesis of HIV-1 infection.” J Infect 48(1): 1-12.

Morgan, D., C. Mahe, et al. (2002). “HIV-1 infection in rural Africa: is there a difference in median time to AIDS and survival compared with that in industrialized countries?” AIDS 16(4): 597-603.

Parsonnet, J. (1999). Microbes and malignancy: infection as a cause of human cancers. New York; Oxford, Oxford University Press.

Ramratnam, B., R. Ribeiro, et al. (2004). “Intensification of antiretroviral therapy accelerates the decay of the HIV-1 latent reservoir and decreases, but does not eliminate, ongoing virus replication.” J Acquir Immune Defic Syndr 35(1): 33-37.

Schneider, M. F., S. J. Gange, et al. (2005). “Patterns of the hazard of death after AIDS through the evolution of antiretroviral therapy: 1984-2004.” AIDS 19(17): 2009-2018.

Schurra, C. and A. Bensimon (2009). “Combing genomic DNA for structural and functional studies.” Methods Mol Biol 464: 71-90.

Siliciano, J. D., J. Kajdas, et al. (2003). “Long-term follow-up studies confirm the stability of the latent reservoir for HIV-1 in resting CD4+ T cells.” Nat Med 9(6): 727-728.

Thomas, C. E., A. Ehrhardt, et al. (2003). “Progress and problems with the use of viral vectors for gene therapy.” Nat Rev Genet 4(5): 346-358.

Tobin, N. H., G. H. Learn, et al. (2005). “Evidence that low-level viremias during effective highly active antiretroviral therapy result from two processes: expression of archival virus and replication of virus.” J Virol 79(15): 9625-9634.

Weiss, R. A. (1993). “How does HIV cause AIDS?” Science 260(5112): 1273-1279.

Zhou, H. S., N. Zhao, et al. (2007). “Site-specific transfer of an intact beta-globin gene cluster through a new targeting vector.” Biochem Biophys Res Commun 356(1): 32-37.

### INCORPORATION BY REFERENCE

Each document, patent, patent application or patent publication cited by or referred to in this disclosure is incorporated by reference in its entirety, especially with respect to the specific subject matter surrounding the citation of the reference in the text. However, no admission is made that any such reference constitutes background art and the right to challenge the accuracy and pertinence of the cited documents is reserved.

1. A method for detecting an infectious viral polynucleotide in a biological sample comprising:
separating, extracting or otherwise obtaining a polynucleotide from said sample,
Molecular Combing said polynucleotide to form a stretched polynucleotide,
contacting said stretched polynucleotide with one or more probes that recognize an infectious viral polynucleotide sequence,
detecting hybridization of the probes to the combed sample; thereby detecting an infectious viral polynucleotide.

2. The method of claim 1, wherein said biological sample is a tissue sample, cell(s), serum, blood, CSF, or synovial fluid sample obtained from a human, a non-human mammal or a bird.

3. The method of claim 1, wherein said polynucleotide is an infectious or a non-infectious viral DNA extracted from tissue, cell(s), serum, blood, CSF, or synovial fluid sample of a human, a non-human mammal, or a bird.

4. The method of claim 1, wherein said polynucleotide is integrated into the DNA or episomal or a biological fluid of a human or a non-human mammal or a bird.

5. The method of claim 1, wherein said polynucleotide is detected with one or more probes that bind to a viral DNA of a double stranded DNA virus.

6. The method of claim 1, wherein said polynucleotide is detected with one or more probes that bind to DNA of a herpes virus, a papilloma virus, a hepatitis virus or a retrovirus.

7. The method of claim 1, wherein said one or more probes comprises a set of probes that bind to at least 1 kb of the genomic DNA of a virus.

8. The method of claim 1, wherein said one or more probes comprises at least two sets of probes that are tagged with different labels selected so as to permit the identification of different portions or segments or rearranged fragments of a viral genome.

9. A method for detecting or identifying an infectious or genomic viral polynucleotide sequence in a mammalian cell, tissue or biological fluid comprising using Molecular Combing to detect the presence or the quantity of infectious viral polynucleotide or genomic viral polynucleotide in a cell, tissue or biological fluid.

10. A method for quantifying an infectious or genomic viral polynucleotide sequence comprising using Molecular Combing to detect quantity of infectious viral polynucleotide or genomic viral polynucleotide in a cell, tissue or biological fluid compared to an uninfected control sample or an otherwise similar sample obtained at a different point in time.

11. A method for detecting or following viral presence or virus replication or viral genomic rearrangements in a mammalian cell comprising Molecular Combing for the presence of latent or replicating viral DNA or rearranged viral DNA in a cell, tissue or biological fluid.

12. A method for evaluating the efficacy of an anti-viral treatment comprising:
detecting using Molecular Combing the presence, arrangement or quantity of infectious or genomic viral DNA in a sample obtained from a subject,
treating said subject with an anti-viral agent, and
re-evaluating using Molecular Combing the presence, arrangement or quantity of infectious viral or genomic viral DNA in said subject.

13. A set of probes covering 5-100% of the HSV or HIV genome.

14. A kit for performing Molecular Combing comprising
a Molecular Combing apparatus and/or reagents,
one or more probes that bind to an infectious viral polynucleotide for detecting or identifying genomic viral DNA in a combed DNA molecule, and
optionally one or more cell, tissue or biological fluid sample(s), and
optionally software for detecting or classifying the infectious sequences.

15. Biological materials HSV-B4 (CNCM I-4298), HSV-B19 (CNCM I-4299), HSV-Sc54 (CNCM I-4300), or HSV-P4 (CNCM I-4301).


SEQUENCE LISTING (24 sequences)

SEQ ID NO: 1 - DNA, 20 bp, Artificial
  Forward Primer HSV-P4
tggttgtgttactgggcaaa

SEQ ID NO: 2 - DNA, 20 bp, Artificial
  Reverse Primer HSV-P4
tcgatcgacgacaccataaa

SEQ ID NO: 3 - DNA, 20 bp, Artificial
  Forward Primer HSV-P5
cagatacgactcccgcagat

SEQ ID NO: 4 - DNA, 20 bp, Artificial
  Reverse Primer HSV-P5
cgacgacctcgacgttattt

SEQ ID NO: 5 - DNA, 20 bp, Artificial
  Forward Primer HSV-P6
cgtgaggtccaaaatcacct

SEQ ID NO: 6 - DNA, 20 bp, Artificial
  Reverse Primer HSV-P6
gacaggcaagctcaaagtcc

SEQ ID NO: 7 - DNA, 19 bp, Artificial
  Forward Primer HSV-P8
agatgtccacgagcaccag

SEQ ID NO: 8 - DNA, 20 bp, Artificial
  Reverse Primer HSV-P8
cctgactttgtggggctaaa

SEQ ID NO: 9 - DNA, 19 bp, Artificial
  Forward Primer HIV-S1
tggaagggctaatttggtc

SEQ ID NO: 10 - DNA, 20 bp, Artificial
  Reverse Primer HIV-S1
tattgctggtgatcctttcc

SEQ ID NO: 11 - DNA, 21 bp, Artificial
  Forward Primer HIV-S2
ccagcaatattccagtgtagc

SEQ ID NO: 12 - DNA, 20 bp, Artificial
  Reverse Primer HIV-S2
tgaaacaaacttggcaatga

SEQ ID NO: 13 - DNA, 20 bp, Artificial
  Forward Primer HIV-S3
catctcctatggcaggaaga

SEQ ID NO: 14 - DNA, 22 bp, Artificial
  Reverse Primer HIV-S3
tgctagagattttccacactga

SEQ ID NO: 15 - DNA, 152261 bp, Herpes simplex HSV-1 (NC_001806.1)
agcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggcagcccgggccccccgcggccgagactagcgagttagacaggcaagcactactcgcctctgcacgcacatgcttgcctgtcaaactctaccaccccggcacgctctctgtctccatggcccgccgccgccgccatcgcggcccccgccgcccccggccgcccgggcccacgggcgccgtcccaaccgcacagtcccaggtaacctccacgcccaactcggaacccgcggtcaggagcgcgcccgcggccgccccgccgccgccccccgccggtgggcccccgccttcttgttcgctgctgctgcgccagtggctccacgttcccgagtccgcgtccgacgacgacgatgacgacgactggccggacagccccccgcccgagccggcgccagaggcccggcccaccgccgccgccccccggccccggcccccaccgcccggcgtgggcccggggggcggggctgacccctcccaccccccctcgcgccccttccgccttccgccgcgcctcgccctccgcctgcgcgtcaccgcggagcacctggcgcgcctgcgcctgcgacgcgcgggcggggagggggcgccggagccccccgcgacccccgcgacccccgcgacccccgcgacccccgcgacccccgcgcgggtgcgcttctcgccccacgtccgggtgcgccacctggtggtctgggcctcggccgcccgcctggcgcgccgcggctcgtgggcccgcgagcgggccgaccgggctcggttccggcgccgggtggcggaggccgaggcggtcatcgggccgtgcctggggcccgaggcccgtgcccgggccctggcccgcggagccggcccggcgaactcggtctaacgttacacccgaggcggcctgggtcttccgcggagctcccgggagctccgcaccaagccgctctccggagagacgatggcaggagccgcgcatatatacgcttggagccagcccgccctcacagggcgggccgcctcgggggcgggactggccaatcggcggccgccagcgcggcggggcccggccaaccagcgtccgccgagtcttcggggcccggcccattgggcgggagttaccgcccaatgggccgggccgcccacttcccggtatggtaattaaaaacttgcaagaggccttgttccgcttcccggtatggtaattagaaactcattaatgggcggccccggccgcccttcccgcttccggcaattcccgcggcccttaatgggcaaccccggtattccccgcctcccgcgccgcgcgtaaccactcccctggggttccgggttatgctaattgcttttttggcggaacacacggcccctcgcgcattggcccgcgggtcgctcaatgaacccgcattggtcccctggggttccgggtatggtaatgagtttcttcgggaaggcgggaagccccggggcaccgacgcaggccaagcccctgttgcgtcggcgggaggggcatgctaatggggttctttgggggacaccgggttgggcccccaaatcgggggccgggccgtgcatgctaatgatattctttgggggcgccgggttggtccccggggacggggccgccccgcggtgggcctgcctcccctgggacgcgcggccattgggggaatcgtcactgccgcccctttggggaggggaaaggcgtggggtataagttagccctggcccgacagtctggtcgcatttgcacctcggcactcggagcgagacgcagcagccaggcagactcgggccgccccctctccgcatcaccacagaagccccgcctacgttgcgacccccagggaccctccgtccgcgaccctccagccgcatacgacccccatggagccccgccccggagcgagtacccgccggcctgagggccgcccccagcgcgaggtgaggggccgggcgccatgtctggggcgccatattggggggcgccatattggggggcgccatgttgggggacccccgacccttacactggaaccggccgccatgttgggggacccccactcatacacgggagccgggcgccatgttggggcgccatgttagggggcgtggaaccccgtgacactatatatacagggaccgggggcgccatgttagggggtgcggaaccccctgaccctatatatacagggaccggggtcgccctgttgggggtcgccatgtgaccccctgactttatatatacagacccccaacacatacacatggcccctttgactcagacgcagggcccggggtcgccgtgggaccccctgactcatacacagagacacgcccccacaacaaacacacaaggaccggggtcgccgtgttgggggcgtggtccccactgactcatacgcaggccccccttactcacacgcatctaggggggtggggaggagccgcccgccatatttgggggacgccgtgggacccccgactccggtgcgtctggagggcgggagaagagggaagaagaggggtcgggatccaaaggacggacccagaccacctttggttgcagacccctttctcccccctcttccgaggccagcaggggggcaggactttgtgaggcgggggggggagagggggaactcgtgggtgctgattgacgcgggaaatccccccccattcttacccgccccccttttttccccttagcccgccccggatgtctgggtgtttccctgcgaccgagacctgccggacagcagcgactctgaggcggagaccgaagtgggggggcggggggacgccgaccaccatgacgacgactccgcctccgaggcggacagcacggacacggaactgttcgagacggggctgctggggccgcagggcgtggatgggggggcggtctcgggggggagccccccccgcgaggaagaccccggcagttgcgggggcgccccccctcgagaggacggggggagcgacgagggcgacgtgtgcgccgtgtgcacggatgagatcgcgccccacctgcgctgcgacaccttcccgtgcatgcaccgcttctgcatcccgtgcatgaaaacctggatgcaattgcgcaacacctgcccgctgtgcaacgccaagctggtgtacctgatagtgggcgtgacgcccagcgggtcgttcagcaccatcccgatcgtgaacgacccccagacccgcatggaggccgaggaggccgtcagggcgggcacggccgtggactttatctggacgggcaatcagcggttcgccccgcggtacctgaccctgggggggcacacggtgagggccctgtcgcccacccacccggagcccaccacggacgaggatgacgacgacctggacgacggtgaggcggggggcggcaaggaccctgggggaggaggaggaggagggggggggagggaggaataggcgggcgggcgaggaaagggcgggccggggagggggcgtaacctgatcgcgccccccgttgtctcttgcagcagactacgtaccgcccgccccccgccggacgccccgcgcccccccacgcagaggcgccgccgcgccccccgtgacgggcggggcgtctcacgcagccccccagccggccgcggctcggacagcgcccccctcggcgcccatcgggccacacggcagcagtaacaccaacaccaccaccaacagcagcggcggcggcggctcccgccagtcgcgagccgcggcgccgcggggggcgtctggcccctccgggggggttggggttggggttggggttgttgaagcggaggcggggcggccgaggggccggacgggcccccttgtcaacagacccgccccccttgcaaacaacagagaccccatagtgatcagcgactcccccccggcctctccccacaggccccccgcggcgcccatgccaggctccgccccccgccccgggccccccgcgtccgcggccgcgtcgggacccgcgcgcccccgcgcggccgtggccccgtgcgtgcgagcgccgcctccggggcccggcccccgcgccccggcccccggggcggagccggccgcccgccccgcggacgcgcgccgtgtgccccagtcgcactcgtccctggctcaggccgcgaaccaagaacagagtctgtgccgggcgcgtgcgacggtggcgcgcggctcgggggggccgggcgtggagggtgggcacgggccctcccgcggcgccgccccctccggcgccgccccgctcccctccgccgcctctgtcgagcaggaggcggcggtgcgtccgaggaagaggcgcgggtcgggccaggaaaacccctccccccagtccacgcgtccccccctcgcgccggcaggggccaagagggcggcgacgcaccccccctccgactcagggccgggggggcgcggccagggtgggcccgggacccccctgacgtcctcggcggcctccgcctcttcctcctctgcctcttcctcctcggccccgacccccgcgggggccgcctcttccgccgccggggccgcgtcctcctccgcttccgcctcctcgggcggggccgtcggtgccctgggagggagacaagaggaaacctccctcggcccccgcgctgcttctgggccgcgggggccgaggaagtgtgcccggaagacgcgccacgcggagacttccggggccgtccccgcgggcggcctcacgcgctacctgcccatctcgggggtctctagcgtggtcgccctgtcgccttacgtgaacaagactatcacgggggactgcctgcccatcctggacatggagacggggaacatcggggcgtacgtggtcctggtggaccagacgggaaacatggcgacccggctgcgggccgcggtccccggctggagccgccgcaccctgctccccgagaccgcgggtaaccacgtgatgccccccgagtacccgacggcccccgcgtcggagtggaacagcctctggatgacccccgtggggaacatgctgttcgaccagggcaccctagtgggcgccctggacttccgcagcctgcggtctcggcacccgtggtccggggagcagggggcgtcgacccgggacgagggaaaacaataagggacgccccccgtgtttgtggggaggggggggtcgggcgctgggtggtctctggccgcgcccactacaccagccaatccgtgtcggggaggggaaaagtgaaagacacgggcaccacacaccagcgggtcttttgtgttggccctaataaaaaaaaactcaggggatttttgctgtctgttgggaaataaaggtttacttttgtatcttttccctgtctgtgttggatgtatcgcggggatgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgcgtgggagtgggggtgccatgttgggcaggctctggtgttaaccacagagccgcggcccgggctgcctgaccaccgatccccgaaagcatcctgccactggcatggagccagaaccacagtgggttgggtgtgggtgttaagtttccgcgagcgcctgcccgcccggactgacctggcctctggccgccacaaagggcggggggggttaactacactatagggcaacaaaggatgggaggggtggcggggcgggacggggcgcccaaaagggggtcggccacaccacagacgtgggtgttggggggtggggcggaggggtgggggggggggagacagaaacaggaacatagttagaaaacaagaatgcggtgcagccagagaatcacaggagacgaggggatgggcgtgttggttaccaacccacacccaggcatgctcggtggtatgaaggagggggggcggtgcttcttagagaccgccgggggacgtggggttggtgtgcaaaggcacgcgcacccgcgcggccaggtgggccggtactccatccccccctcccccgacccttcccacccccgcgtgccagagatcaccccggtcccccggcacccgccactcctccgtatcctcgctttaggaacaactttagggggggtacacacgcgccgtgcatttccttccacaccccccctcccccgcactccccccccccaggcagtaagacccaagcatagagagccaggcacaaaaacacaggcggggtgggacacatgccttcttggagtacgtgggtcattggcgtggggggttacagcgacaccggccgaccccctggcggtcttccagccggcccttagataagggggcagttggtggtcggacgggtaagtaacagagtctgactaagggtgggagggggggaaaagaacgggctggtgtgctgtaacacgagcccacccgcgagtggcgtggccgaccttagcctctggggcgccccctgtcgtttgggtcccccccctctattggggagaagcaggtgtctaacctacctggaaacgcggcgtctttgttgaacgacaccggggcgccctcgacgagtgggataacgggggaggaagggagggaggagggtactgggggtgaagaaggggggggggagaagcgagaacaggaaaggcgatggagcccggcagaacaccgaggaaaaaaaaaccacagcgcatgcgccgggccgttgtggggccccgggccggggccccttgggtccgccggggccccgggccgggccgccacgggggccggccgttggcggtaaccccgagtgttcatctcaggccccgggccgggaacccggaaaagcctccggggggcctttttcgcgtcgcgtgccggcgagcgggtccggacggggcccggaccgccgcggtcgggggcccctcgtcccgggccgtacgcggccttcgccccgtgaggggacagacgaacgaaacattccggcgacggaacgaaaaacaccccagacgggttaaagaaacagaaaccgcaacccccaccacccccgaaacggggaaaacgaaaaaacagaccagcggccggccggcgcttagggggaggatgtcgccgacgccccttggccgccccggctgcaggggggcccggagagccgcggcacccggacgcgcccggaaagtctttcgcaccacccgcgatcggcacggccgcgcccccgcttttataaaggctgagatgacgcagcaaaaacaggccacagcaccacgtgggtaggtgatgtaattttattttcctcgtctgcggcctaatggatttccgggcgcggtgcccctgtctgcagagcacttaacggattgatatctcgcgggcacgcgcgcccttaatggaccggcgcggggcggggggccggatacccacacgggcggggggggggtgtcgcgggccgtctgctggcccgcggccacataaacaatgactctgggcctttctgcctctgccgcttgtgagtgcgcgcgccggctctgcggtgtcggcggcggctgcggcggctgcggcggccgccgtgttcggtctcggtagccggccggcgggtggactcgcggggggccggagggtggaaggcaggggggtgtaggatgggtatcaggacttccacttcccgtccttccatcccccgttcccctcggttgttcctcgcctcccccaacaccccgccgctttccgttggggttgttattgttgtcgggatcgtgcgggccgggggtcgccggggcaggggcgggggcgtgggcgggggtgctcgtcgatcgaccgggctcagtgggggcgtggggtgggtgggagaaggcgaggagactggggtgggggtgtcggtgggtggttgttttttgtggttgtttttgtgtctgttcccgtcccccgtcaccccctccctccgtcccctccgtccccccgtcgcgggtgtttgtgtttgtttattccgacattggtttatttaaataaacacagccgttctgcgtgtctgttcttgcgtgtggctgggggcttatatgtggggtcccgggggcgggatggggtttagcggcggggggcggcgcgccggacggggcgctggagataacggcccccggggaacgggggaccggggctgggtatcccgaggtgggtgggtgggcggcggtggccgggccgggccgggccgggccgggccgggtgggcggggtttggaaaaacgaggaggaggaggagaaggcgggggggggggagacggggggaaagcaaggacacggcccggggggtgggagcgcgggccgggccgctcgtaagagccgcgacccggccgccggggagcgttgtcgccgtcggtctgccggcccccgtccctcccttttttgaccaaccagcgccccccccccccctcaccaccattcctactaccaccaccaccaccaccaccgacacctcccgcgcacccccgcccacatccccccccaacccgcaccaccagcacgggttgggggtagcaggggatcaaaggggggcaaagcggcggggcggttcggggggggggggggggggcgggaaaccaagtaggcccgcccatccgcggcccctcccggcagccacgcccccagcgtcgggtgtcacggggaaagagcagaggggagaggggagagggggggagaggggagagggggggagaggggagagggggggagaggggagagggggggagaggggagagggggggagaggggagagggggggagaggggagagggggggagaggggagagggggggagaggggagagggggggagaggggagagggggggagagggggtatataaaccaacgaaaagcgcgggaacggggatacggggcttgtgtggcacgacgtcgtggttgtgttactgggcaaacacttggggactgtaggtttctgtgggtgccgaccctaggcgctatggggattttgggttgggtcgggcttattgccgttggggttttgtgtgtgcgggggggcttgccttcaaccgaatatgttattcggagtcgggtggctcgagaggtgggggatatattaaaggtgccttgtgtgccgctcccgtctgacgatcttgattggcgttacgagaccccctcggctataaactatgctttgatagacggtatatttttgcgttatcactgtcccggattggacacggtcttgtgggataggcatgcccagaaggcatattgggttaacccctttttatttgtggcgggttttttggaggacttgagttaccccgcgtttcctgccaacacccaggaaacagaaacgcgcttggccctttataaagagatacgccaggcgctggacagtcgcaagcaggccgccagccacacacctgtgaaggctgggtgtgtgaactttgactattcgcgcacccgccgctgtgtagggcgacaggatttgggacctaccaacggaacgtctggacggaccccggttctgccgccggacgatgaagcgggcctgcagccgaagcccctcaccacgccgccgcccatcatcgccacgtcggaccccaccccgcgacgggacgccgccacaaaaagcagacgccgacgaccccactcccggcgcctctaacgatgcctcgacggaaacccgtccgggttcggggggcgaaccggccgcctgtcgctcgtcagggccggcggcgctcctcgccgccctagaggctggtcccgctggtgtgacgttttcctcgtccgcgccccccgaccctcccatggatttaacaaacgggggggtgtcgcctgcggcgacctcggcgcctctggactggaccacgtttcggcgtgtgtttctgatcgacgacgcgtggcggcccctgatggagcctgagctggcgaaccccttaaccgcccacctcctggccgaatataatcgtcggtgccagaccgaagaggtgctgccgccgcgggaggatgtgttttcgtggactcgttattgcacccccgacgaggtgcgcgtggttatcatcggccaggacccatatcaccaccccggccaggcgcacggacttgcgtttagcgtgcgcgcgaacgtgccgcctcccccgagtcttcggaatgtcttggcggccgtcaagaactgttatcccgaggcacggatgagcggccacggttgcctggaaaagtgggcgcgggacggcgtcctgttactaaacacgaccctgaccgtcaagcgcggggcggcggcgtcccactctagaatcggttgggaccgtttcgtgggcggagttatccgccggttggccgcgcgccgccccggcctggtgtttatgctctggggcacacacgcccagaatgccatcaggccggaccctcgggtccattgcgtcctcaagttttcgcacccgtcgcccctctccaaggttccgttcggaacctgccagcatttcctcgtggcgaaccgatacctcgagacccggtcgatttcacccatcgactggtcggtttgaaaggcatcgacgtccggggtttttgtcggtgggggcttttgggtatttccgatgaataaagacggttaatggttaaacctctggtctcatacgggtcggtgatgtcgggcgtcgggggagagggagttccctctgcgcttgcgattctagcctcgtggggctggacgttcgacacgccaaaccacgagtcggggatatcgccagatacgactcccgcagattccattcggggtgccgctgtggcctcacctgaccaacctttacacgggggcccggaacgggaggccacagcgccgtctttctccccaacgcgcgcggatgacggcccgccctgtaccgacgggccctacgtgacgtttgataccctgtttatggtgtcgtcgatcgacgaattagggcgtcgccagctcacggacaccatccgcaaggacctgcggttgtcgctggccaagtttagcattgcgtgcaccaagacctcctcgttttcgggaaacgccccgcgccaccacagacgcggggcgttccagcgcggcacgcgggcgccgcgcagcaacaaaagcctccagatgtttgtgttgtgcaaacgcgcccacgccgctcgagtgcgagagcagcttcgggtcgttattcagtcccgcaagccgcgcaagtattacacgcgatcttcggacgggcggctctgccccgccgtccccgtgttcgtccacgagttcgtctcgtccgagccaatgcgcctccaccgagataacgtcatgctggcctcgggggccgagtaaccgccccccccccatgccaccctcactgcccgtcgcgcgtgtttgatgttaataaataacacataaatttggctggttgtttgttgtctttaatggaccgcccgcaagggggggggggcatttcagtgtcgggtgacgagcgcgatccggccgggatcctaggaccccaaaagtttgtctgcgtattccagggcggggctcagttgaatctcccgcagcacctctaccagcaggtccgcggtgggctggagaaactcggccgtcccggggcaggcggttgtcgggggtggaggcgcggcgcccaccccgtgtgccgcgcctggcgtctcctctgggggcgacccgtaaatggttgcagtgatgtaaatggtgtccgcggtccagaccacggtcaaaatgccggccgtggcgctccgggcgctttcgccgcgcgaggagctgacccaggagtcgaacggatacgcgtacatatgggcgtcccacccgcgttcgagcttctggttgctgtcccggcctataaagcggtaggcacaaaattcggcgcgacagtcgataatcaccaacagcccaatgggggtgtgctggataacaacgcctccgcgcggcaggcggtcctggcgctcccggccccgtaccatgatcgcgcgggtgccgtactcaaaaacatgcaccacctgcgcggcgtcgggcagtgcgctggtcagcgaggccctggcgtggcataggctatacgcgatggtcgtctgtggattggacatctcgcggtgggtagtgagtcccccgggccgggttcggtggaactgtaaggggacggcgggttaatagacaatgaccacgttcggatcgcgcagagccgatagtatgtgctcactaatgacgtcatcgcgctcgtggcgctcccggagcggatttaagttcatgcgaaggaattcggaggaggtggtgcgggacatggccacgtacgcgctgttgaggcgcaggttgccgggcgtaaagcagatggcgaccttgtccaggctaaggccctgggagcgcgtgatggtcatggcaagcttggagctgatgccgtagtcggcgtttatggccatggccagctccgtagagtcaatggactcgacaaactcgctgatgttggtgttgacgacggacatgaagccgtgttggtcccgcaagaccacgtaaggcaggggggcctcttccagtaactcggccacgttggccgtcgcgtgccgcctccgcagctcgtccgcaaaggcaaacacccgtgcgtacgtgtatcccatgagcgtataattgtccgtctgcagggcgacggacatcagccccccgcgcggcgagccggtcagcatctcgcagccccggaagataacgttgtccacgtacgtgctaaagggggcgacttcaaatgcctccccgaagagctcttggaggattcggaatctcccgaggaaggcccgcttcagcagcgcaaactgggtgtgaacggcggcggtggtctccggttccccgggggtgtagtggcagtaaaacacgtcgagctgttgttcgtccagccccgcgaaaataacgtcgaggtcgtcgtcgggaaaatcgtccgggcccccgtcccgcggccccagttgcttaaaatcaaacgcacgctcgccgggggcgcctgcgtcggccattaccgacgcctgcgtcggcacccccgaagatttggggcgcagagacagaatctccgccgttagttctcccatgcgggcgtaggcgagggtcctctgggtcgcatccaggcccgggcgctgcagaaagttgtaaaaggagataagcccgctaaatatgagccgcgacaggaacctgtaggcaaactccaccgaagtctccccctgagtctttacaaagctgtcgtcacgcaacactgcctcgaaggcccggaacgtcccactaaacccaaaaaccagttttcgcaggcgcgcggtcaccgcgatctggctgttgaggacgtaagtgacgtcgttgcgggccacgaccagctgctgtttgctgtgcacctcgcagcgcatgtgccccgcgtcctggtcctggctctgcgagtagttggtgatgcggctggcgttggccgtgagccacttttcaatcgtcaggccgggctggtgtgtcagccgtcggtattcgtcaaactccttgaccgacacgaacgtaagcacggggagggtgaacacgacgaactccccctcacgggtcaccttcaggtaggcgtggagcttggccatgtacgcgctcacctctttgtgggaggagaacagccgcgtccagccggggaggttggcggggttggtgatgtagttttccgggacgacgaagcgatccacgaactgcatgtgctcctcggtgatgggcaggccgtactccagcaccttcatgaggttaccgaactcgtgctcgacgcaccgtttgttgttaataaaaatggcccagctatacgagaggcgggcgtactcgcgcagcgtgcggttgcagatgaggtacgtgagcacgttctcgctctggcggacggaacaccgcagtttctggtgctcgaaggtcgactccagggacgccgtctgcgtcggcgagcccacacacaccaacacgggccgcaggcgggccgcgtactggggggtgtggtacagggcgttaatcatccaccagcaatacaccacggccgtgaggaggtgacgcccaaggagcccggcctcgtcgatgacgatcacgttgctgcgggtaaaggccggcagcgccccgtgggtggccggggccaaccgcgtcagggcgccctcggccaaccccagggtccgttccagggcggccagggcgcgaaactcgttccgcaactcctcgcccccggaggcggccagggcgcgcttcgtgaggtccaaaatcacctcccagtagtacgtcagatctcgtcgctgcaggtcctccagcgaggcggggttgctggtcagggtgtacgggtactgtcccagttgggcctggacgtgattcccgcgaaacccaaattcatgaaagatggtgttgatgggtcggctgagaaaggcgcccgagagtttggcgtacatgttttgggccgcaatgcgcgtggcgcccgtcaccacacagtccaagacctcgttgattgtctgcacgcacgtgctctttccggagccagcgttgccggtgataagatacaccgcgaacggaaactccctgaggggcaggcctgcgggggactctaaggccgccacgtcccggaaccactgcagatggggcacttgcgctccgtcgagctgttgttgcgagagctctcggatgcgcttaaggattggctgcaccccgtgcatagacgtaaaatttaaaaaggcctcggccctccctggaacggctggtcggtccccgggttgctgaaggtgcggcgggccgggtttctgtccgtctagctggcgctccccgccggccgccgccatgaccgcaccacgctcgcgggcccccactacgcgtgcgcggggggacacggaagcgctgtgctcccccgaggacggctgggtaaaggttcaccccagccccggtacgatgctgttccgcgagattctccacgggcagctggggtataccgagggccagggggtgtacaacgtcgtccggtccagcgaggcgaccacccggcagctgcaggcggcgatctttcacgcgctcctcaacgccaccacttaccgggacctcgaggcggactggctcggccacgtggcggcccgcggtctgcagccccaacggctggttcgccggtacaggaacgcccgggaggcggatatcgccggggtggccgagcgggtgttcgacacgtggcggaacacgcttaggacgacgctgctggactttgcccacgggttggtcgcctgctttgcgccgggcggcccgagcggcccgtcaagcttccccaaatatatcgactggctgacgtgcctggggctggtccccatattacgcaagcgacaagaagggggtgtgacgcagggtctgagggcgtttctcaagcagcacccgctgacccgccagctggccacggtcgcggaggccgcggagcgcgccggccccgggttttttgagctggcgctggccttcgactccacgcgcgtggcggactacgaccgcgtgtatatctactacaaccaccgccggggcgactggctcgtgcgagaccccatcagcgggcagcgcggagaatgtctggtgctgtggccccccttgtggaccggggaccgtctggtcttcgattcgcccgtccagcggctgtttcccgagatcgtcgcgtgtcactccctccgggaacacgcgcacgtctgccggctgcgcaataccgcgtccgtcaaggtgctgctggggcgcaagagcgacagcgagcgcggggtggccggtgccgcgcgggtcgttaacaaggtgttgggggaggacgacgagaccaaggccgggtcggccgcctcgcgcctcgtgcggcttatcatcaacatgaagggcatgcgccacgtaggcgacattaacgacaccgtgcgttcctacctcgacgaggccggggggcacctgatagacgccccggccgtcgacggtaccctccctggattcggcaagggcggaaacagccgcgggtctgcgggccaggaccagggggggcgggcgccgcagcttcgccaggccttccgcacggccgtggttaacaacatcaacggcgtgttggagggctatataaataacctgtttggaaccatcgagcgcctgcgcgagaccaacgcgggcctggcgacccaattgcaggagcgcgaccgcgagctccggcgcgcaacagcgggggccctggagcgccagcagcgcgcggccgacctggcggccgagtccgtgaccggtggatgcggcagccgccctgcgggggcggacctgctccgggccgactatgacattatcgacgtcagcaagtccatggacgacgacacgtacgtcgccaacagctttcagcacccgtacatcccttcgtacgcccaggacctggagcgcctgtcgcgcctctgggagcacgagctggtgcgctgttttaaaattctgtgtcaccgcaacaaccagggccaagagacgtcgatctcgtactccagcggggcgatcgccgcattcgtcgccccctactttgagtcagtgcttcgggccccccgggtaggcgcgcccatcacgggctccgatgtcatcctgggggaggaggagttatgggatgcggtgtttaagaaaacccgcctgcaaacgtacctgacagacatcgcggccctgttcgtcgcggacgtccagcacgcagcgctgcccccgcccccctccccggtcggcgccgatttccggcccggcgcgtccccgcggggccggtccagatcgcggtcgcccggaagaactgcgcgaggcgcgccggaccagggcgggggcatcgggcaccgggatggccgccgcgacggccgacgatgaggggtcggccgccaccatcctcaagcaggccatcgccggggaccgcagcctggtcgaggcggccgaggcgattagccagcagacgctgctccgcctggcctgcgaggtgcgccaggtcggcgaccgccagccgcggtttaccgccaccagcatcgcgcgcgtcgacgtcgcgcctgggtgccggttgcggttcgttctggacgggagtcccgaggacgcctatgtgacgtcggaggattactttaagcgctgctgcggccagtccagttatcgcggcttcgcggtggcggtcctgacggccaacgaggaccacgtgcacagcctggccgtgccccccctcgttctgctgcaccggttctccctgttcaaccccagggacctcctggactttgagcttgcctgtctgctgatgtacctggagaactgcccccgaagccacgccaccccgtcgacctttgccaaggttctggcgtggctcggggtcgcgggtcgccgcacgtccccattcgaacgcgttcgctgccttttcctccgcagttgccactgggtcctaaacacactcatgttcatggtgtacgtaaaaccgttcgacgacgagttcgtcctgccccactggtacatggcccggtacctgctggccaacaacccgccccccgttctctcggccctgttctgtgccaccccgacgagctcctcattccggctgccggggccgcccccccgctccgactgcgtggcctataaccccgccgggatcatggggagctgctgggcgtcggaggaggtgcgcgcgcctctggtctattggtggctttcggagaccccaaaacgacagacgtcgtcgctgttttatcagttttgttgaattttaggaaataaacccggttttgtttctgtggcctcccgacggatgcgcgtgtccttactccgtcttggtgggtgggtggctgtgtatggcgtcccatctgtgcggggaggggggcaagtcggcacgtattcggacagactcaagcacacacgggggagcgctcttgtctcagggcaatgtttttattggtcaaactcaggcaaacagaaacgacatcttgtcgtcaaagggatacacaaacttccccccctcgccccatactcccgccagcaccccggtaaacaccaactcaatctcgcgcaggatttcgcgcaggtgatgagcgcagtccacgggggggagcacaaggggccgcgggtatagatcgacggggacgccgaccgactccccgcctccgggacagacacgcacgacgcgccgccagtagtgctctgcgtccagcaaggcgccgccgcggaaggcagtggggggcaaggggtcgctggcctcaaagggggacacccgaacgctccagtactccgcgtccaaccgtttattaaacgcgtccaagataaggcggtcgcaggcgtcctccataaggccccgggccgtgagtgcgtcctcctccggcacgcatgccgttgtcaggcccaggacccgtcgcagcgtgtcgcgtacgacccctgccgccgtggtgtacgcgggcccgcggagaggaaatcccccaagatggtcagtgttgtcgcgggagttccagaaccacactcccgcctggctccaggcgactgcgtgggtgtagacgccctcgagggccaggcacagtgggtgccgcagccggacggcgttggccctaagcacggctcccacggccgtctcgatggcccgccgggcgtcctcgatcaccccggaagccgcatccgcgtcttgggggtccacgttaaagacaccccagaacgcacccccatcgcccccgcagaccgcgaacttcaccgagctggccgtctcctcgatctgcaggcagacggcggccattaccccacccaggagctgccgcagcgcagggcaggcgttgcacgtgtccgggaccaggcgctccaagacggccccggcccagggctctgagggagcggccaccaccagcgcgtccagtcttgctaggcccgtccggccgtgggggtccgccagcccgctccccccgaggtcggccagggccgccaggagctgggcgcgaagtccggggaagcaaaaccgcgccgtccagacgggcccgacggccgcgggcgggtctaacagttggatgattttagtggcgggatgccaccgcgccaccgcctcccgcaccgcgggcaggaggcatccggctgccgccgaggccacgccgggccaggctcgcggggggaggacgaccctggcccccaccgcgggccaggcccccaggagcgcggcgtaagcggccgcggccccgcgcaccaggtcccgtgccgactcggccgtggccggcacggtgaacgtgggccaacccggaaaccccaggacggcaaagtacgggacgggtcccccccggacctcaaactcgggccccagaaaggcaaagacgggggccagggccccgggggcggcgtggaccgtggtatgccactgccggaaaagggcgacgagcgccggcgcggagaacttctcgccggcgcttacaaagtagtcgtaatcgcggggcagcagcacccgtgccgtgactcgttgcgggtgcccgcgtggccgcaggcccacctcgcacacctcgaccaggtccccgaacgcgccctccttcttgatcggcggaaacgcaagagtctggtattcgcgcgcaaatagcgcggttccggtggtgatgttaacggtcagcgaagcggcggacgcgcactggggggtgtcgcgaatggccgccaggcgcgcccacgccagccgcgcgtcgggatgctcggcaacgcgcgccgccagggccatagggtcgatgtcaatgttggcctccgcgaccaggagagcggcgcgaggggcggcgggcgggccccacgacgctctctcaactttcaccaccagtcccgtgcgtgggtccgagccgatacgcagcggggcgaacagggccaccggcccggtctggcgctccagggccgccaggacgcacgcgtacagcgcccgccacagagtcgggttctccaggggctccagcggggaggcggccggcgtcgtcgcggcgcgggcggccgccacgacggcctggacggagacgtccgcggagccgtagaaatcccgcagctccgtcgcggtgacggagacctccgcaaagcgcgcgcgaccctcccctgcggcgttgcgacatacaaaatacaccagggcgtggaagtactcgcgagcgcgggggggcagccataccgcgtaaagggtaatggcgctgacgctctcctccacccacacgatatctgcggtgtccatcgcacggcccctaaggatcacgggcggtctgtgggtcccatgctgccgtgcctggccgggcccggtgggtcgcggaaaccggtgacggggggggggcggtttttggggttggggtgggggtgggaaacggcccgggtccgggggccaacttggcccctcggtgcgttccggcaacagcgccgccggtccgcggacgaccacgtaccgaacgagtgcggtcccgagacttatagggtgctaaagttcaccgccccctgcatcatgggccaggcctcggtggggagctccgacagcgccgcctccaggatgatgtcagcgttggggttggcgctggatgagtgcgtgcgcaaacagcgcccccacgcgggcacgcgtagcttgaagcgcgcgcccgcaaactcccgcttgtgggccataagcagggcgtacagctgcctgtgggtccggcaggcgctgtggtcgatgtggtgggcgtccaacaaccccacgattgtctgtttggtgaggtttttaacgcgccccgccccgggaaacgtctgcgtgcttttggccatctgcacgccaaacagttcgccccagattatcttgaacagcgccaccgcgtggtccgtctcgctaacggacccgcgcgggggacagccgcttagggcgtcggcgacgcgcttgacggcttcctccgagagcagaagtccgtcggttacgttacagtggcccagttcgaacaccagctgcatgtagcggtcgtagtggggggtcagtaggtccagcacgtcatcggggccgaaggtcctcccagatcccccggccgccgagtcccaatgcaggcgcgcggccatggtgctgcacaggcacaacagctcccagacgggggttacgttcagggtggggggcagggccacgagctccagctctccggtgacgttgatcgtggggatgacgcccgtggcgtagtggtcatagatccgccgaaatatggcgctgctgcgggtggccatgggaacgcggagacaggcctccagcaacgccaggtaaataaaccgcgtgcgtcccatcaggctgttgaggttgcgcatgagcgcgacaatttccgccggcgcgacatcggaccggaggtatttttcgacgaaaagacccacctcctccgtctcggcggcctgggccggcagcgacgcctcgggatcccggcaccgcagctcccgtagatcgcgctgggccctgagggcgtcgaaatgtacgccccgcaaaaacagacagaagtcctttggggtcagggtatcgtcgtgtccccagaagcgcacgcgtatgcagtttagggtcagcagcatgtgaaggatgttaaggctgtccgagagacacgccagcgtgcatctctcaaagtagtgtttgtaacggaatttgttgtagatgcgcgacccccgccccagcgacgtgtcgcatgccgacgcgtcacagcgccccttgaaccggcgacacagcaggtttgtgacctgggagaactgcgcgggccactggccgcaggaactgaccacgtgattaaggagcatgggcgtaaagacgggctccgagcgcgccccggagccgtccatgtaaatcagtagctcccccttgcggagggtgcgcacccgtcccagggactggtacacggacaccatgtccggtccgtagttcatgggtttcacgtaggcgaacatgccatcaaagtgcaggggatcgaagctgaggcccacggttacgaccgtcgtgtatataaccacgcggtattggccccacgtggtcacgtccccgaggggggtgagcgagtgaagcaacagcacgcggtccgtaaactgacggcagaaccgggccacgatctccgcgaaggagaccgtcgacgaaaaaatgcagatgttatcgcccccgccaaggcgcgcttccagctccccaaagaacgtggccccccgggcctccggagaggcgtccggagacgggccgctcggcggcccgggcgggcgcagggcagcctgcaggagctcggtccccagacgcgggagaaacaggcaccggcgcgccgaaaacccgggcatggcgtactcgccgaccaccacatgcacgtttttttcgccccggagaccgcacaggaagtccaccaactgcgcgttggcggttgcgtccatggcgatgatccgaggacagatgcgcagcaggcgtagcattaacgcatccacgcggcccagttgctgcatcgttggcgaatagagctggcccagcgtcgacataacctcgtccagaacgaggacgtcgtagttgttcagaaggttggggcccacgcgatgaaggctttccacctggacgataagtcggtggaaggggcggtcgttcataatgtaattggtggatgagaagtaggtgacaaagtcgaccaggcctgactcagcgaaccgcgtcgctagggtctgggtaaaactccgacgacaggagacgacgagcacactcgtgtccggagagtggatcgcttcccgcagccagcggatcagcgcggtagtttttcccgaccccattggcgcgcggaccacagtcacgcacctggccgtcggggcgctcgcgttggggaaggtgacgggtccgtgctgctgccgctcgatcgttgttttcgggtgaacccggggcacccattcggccaaatcccccccgtacaacatccgcgctagcgatacgctcgacgtgtactgttcgcactcgtcgtccccaatgggacgcccggcccccagaggatctcccgactccgcgccccccacgaaaggcatgaccggggcgcggacggcgtggtgggtctggtgtgtgcaggtggcgacgtttgtggtctctgcggtctgcgtcacggggctcctcgtcctggcctctgtgttccgggcacggtttccctgcttttacgccacggcgagctcttatgccggggtgaactccacggccgaggtgcgcgggggtgtagccgtgcccctcaggttggacacgcagagccttgtgggcacttatgtaatcacggccgtgttgttgttggccgtggccgtgtatgccgtggtcggcgccgtgacctcccgctacgaccgcgccctggacgcgggccgccgtctggctgcggcccgcatggccatgccgcacgccacgctgatcgccggaaacgtctgctcttggttgctgcagatcaccgtcctgttgctggcccatcgcatcagccagctggcccacctggtttacgtcctgcactttgcgtgtctggtgtattttgcggcccatttttgcaccaggggggtcctgagcgggacgtatctgcgtcaggtgcacggcctgatggagctggccccgacccatcatcgcgtcgtcggcccggctcgcgccgtgctgacaaacgccttgctgttgggcgtcttcctgtgcacggccgacgccgcggtatccctgaataccatcgccgcgttcaactttaatttttcggccccgggcatgctcatctgcctgaccgtgctgttcgccattctcgtcgtatcgctgttgttggtggtcgagggggtgttgtgtcactacgtgcgcgtgttggtgggcccccacctgggggccgtggccgccacgggcatcgtcggcctggcctgcgagcactattacaccaacggctactacgttgtggagacgcagtggccgggggctcagacgggagtccgcgtcgccctcgccctggtcgccgcctttgccctcggcatggccgtgctccgctgcacccgcgcctatctgtatcacaggcggcaccacaccaaattttttatgcgcatgcgcgacacgcgacaccgcgcacattccgccctcaagcgcgtacgcagttccatgcgcggatcgcgagacggccgccacaggcccgcacccggcagcccgcccgggattcccgaatatgcggaagacccctacgcgatctcatacggcggccagctcgaccggtacggagattccgacggggagccgatttacgacgaggtggcggacgaccaaaccgacgtattgtacgccaagatacaacacccgcggcacctgcccgacgacgatcccatctatgacaccgttggggggtacgaccccgagcccgccgaggaccccgtgtacagcaccgtccgccgttggtagctgtttggttccgttttaataaaccgtttgtgtttaacccgaccgtggtgtatgtctggtgtgtggcgtccgatcccgttactatcaccgtccccccccccccctcaaccccggcgattgtgggttttttaaaaacgacacgcgtgcgaccgtatacagaacattgttttggtttttattcgctatcggacatggggggtggaaactgggtggcggggcaggcgcctccgggggtccgccggtgagtgtggcgcgagggggggtccgatgaacgcaggcgctgtctccccggggcccgcgtaaccccgcgcatatccgggggcacgtagaaattaccttcctcttcggactcgatatccacgacgtcaaagtcgtgggcggtcagcgagacgacctccccgtcgtcggtgatgaggacgttgtttcggcagcagcagggccgggccccggagaacgagaggcccatagctcggcgagcgtgtcgtcgaatgccaggcggctgcttcgctggatggccttatagatctccggatcgatgcggacgggggtaatgatcagggcgatcggaacggcctggttcgggagaatggacgccttgctgggtcctgcggccccgagagccccggcgccgtcctccaggcggaacgttacgccctcctccgcgctggtgcggtgcctgccgataaacgtcaccagatgcgggtggggggggcagtcggggaagtggctgtcgagcacgtagccctgcaccaagatctgcttaaagttcgggtgacgggggttcgcgaagacgggctcgcggcggaccagatccccggagctccaggacacgggggagatggtgtggcgtccgaggtcgggggcgccaaacagaagcacctccgagacaacgccgctatttaactccaccaaggcccgatccgcggcggagcaccgccttttttcgcccgaggcgtgggcctctgaccaggcctggtcttgcgtgacgagagcctcctccgggccggggacgcgcccgggcgcgaagtatcgcacgctgggcttcgggatcgaccggataaatgcccggaacgcctccggggaccggtgtgccatcaagtcctcgtacgcggaggccgtggggtcgctggggtccatggggtcgaaagcgtacttggcccggcatttgacctcgtaaaaggccaggggggtcttggggactggggccaggtagccgtgaatgtcccgaggacagacgagaatatccagggacgccccgaccatccccgtgtgaccgtccatgaggaccccacacgtatgcacgttctcttcggcgaggtcgctgggttcgtggaagataaagcgccgcgtgtcggcgccggcctcgccgccgtcgtccgcgcggcccacgcagtagcgaaacagcaggcttcgggccgtcggctcgttcacccgcccgaacatcaccgccgaagactgtacatccggccgcaggctggcgttgtgcttcagccactggggcgagaaacacggaccctgggggccccagcggagggtggatgcggtcgtgaggccccgccggagcagggcccatagctggcagtcggcctggttttgcgtggccgcctcgtaaaaccccatgaggggccggggcgccacggcgtccgcggcggccgggggcccgcggcgcgtcaggcgccataggtgccgaccgagtccgcggtccaccatacccgcctcctcgaggaccacggccagggaacacagataatccaggcgggcccagaggggaccgatggccagaggggcgcggacgccgcgcagcaacccgcgcaggtggcgctcgaacgtctcggctagtatatgggagggcagcgcgttggggatcaccgacgccgaccacatagagtcaaggtccggggagtcgggatcggcgtccgggtcgcgggcgtgggtgcccccaggagatagcggaatgtctggggtcggaggccctgaggcgtcagaaagtgccggcgacgcggcccggggcttttcgtctgcggtgtcggtggcgtgctgatcacgtggggggttaacgggcgaatgggagctcgggtccacagctgatgtcgtctggggtggggggggcaggggacggaaggtggttgtcagcggaagactgttagggcgggggcgcttgggggggctgtcggggccacgaggggtgtcctcggccagggcccagggacgcttagtcacggtgcgtcccggcggacatgctgggcctaccgtggactccatttccgagacgacgtggggggagcggtggttgagcgcgccgccgggtgaacgctgattctcacgacagcgcgtgccgcgcgcacgggttggtgtgacacaggcgggacaccagcaccaggagaggcttaagctcgggaggcagcgccaccgacgacagtatcgccttgtgtgtgtgctggtaatttatacaccgatccgtaaacgcgcgccgaatcttgggattgcggaggtggcgccggatgccctctgggacgtcatacgccaggccgtgggtgttggtctcggccgagttgacaaacagggctgggtgcagcacgcagcgataggcgagcagggccagggcgaagtccggcgacagctggttgttaaaatactggtaaccgggaaaccgggtcacgggtacgcccaggctcggggcgacgtacacgctaaccaccaactccagcagcgtctggcccagggcgtacaggtcaaccgctaacccgacgtcgtgcttcaggcggtggttggtaaattcggcccgttcgttgttaaggtatttcaccaacagctccgggggctggttatacccgtgacccaccagggtgtgaaagttggctgtggttagggcggtgggcatgccaaacatccggggggacttgaggtccggctcctggaggcaaaactgcccccgggcgatcgtggagttggagttgagggtgacgaggctaaagtcggcgaggacggcccgccggagcgagacggcgtccgaccgcagcatgacgaggatgttggcgcacttgatatccaggtggctgatcccgcaggtggtgtttaaaaacacaacggcgcgggccagctccgtgaagcactggtggagggccgtcgagaccgaggggtttgttgtgcgcagggacgccagttggccgatatacttaccgaggtccatgtcgtacgcggggaacactatctgtcgttgttgcagcgagaacccgaggggcgcgatgaagccgcggatgttgtgggtgcggccggcgcgtagaacgcactccccgaccaacagggtcgcgatgagctcaacggcaaaccactccttttcctttatggtcttaacggcaagcttatgttcgcgaatcagttggacgtcaccgtatcccccagaccccccgaagcttcgggccccggggatctcgagggtcgtgtagtgtagggcggggttgatggcgaacacggggctgcatagcttgcggatgcgcgtgagggtgaggatgtgcgagggggacgaggggggtgcggttaacgccgcctgggatctgcgcaggggcgggcggttcagtttggccgccgtaccgggcgtctcgggggacgcgcggcgatgagacgagcggctcattcgccatcgggatagtcccgcgcgaagccgctcgcggaggccggatcggtggcgggacccgtgggaggagcgggagacggcggcgtcctggagagaggggccgctggggcgcccggaggccccgtgggggttggagtgtacgtaggatgcgagccaatccttgaaggaccgttggcgtgcaccttgggggctgaggttagctgccacatgaccagcaggtcgctgtctgcgggactcatccatccttcggccaggtcgccgtctccccacagagaagcgttggtcgctgcttcctcgagttgctcctcctggtccgcaagacgatcgtccacggcgtccaggcgctcaccaagcgccggatcgaggtaccgtcggtgtgcggttagaaagtcacgacgcgccgcttgctcctccacgcgaattttaacacaggtcgcgcgctgtcgcatcatctctaagcgcgcgcgggactttagccgcgcctccaattccaagtgggccgcctttgcagccataaaggcgccaacaaaccgaggatcttgggtgctgacgccctcccggtgcagctgcagggtctggtccttgtaaatctcggctcggaggtgcgtctcggccaggcgtcggcgcagggccgcgtgggcggcatctcggtccattccgccaccctgcgggcgacccggggggtgctctgatagtctcgcgtgcccaaggcccgtgatcggggtacttcgccgccgcgacccgccacccggtgtgcgcgatgtttggtcagcagctggcgtccgacgtccagcagtacctggagcgcctcgagaaacagaggcaacttaaggtgggcgcggacgaggcgtcggcgggcctcaccatgggcggcgatgccctacgagtgccctttttagatttcgcgaccgcgacccccaagcgccaccagaccgtggtccctggcgtcgggacgctccacgactgctgcgagcactcgccgctcttctcggccgtggcgcggcggctgctgtttaatagcctggtgccggcgcaactaaaggggcgtgatttcgggggcgaccacacggccaagctggaattcctggcccccgagttggtacgggcggtggcgcgactgcggtttaaggagtgcgcgccggcggacgtggtgcctcagcgtaacgcctactatagcgttctgaatacgtttcaggccctccaccgctccgaagcctttcgccagctggtgcactttgtgcgggactttgcccagctgctcaaaacctccttccgggcctccagcctcacggagaccacgggcccccccaaaaaacgggccaaggtggacgtggccacccacggccggacgtacggcacgctggagctgttccaaaaaatgatccttatgcacgccacctactttctggccgccgtgctcctcggggaccacgcggagcaggtcaacacgttcctgcgtctcgtgtttgagatccccctgtttagcgacgcggccgtgcgccacttccgccagcgcgccaccgtgtttctcgtcccccggcgccacggcaagacctggtttctggtgcccctcatcgcgctgtcgctggcctcctttcgggggatcaagatcggctacacggcgcacatccgcaaggcgaccgagccggtgtttgaggagatcgacgcctgcctgcggggctggttcggttcggcccgagtggaccacgttaaaggggaaaccatctccttctcgtttccggacgggtcgcgcagtaccatcgtgtttgcctccagccacaacacaaacgtaagtcctcttttctttcgcatggctctcccaaggggccccgggtcgacccgacccacacccacccacccacatacacacacaaccagacgcgggaggaaagtctgccccgtgggcactgatttttattcgggatcgcttgaggaggcccgggcaacggcccgggcaacggtggggcaactcgtagcaaataggcgactgatgtacgaagagaagacacacaggcgccacccggcgctggtcggggggatgttgtccgcgccgcaccgtcccccgacgacctcttgcagacggtccgtgatgcaaggacggcggggggcctgcagcagggtgaccgtatccacgggatggccaaagagaagcggacacaggctagcatccccctggaccgccagggtacactgggccatcttggcccacagacacggggcgacgcagggacaggactccgttacgacggaggagagccacagtgcgttggcggaatcgatgtggggcggcggggcgcaggactcgcagccccccgggtggttggtgatcctggccaggagccatcccagatggcgggccctgcttcccggtggacagagcgaccccaggtcgctgtccatggcccagcagtagatctggccgctggggaggtgccaccaggcccccgggcccaaggcgcagcacgcgcccggctccgggggggtcttcgcggggaccagatacgcgccatccagctcgccgaccactggctcctccgcgagctgttcggtggttgggtcgggggtttcctccgggggggtggccgcccgtatgcgtgcgaacgtgagggtgcacaggagcggggtcagggggtgcgtcacgctccggaggtggacgatcgcgcagtagcggcgctcgcggttaaagaaaaagagggcaaagaaggtgttcgggggcaaccgcagcgccttggggcgcgtcagatacagaaaaatctcgcagaagagggcgcgcccggggtctgggttaggaagggccacctgacacagaggctcggtgaggaccgttagacaccgaaagatcttgagccgctcgtccgcccgaacgacgcgccacacaaagacggagttgacaatgcgcgcgatagagtcgacgtccgtccccaggtcgtcgactctatcgcgcgtgccgcgagctccggcccgggaatccggccggggcaaggtccccgggggaccaggcggcgccaggggccgccggggtcccagctgcgccatgccgggggcggggggagggcaaaccccagaggcgggggccaacggcgcggggaggagtgggtgggcgaggtggccgggggaaggcgcccgctagcgagaccggccgttcccggacgacaccttgcgacaaaacctaaggacagcggcccgcgcgacggggtccgagaggctaaggtaggccgcgatgttaatggtgaacgcaaagccgccgggaaagacaactatgccacagaggcggcgattaaaccccaggcagaggtaggcgtagctttccccgggcaggtattgctcgcagaccctgcgtggggctgtggaggggacggcctccatgaagcgacatttactctgctcgcgtttactgacgtcaccatccatcgccacggcgattggacgattgttaagccgcagcgtgtctccgcttgtgctgtagtagtcaaaaacgtaatggccgtcggagtcggcaaagcgggccgggaggtcgtcgccgagcgggacgacccgccgcccccgaccgccccgtccccccaggtgtgccaggacggccagggcatacgcggtgtgaaaaaaggcgtcgggggcggtcccctcgacggcgcgcatcaggttctcgaggagaatggggaagcgcctggtcacctcccccaaccacgcgcgttggtcggggccaaagtcatagcgcaggcgctgtgagattcgcgggccgccctgaagcgcggcccggatggcctggcccagggcccggaggcacgccagatgtatgcgcgcggtaaaggcgacctcggcggcgatgtcaaagggcggcaggacggggcgcgggtggcgcaggggcacctcgagcgcgggaaagcgtagcagcagctccgcctgcccagcgggagacagctggtgggggcgcacgacgcgttctgcggcgcaggcctcggtcagggccgtggccagcgccgaggacagcagcggagggcgggcgcgtcgcccgccccacgccacggagttctcgtaggagacgacgacgaagcgctgcttggttccgtagtggtggcgcaggaccacggagatagaacgacggctccacagccagtccggccggtcgccgccggccagggcttcccatccgcgatccaaccactcgaccagcgaccgcggctttgcggtaccaggggtaagggttagaacgtcgttcaggatgtcctcgcccccgggcccgtggggcgctggggccacaaagcggcccccgccggggggctccagacccgccagcaccgcatctgcgtcagccgcccccatggcgcccccgctgacggcctggtgaaccagggcgccctggcgtagccccgatgcaacgccacaggccgcacgcccggtccgcgctcggaccgggtggcggcgggtgacgtcctgcactgcccgctgaaccaacgcgaggatctcctcgttctcctgtgcgatggacacgtcctgggccgcggtcgtgtcgccgccgggggccgtcagctgctcctccggggagatgggggggtcggacgccccgacgatgggcgggtctgcgggcgcccccgcgtggggccgggccaagggctgcggacgcggggacgcgctttcccccagacccatggacaggtgggccgcagcctccttcgcggccggcggggcggcggcgccaagcagagcgacgtagcggcacaaatgccgacagacgcgcatgatgcgcgtgctgtcggccgcgtagcgcgtgttgggggggacgagctcgtcgtaactaaacagaatcacgcgggcacagctcgcccccgagccccacgcaaggcgcagcgccgccacggcgtacgggtcatagacgccctgcgcgtcacacaccacgggcagggagacgaacaaccccccggcgctggacgcacgcggaaggaggccagggtgtgccggcacgacgggggccagaagctcccccaccgcatccgcgggcacgtaggcggcaaacgccgtgcaccacggggtacagtcgccggtggcatgagcccgagtctggatttcgacctggaagtttgcggccgtcccgagtccggggcggccgcgcatcagggcggccagagggattcccgcggccgccaggcactcgctggatatgatgacgtgaaccaaagaccgagggccgacccgggccgtggccgagatcgtctggacctcgttggccaagtgcgcgttcatggttcgggggtgggtgtgggtgtgtaggcgatgcgggtcccccgagtccgcgggaagggcgtgggtttggcgcgcgtatgcgtattcgccaacggaggcgtgcgtgcttatgcgcggcgcgtttcttctgtctctagggaatccgaggccaggactttaacctgctctttgtcgacgaggccaactttattcgcccggatgcggtccagacgattatgggctttctcaaccaggccaactgcaagattatcttcgtgtcgtccaccaacaccgggaaggccagtacgagctttttgtacaacctccgcggggccgcagacgagcttctcaacgtggtgacctatatatgcgatgatcacatgccgagggtggtgacgcacacaaacgccacggcctgttcttgttatatcctcaacaagcccgttttcatcacgatggacggggcggttcgccggaccgccgatttgtttctggccgattccttcatgcaggagatcatcgggggccaggccagggagaccggcgacgaccggcccgttctgaccaagtctgcgggggagcggtttctgttgtaccgcccctcgaccaccaccaacagcggcctcatggcccccgatttgtacgtgtacgtggatcccgcgttcacggccaacacccgagcctccgggaccggcgtcgctgtcgtcgggcggtaccgcgacgattatatcatcttcgccctggagcacttttttctccgcgcgctcacgggctcggcccccgccgacatcgcccgctgcgtcgtccacagtctgacgcaggtcctggccctgcatcccggggcgtttcgcggcgtccgggtggcggtcgagggaaatagcagccaggactcggccgtcgccatcgccacgcacgtgcacacagagatgcaccgcctactggcctcggagggggccgacgcgggctcgggccccgagcttctcttctaccactgcgagcctcccgggagcgcggtgctgtacccctttttcctgctcaacaaacagaagacgcccgcctttgaacactttattaaaaagtttaactccgggggcgtcatggcctcccaggagatcgtttccgcgacggtgcgcctgcagaccgacccggtcgagtatctgctcgagcagctaaataacctcaccgaaaccgtctcccccaacactgacgtccgtacgtattccggaaaacggaacggcgcctcggatgaccttatggtcgccgtcattatggccatctacctcgcggcccaggccggacctccgcacacattcgctcctatcacacgcgtctcgtgagcgcccaataaacacacccaggtatgctacgcacgaccacggtgtcgtctgttaaggggggggggggaagggggtgttggcgggaagcgtgggaacacgggggattctctcacgaccggcaccagtaccacccccctgtgaacacagaaaccccaacccaaatcccataaacatacgacacacaggcatattttggaatttcttaggtttttatttatttaggtatgctggggtttctccctggatgcccacccccacccccccgtgggtctagccgggccttagggatagcgtataacgggggccatgtctccggaccgcacaacggccgcgccgtcaaaggtgcacacccgaaccacgggagccagggccaaggtgtctcctagttggcccgcgtgggtcagccaggcgacgagcgcctcgtaaagcggcagccttcgctctccatcctgcatcagggccggggcttcggggtgaatgagctgggcggcctcccgcgtgacactctgcatctgcagtagagcgttcacgtacccgtcctgggcacttagcgcaaagagccgggggattagcgtaaggatgatggtggttccctccgtgatcgagtaaaccatgttaaggaccagcgatcgcagctcggcgtttacgggaccgagttgttggacgtccgccagcagcgagaggcgactcccgttgtagtacagcacgttgaggtctggcagccctccggggtttctggggctggggttcaggtcccggatgcccctggccacgagccgcgccacgatttcgcgcgccaggggcgatggaagcggaacgggaaaccgcaacgtgaggtccagcgaatccaggcgcacgtccgtcgcttggccctcgaacacgggcgggacgaggctgatggggtccccgttacagagatctacgggggaggtgttgcgaaggttaacggtgccggcgtgggtgaggcccacgtccagggggcaggcgacgattcgcgtgggaagcacccgggtgatgaccgcggggaagcgccttcggtacgccagcaacaaccccaacgtgtcgggactgacgcctccggagacgaaggattcgtgcgccacgtcggccagcgtcagttgccggcggatggtcggcaggaataccacccgcccttcgcagcgctgcagcgccgccgcatcggggcgcgagatgcccgagggtatcgcgatgtcagtttcaaagccgtccgccagcatggcgccgatccacgcggcagggagtgcagtggtggttcgggtggcgggaggagcgcggtgggggtcagcggcgtagcagagacgggcgaccaacctcgcataggacggggggtgggtcttagggggttgggaggcgacagggaccccagagcatgcgcggggaggtctgtcgggcccagacgcaccgagagcgaatccgtccgcggagtcccggcttgggttttatggggcccggccctcggaatcgcggcttgtcggcggggacaaagggggcggggctaggggcttgcggaaacagaagacgcgtgggataaaagaatcgcactaccccaaggaagggcggggcggtttattacagagccagtcccttgagcggggatgcgtcatagacgagatactgcgcgaagtgggtctcccgcgcgtgggcttccccgttgcgggcactgcggaggagggcggggtcgctggcgcaggtgagcgggtaggcctcctgaaacaggccacacgggtcctccacgagttcgcggcaccccggggggcgcttaaactgtacgtcgctggcggcggtggccgtggacaccgccgaacccgtctccacgatcaggcgctccaggcagcgatgtttggcggcgatgtcggccgacgtaaagaacttaaagcaggggctgagcaccggcgaggccccgttgaggtggtaggccccgttatagagcaggtccccgtacgaaaatcgctgcgacgcccacgggttggccgtggccgcgaaggcccgggacgggtcgctctggccgtggtcgtacatgagggcggtgacatccccctccttgtcccccgcgtaaacgcccccggcggcgcgtccccgggggttgcagggccggcggaagtagttgacgtcggtcgacacgggggtggcgataaactcacacacggcgtcctggccgtggtccatccctgcgcgccgcggcacctgggcgcacccgaacacggggacgggctgggccggccccaggcggtttcccgccacgaccgcgttccgcaggtacacggctgccgcgttgtccaggagagggggagccccgcggcccaggtaaaagttttggggaaggttgcccatgtcggtgacggggttgcggacggttgccgtggccacgacggcggtgtagcccacgcccaggtccacgttcgcgcgcggctgggtgagcgtgaagtttacccccccgccagtttcgtgccgggccacctggagctggcccaggaagtacgcctccgacgcgcgctccgagaacagcacgttctcagtcacaaagcggtcctgtcggacgacggtgaacccaaacccgggatggaggcccgtcttgagctgatgatgcaaggccacgggactgatcttgaagtaccccgccatgagcgcgtaggtcagcgcgttctccccggccgcgctctcgcggacgtgctgcacgacgggctgtcggatcgacgaaaagtagttggcccccagagccggggggaccagggggacctgccgcgacaggtcgcgcagggccggggggaaattgggcgcgttcgccacgtggtcggccccggcgaacagcgcgtggacggggagggggtaaaaatagtcgccattttggatggtatggtccagatgctggggggccatcagcaggattccggcgtgcaacgccccgtcgaatatgcgcatgttggtggtggacgcggtgttggcgcccgcgtcgggcgccgccgagcagagcagcgccgttgtgcgttcggccatgttgtgggccagcacctgcagcgtgagcatggcgggcccgtccactaccacgcgcccgttgtgaaacatggcgttgaccgtgttggccaccagattggccgggtgcagggggtgcgcggggtccgtcacggggtcgctggggcactcctcgccgggggcgatctccgggaccaccatgttctgcagggtggcgtatacgcggtcgaagcgaacccccgcggtgcagcagcggccccgcgagaaggcgggcaccatcacgtagtagtaaatcttgtggtgcacggtccagtccgccccccggtgcggccggtcatccgcggcgtccgcggctcgggcctgggtgttgtgcagcagctggccgtcgttgcggttgaagtccgcggtcgccacgttacatgccgccgcgtacacggggtcgtggccccccgcgctaacccggcagtcgcgatggcggtccagggccgcgcgccgcatcagggcgtcacagtcccacacgaggggtggcagcagcgccgggtctcgcattaggtgattcagctcggcttgcgcctgcccgcccagctccgggccggtcagggtaaagtcatcaaccagctgggccagggcctcgacgtgcgccaccaggtcccggtacacggccatgcactcctcgggaaggtctcccccgaggtaggtcacgacgtacgagaccagcgagtagtcgttcacgaacgccgcgcaccgcgtgttgttccagtagctggtgatgcactggaccacgagccgggccagggcgcagaagacgtgctcgctgccgtgtatggcggcctgcagcaggtaaaacaccgccgggtagttgcggtcgtcgaacgccccgcgaacggcggcgatggtggcgggggccatggcgtggcgtcccacccccagctccaggccccgggcgtcccggaacgccgccggacatagcgccaggggcaagttgccgttcaccacgcgccaggtggcctggatctcccccgggccggccgggggaacgtccccccccggcagctccacgtcggccacccccacaaagaagtcgaacgcggggtgcagctcaagagccaggttggcgttgtcgggctgcataaactgctccggggtcatctggccttccgcgacccatcggacccgcccgtgggccaggcgctgcccccaggcgttcaaaaacagctgctgcatgtctgcggcggggccggccggggccgccacgtacgccccgtacggattggcggcttcgacggggtcgcggttaaggcccccgaccgccgcgtcaacgttcatcagcgaagggtggcacacggtcccgatcgcgtgttccagagacaggcgcagcacctggcggtccttcccccaaaaaaacagctggcggggcgggaaggcgcggggatccgggtggccgggggcggggactaggtccccggcgtgcgcggcaaaccgttccatgaccggattgaacaggcccaggggcaggacgaacgtcaggtccatggcgcccaccagggggtagggaacgttggtggcggcgtagatgcgcttctccagggcctccagaaagaccagcttctcgccgatggacaccagatccgcgcgcacgcgcgtcgtctggggggcgctctcgagctcgtccagcgtctgccggttcaggtcgagctgctcctcctgcatctccagcaggtggcggcccacgtcgtccagacttcgcacggccttgcccatcacgagcgccgtgaccaggttggccccgttcaggaccatctcgccgtacgtcaccggcacgtcggcttcggtgtcctccactttcaggaaggactgcaggaggcgctgtttgatcggggcggtggtgacgagcaccccgtcgaccggccgcccgcgcgtgtcggcatgcgtcagacggggcacggccacggagggctgcgtggccgtggtgaggtccacgagccaggcctcgacggcctcccggcggtggcccgccttgcccaggaaaaagctcgtctcgcagaagcttcgctttagctcggcgaccagggtcgcccgggccaccctggtggccaggcggccgttgtccaggtatcgttgcatcggcaacaacaaagccaggggcggcgccttttccagcagcacgtgcagcatctggtcggccgtgccgcgctcaaacgccccgaggacggcctggacgttgcgagcgagctgttggatggcgcgcaactggcgatgcgcgccgatacccgtcccgtccagggcctcccccgtgagcagggcgatggcctcggtggccaggctgaaggcggcgttcagggcccggcggtcgataatcttggtcatgtaattgtgtgtgggttgctcgatggggtgcgggccgtcgcgggcaatcagcggctggtggacctcgaactgtacgcgcccctcgttcatgtaggccagctccggaaacttggtacacacgcacgccaccgacaacccgagctccagaaagcgcacgagcgacagggtgttgcaatacgaccccagcagggcgtcgaactcgacgtcgtacaggctgtttgcatcggagcgcacgcgggaaaaaaaatcaaacaggcgtcgatgcgacgccacctcgatcgtgctaaggagggacccggtcggcaccatggccgcggcataccggtatcccggagggtcgcggttgggagcggccatggggtcgcgtggagatcggctgtctctagcgatattggcccggggaggctaagatccaccccaacgcccggccacccgtgtacgtgcccgacggcccaaggtccaccgaaagacacgacgggcccggacccaaaaaggcgggggatgctgtgtgagaggccgggtgccggtcgggggggaaaggcaccgggagaaggctgcggcctcgttccaggagaacccagtgtccccaacagacccggggacgtgggatcccaggccttatatacccccccccccgccccacccccgttagaacgcgacgggtgcattcaagatggccctggtccaaaagcgtgccaggaagaaattggcagaggcggcaaagctgtccgccgccgccacccacatcgaggccccggccgcgcaggctatccccagggcccgtgtgcgcaggggatcggtgggcggcagcatttggttggtggcgataaagtggaaaagcccgtccggactgaaggtctcgtgggcggcggcgaacaaggcacacagggccgtgcctcccaaaaacacggacatcccccaaaacacgggcgccgacaacggcagacgatccctcttgatgttaacgtacaggaggagcgcccgcaccgcccacgtaacgtagtagccgacgatggcggccaggatacaggccggcgccaccacccttccggtcagcccgtaatacatgcccgctgccaccatctccaacggcttcaggaccaaaaacgaccaaaggaacagaatcacgcgctttgaaaagaccggctgggtatggggcggaagacgcgagtatgccgaactgacaaaaaaatcagaggtgccgtacgaggacaatgaaaactgttcctccagcggcagttctccctcctcccccccgaaggcggcctcgtcgaccagatctcgatccaccagaggaaggtcatcccgcatggtcatggggtgtgcggtggaggtggggagaccgaaaccgcaaagggtcgcttacgtcagcaggatcccgagatcaaagacacccgggttcttgcacaaacaccacccgggttgcatccgcggaggcgagtgttttgataaggccgttccgcgccttgatataacctttgatgttgaccacaaaacccggaatttacgcctacgccccaatgcccacgcaagatgaggtaggtaacccccccgtgggtgtgacgttgcgtttagttcattggaggccaaggggaaaaatggggtggggaggaaacggaaaacccagtaggccgtgtcgggaacacgcccggggttgtcctcaaaaggcagggtccatactacggaagccgtcgttgtattcgagacctgcctgtgcaacgcacgtcggggttgcctgtgtccggttcggcccccaccgcgtgcggcacgcacgaggacgagtccgcgtgctttattggcgttccaagcgttgccctccagtttctgttgtcggtgttcccccatacccacgcccacatccaccgtagggggcctctgggccgtgttacgtcgccgcccgcgatggagcttagctacgccaccaccatgcactaccgggacgttgtgttttacgtcacaacggaccgaaaccgggcctactttgtgtgcggggggtgtgtttattccgtggggcggccgtgtgcctcgcagcccggggagattgccaagtttggtctggtcgttcgagggacaggcccagacgaccgcgtggtcgccaactatgtacgaagcgagctccgacaacgcggcctgcaggacgtgcgtcccattggggaggacgaggtgtttctggacagcgtgtgtcttctaaacccgaacgtgagctccgagctggatgtgattaacacgaacgacgtggaagtgctggacgaatgtctggccgagtactgcacctcgctgcgaaccagcccgggtgtgctaatatccgggctgcgcgtgcgggcgcaggacagaatcatcgagttgtttgaacacccaacgatagtcaacgtttcctcgcactttgtgtataccccgtccccatacgtgttcgccctggcccaggcgcacctcccccggctcccgagctcgctggaggccctggtgagcggcctgtttgacggcatccccgccccacgccagccacttgacgcccacaacccgcgcacggatgtggttatcacgggccgccgcgccccacgacccatcgccgggtcgggggcggggtcggggggcgcgggcgccaagcgggccaccgtcagcgagttcgtgcaagtcaaacacattgaccgcgtgggccccgctggcgtttcgccggcgcctccgccaaacaacaccgactcgagttccctggtgcccggggcccaggattccgccccgcccggccccacgctaagggagctgtggtgggtgttttatgccgcagaccgggcgctggaggagccccgcgccgactctggcctcacccgcgaggaggtacgtgccgtacgtgggttccgggagcaggcgtggaaactgtttggctccgcgggggccccgcgggcgtttatcggggccgcgttgggcctgagccccctccaaaagctagccgtttactactatatcatccaccgagagaggcgcctgtcccccttccccgcgctagtccggctcgtaggccggtacacacagcgccacggcctgtacgtccctcggcccgacgacccagtcttggccgatgccatcaacgggctgtttcgcgacgcgctggcggccggaaccacagccgagcagctcctcatgttcgaccttctccccccaaaggacgtgccggtgggaagcgacgtgcaggccgacagcaccgctctgctgcgctttatagaatcgcaacgtctcgccgtccccgggggggtgatctcccccgagcacgtcgcgtaccttggtgcgttcctgagcgtgctgtacgctggccgcgggcgcatgtccgcagccacgcacaccgcgcggctgacaggggtgacctccctggtgctagcggtgggtgacgtggaccgtctttccgcgtttgaccgcggagcggcgggcgcggccagccgcacgcgggccgccgggtacctggatgtgcttcttaccgttcgtctcgctcgctcccaacacggacagtctgtgtaaaagaccccaataaacgtatatcgctactacacccttgtgtgtcaatggacgcctctccgggggggggggagggaaagcaaagaggggctgggggagcggcaccaccggggcctgaacaaacaaaccacagacacggttacagtttattcggtcgggcggagaaacggccgaagccacgcccactttattcgcgtctccaaaaaaacgggacacttgtccggagaacctttaggatgccagccagggcggcggtaatcataaccacgcccagcgcagaggcggccagaaacccgggcgcaattgcggccacgggctgcgtgtcaaaggctagcaaatgaatgacggttccgtttggaaatagcaacaaggccgtggacggcacgtcgctcgaaaacacgcttggggcgccctccgtcggcccggcggcgatttgctgctgtgtgttgtccgtatccaccagcaacacagacatgacctccccggccggggtgtagcgcataaacacggcccccacgagccccaggtcgcgctggttttgggtgcgcaccagccgcttggactcgatatcccgggtggagccttcgcatgtcgcggtgaggtaggttaggaacagtgggcgtcggacgtcgacgccggtgagcttgtagccgatcccccggggcagaggggagtgggtgacgacgtagctggcgttgtgggtgatgggtaccaggatccgtggctcgacgttggcagactgccccccgcaccgatgtgaggcctcagggacgaaggcgcggatcagggcgttgtagtgtgcccaacgcgtcagggtcgaggcgaggccgtgggtctgctgggccaggacttcgaccggggtctcggatcgggtggcttgagccagcgcgtccaggataaacacgctctcgtctagatcaaagcgcagggaggccgcgcatggcgaaaagtggtccggaagccaaaagagggttttctggtggtcggcccgggccagcgcggtccggaggtcggcgttggtcgctgcggcgacgtcggacgtacacagggccgaggctatcagaaggctccggcgggcgcgttcccgctgcaccgccgaggggacgccagccaagaacggctgccggaggacagccgaggcgtaaaatagcgcccggtggacgaccggggtggtcagcacgcggccccctagaaactcggcatacagggcgtcgatgagatgggctgcgctgggcgccactgcgtcgtacgccgaggggctatccagcacgaaggccagctgatagcccagcgcgtgtaatgccaagctctgttcgcgctccagaatctcggccaccaggtgctggagccgagcctctagctgcaggcgggccgtgggatccaagactgacacattaaaaaacacagaatccgcggcacagcccgcggccccgcgggcggccaacccggcaagcgcgcgcgagtgggccaaaaagcctagcaggtcggagaggcagaccgcgccgtttgcgtgggcggcgttcacgaaagcaaaacccgacgtcgcgagcagccccgttaggcgccagaagagaggggggcgcgggccctgctcggcgcccgcgtcccccgagaaaaactccgcgtatgcccgcgacaggaactgggcgtagttcgtgccctcctccgggtagccgcccacgcggcggagggcgtccagcgcggagccgttgtcggcccgcgtcagggaccctaggacaaagacccgataccgggggccgcccgggggcccgggaagagcccccggggggttttcgtccgcggggtccccgacccgatctagcgtctggcccgcggggaccaccatcacttccaccggagggctgtcgtgcatggatatcacgagccccatgaattcccgcccgtagcgcgcgcgcaccagcgcggcatcgcacccgagcaccagctcccccgtcgtccagatgcccacgggccacgtcgaggccgacggggagaaatacacgtacctacctggggatctcaacaggccccgggtggccaaccaggtcgtggacgcgttgtgcaggtgcgtgatgtccagctccgtcgtcgggtgccgccgggccccaaccggcggtcgggggggcggtgtatcacgcggcccgctcgggtggctcgccgtcgccacgttgtctccccgcgggaacgtcagggcctcggggtcagggacggccgaaaacgttacccaggcccgggaacgcagcaacacggaggcggctggattgtgcaagagacccttaaggggggcgaccgaggggggaggctgggcggtcggctcgaccgtggtgggggcgggcaggctcgcgttcgggggccggccgagcaggtaggtcttcgggatgtaaagcagctggccggggtcccgcggaaactcggccgtggtgaccaatacaaaacaaaagcgctcctcgtaccagcgaagaaggggcagagatgccgtagtcaggtttagttcgtccggcggcgccagaaatccgcgcggtggtttttgggggtcgggggtgtttggcagccacagacgcccggtgttcgtgtcgcgccagtacatgcggtccatgcccaggccatccaaaaaccatgggtctgtctgctcagtccagtcgtggacctgaccccacgcaacgcccaaaataataacccccacgaaccataaaccattccccatgggggaccccgtccctaacccacggggcccgtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggcagccggcgggcgattggtcgtaatccaggataaagacatgcatgggacggaggcgtttggccaagacgtccaaagcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgcccccgaaagctgtccccaatcctcccgccacgacccgccgccctgcagataccgcaccgtattggcaagcagcccataaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccaacacccgccagtaagtcatcggctcgggtacgtagacgatatcgtcgcgcgaacccagggccaccagcagttgcgtggtggtggttttccccatcccgtggggaccgtctatataaacccgcagtagcgtgggcattttctgctccaggcggacttccgtggctttttgttgccggcgagggcgcaacgccgtacgtcggttgttatggccgcgagaacgcgcagcctggtcgaacgcagacgcgtgttgatggcaggggtacgaagccatacgcgcttctacaaggcgctggccgaagaggtgcgggagtttcacgccaccaagatctgcggcacgctgttgacgctgttaagcgggtcgctgcagggtcgctcggtattcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagctcctgcagtccctcgcgcctccgggtgacaagatagtgtacctgtgccccgtcctggtgtttgtcgcccaacggacgctccgcgtcagccgcgtgacccggctcgtcccgcagaaggtctccggtaatatcaccgcagtcgtgcggatgctccagagcctgtccacgtatacggtccccattgagcctaggacccagcgagcccgtcgccgccgcggcggcgccgcccgggggtctgcgagcagaccgaaaaggtcacactctggggcgcgcgacccgcccgagtcagcggcccgccagttaccacccgccgaccaaacccccacctccacggagggcgggggggtgcttaagaggatcgcggcgctcttctgcgtgcccgtggccaccaagaccaaaccccgagccgcctccgaatgagagtgtttcgttccttccccctccccccgcgtcagacaaaccctaaccaccgcttaagcggcccccgcgaggtccgaagactcatttggatccggcgggagccacccgacaacagcccccgggttttcccacgccagacgccggtccgctgtgccatcgcgccccctcatcccaccccccatcttgtccccaaataaaacaaggtctggtagttaggacaacgaccgcagttctcgtgtgttattttcgctctccgcctctcgcagatggacccgtactgcccatttgacgctctggacgtctgggaacacaggcgcttcatagtcgccgattcccgaaacttcatcacccccgagttcccccgggacttttggatgtcgcccgtctttaacctcccccgggagacggcggcggagcaggtggtcgtcctacaggcccagcgcacagcggctgccgctgccctggagaacgccgccatgcaggcggccgagctccccgtcgatatcgagcgccggttacgcccgatcgaacggaacgtgcacgagatcgcaggcgccctggaggcgctggagacggcggcggccgccgccgaagaggcggatgccgcgcgcggggatgagccggcgggtgggggcgacgggggggcgcccccgggtctggccgtcgcggagatggaggtccagatcgtgcgcaacgacccgccgctacgatacgacaccaacctccccgtggatctgctacacatggtgtacgcgggccgcggggcgaccggctcgtcgggggtggtgttcgggacctggtaccgcactatccaggaccgcaccatcacggactttcccctgaccacccgcagtgccgactttcgggacggccgtatgtccaagaccttcatgacggcgctggtactgtccctgcaggcgtgcggccggctgtatgtgggccagcgccactattccgccttcgagtgcgccgtgttgtgtctctacctgctgtaccgaaacacgcacggggccgccgacgatagcgaccgcgctccggtcacgttcggggatctgctgggccggctgccccgctacctggcgtgcctggccgcggtgatcgggaccgagggcggccggccacagtaccgctaccgcgacgacaagctccccaagacgcagttcgcggccggcgggggccgctacgaacacggagcgctggcgtcgcacatcgtgatcgccacgctgatgcaccacggggtgctcccggcggccccgggggacgtcccccgggacgcgagtacccacgttaaccccgacggcgtggcgcaccacgacgacataaaccgcgccgccgccgcgttcctcagccggggccacaacctattcctgtgggaggaccagactctgctgcgggcaaccgcgaacaccataacggccctgggcgttatccagcggctcctcgcgaacggcaacgtgtacgcggaccgcctcaacaaccgcctgcagctgggcatgctgatccccggagccgtcccttcggaggccatcgcccgtggggcctccgggtccgactcgggggccatcaagagcggagacaacaatctggaggcgctatgtgccaattacgtgcttccgctgtaccgggccgacccggcggtcgagctgacccagctgtttcccggcctggccgccctgtgtcttgacgcccaggcggggcggccggtcgggtcgacgcggcgggtggtggatatgtcatcgggggcccgccaggcggcgctggtgcgcctcaccgccctggaactcatcaaccgcacccgcacaaaccccacccctgtgggggaggttatccacgcccacgacgccctggcgatccaatacgaacaggggcttggcctgctggcgcagcaggcacgcattggcttgggctccaacaccaagcgtttctccgcgttcaacgttagcagcgactacgacatgttgtactttttatgtctggggttcattccacagtacctgtcggcggtttagtgggtggtgggcgaggggggagggggcattagggagaaaaaacaagagcctccgttgggttttctttgtgcctgtactcaaaaggtcataccccgtaaacggcgggctccagtcccggcccggcggttggcgtgaacgcaacggcgggagctgggttagcgtttagtttagcattcgctctcgcctttccgcccgccccccgaccgttgcgcctttttttttttcgtccaccaaagtctctgtgggtgcgcgcatggcagccgatgccccgggagaccggatggaggagcccctgcccgacagggccgtgcccatttacgtggctgggtttttggccctgtatgacagcggggactcgggcgagttggcattggatccggatacggtgcgggcggccctgcctccggataacccactcccgattaacgtggaccaccgcgctggctgcgaggtggggcgggtgctggccgtggtcgacgacccccgcgggccgttttttgtggggctgatcgcctgcgtgcagctggagcgcgtcctcgagacggccgccagcgctgcgattttcgagcgccgcgggccgccgctctcccgggaggagcgcctgttgtacctgatcaccaactacctgccctcggtctccctggccacaaaacgcctggggggcgaggcgcaccccgatcgcacgctgttcgcgcacgtcgcgctgtgcgcgatcgggcggcgcctcggcactatcgtcacctacgacaccggtctcgacgccgccatcgcgccctttcgccacctgtcgccggcgtctcgcgagggggcgcggcgactggccgccgaggccgagctcgcgctgtccgggcgcacctgggcgcccggcgtggaggcgctgacccacacgctgctttccaccgccgttaacaacatgatgctgcgggaccgctggagcctggtggccgagcggcggcggcaggccgggatcgccggacacacctacctccaggcgagcgaaaaattcaaaatgtggggggcggagcctgtttccgcgccggcgcgcgggtataagaacggggccccggagtccacggacataccgcccggctcgatcgctgccgcgccgcagggtgaccggtgcccaatcgtccgtcagcgcggggtcgccttgtccccggtactgccccccatgaaccccgttccgacatcgggcaccccggcccccgcgccgcccggcgacgggagctacctgtggatcccggcctcccattacaaccagctcgtcgccggccatgccgcgccccaaccccagccgcattccgcgtttggtttcccggctgcggcggggtccgtggcctatgggcctcacggtgcgggtctttcccagcattaccctccccacgtcgcccatcagtatcccggggtgctgttctcgggacccagcccactcgaggcgcagatagccgcgttggtgggggccatagccgcggaccgccaggcgggcggtcagccggccgcgggagaccctggggtccgggggtcgggaaagcgtcgccggtacgaggcggggccgtcggagtcctactgcgaccaggacgaaccggacgcggactacccgtactaccccggggaggctcgaggcgcgccgcgcggggtcgactcccggcgcgcggcccgccattctcccgggaccaacgagaccatcacggcgctgatgggggcggtgacgtctctgcagcaggaactggcgcacatgcgggctcggaccagcgccccctatggaatgtacacgccggtggcgcactatcgccctcaggtgggggagccggaaccaacaacgacccacccggccctttgtcccccggaggccgtgtatcgccccccaccacacagcgccccctacggtcctccccagggtccggcgtcccatgcccccactcccccgtatgccccagctgcctgcccgccaggcccgccaccgcccccatgtccttccacccagacgcgcgcccctctaccgacggagcccgcgttcccccccgccgccaccggatcccaaccggaggcatccaacgcggaggccggggcccttgtcaacgccagcagcgcagcacacgtggacgttgacacggcccgcgccgccgatttgttcgtctctcagatgatgggggcccgctgattcgccccggtctttggtaccatgggatgtcttactgtatatctttttaaataaaccaggtaataccaaataagacccattggtgtatgttctttttttattgggaggcgcgggtaggcgggtagctttacaatgcaaaagccttcgacgtggaggaaggcgtggggggggggggaatcggcactgaccaagggggtccgttttgtcacgggaaaggaaagaggaaacaggccgcggacacccgggggagtttgtgttcccttttctttcttcccacacacacaaaaggcgtaccaaacaaacaaaccaaaagatgcacatgcggtttaacacccgtggtttttatttacaacaaaccccccatcacaggtcgtcctcgtcggcgtcaccgtctttgttgggaacttgggtgtagttggtgttgcggcgcttgcgcatgaccatgtcggtgaccttggcgctgagcagcgcgctcgtgcccttcttcttggccttgtgttccgtgcgctccatggcagacaccagggccatgtaccgtatcatctcccgggcctcggctagcttggcctcgtcaaagtcgccgccctcctcgccctccccggacgcgtccgggttggtggggttcttgagctccttggtggttagcgggtacagggccttcatggggttgctctgcagccgcatgacgtagcgaaaggcgaagaaggccgccgccaggccggccaggaccaacagacccacggccagcgccccaaaggggttggacatgaaggaggacacgcccgacacggccgataccacgccgcccacgatgcccatcaccaccttgccgaccgcgcgccccaggtcgcccatcccctcgaagaacgcgcccaggcccgcaaacatggcggcgttggcgtcggcgtggatgaccgtgtcgatgtcggcgaagcgcaggtcgtgcagctggttgcggcgctggacctccgtgtagtccagcaggccgctgtccttgatctcgtggcgggtgtacacctccagggggacaaactcgtgatcctccagcatggtgatgttgaggtcgatgaaggtgctgacggtggtgatgtcggcgcggctcagctggtgggagtacgcgtactcctcgaagtacacgtagcccccaccgaaggtgaagtagcgccggtgtcccacggtgcacggctcgatcgcatcgcgcgtcagccgcagctcgttgttctcccccagctgcccctcgaccaacgggccctggtcttcgtaccgaaagctgaccagggggcggctgtagcaggccccgggccgcgagctgatgcgcatcgagttttggacgatcacgttgtccgcggcgaccggcacgcacgtggagacggccatcacgtcgccgagcatccgcgcgctcacccgccggcccacggtggccgaggcgatggcgttggggttcagcttgcgggcctcgttccacagggtcagctcgtgattctgcagctcgcaccacgcgatggcaacgcggcccaacatatcgttgacatggcgctgtatgtggttgtacgtaaactgcagcctggcgaactcgatggaggaggtggtcttgatgcgctccacggacgcgttggcgctggccccgggcggcgggggcgtggggtttgggggcttgcggctctgctcgcggaggtgttcccgcacgtacagctccgcgagcgtgttgctgagaaggggctggtacgcgatcagaaagcccccattggccaggtagtactgcggctggcccaccttgatgtgcgtcgcgttgtacctgcgggcgaagatgcggtccatggcgtcgcgggcgtccttgccgatgcagtcccccaggtccacgcgcgagagcgggtactcggtcaggttggtggtgaaggtggtggatatggcgtcggaagagaatcggaaggagccgccgtactcggagcgcagcatctcgtccacctcctgccacttggtcatggtgcagaccgacgggcgctttggcacccagtcccaggccacggtgaacttgggggtcgtgagcaggttccgggtggtcggcgccgtggcccgggccttggtggtgaggtcgcgcgcgtagaagccgtcgacctgcttgaagcggtcggcggcgtagctggtgtgttcggtgtgcgacccctcccggtagccgtaaaacggggacatgtacacaaagtcgccagtcgccaacacaaactcgtcgtacgggtacaccgagcgcgcgtccacctcctcgacgatgcagtttaccgtcgtcccgtaccggtggaacgcctccacccgcgaggggttgtacttgaggtcggtggtgtgccagccccggctcgtgcgggtcgcggcgttggccggtttcagctccatgtcggtctcgtggtcgtcccggtgaaacgcggtggtctccaggttgttgcgcacgtacttggccgtggaccgacagacccccttggcgttgatcttgtcgatcacctcctcgaaggggacgggggcgcggtcctcaaagatccccataaactgggagtagcggtggccgaaccacacctgcgaaacggtgacgtctttgtagtacatggtggccttgaacttgtacggggcgatgttctccttgaagaccaccgcgatgccctccgtgtagttctgaccctcgggccgggtcgggcagcggcgcggctgctcgaactgcaccaccgtggcgcccgtggggggtgggcacacgtaaaagtttgcatcggtgttctccgccttgatgtcccgcaggtgctcgcgcagggtggcgtggcccgcggcgacggtcgcgttgtcgccggcggggcgcggcggctttgggggtttcggttttctgttcttcttcggtttcgggtcccccgttgggggggcgccaggggcgggcggcgccggagtggcagggcccccgttcgccgcctgggtcgcggccgcgaccccaggcgtgccgggggaactcggagccgccgacgccaccaggacccccagcgtcaaccccaagagcgcccatacgacgaaccaccggcgcccccgcgcgggggcgccctggcgcatggcgggactacgggggcccgtcgtgccccccgtcaggtagcctgggggcgaggtgctggaggaccgagtagaggatcgagaaaacgtctcggtcgtagaccacgaccgaccgggggccgatacagccgtcgggggcgctctcgacgatggccaccagcggacagtcggagtcgtacgtgagatatacgccgggcgggtaacggtaacgaccttcggaggtcgggcggctgcagtccgggcggcgcaactcgagctccccgcaccggtagaccgaggcaaagagtgtggtggcgataatcagctcgcgaatatatcgccaggcggcgcgctgagtgggcgttattccggaaatgccgtcaaaacagtaaaacctctgaaattcgctgacggcccaatcagcacccgagccccccgcccccatgatgaaccgggcgagctcctccttcaggtgcggcaggagccccacgttctcgacgctgtaatacagcgcggtgttggggggctgggcgaagctgtgggtggagtgatcaaagaggggcccgttgacgagctcgaagaagcgatgggtgatgctggggagcagggccgggtccacctggtgtcgcaggagagacgctcgcatgaaccggtgcgcgtcgaacacgcccggcgccgagcggttgtcgatgaccgtgcccgcgcccgccgtcagggcgcagaagcgcgcgcgcgccgcaaagccgttggcgaccgcggcgaacgtcgcgggcagcacctcgccgtggacgctgacccgcagcatcttctcgagctccccgcgctgctcgcggacgcagcgccccaggctggccaacgaccgcttcgtcaggcggtccgcgtacagccgccgtcgctcccgcacgtccgcggccgcttgcgtggcgatgtccccccacgtctcgggcccctgccccccgggcccgcggcgacggtcttcgtcctcgcccccgcccccgggagctcccaacccccgtgccccttcctctacggcgacacggtccccgtcgtcgtcggggcccgcgccgcccttgggcgcgtccgccgcgccccccgcccccatgcgcgccagcacgcgacgcagcgcctcctcgtcgcactgttcggggctgacgaggcgccgcaagagcggcgtcgtcaggtggtggtcgtagcacgcgcggatgagcgcctcgatctgatcgtcgggtgacgtggcctgaccgccgattattagggcgtccaccatatccagcgccgccaggtggctcccgaacgcgcgatcgaaatgctccgcccgccgcccgaacagcgccagttccacggccaccgcggcggtctcctgctgcaactcgcgccgcgccagcgcggtcaggttgctggcaaacgcgtccatggtggtctggccggcgcggtcgccggacgcgagccagaatcgcaattcgctgatggcgtacaggccgggcgtggtggcctgaaacacgtcgtgcgcctccagcagggcgtcggcctccttgcggaccgagtcgttctcgggcgacgggtggggctgcccgtcgccccccgcggtccgggccagcgcatggtccaacacggagagcgcccgcgcgcggtcggcgtccgacagcccggcggcgtggggcaggtaccgccgcagctcgttggcgtccagccgcacctgcgcctgctgggtgacgtggttacagatacggtccgccaggcggcgggcgatcgtcgccccctggttcgccgtcacacacagttcctcgaaacagaccgcgcaggggtgggacgggtcgctaagctccggggggacgataaggcccgaccccaccgcccccaccataaactcccgaacgcgctccagcgcggcggtggcgccgcgcgagggggtgatgaggtggcagtagtttagctgctttagaaagttctcgacgtcgtgcaggaaacacagctccatatggacggtcccgccatacgtatccagcctgacccgttggtgatacggacagggtcgggccaggcccatggtctccgtgaaaaacgccgcgacgtctcccgcggtcgcgaacgtctccaggctgcccaggagccgctcgccctcgcgccacgcgtactctagcagcaactccagggtgaccgacagcggggtgagaaaggccccggcctgggcctccaggcccggcctcagacgacgccgcagcgcccgcacctgaagcgcgttcagcttcagttgggggagcttcccccgtccgatgtgggggtcgcaccgccggagcagctctatctgaaacacataggtctgcacctgcccgagcagggctaacaacttttgacgggccacggtgggctcggacaccggggcggccatctcgcggcgccgatctgtaccgcggccggagtatgcggtggaccgaggcggtccgtacgctacccggcgtctggctgagccccggggtccccctcttcggggcggcctcccgcgggcccgccgaccggcaagccgggagtcggcggcgcgtgcgtttctgctctattcccagacaccgcggagaggaatcacggcccgcccagagatatagacacggaacacaaacaagcacggatgtcgtagcaataatttattttacacacattccccgccccgccctaggttcccccaccccccaacccctcacagcatatccaacgtcaggtctccctttttgtcggggggcccctccccaaacgggtcatccccgtggaacgcccgtttgcggccggcaaatgccggtcccggggcccccgggccgccgaacggcgtcgcgttgtcgtcctcgcagccaaaatccccaaagttaaacacctccccggcgttgccgagttggctgactagggcctcggcctcgtgcgccacctccagggccgcgtccgtcgaccactcgccgttgccgcgctccagggcacgcgcggtcagctccatcatctcctcgcttaggtactcgtcctccaggagcgccagccagtcctcgatctgcagctgctgggtgcggggccccaggcttttcacggtcgccacgaacacgctactggcgacggccgccccgccctcggagataatgccccggagctgctcgcacagcgagctttcgtgcgctccgccgccgaggcttgaggccgcgcacacaaacccggcccggggacaggccaggacgaacttgcgggtgcggtcaaaaataaggagcgggcacgcgtttttgccgcccatcaggctggcccagttcccggcctgaaacacacggtcgttgccggccatgccgtagtacttgctgatgctcaaccccaacacgaccatggggcgcgccgccatgacgggccgcagcaggttgcagctggcgaacatggacgtccacgcgcccggatgcgcgtccacggcgtccatcagcgcgcgggccccggcctccaggcccgccccgccctgcgcggaccacgcggccgcagcctgcacgctggggggacggcgggaccccgcgatgatggccgtaagggtgttgatgaagtatgtcgagtgatcgcagtaccgcagaatctggtttgccatgtagtacatcgccagctcgctcacgttgttgggggccaggttaataaagtttatcgcgccgtagtccagggaaaactttttaatgaacgcgatggtctcgatgtcctcgcgcgacaggagccgggcgggaagctggttgcgttggagggccgtccagaaccactgcgggttcggctggttggaccccgggggcttgccgttggggaagatggccgcgtggaactgcttcagcagaaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagctccttgggaaaccccagggtcacgcgggcaacgtcgccctcgaagctgctctcggcgggggccgtctggccggccgttaggctgggggcgcagatagccgccccctccgagagcgcgaccgtcagcgttttggccgacagaaacccgttgttaaacatgtccatcacgcgccgccgcagcaccggttggaattgattgcgaaagttgcgcccctcgaccgactgcccggcgaacaccccgtggcactgactcagggccaggtcctggtacacggcgaggttggatcgccgcccgagaagctgaagcagggggcacggcccgcacgcgtacgggtccagcgtcagggacatggcgtggttggcctcgcccagaccgtcgcgaaacttgaagttcctcccctccaccaggttgcgcatcagctgctccacctcgcggtccacgacctgcctgacgttgttcaccaccgtatgcagggcctcgcggttggtgatgatggtctccagccgccccatggccgtggggaccgcctggtccacgtactgcagggtctcgagttcggccatgacgcgctcggtcgccgcgcggtacgtctcctgcatgatggtccgggcggtctcggatccgtccgcgcgcttcagggccgagaaggcggcgtagtttcccagcacgtcgcagtcgctgtacatgctgttcatggtcccgaagacgccgatggctccgcgggcggcgctggcgaactttggatggcgcgcccggaggcgcatgagcgtcgtgtgtacgcaggcgtggcgcgtgtcgaaggtgcataggttacagggcacgtcggtctggttggagtccgcgacgtatcgaaacacgtccatctcctggcgcccgacgatcacggcgccgtcgcagcgctccaggtaaaacagcatcttggccagcagcgccggggaaaacccacacagcatggccaggtgctcgccggcaaattcctgggttccgccgacgaggggcgcggtgggccgaccctcgaacccgggcaccacgtgtccctcgcggtccacctgtgggttggccgccacgtgggtcccgggcacgaggaagaagcggtaaaaggagggtttgctgtggtcctttgggtccgccgggccggcgtcgtccacctcggtgagatggagggccgagttggtgctaaataccatggcccccacgagtcccgcggcgcgcgccaggtacgccccgacggcgttggcgcgggccgcggccgtgtcctggccctcgaacagcggccacgcggagatgtcggtgggcggctcgtcaaagacggccatcgacacgatagactcgagggccagggcggcgtctccggccatgacggaggccaggcgctgttcgaacccgcccgccgcgcccttgccgccgccgtcgcgcccgccccgcggggtcttaccctggctggcttcgaaggccgtgaacgtaatgtcggcggggagggcggcgccctcgtggttttcgtcaaacgccaggtgggcggccgcgcgggccacggcgtccacgtttcggcatcgcagtgccacggcggcgggtcccacgaccgcctcgaacaggaggcggttgagggggcggttaaaaaacggaagcgggtaggtaaatttctccccgatcgatcggtggttggcgttgaacggctctgcgatgacacggctaaaatccggcatgaacagctgcaacgggtacacgggtatgcggtgcacctccgccccgcctatggttaccttgtccgagcctcccaggtgcagaaaggtgttgttgatgcacacggcctccttgaagccctcggtaacgaccagatacaggagggcgcggtccgggtccaggccgaggcgctcacacagcgcctcccccgtcgtctcgtgtttgaggtcgccgggccggggggtgtagtccgaaaagccaaaatggcggcgtgcccgctcgcaaagtcgcgtcaggttcggggcctgggtgctggggtccaggtgccggccgccgtgaaagacgtacacggacgagctgtagtgcgagggcgtcagtttcagggacaccgcggtacccccgagccccgtcgtgcgagaacccacgaccacggccacgttggcctcaaagccgctctccacggtcaggcccacgaccaggggcgccacggcgacgtcggaatcgccgctgcgtgccgacagtaacgccagaagctcgatgccttcggacggacacgcgcgagcgtacacgtatcccaggggcccgggggggaccttgatggtggttgccgtcttgggctttgtctccatgtccttttgtcaatcggtccgcgaacggaggtaatcccggcacgacgacggacgcccgacaaggtatgtctcccgagcgtcaaaatccgggggggggcggcgacggtcaaggggagggttggagaccggggttggggaatgaatccctccccttcaccgacaaccccccgggtaaccacggggtcgccgatgaaccccggcggccggcaacgcggggtccctgcgagaggcacagatgcttacggtcaggtgctccgggtcgggtgcgtctggtatgcggttggtatatgtacactttacctgggggcgtgccggtccgccccagcccctcccacgccccgcgcgtcatcagccggtgggcgtggccgctattataaaaaaagtgagaacgcgaagcgttcgcactttgtcctaataatatatatattattaggacaaagtgcgaacgcttcgcgttctcactttttttataatagcggccacgcccaccggctacgtcactctcctgtcggccgccggcggtccataagcccggccggccgggccgacgcgaataaaccgggccgccggccggggcgccgcgcagcagctcgccgcccggatccgccagacaaacaaggcccttgcacatgccggcccgggcgagcctgggggtccggtaattttgccatcccacccaagcggctttttgggtttttctcttcccccctccccacattcccctctttaggggttcgggtgggaacaaccgcgatgttttccggtggcggcggcccgctgtcccccggaggaaagtcggcggccagggcggcgtccgggttttttgcgcccgccggccctcgcggagccagccggggacccccgccttgtttgaggcaaaacttttacaacccctacctcgccccagtcgggacgcaacagaagccgaccgggccaacccagcgccatacgtactatagcgaatgcgatgaatttcgattcatcgccccgcgggtgctggacgaggatgcccccccggagaagcgcgccggggtgcacgacggtcacctcaagcgcgcccccaaggtgtactgcgggggggacgagcgcgacgtcctccgcgtcgggtcgggcggcttctggccgcggcgctcgcgcctgtggggcggcgtggaccacgccccggcggggttcaaccccaccgtcaccgtctttcacgtgtacgacatcctggagaacgtggagcacgcgtacggcatgcgcgcggcccagttccacgcgcggtttatggacgccatcacaccgacggggaccgtcatcacgctcctgggcctgactccggaaggccaccgggtggccgttcacgtttacggcacgcggcagtacttttacatgaacaaggaggaggtcgacaggcacctacaatgccgcgccccacgagatctctgcgagcgcatggccgcggccctgcgcgagtccccgggcgcgtcgttccgcggcatctccgcggaccacttcgaggcggaggtggtggagcgcaccgacgtgtactactacgagacgcgccccgctctgttttaccgcgtctacgtccgaagcgggcgtgtgctgtcgtacctgtgcgacaacttctgcccggccatcaagaagtacgagggtggggtcgacgccaccacccggttcatcctggacaaccccgggttcgtcaccttcggctggtaccgtctcaaaccgggccggaacaacacgctagcccagccggcggccccgatggccttcgggacatccagcgacgtcgagtttaactgtacggcggacaacctggccatcgaggggggcatgagcgacctaccggcatacaagctcatgtgcttcgatatcgaatgcaaggcggggggggaggacgagctggcctttccggtggccgggcacccggaggacctggtcatccagatatcctgtctgctctacgacctgtccaccaccgccctggagcacgtcctcctgttttcgctcggttcctgcgacctccccgaatcccacctgaacgagctggcggccaggggcctgcccacgcccgtggttctggaattcgacagcgaattcgagatgctgttggccttcatgacccttgtgaaacagtacggccccgagttcgtgaccgggtacaacatcatcaacttcgactggcccttcttgctggccaagctgacggacatttacaaggtccccctggacgggtacggccgcatgaacggccggggcgtgtttcgcgtgtgggacataggccagagccacttccagaagcgcagcaagataaaggtgaacggcatggtgaacatcgacatgtacgggattataaccgacaagatcaagctctcgagctacaagctcaacgccgtggccgaagccgtcctgaaggacaagaagaaggacctgagctatcgcgacatccccgcctactacgccgccgggcccgcgcaacgcggggtgatcggcgagtactgcatacaggattccctgctggtgggccagctgttttttaagtttttgccccatctggagctctcggccgtcgcgcgcttggcgggtattaacatcacccgcaccatctacgacggccagcagatccgcgtctttacgtgcctgctgcgcctggccgaccagaagggctttattctgccggacacccaggggcgatttaggggcgccgggggggaggcgcccaagcgtccggccgcagcccgggaggacgaggagcggccagaggaggagggggaggacgaggacgaacgcgaggagggcgggggcgagcgggagccggagggcgcgcgggagaccgccggcaggcacgtggggtaccagggggccagggtccttgaccccacttccgggtttcacgtgaaccccgtggtggtgttcgactttgccagcctgtaccccagcatcatccaggcccacaacctgtgcttcagcacgctctccctgagggccgacgcagtggcgcacctggaggcgggcaaggactacctggagatcgaggtgggggggcgacggctgttcttcgtcaaggctcacgtgcgagagagcctcctcagcatcctcctgcgggactggctcgccatgcgaaagcagatccgctcgcggattccccagagcagccccgaggaggccgtgctcctggacaagcagcaggccgccatcaaggtcgtgtgtaactcggtgtacgggttcacgggagtgcagcacggactcctgccgtgcctgcacgttgccgcgacggtgacgaccatcggccgcgagatgctgctcgcgacccgcgagtacgtccacgcgcgctgggcggccttcgaacagctcctggccgatttcccggaggcggccgacatgcgcgcccccgggccctattccatgcgcatcatctacggggacacggactccatctttgtgctgtgccgcggcctcacggccgccgggctgacggccgtgggcgacaagatggcgagccacatctcgcgcgcgctgtttctgccccccatcaaactcgagtgcgaaaagacgttcaccaagctgctgctgatcgccaagaaaaagtacatcggcgtcatctacgggggtaagatgctcatcaagggcgtggatctggtgcgcaaaaacaactgcgcgtttatcaaccgcacctccagggccctggtcgacctgctgttttacgacgataccgtctccggagcggccgccgcgttagccgagcgccccgcggaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgccccccccgcggccctgccctccccggccaagcgcccccgggagacgccgtcgcctgccgaccccccgggaggcgcgtccaagccccgcaagctgctggtgtccgagctggccgaggatcccgcatacgccattgcccacggcgtcgccctgaacacggactattacttctcccacctgttgggggcggcgtgcgtgacattcaaggccctgtttgggaataacgccaagatcaccgagagtctgttaaaaaggtttattcccgaagtgtggcaccccccggacgacgtggccgcgcggctccggaccgcagggttcggggcggtgggtgccggcgctacggcggaggaaactcgtcgaatgttgcatagagcctttgatactctagcatgagccccccgtcgaagctgatgtccctcattttacaataaatgtctgcggccgacacggtcggaatctccgcgtccgtgggtttctctgcgttgcgccggaccacgagcacaaacgtgctctgccacacgtgggcgacgaaccggtaccccgggcacgcggtgagcatccggtctatgagccggtagtgcaggtgggcggacgtgccgggaaagatgacgtacagcatgtggcccccgtaagtggggtccgggtaaaacaacagccgcgggtcgcacgccccgcctccgcgcaggatcgtgtggacgaaaaaaagctcgggttggccaagaatcccggccaagaggtcctggaggggggcgttgtggcggtcggccaacacgaccaaggaggccaggaaggcgcgatgctcgaatatcgtgttgatctgctgcacgaaggccaggattagggcctcgcggctggtggcggcgaaccgcccgtctcccgcgttgcacgcgggacagcaacccccgatgcctaggtagtagcccatcccggagagggtcaggcagttgtcggccacggtctggtccagacagaagggcagcgagacgggagtggtcttcaccaggggcaccgagagcgagcgcacgatggcgatctcctcggagggcgtctgggcgagggcggcgaaaaggccccgatagcgctggcgctcgtgtaaacacagctcctgtttgcgggcgtgaggcggcaggctcttccgggaggcccgacgcaccacgcccagagtcccgccggccgcagaggagcgcgaccgccggcgctccttgccgtgatagggcccgggccgggagccgcggcgatgggggtcggtgtcatacataggtacacagggtgtgctccagggacaggagcgagatcgagtggcgtctaagcagcgcgcccgcctcacggacaaatgtggcgagcgcggtgggctttggtacaaatacctgatacgtcttgaaggtgtagatgagggcacgcaacgctatgcagacacgcccctcgaactcgttcccgcaggccagcttggccttgtggagcagcagctcgtcgggatgggtggcggggggatggccgaacagaacccaggggtcaacctccatctccgtaatggcgcacatggggtcacagaacatgtgcttaaagatggcctcgggccccgcggcccgaagcaggctcacaaaccggcccccgtccccgggctgcgtctcggggtcagcctcgagctggtcgacgacgggtacgatacagtcgaagaggctcgtgttgttttccgagtagcggaccacggaggcccggagtctgcgcagggccagccagtaagcacgcaccagtaacaggttacacagcaggcattctccgccggtgcgcccgcgcccccggccgtgtttcagcacggtggccatcagagggcccaggtcgaggtcgggctgggcatcgggttcggtaaactgcgcaaagcgcggagccacgtcgcgcgtgcgtgccccgcgatgcgcttcccaggactggcggaccgtggcgcgacgggcctccgcggcagcgcgcagctggggccccgactcccagacggcgggggtgccggcgaggagcaacaggaccagatccgcgtacgcccacgtatccggcgactcctccggctcgcggtccccggcgaccgtctcgaattccccgttgcgagcggcggcgcgagtacagcagctgtccccgcccccgcgccgaccctccgtgcagtccaggagacgggcgcaatccttccagttcatcagcgcggtggtgagcgacggctgcgtgccggatcccgccgccgaccccgccccctcctcgcccccggaggccaaggttccgatgagggcccgggtggcagactgcgccaggaacgagtagttggagtactgcaccttggcggctcccggggagggcgagggcttgggttgcttctgggcatgccgcccgggcaccccgccgtcggtacggaagcagcagtggagaaaaaagtgccggtggatgtcgtttatggtgagggcaaagcgtgcgaaggagccgaccagggtcgccttcttggtgcgcagaaagtggcggtccatgacgtacacaaactcgaacgcggccacgaagatgctagcggcgcagtggggcgcccccaggcatttggcacagagaaacgcgtaatcggccacccactgaggcgagaggcggtaggtttgcttgtacagctcgatggtgcggcagaccagacagggccggtccagcgcgaaggtgtcgatggccgccgcggaaaagggcccggtgtccaaaagcccctccccacagggatccgggggcgggttgcggggtcctccgcgcccgcccgaaccccctccgtcgcccgcccccccgcgggcccttgagggggcggtgaccacgtcggcggcgacgtcctcgtcgagcgtaccgacgggcggcacacctatcacgtgactggccgtcaggagctcggcgcagagagcctcgttaagagccaggaggctgggatcgaaggccacatacgcgcgctcgaacgcccccgccttccagctgctgccgggggactcttcgcacaccgcgacgctcgccaggaccccggggggcgaagttgccatggctgggcgggaggggcgcacgcgccagcgaactttacgggacacaatccccgactgcgcgctgcggtcccagaccctggagagtctagacgcgcgctacgtctcgcgagacggcgcgcatgacgcggccgtctggttcgaggatatgacccccgccgagctggaggttgtcttcccgactacggacgccaagctgaactacctgtcgcggacgcagcggctggcctccctcctgacgtacgccgggcctataaaagcgcccgacgacgccgccgccccgcagaccccggacaccgcgtgtgtgcacggcgagctgctcgccgccaagcgggaaagattcgcggcggtcattaaccggttcctggacctgcaccagattctgcggggctgacgcgcgtgctgttgggcgggacggttcgcgaaccctttggtgggtttacgcgggcacgcacgctcccatcgcgggcgccatggcgggactgggcaagccctacaccggccacccaggtgacgccttcgagggtctcgttcagcgaattcggcttatcgtcccatctacgttgcggggcggggacggggaggcgggcccctactctccctccagcctcccctccaggtgcgcctttcagtttcatggccatgacgggtccgacgagtcgtttcccatcgagtatgtactgcggcttatgaacgactgggccgaggtcccgtgcaacccttacctgcgcatacagaacaccggcgtgtcggtgctgtttcaggggttttttcatcgcccacacaacgcccccgggggcgcgattacgccagagcggaccaatgtgatcctgggctccaccgagacgacggggctgtccctcggcgacctggacaccatcaaggggcggctcggcctggatgcccggccgatgatggccagcatgtggatcagctgctttgtgcgcatgccccgcgtgcagctcgcgtttcggttcatgggccccgaagatgccggacggacgagacggatcctgtgccgcgccgccgagcaggctattacccgtcgccgccgaacccggcggtcccgggaggcgtacggggccgaggccgggctgggggtggccggaacgggtttccgggccaggggggacggttttggcccgctccccttgttaacccaagggccctcccgcccgtggcaccaggccctgcggggtcttaagcacctacggattggcccccccgcgctcgttttggcggcgggactcgtcctgggggccgctatttggtgggtggttggtgctggcgcgcgcctataaaaaaggacgcaccgccgccctaatcgccagtgcgttccggacgccttcgccccacacagccctcccgaccgacacccccatatcgcttcccgacctccggtcccgatggccgtcccgcaatttcaccgccccagcaccgttaccaccgatagcgtccgggcgcttggcatgcgcgggctcgtcttggccaccaataactctcagtttatcatggataacaaccacccgcacccccagggcacccaaggggccgtgcgggagtttctccgcggtcaggcggcggcgctgacggaccttggtctggcccacgcaaacaacacgtttaccccgcagcctatgttcgcgggcgacgccccggccgcctggttgcggcccgcgtttggcctgcggcgcacctattcaccgtttgtcgttcgagaaccttcgacgcccgggaccccgtgaggcccggggagttccttctggggtgttttaatcaataaaagaccacaccaacgcacgagccttgcgtttaatgtcgtgtttattcaagggagtgggatagggttcgacggttcgaaacttaacacaccaaataatcgagcgcgtctagcccagtaacatgcgcacgtgatgtaggctggtcagcacggcgtcgctgtgatgaagcagcgcccggcgggtccgctgtaactgctgttgtaggcggtaacaggcgcggatcagtaccgccagggcgctacgaccggtgcgttgcacgtagcgtcgcgacagaactgcgtttgccgatacgggcggggggccgaattgtaagcgcgtcacctcttgggagtcatcggcggataacgcactgaatggttcgttggttatgggggagtgtggttccccagggagtgggtcgagcgcctcggcctcggaatccgagaggaacaacgaggtggcgtcggagtcttcgtcgtcagagacatacagggtctgaagcagcgacacgggcgggggggtagcgtcgatgtgtagcgcgagggaggatgcccacgaagacaccccagacaaggagctgcccgtgcgtggatttgtggaagacgcggaagccgggacggatgggcggttttgcggtgcccggaaccgaaccgccggatactccccgggtgctacatgcccgttttggggctggggttggggctggggttggggctggggttggggctggggttggggctggggttggggctggggttggggttggggttggggctggggttggggttggggctggggctggggctggggctggggctggggctggggctggggctggggctggggctggggctggggctggggctggggctggggctggggctggggttggggcgcggacaggcggctgacggtcaaatgcccccgggggcgcgcagatgtggtgggcgtggccaccggctgccgtgtagtggggcggcgggaaaccgggcctccgggcgcaacaccgccctccagcgtcaagtatgtggggggcgggcctgacgtcgggggcggggcgacgggttggaccgcgggaggcgggggagagggacctgcgggagaggatgaggtcggctcggccgggttgcggcctaaaacaggggccgtggggtcggcggggtcccagggtgaagggagggattcccgcgattcggacagcgacgcgacagcggggcgcgtaaggcgccgctgcggcccgcctacgggaaccctggggggggttggcgcgggacccgaggttagcggggggcggcggttttcgcccccgggcaaaaccgtgccggttgcgaccgggggcggaacgggatcgatagggagagcgggagaagcctggccggcggcctggggcccgagcgggaggggcacaccagacaccaaagcgtggggcgctggctctgggggtttgggaggggccggggggcgcgcgaaatcggtaaccggggcgaccgtgtcggggagggcaggcggccgccaaccctgggtggtcgcggaagcctgggtggcgcgcgccagggagcgtgcccggcggtgtcggcgcgcgcgcgacccggacgaagaagcggcagaagcgcgggaggaggcgggggggcggggggcggtggcatcggggggcgccggggaactttggggggacggcaagcgccggaagtcgtcgcgggggcccacgggcgccggccgcgtgctttcggccgggacgcccggtcgtgcttcgcgagccgtgactgccggcccagggggccgcggtgcacactgggacgtggggacggactgatcggcggtgggcgaaagggggtccggggcaaggaggggcgcggggccgccggagtcgtcagacgcgagctcctccaggccgtgaatccatgcccacatgcgaggggggacgggctcgccgggggtggcgtcggtgaatagcgtgggggccaggcttccgggccccaacgagccctccgccccaacaaggtccacagggccgggggtcgggtttgggaccgaggggctctggtcgtcgggggcgcgctggtacaccggatgccccgggaatagctcccccgacaggagggaggcgtcgaacggccgcccgaggatagctcgcgcgaggaaggggtcctcgtcggtggcgctggcggcgaggacgtcctcgccgcccgccacaaacgggagctcctcggtggcctcgctgccaacaaaccgcacgtcgggggggccgggggggtccgggttttcccacaacaccgcgaccggggtcatggagatgtccacgagcaccagacacggcgggccccgggcgaggggccgctcggcgatgagcgcggacaggcgcgggagctgtgccgccagacacgcgttttcgatcgggttcaggtcggcgtgcaggaggcggacggcccacgtctcgatgtcggacgacacggcatcgcgcaaggcggcgtccggcccgcgagcgcgtgagtcaaacagcgtgagacacagctccagctccgactcgcgggaaaaggccgtggtgttgcggagcgccacgacgacgggcgcgcccaggagcactgccgccagcaccaggtccatggccgtaacgcgcgccgcgggggtgcggtgggtggcggcggccggcacggcgacgtgctggcccgtgggccggtagagggcgttggggggagcggggggtgacgcctcgcgcccccccgaggggctcagcgtctgcccagattccagacgcgcggtcagaagggcgtcgaaactgtcatactctgtgtagtcgtccggaaacatgcaggtccaaagagcgaccagagcggtgcttgggagacacatgcgcccgaggacgctcaccgccgccagcgcctgggcgggactcagctttcccagcgcggcgccgcgctcggttcccagctcggggaccgagcgccagggcgccagggggtcggtttcggacaacttgccgcggcgccagtctgccagccgcgtgccgaacatgaggccccgggtcggagggcctccggccgaaaacgctggcagcacgcggatgcgggcgtctggatgcggggtcaggcgctgcacgaatagcatggaatctgctgcgttctgaaacgcacgggggagggtgagatgcatgtactcgtgttggcgaaccagatccaggcgccaaaaggtgtaaatgtgttccggggagctggccaccagcgccaccagcacgtcgttctcgttaaaggaaacgcggtgcctagtggagctctggggtccgagcggcggccccggggccgccgcgtcacccccccattccagctgggcccagcgacacccaaactcgcgcgtgagagtggtcgcgacgagggcgacgtagagctcggccgccgcatccatcgaggccccccatctcgcctggcggtggcgcacaaagcgtccgaagagctgaaagttggcggcctgggcgtcgctgagggccagctgaagccggttgatgacggtgaggacgtacatggccgtgacggtcgaggccgactccagggtgtccgtcggaagcggggggcgaatgcatgccgcctcgggacacatcagcagcgcgccgagcttgtcggtcacggccgggaagcagagcgcgtactgcagtggcgttccatccgggaccaaaaagctgggggcgaacggccgatccagcgtactggtggcctcgcgcagcaccaggggccccgggcctccgctcactcgcaggtacgcctcgccccggcggcgcagcatctgcgggtcggcctcttggccgggtggggcggacgcccgggcgcgggcgtctagggcgcgaagatccacgagcaggggcgcgggcgcggccgccgcgcccgcgcccgtctggcctgtggccttggcgtacgcgctatataagcccatgcggcgttggatgagctcccgcgcgccccggaactcctccaccgcccatggggccaggtccccggccaccgcgtcgaattccgccaacaggccccccagggtgtcaaagttcatctcccaggccacccttggcaccacctcgtcccgcagccgggcgctcaggtcggcgtgttgggccacgcgccccccgagctcctccacggccccggcccgctcggcgctcttggcgcccaggacgccctggtacttggcgggaaggcgctcgtagtcccgctgggctcgcagccccgacacagtgttggtggtgtcctgcagggcgcgaagctgctcgcatgccgcgcgaaatccctcgggcgatttccaggcccccccgcgaacgcggccgaagcgaccccatacctcgtcccactccgcctcggcctcctcgagagacctccgcagggcctcgacgcggcgacgggtgtcgaagagcgcctgcaggcgcgcgccctgtcgcgtcaggaggcccgggccgtcgccgctggccgcgcttagcgggtgcgtctcaaaggtacgctgggcatgttccaaccaggcgaccgcctgcacgtcgagctcgcgcgccttctccgtctggtccaccagaatttcgacctgatccgcgatctcctccgccgagcgcgcctggtccagcgtcttggccacggtcgccgggacggcgaccaccttcagcagggtcttcagattggccagaccctcggcctcgagctgggcccggcgctcgcgcgcggccagcacctcccgcagccccgccgtgacccgctcggtggcttcggcgcgctgctgtttggcgcgcaccacggcgtccttggtatcggccaggtcctgtcgggtcacgaatgcgacgtagtgcgcgtacgccgtgtccttcacggggctctggtccacgcgctccagcgccgccacgcacgccaccagcgcgtcctcgctcgggcagggcagggtgacccctgcccggacaagctcggcggccgccgccgggtcgttgcgcaccgcggatatctcctccgcggcggcggccaggtccagcgccacgcttccgatcgcgcgccgcgcgtcggcccggagggcgtccaggcgatcgcggatatccacgtactcggcgtagcccttttgaaaaaacggcacgtactggcgcagggccggcacgccccccaagtcttccgacaggtgtaggacggcctcgtggtagtcgataaacccgtcgttcgcctgggcccgctccagcagcccccccgccagccgcagaagccgcgccaggggctcggtgtccacccgaaacatgtcggcgtacgtgtcggccgcggccccgaaggccgcgctccagtcgatgcggtgaatggctgcgagcggggggagcatggggtggcgctggttctcgggggtgtatgggttaaacgcaagggccgtctccagggcaagggtcaccgccttggcgttggttcccagcgcctgttcggcccgctttcggaagtcccgggggttgtagccgtgcgtgcccgccagcgcctgcaggcgacggagctcgaccacgtcaaactcggcaccgctttccacgcggtccagcacggcctccacgtcggcggcccagcgctcgtggctactgcgggcgcgctgggccgccatcttctctctgaggtcggcggtggcggcctcaagttcgtcggcgcggcgtcgcgtggcgccgatgacctttcccagctcctgcagggcgcgcccgctgggggagtggtccccggccgtcccttcggcgtgcaacaggcccccgaacctgccctcgtggcccgcgaggctttcccgcgcgccggtggtcgcgcgcgtcgcggcctggatcagggaggcatgctctccctccggttggttggcggcccggcgcacctggacgacaaggtcggcggcagccgaccctaaggtcgtgagctgggcgatggccacccgcgcgtccagggccaaccgagtcgccttgacgtatcccgcggcgctgtcggccatggccgctaggaaggccaggggggaggccgggtcgctggcggccgcgcccagggccgtcaccgcgtcgaccaggacgcggtgcgcccgcacggccgcatccaccgtcgacgcagggtctgccgttgcgacggcggcgctgccggcgttgatggcgttcgagacggcgtgggctatgatcggggcgtgatcggcgaagaactgcaagagaaacggagtctctggggcgtcggcgaacaggttcttcagcaccaccacgaagctgggatgcaagccagacagagccgtcgccgtgtccggagtcgggtgctccagggcatctcggtactgccccagcagcccccacatgtccgcccgcagcgccgccgtaacctcagggggcgccccccgaacggcctcggggaggtccgaccagcccgccggcagggaggcccgcagggtcgccaggacggccggacaggcctttagccccacaaagtcagggagggggcgcaggaccccctggagtttgtgcaagaacttctcccgggcgtcgcgggccaccttcgcccgctcccgcgctccctcgagcattgcctccagggagcgcgcgcgctcccgcaaacgggcacgcgcatcgggggcgagctctgccgtcagcttggcggcatccatggcccgcgcctgccgcagcgcttcctcggccatgcgcgtggcctctggcgacagcccgccgtcgtcggggtagggcgacgcgccgggcgcaggaacaaaggccgcgtcgctgtccagctgctggcccagggccgcatctagggcgtcgaagcgccgcagctcggccagacccgagctgcggcgcgcctgttggtcgttaatgtcgcggatgctgcgcgccagctcgtccagtggcttgcgttctatcagcccttggttggcggcgtccgtcaggacggagagccaggccgccaggtcctcgggggcgtccagcgtctggccccgctggatcagatcccgcaacaggatggccgtggggctggtcgcgatcgggggcggggcgggaatggcggcgcgctgcgcgatgtcccgcgtgtgctggtcgaagacaggcagggactcgagcagctggaccacgggcacgacggcggccgaagccacgtgaaaccggcggtcgttgttgtcgctggcctgtagagccttggcgctgtatacggccccccggtaaaagtactccttaaccgcgccctcgatcgcccgacgggcctgggtccgcacctcctccagccgaacctgaacggcctcggggcccagggggggtgggcgcggagccccctgcggggccgccccggccggggcgggcattacgccgaggggcccggcgtgctgtgagaccgcgtcgaccccgcgagcgagggcgtcgagggcctcgcgcatctggcgatcctccgcctccaccctaatctcttcgccacgggcaaatttggccagagcctggactctatacagaagcggttctgggtgcgtcggggtggcgggggcaaaaagggtgtccgggtgggcctgcgagcgctccagaagccactcgccgaggcgtgtatacagattggccggcggggccgcgcgaagctgcagctccaggtccgcgagttccccgtaaaaggcgtccgtctcccgaatgacatccctagccacaaggatcagcttcgccagcgccaggcgaccgatcagagagttttcgtccagcacgtgctggacgaggggcagatgggcggccacgtcggccaggctcaggcgcgtggaggccagaaagtcccccacggccgttttccagggcagcatgttcagggtaaactccagcagggcggcggccgggccggccaccccggcctgggtgtgcgtccgggccccgttctcgatgagaaaggcgaggacgcgttcaaagaaaaaaataacacagagctccagcagccccggagaggccggatacggcgaccgtaaggcgctgatggtgagccgcgaacacgcggcgacctcgcgggccagggcggcggagcacgcggtgaacttaaccgccgtggcggccacgtttgggtgggcctcgaacagctgggcgaggtctgcgcccgggggctcgggcgagcggcgagtcttcagcgcctcgagggcctgtgaggacgccggaaccgtgggcccgtcgtcctcgcccgcctcggcgaccggcggcccggccgggtcggggggtgccgaggcgaggacaggctccggaacggaggcggggaccgcggccccgacgggggttttgcctttgggggtggatttcttcttggttttggcagggggggccgagcgtttcgttttctcccccgaagtcaggtcttcgacgctggaaggcggagtccaggtgggtcggcggcgcttgggaaggccggccgagtagcgtgcccggtgccgaccaaccgggacgacgcccatctccaggacccgcatgtcgtcgtcatcttcttcggccgcctctgcggcgggggtcttgggggcggagggaggcggtggtgggatcgcggagggtgggtcggcggagggtgggtcggcggaggggggatccgtgggtggggtacccttcagggccaccgcccatacatcgtcgggcgcccgattcgggcgcttggcctctggttttgccgacggaccggccgtcccccgggatgtctcggaggccctgtcgtcgcgacgggcccgggtcggtggcggcgactgggcggctgtgggcgggtgtggccccgggccccctaccccctcccgggggcccacgccgacgcagggctcccccaggcccgcgatctcgccccgcagggggtgcgtgatggccacgcgccgttcgctgaacgcttcgtcctgcaggtaagtctcgctggccccgtaaagatgcagagccgcggccgtcaagtccgcaggagccgcgggttccgggcccgacggcacgaaaaacaccatggctcccgcccaccgtacgtccgggcgatcgcgggtgtaatacgtcaggtatggatacatgtcccccgcccgcactttggcgatgaacgcgggggtgccctccggaaggccgtgcgggtcaaaaaggtatgcggtgtcgccgtccctgaacagccccatccctagggggccaatggttaggagcgtgtacgacagggggcgcagggcccacgggccggcgaagaacgtgtgtgcggggcattgtgtctccagcaggcccgccgcgggctccccgaagaagcccacctcgccgtatacgcgcgagaagacacagcgcagtccgccgcgcgcccctgggtactcgaggaagttggggagctcgacgatcgaacacatgcgcggcggcccagggcccgcggtcgcgcgcgtccactcgcccccctcgaccaaacatccctcgatggcctccgcggacaggacgtcgcgagggcccacatcaaatatgaggctgagaaaggacagcgacgagcgcatgcacgataccgacccccccggctccaggtcgggcgcgaactggttccgagcaccggtgaccacgatgtcgcgatcccccccgcgttccatcgtggagtgcggtggggtgcccgcgatcatatgtgccctgctggccagagacccggcctgtttatggaccggacccccggggttagtgttgtttccgccacccatgcccccgtaccatggccccggttcccctgattaggctacgagtcgcggtgatcgcttcccaaaaaccgagctgcgtttgtctgtcttggtcttccacccccccccccgcccgcccgcacaccataacaccgagaacaacacacgggggtgggcgtaacataataaagctttattggtaactagttaacggcaagtccgtgggtggcgcgacggtgtcctccgggatcatctcgtcgtcctcgacgggggtgttggaatgaggcgccccctcgcggtccgcctggcgtgggccgtgcccataggcctccggcttctgtgcgtccatgggcataggcgcggggagactgtttccggcgtcgcggacctccaggtccctgggagactccggtccggctaacggacgaaacgcggaagcgcgaaacacgccgtcggtgacccgcaggagctcgttcatcagtaaccaatccatactcagcgtaacggccagcccctggcgagacagatccacggagtccggaaccgcggtcgtctggcccagggggccgaggctgtagtccccccaggcccctaggtcgcgacggctcgtaagcacgacgcggtcggccgcggggctttgcgggggggcgtcctcgggcgcatgcgccattacctctcggatggccgcggcgcgctggtcggccgagctgaccaagggcgccacgaccacggcgcgctccgtctgcaggcccttccacgtgtcgtggagttcctggacaaactcggccacgggctcgggtcccgcggccgcgcgcgcggcttgatagcaggccgacagacgccgccagcgcgctagaaactgacccatgaagcaaaacccggggacctggtctcccgacagcagcttcgacgcccgggcgtgaatgccggacacgacggacagaaacccgtgaatttcgcgccggaccacggccagcacgttgtcctcgtgcgacacctgggccgccagctcgtcgcacacccccaggtgcgccgtggtttcggtgatgacggaacgcaggctcgcgagggacgcgaccagcgcgcgcttggcgtcgtgatacatgctgcagtactgactcaccgcgtcccccatggcctcggggggccagggccccaggcggtcgggcgtgtccccgaccaccgcatacaggcggcgcccgtcgctctcgaaccgacactcgaaaaaggcggagagcgtgcgcatgtgcagccgcagcagcacgatggcgtcctccagttggcgaatcagggggtctgcgcgctcggcgaggtcctgcagcaccccccgggcggccagggcgtacatgctaatcaacaggaggctggtgcccacctcggggggcggggggggctgcagctggaccaggggccgcagctgctcgacggcacccctggagatcacgtacagctcccggagcagctgctctatgttgtcggccatctgcatagtggggccgaggccgccccgggcggccggttcgaggagggtgatcagcgcgcccagtttggtgcgatggccctcaaccgtggggagatagcccagcccaaagtcccgggcccaggccaacacacgcagggcgaactcgaccgggcggggaaggtaggccgcgctacacgtggccctcagcgcgtccccgaccaccagggccagaacgtaggggacgaagcccgggtcggcgaggacgttggggtgaatgccctcgagggcggggaagcggatctgggtcgccgcggccaggtggacagagggggcgtggctgggctgcccgacggggagaagcgcggacagcggcgtggccggggtggtgggggtgatgtcccagtgggtctgaccatacacgtcgatccagatgagcgccgtctcgcggagaaggctgggttgaccggaactaaagcggcgctcggccgtctcaaactcccccacgagcgcccgccgcaggctcgccagatgttccgtcggcacggccggacccatgatacgcgccagcgtctggcttagaacgccccccgacaggccgaccgcctcgcagagccgcccgtgcgtgtgctcgctggcgccctggacccgcctgaaagtttttacgtagttggcatagtacccgtattcccgcgccaacccaaacacgttcgaccccgcgagggcaatgcacccaaagagctgctggacttcgccgagtccgtggccggtgggcgtccgcgcggggacgcccgccgccagaaacccctccagggccgaaaggtagtgcgtgcagtgcgagggcgtgaacccagcgtcgatcagggtgttgatcaccacggagggcgaattggtattctggatcaacgtccacgtctgctgcagcagagccagcagccgctgctgggcgccggcggagggctgctccccgagctgcagcaggctggagacggcaggctggaagactgccagtgccgacgaactcaggaacggcacgtcgggatcaaacacggccacgtccgtccgcacgcgcgccattagcgtccccgggggcgcacaggccgagcgcgggctgacgcggctgagggccgtcgacacgcgcacctcctcgcggctgcgaaccatcttgttggcctcgagcggcggaatcattatggccgggtcgatctcccgcacggtgtgctgaaactgcgccaacaggggcggcgggaccacagccccccgctcgggggtcgtcaggtactcgtccaccagggccaacgtaaagagggcccgtgtgaggggagtgagggtcgcgtcgtctatgcgctggaggtgcgccgagaacagcgtcacccgattactcaccagggccaagaaccggaggccctcttgcacgaacggggcggggaagagcaggctgtacgccggggtggtaaggttcgcgctgggctgccccaacgggaccggcgccatcttgagcgacgtctccccaagggcctcgatggaggtccgcgggctcatggccaagcagctcttggtgacggtttgccagcggtctatccactccacggcgcactggcggacgcggaccggccccagggccgccgcggtgcgcaggccggcggaatccagcgcatgggacgtgtcggagccggtgaccgcgaggatggtgtccttgatgacctccatctcccggaaggcctggtcgggggcctcggggagagccaccaccaagcggtgtacgagcaacccggggaggttctcggccaagagcgccgtctccggaagcccgtgggcccggtggagcgcgcacaggtgttccagcagcggccgccagcagtcccgcgcgtctgccggggcgatggccgttcccgacaacagaaacgccgccatggcggcgcgcagcttggccgtggccagaaacgccgggtcgtccgccccgtttgccgtctcggccgtgggggttggcggttggcgaaggccggctaggctcgccaataggcgctgcataggtccgtccgagggcggaccggcgggtgaggtcgtgacgacgggggcctcggacgggagaccgcggtctgccatgacgcccggctcgcgtgggtgggggacagcgtagaccaacgacgagaccgggcgggaatgactgtcgtgcgctgtagggagcggcgaattatcgatcccccgcggccctccaggacccccgcaggcgttgcgagtaccccgcgtcttcgcggggtgttatacggccacttaagtcccggcatcccgttcgcggacccaggcccgggggattgtccggatgtgcgggcagcccggacggcgtgggttgcggactttctgcggggcggcccaaatggccctttaaacgtgtgtatacggacgcgccgggccagtcggccaacacaacccaccggaggcggtagccgcgtttggctgtggggtgggtggttccgccttgcgtgagtgtcctttcgacccccccccccctccctcccccgggtcttgctaggtcgcgatctggggtcgcaatgaagaccaatccgctacccgcaaccccttccgtgtggggcgggagtaccgtggaactcccccccacaacacgcgataccgcgggacagggcctgcttcggcgcgtcctgcgccccccgatctctcgccgcgacggcccagggctccccagggggtcgggaccccggagggcggccagcacgctgtggttgcttggcctggacggcacagacgcgccccctggggcgctgacccccaacgacgataccgaacaggccctggacaagatcctgcggggcaccatgcgcgggggggcggccctgatcggctccccgcgccatcatctaacccgccaagtgatcctgacggatctgtgccaacccaacgcggatcgtgctgggacgctgcttctggcgctgcggcaccccgccgacctgcctcacctggcccaccagcgcgccccgccaggccggcagaccgagcggctgggcgaggcctggggccagctgatggaggcgaccgccctggggtcggggcgagccgagagcgggtgcacgcgcgcgggcctagtgtcgtttaacttcctggtggcggcgtgtgccgcctcgtacgacgcgcgcgacgccgccgatgcggtacgggcccacgtcacggccaactaccgcgggacgcgggtgggggcgcgcctggatcgtttttccgagtgtctgcgcgccatggttcacacgcacgtcttcccccacgaggtcatgcggtttttcggggggctggtgtcgtgggtcacccaggacgagctagcgagcgtcaccgccgtgtgcgccgggccccaggaggcggcgcacaccggccacccgggccggccccgctcggccgtgatcctcccggcatgtgcgttcgtggacctggacgccgagctggggctggggggcccgggcgcggcgtttctgtacctggtattcacttaccgccagcgccgggaccaggagctgtgttgtgtgtacgtgatcaagagccagctccccccgcgcgggttggagccggccctggagcggctgtttgggcgcctccggatcaccaacacgattcacggcaccgaggacatgacgcccccggccccaaaccgaaaccccgacttccccctcgcgggcctggccgccaatccccaaaccccgcgttgctcggctggccaggtcacgaacccccagttcgccgacaggctgtaccgctggcagccggacctgcgggggcgccccaccgcacgcacctgtacgtacgccgcctttgcagagctcggcatgatgcccgaggatagtccccgctgcctgcaccgcaccgagcgctttggggcggtcagcgtccccgttgtcattctggaaggcgtggtgtggcgccccggcgagtggcgggcatgcgcgtgagcgtagcaaacgccccgcccacacaacgctccgcccccaaccccttccccgctgtcactcgttgttcgttgacccgggcgtccgccaaataaagccactgaaacccgaaacgcgagtgttgtaacgtcctttgggcgggaggaagccacaaaatgcaaatgggatacatggaaggaacacacccccgtgactcaggacatcggtgtgtccttttgggtttcactgaaactggcccgcgccccacccctgcgcgatgtggataaaaagccagcgcgggtggtttagggtaccacaggtgggtgctttggaaacttgccggtcgccgtgctcctgtgagcttgcgtccctccccggtttcctttgcgctcccgccttccggacctgctctcgcctactcttctttggctctcggtgcgattcgtcaggcagcggccttgtcgaatctcgaccccaccactcgccggacccgccgacgtcccctctcgagcccgccgaaacccgccgcgtctgttgaaatggccagccgcccagccgcatcctctcccgtcgaagcgcgggccccggttgggggacaggaggccggcggccccagcgcagccacccagggggaggccgccggggcccctctcgcccacggccaccacgtgtactgccagcgagtcaatggcgtgatggtgctttccgacaagacgcccgggtccgcgtcctaccgcatcagcgataacaactttgtccaatgtggttccaactgcaccatgatcatcgacggagacgtggtgcgcgggcgcccccaggacccgggggccgcggcatcccccgctcccttcgttgcggtgacaaacatcggagccggcagcgacggcgggaccgccgtcgtggcattcgggggaaccccacgtcgctcggcggggacgtctaccggtacccagacggccgacgtccccaccgaggcccttgggggcccccctcctcctccccgcttcaccctgggtggcggctgttgttcctgtcgcgacacacggcgccgctctgcggtattcgggggggagggggatccagtcggccccgcggagttcgtctcggacgaccggtcgtccgattccgactcggatgactcggaggacacggactcggagacgctgtcacacgcctcctcggacgtgtccggcggggccacgtacgacgacgcccttgactccgattcgtcatcggatgactccctgcagatagatggccccgtgtgtcgcccgtggagcaatgacaccgcgcccctggatgtttgccccgggacccccggcccgggcgccgacgccggtggtccctcagcggtagacccacacgcgccgacgccagaggccggcgctggtcttgcggccgatcccgccgtggcccgggacgacgcggaggggctttcggacccccggccacgtctgggaacgggcacggcctaccccgtccccctggaactcacgcccgagaacgcggaggccgtggcgcgctttctgggagatgccgtgaaccgcgaacccgcgctcatgctggagtacttttgccggtgcgcccgcgaggaaaccaagcgtgtcccccccaggacattcggcagcccccctcgcctcacggaggacgactttgggcttctcaactacgcgctcgtggagatgcagcgcctgtgtctggacgttcctccggtcccgccgaacgcatacatgccctattatctcagggagtatgtgacgcggctggtcaacgggttcaagccgctggtgagccggtccgctcgcctttaccgcatcctgggggttctggtgcacctgcggatccggacccgggaggcctcctttgaggagtggctgcgatccaaggaagtggccctggattttggcctgacggaaaggcttcgcgagcacgaagcccagctggtgatcctggcccaggctctggaccattacgactgtctgatccacagcacaccgcacacgctggtcgagcgggggctgcaatcggccctgaagtatgaggagttttacctaaagcgttttggcgggcactacatggagtccgtcttccagatgtacacccgcatcgccggctttttggcctgccgggccacgcgcggcatgcgccacatcgccctggggcgagaggggtcgtggtgggaaatgttcaagttctttttccaccgcctctacgaccaccagatcgtaccgtcgacccccgccatgctgaacctggggacccgcaactactacacctccagctgctacctggtaaacccccaggccaccacaaacaaggcgaccctgcgggccatcaccagcaacgtcagtgccatcctcgcccgcaacgggggcatcgggctatgcgtgcaggcgtttaacgactccggccccgggaccgccagcgtcatgcccgccctcaaggtccttgactcgctggtggcggcgcacaacaaagagagcgcgcgtccgaccggcgcgtgcgtgtacctggagccgtggcacaccgacgtgcgggccgtgctccggatgaagggggtcctcgccggcgaagaggcccagcgctgcgacaatatcttcagcgccctctggatgccagacctgtttttcaagcgcctgattcgccacctggacggcgagaagaacgtcacatggaccctgttcgaccgggacaccagcatgtcgctcgccgactttcacggggaggagttcgagaagctctaccagcacctcgaggtcatggggttcggcgagcagatacccatccaggagctggcctatggcattgtgcgcagtgcggccacgaccgggagccccttcgtcatgttcaaagacgcggtgaaccgccactacatctacgacacccagggggcggccatcgccggctccaacctctgcaccgagatcgtccatccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggaatcggcatgcagggcctgcacacggcctgcctgaagctggggctggatctggagtctgccgaatttcaggacctgaacaaacacatcgccgaggtgatgctgctgtcggcgatgaagaccagcaacgcgctgtgcgttcgcggggcccgtcccttcaaccactttaagcgcagcatgtatcgcgccggccgctttcactgggagcgctttccggacgcccggccgcggtacgagggcgagtgggagatgctacgccagagcatgatgaaacacggcctgcgcaacagccagtttgtcgcgctgatgcccaccgccgcctcggcgcagatctcggacgtcagcgagggctttgcccccctgttcaccaacctgttcagcaaggtgacccgggacggcgagacgctgcgccccaacacgctcctgctaaaggaactggaacgcacgtttagcgggaagcgcctcctggaggtgatggacagtctcgacgccaagcagtggtccgtggcgcaggcgctcccgtgcctggagcccacccaccccctccggcgattcaagaccgcgtttgactacgaccagaagttgctgatcgacctgtgtgcggaccgcgccccctacgtcgaccatagccaatccatgaccctgtatgtcacggagaaggcggacgggaccctcccagcctccaccctggtccgccttctggtccacgcatataagcgcggactaaaaacagggatgtactactgcaaggttcgcaaggcgaccaacagcggggtctttggcggcgacgacaacattgtctgcatgagctgcgcgctgtgaccgacaaaccccctccgcgccaggcccgccgccactgtcgtcgccgtcccacgctctcccctgctgccatggattccgcggccccagccctctcccccgctctgacggcccttacggaccagagcgcgacggcggacctggcgatccagattccaaagtgccccgaccccgagaggtacttctacacctcccagtgtcccgacattaaccacctgcgctccctcagcatccttaaccgctggctggaaaccgagcttgttttcgtgggggacgaggaggacgtctccaagctttccgagggcgagctcagcttttaccgcttcctcttcgctttcctgtcggccgccgacgacctggttacggaaaacctgggcggcctctccggcctgtttgagcagaaggacattctccactactacgtggagcaggaatgcatcgaagtcgtacactcgcgcgtgtacaacatcatccagctggtgcttttccacaacaacgaccaggcgcgccgcgagtacgtggccggtaccatcaaccacccggccatccgcgccaaggtggactggttggaagcgcgggtgcgggaatgcgcctccgttccggaaaagttcattctcatgatcctcatcgagggcatcttttttgccgcctcgtttgccgccatcgcctaccttcgcaccaacaaccttctgcgggtcacctgccagtcaaacgacctcatcagccgggacgaggccgtgcacacgacggcctcgtgttacatctacaacaactacctcggcgggcacgccaagcccccgcccgaccgcgtgtacgggctgttccgccaggcggtcgagatcgagatcggatttatccgatcccaggcgccgacggacagccatatcctgagcccggcggcgctggcggccatcgaaaactacgtgcgattcagcgcggatcgcctgttgggccttatccacatgaagccactgttttccgccccaccccccgacgccagctttccgctgagcctcatgtccaccgacaaacacaccaattttttcgagtgtcgcagcacctcctacgccggggcggtcgtcaacgatctgtgagtgtcgcggcgcgcttctacccgtgtttgcccataataaacctctgaaccaaactttgggtctcattgtgattcttgtcagggacgcgggggtgggagaggataaaaggcggcgcaaaaagcagtaaccaggtccgtccagattctgcgggcataggataccataattttattggtgggtcgtttgttcggggacaagcgcgctcgtctgacgtttgggctactcgtcccagaatttggccaggacgtccttgtagaacgcgggtgggggggcctgggtccgcaactgctccagaaacctgtcggcgatatcaggggccgtgatatgccgggtcacgatagatcgcgccaggttttcgtcgcggatgtcctggtagataggcaggcgtttcagaagagtccacggcccccgctccttggggccgataagcgatatgacgtacttaatgtagcggtgttccaccagctcggtgatggtcatgggatcggggagccagtccagggactctggggcgtcgtggatgacgtggcgtcgcgggttggccacataactgcggtgctcttccagcagctgcgcgttcgggacctggacgagctcgggcggggtgagtatctccgaggaggacgacctggggccggggtggcccccggtaacgtcccggggatccagggggaggtcctcgtcgtcttcgtatccgccggcgatctgttgggttagaatttcggtccacgagacgcgcgtctcggtgccgccggcggccggcggcagagggggcctggtttccgtggagcgcgagctggtgtgttcccggcggatggcccgccgggtctgagagcgactcgggggggtccagtgacattcgcgcagcacatcctccacggaggcgtaggtgttattgggatggaggtcggtgtggcagcggacaaagagggccaggaactgggggtagctcatcttaaagtactttagtatatcgcgacagttgatcgtgggaatgtagcaggcgctaatatccaacacaatatcacagcccatcaacaggaggtcagtgtccgtggtgtacacgtacgcgaccgtgttggtgtgatagaggttggcgcaggcatcgtccgcctccagctgacccgagttaatgtaggcgtaccccagggcccggagaacgcgaatacagaacagatgcgccagacgcagggccggcttcgagggcgcggcggacggcagcgcggctccggacccggccgtcccccgggtccccgaggccagagaggtgccgcgccggcgcatgttggaaaaggcagagctgggtctggagtcggtgatgggggaaggcggtggagaggcgtccacgtcactggcctcctcgtccgtccggcactgggccgtcgtgcgggccaggatggccttggctccaaacacaaccggctccatacaattgaccccgcgatcggtaacgaagatggggaaaagggacttttgggtaaacacctttaataagcgacagaggcagtgtagcgtaatggcctcgcggtcgtaactggggtatcggcgctgatatttgaccaccaacgtgtacatgacgttccacaggtccacggcaatgggggtgaagtacccggccggggccccaaggccccggcgcttgaccagatggtgtgtgtgggcaaacttcatcatcccgaacaaacccatgtcaggtcaattgtaactgcggatcggcctaactaaggcgtggttggtgcgacggtccgggacacccgagcctgtctctctgtgtatggtgacccagacaacaacaccgacacaagaggacaataatccgttaggggacgctctttataatttcgatggcccaactccacgcggattggtgcagcaccctgcatgcgccggtgcgggccaaccttccccccgctcattgcctcttccaaaagggtgtggcctaacgagctgggggcgtatttaatcaggctagcgcggcgggcctgccgtagtttctggctcggtgagcgacggtccggttgcttgggtcccctggctgccatcaaaaccccaccctcgcagcggcatacgccccctccgcgtcccgcacccgagaccccggcccggctgccctcaccaccgaagcccacctcgtcactgtggggtgttcccagcccgcgttgggatgacggattcccctggcggtgtggcccccgcctcccccgtggaggacgcgtcggacgcgtccctcgggcagccggaggagggggcgccctgccaggtggtcctgcagggcgccgaacttaatggaatcctacaggcgtttgccccgctgcgcacgagccttctggactcgcttctggttatgggcgaccggggcatccttatccataacacgatctttggggagcaggtgttcctgcccctggaacactcgcaattcagtcggtatcgctggcgcggacccacggcggcgttcctgtctctcgtggaccagaagcgctccctcctgagcgtgtttcgcgccaaccagtacccggacctacgtcgggtggagttggcgatcacgggccaggccccgtttcgcacgctggttcagcgcatatggacgacgacgtccgacggcgaggccgttgagctagccagcgagacgctgatgaagcgcgaactgacgagctttgtggtgctggttccccagggaacccccgacgttcagttgcgcctgacgaggccgcagctcaccaaggtccttaacgcgaccggggccgatagtgccacgcccaccacgttcgagctcggggttaacggcaaattttccgtgttcaccacgagtacctgcgtcacctttgctgcccgcgaggagggcgtgtcgtccagcaccagcacccaggtccagatcctgtccaacgcgctcaccaaggcgggccaggcggccgccaacgccaagacggtgtacggggaaaatacccatcgcaccttctctgtggtcgtcgacgattgcagcatgcgggcggtgctccggcgactgcaggtcggcgggggcaccctcaagttcttcctcacgacccccgtccccagtctgtgcgtcaccgccaccggtcccaacgcggtatcggcggtatttctcctgaaaccccagaagatttgcctggactggctgggtcatagccaggggtctccttcagccgggagctcggcctcccgggcctctgggagcgagccaacagacagccaggactccgcgtcggacgcggtcagccacggcgatccggaagacctcgatggcgctgcccgggcgggagaggcgggggccttgcatgcctgtccgatgccgtcgtcgaccacgcgggtcactcccacgaccaagcgggggcgctcggggggcgaggatgcgcgcgcggacacggccctaaagaaacctaagacggggtcgcccaccgcacccccgcccgcagatccagtccccctggacacggaggacgactccgatgcggcggacgggacggcggcccgtcccgccgctccagacgcccggagcggaagccgttacgcgtgttactttcgcgacctcccgaccggagaagcaagccccggcgccttctccgccttccgggggggcccccaaaccccgtatggttttggattcccctgacggggcggggccttggcggccgcccaactctcgcaccatcccgggttaatgtaaataaacttggtattgcccaacactttcccgcgtgtcgcgtgtggttcatgtgtgtgcctggcgcccccaccctcgggttcgtgtatttcctttccctgtccttataaaagccgtatgtggggcgtgacggaaccaccccgcgtgccatcacggccaaggcgcgggatgctccgcaacgacagccaccgggccgtgtccccggaggacggccagggacgggtcgacgacggacggccacacctcgcgtgcgtgggggccctggcgcgggggttcatgcatatctggcttcaggccgccacgctgggttttgcgggatcggtcgttatgtcgcgcgggccgtacgcgaatgccgcgtctggggcgttcgccgtcgggtgcgccgtgctgggctttatgcgcgcaccccctcccctcgcgcggcccaccgcgcggatatacgcctggctcaaactggcggccggtggagcggcccttgttctgtggagtctcggggagcccggcacgcagccgggggccccggccccgggcccggccacccagtgcctggcactgggcgccgcctatgcggcgctcctggtgctcgccgatgacgtctatccgctctttctcctcgccccggggcccctgttcgtcggcaccctggggatggtcgtcggcgggctgacgatcggaggcagcgcgcgctactggtggatcggtgggcccgccgcggccgccctggccgcggcggtgttggcgggcccgggggcgaccaccgccagggactgcttttccagggcttgccccgaccaccgccgcgtctgtgtcatcaccgcaggcgagtctctttcccgccgccccccggaggacccagagcgacccggggttcccgggcccccgtcccccccgaccccccaacgatcccacgggccgccggccgatgaggtcgcaccggccagggtcgcgcggcccgaaaacgtctgggtgcccgtggtcacctttctgggggcgggcgcgcttgccgtcaagacggtgcgagaacatgcccggggaacgccgggcccgggcctgccgctgtggccccaggtgtttctcggaggccatgtggcggtggccctgacggagctgtgtcaggcgcttccgccctgggaccttacggacccgctgctgtttgttcacgccggactgcaggtcatcaacctcgggttggtgtttcggttttccgaggttgtcgtgtatgcggcgctagggggtgccgtgtggatttcgttggcgcaggtgctggggctccggcgtcgcctgcacaggaaggaccccggggacggggcccggttggcggcgacgcttcggggcctcttcttctccgtgtacgcgctggggtttggggtgggggtgctgctgtgccctccggggtcaacgggcgggcggtcgggcgattgatatatttttcaataaaaggcattagtcccgaagaccgccggtgtgtgatgatttcgccataacacccaaaccccggatggggcccgggtataaattccggaaggggacacgggctaccctcactatcgagggcgcttggtcgggaggccgcatcgaacgcacacccccatccggtggtccgtgtggaggtcgttttcatgcccggtctcgctttgccgggaacgctagccgatccctcgcgagggggaggcgtcgggcatggccccggggcgggtgggccttgccgtggtcctgtggagcctgttgtggctcggggcgggggtgtccgggggctcggaaactgcctccaccgggcccacgatcaccgcgggagcggtgacgaacgcgagcgaggcccccacatcggggtcccccgggtcagccgccagcccggaggtcacccccacatcgaccccaaaccccaacaatgtcacacaaaacaaaaccacccccaccgagccggccagccccccaacaacccccaagcccacctccacgcccaaaagcccccccacgtccacccccgaccccaaacccaagaacaacaccacccccgccaagtcgggccgccccactaaaccccccgggcccgtgtggtgcgaccgccgcgacccattggcccggtacggctcgcgggtgcagatccgatgccggtttcggaattccacccgcatggagttccgcctccagatatggcgttactccatgggtccgtcccccccaatcgctccggctcccgacctagaggaggtcctgacgaacatcaccgccccacccgggggactcctggtgtacgacagcgcccccaacctaacggacccccacgtgctctgggcggagggggccggcccgggcgccgaccctccgttgtattctgtcaccgggccgctgccgacccagcggctgattatcggcgaggtgacgcccgcgacccagggaatgtattacttggcctggggccggatggacagcccgcacgagtacgggacgtgggtgcgcgtccgcatgttccgccccccgtctctgaccctccagccccacgcggtgatggagggtcagccgttcaaggcgacgtgcacggccgccgcctactacccgcgtaaccccgtggagtttgtctggttcgaggacgaccaccaggtgtttaacccgggccagatcgacacgcagacgcacgagcaccccgacgggttcaccacagtctctaccgtgacctccgaggctgtcggcggccaggtccccccgcggaccttcacctgccagatgacgtggcatcgcgactccgtgacgttctcgcgacgcaatgccaccgggctggccctggtgctgccgcggccaaccatcaccatggaatttggggtccgcattgtggtctgcacggccggctgcgtccccgagggcgtgacgtttgcctggttcctgggggacgacccctcaccggcggctaagtcggccgttacggcccaggagtcgtgcgaccaccccgggctggctacggtccggtccaccctgcccatttcgtacgactacagcgagtacatctgtcggttgaccggatatccggccgggattcccgttctagaacaccacggcagtcaccagcccccacccagggaccccaccgagcggcaggtgatcgaggcgatcgagtgggtggggattggaatcggggttctcgcggcgggggtcctggtcgtaacggcaatcgtgtacgtcgtccgcacatcacagtcgcggcagcgtcatcggcggtaacgcaagacccccccgttacctttttaatatctatatagtttggtcccccctctatcccgcccaccgctgggcgctataaagccgccaccctctcttccctcaggtcatccttggtcgatcccgaacgacacacggcgtggagcaaaacgcctccccctgagccgctttcctaccaacacaacggcatgcctctgcgggcatcggaacacgcctaccggcccctgggccccgggacaccccccatgcgggctcggctccccgccgcggcctgggttggcgtcgggaccatcatcgggggagttgtgatcattgccgcgttggtcctcgtgccctcgcgggcctcgtgggcactttccccatgcgacagcggatggcacgagttcaacctcgggtgcatatcctgggatccgacccccatggagcacgagcaggcggtcggcggctgtagcgccccggcgaccctgatcccccgcgcggctgccaaacagctggccgccgtcgcacgcgtccagtcggcaagatcctcgggctactggtgggtgagcggagacggcattcgggcctgcctgcggctcgtcgacggcgtcggcggtattgaccagttttgcgaggagcccgcccttcgcatatgctactatccccgcagtcccgggggctttgttcagtttgtaacttcgacccgcaacgcgctggggctgccgtgaggcgcgtgtactgcggtctgtctcgtctcctcttctccccttccctccccctccgcatcccaggatcacaccggtcaacgagggttgggggggtccggcacggacccaaaataataaacacacaatcacgtgcgataaaaagaacacgcggtcccctgtggtgtttttggttatttttattaaatctcgtcgacaaacagggggaaaggggcgtggtctagcgacggcagcacgggcggaggcgttcaccggctccggcgtccttcgcgtttaagcttggtcaggagggcgctcagggcggcgacgttggtcgggccgtcgttggtcagggcgttggctcgatggcgggcgaggacgggcgaggggctcaacggcgggggcgggggcccggtgcggcccgggggggaaaatagggcggatcccccccagtcgtacaggggattttccgcctcaatgtacggggaggccggcgctgcattcgccgtgttcacgcagacgttttcgtagacccgcatccatggtatttcctcgtagacacgccccccgtcctcgctcaccgtctcgtatattgactcgtcgtcctcgtagggggcgtgccgttcgcgggccgaggcggcgtgggtggctttgcggcgggcgtcgtcgtcgtcgtcgtcggccgtcagatacgtggcttccatctggtcgggttctccctccggggcgggtccccacacccgtggccgatcgaggctccccagagacgcgcgccggacgaggagggggcacgtcgccgccggcggtcgcctgtcgggtcccgcgacgttacgggccgggaggcgcgggggcacctcccccatgtgcgtgtaatacgtggccggctgtgtggccgcagcggggggctcggcgaccgggtcgttcgcatccggaagcgggggccccgcgccgtccgcgcggcgcctccggaacctccgggtggacgcgggggtcgagtgtaggcgaggtcgggggaggggcgggggctcgttgtcgcgccgcgcccgctgaatcttttcccgacaggtcccaccccccgcgcgatgcccccccgggccgctggccatgtcgtccgggggaggccccgcggaccacgtcgtccggcgagacgccacgagccgcaggatggactcgtagtggagcgacggcgccccgttgcggagcagatccgcggccagggcggccccgaaccaagccttgatgctcaactccatccgggcccagctgggggcggtcatcgtggggaacaggggggcggtggtccgacagaaacgctcctggctgtccaccgcggcccgcagatactcgttgttcaggctgtcggtggcccagacgccgtacccggtgagggtcgcgttgatgatatactgggcgtggtgatggacgatcgacagaacctccaccgtggatacgacggtatccacggtcccgtacgtaccgccgctccgcttgccggtctgccacaggttggctaggcgcgtcaggtggcccaggacgtcgctgaccgccgccctgagcgccatgcactgcatggagccggtcgtgccgctgggaccccggtccagatggcgcgcgaacgtttccgcgggcgcctccgggctgccgccgagcgggaggaaccggcgattggagggactcagccggtgacatacgtgcttgtccgtcgtccacagcatccaggacgcccaccggtacagcacggagacgtaggccaggagctcgttgagccgcagtgcggtgtcggtgctggggcggcttgggtccgccgggcgcataaagaacatgtactgctgaatccgatggagggcgtcgcgcaggccggccacggtggcggcgtacttggccgccacggccccgctcttgaacggggtgcgcgccagcagctttggcgccagggtgggccgcagcagcacgtgaaggctggggtcgcagtcgcccacggggtcctcggggacgtccaggccgctgggcaccaccgtctgcaggtacttccagtactgcgtgaggatggcgcggctcaactggccgccgggcagctccacctcgcccagcgcctgggtggcggccgaagcgtagtgccggatgtactcgtagtgcgggtcgctggcgagcccgtccacgatcaaactctcgggaaccgtgttgtgttgccgcgcggccaaccggacgctgcgatcggtgcaggtcagaaacgccggctgcgcgtcgtcggagcgctgccgcaaggcgcccacggccgcgctaaggagcccctccggggtggggagcagacacccgccgaagatgcgccgctcgggaacgcccgcgttgtcgccgcggatcaggttggcaggcgtcaggcaccgcgccagccgcagggagctcgcgccgcgcgtccggcgctgcatggtgacgcccgttcggtcgggacccgccggtcggagttatgccgcgtccagggccatcggggcgctttttatcgggaggagcttatgggcgtggcgggcctcccagcccggtcgcgcgcctccccgacacgtgcgcccgcagggcggcggccccctcgtctcccatcagcagtttcctaaactgggacatgatgtccaccacgcggacccgcgggcccaacacggacccgccgcttacgggggcgggggggaagggctccaggtccttgagaagaaaggcggggtctgccgtcccggacacgggggcccggggcgctgaggaggcggggcgcagatccacgtgctccgcggccgcgcggacgtccgcccagaacttggcgggggtggtgcgcgcgtacaggggctgggtcgctcggaggacgcacgcgtagcgcaggggggtgtacgtgcccacctcgggggccgtgaatcccccgtcaaacgcggccagtgtcacgcacgccaccacggtgtcggcaaagcccagcagccgctgcaggacgagcccggcggccagaatggcgcgcgtggccgccgcgtcgtcccggcgccggtgcgcgtccccgcacgcccgggcgtactttaaggtcacggtcgccagggccgtgtgcagcgcgtacaccgcagcgcccagcacggcgttgagcccgctgttggcgagcagccggcgcgctgcggtgtcgcccagcgcctcgtgctcggcccccacgaccgcggggcttcccaggggcagggcgcgaaacagctcctcccgcgccacgtccgcaaaggcggggtggtgcacgtgcgggtgcaggcgcgcccccacgaccaccgagagccactggaccgtctgctccgccatcaccgccagcacatccagcacgcgccccaggaaggcggcctcccgcgtcaaaacgcaccggacggcgtcgggattgaagcgggcgagcagggccccggtggccaggtacgtcatgcggccggcatagcgggcggccacgcgacagtcgcggtccagcagcgcgcgcaccccgggccagtacagcagggaccccagcgagctgcggaacaccgcggcgtcggggccggattggggggacactaacccccccgcgctcagtaacggcacggccgcggccccgacgggacgcaacgccgtgaggctcgcgaactgccgcctcagctcggccgccctgtcgtccaggtcagacccgcgcgcctccgcgtgaaggcgcgtcccgcacacccacccgttgatggccagccgcacgacggcatccgccaaaaagctcatcgcctgggcggggctggtttttgttcgacgatccgtcaggtcaagaatcccatcgcccgtgatataccaggccaacgcctcgccctgctgcagggtttggcggaaaaacaccgcggggttgtcgggggaggcgaagtgcatgacccccacgcgcgataacccgaacgcgctatccggacacgggtaaaacccggccggatgccccagggctagggcggagcgcacggactcgtcccacacggcaacctgaggggccagtcgatccaacgggaatgccgcccggagctccgggcccggcacgcgtccctccagaacctccaccttgggcggggaacgggccccgccgccgtcctccggcccgacggcttccgggtagtcgtcctcctcgtactgcagctcctctaggaacagcggcgacggcgccacccgcgaaccgccgacccgccccaaaatagcccgcgcgtcgacgggacccaggtatcccccctgccgggcctgcggaggaccgcggggaacctcatcatcatcgtccaggcgaccgcgcaccgactggctacgggccgcatcgggcccggggcgctgccgggacgctcggcgatgggatgtgggcggggcttccgacgcgcgccgtcgtcgggctcgcgggccttcccgtcgacggcgcacgggcggctcgtcgcccgccatctcctccagagcctctagctcgctgtcgtcatccccgcggaacaccgcacgcaggtaccccatgaaccccaccccatcgcccgctggctcgtccgccacgggcgaggcgcgggggcgggtggatgcgcgcctcctgcgccccgcgggttcgcgagccgacatggtggcgatagacgcgggttatcggatgtccgctaccccccaaaaaagaaaaagaccccacagcgcggatggaggccggggtaggtgccgccggaccccctcgcgatgggaatggacgggagcgacggggccggcgcaaaaaaacgcagtatctcccgcgaaggctacccgccgccccagcccccggccaaatgcggaaacggtcccgcgctctcgcctttatacgcgggccgccctgcgacacaatcacccgtccgtggtttcgaatctacacgacaggcccgcagacgcggctaacacacacgccggcaacccagaccccagtgggttggttgcgcggtcccgtctcctggctagttctttcccccaccaccaaataatcagacgacaaccgcaggttttgtaatgtatgtgctcgtgtttattgtggatacgaaccggtgacgggaggggaaaacccagacgggggatgcgggtccggtcgcgccccctacccaccgtactcgtcaattccaagggcatcggtaaacatctgctcaaactcgaagtcggccatatccagagcgccgtagggggcggagtcgtggggggtaaatcccggccccggggaatccccgtcccccaacatgtccagatcgaaatcgtctagcgcgtcggcatgcgccatcgccacgtcctcgccgtctaagtggagctcgtcccccaggctgacatcggtcgggggggccgtcgacagtctgcgcgtgtgtcccgcggggagaaaggacaggcgcggagccgccagccccgcctcttcgggggcgtcgtcgtccgggagatcgagcaggccctcgatggtagacccgtaattgtttttcgtacgcgcgcggctgtacgcgtgttcccgcatgaccgcctcggagggcgaggtcgtgaagctggaatacgagtccaacttcgcccgaatcaacaccataaagtacccagaggcgcgggcctggttgccatgcagggtgggaggggtcgtcaacggcgcccctggctcctccgtagccgcgctgcgcaccagcgggaggttaaggtgctcgcgaatgtggtttagctcccgcagccggcgggcctcgattggcactccccggacggtgagcgctccgttgacgaacatgaagggctggaacagacccgccaactgacgccagctctccaggtcgcaacagaggcagtcaaacaggtcgggccgcatcatctgctcggcgtacgcggcccataggatctcgcgggtcaaaaatagatacaaatgcaaaaacaaaacacgcgccagacgagcggtctctcggtagtacctgtccgcgatcgtggcgcgcagcatttctcccaggtcgcgatcgcgtccgcgcatgtgcgcctggcggtgcagctgccggacgctggcgcgcaggtaccggtacagggccgagcagaagttggccaacacggttcgatagctctcctcccgcgcccgtagctcggcgtggaagaaacgagagagcgcttcgtagtagagcccgaggccgtcgcgggtggccggaagcgtagggaaggccacgtcgccgtgggcgcgaatgtcgatttgggtgcgttcggggacgtacgcgtccccccattccaccacatcgctgggcagcgttgataggaatttacactcccggtacaggtcggcgttggtcggtagcgccgaaaacagatcctcgttccaggtatcgagcatggtacatagcgcggggcccgcgctaaagcccaagtcgtcgaggagacggttaaagagggcggcgggggggacgggcatgggtggggagggcatgagctgggcctggctcaggcgccccgttgcgtacagcgggggggccgccggggtgtttttgggacccccggccgggcgggggggcggtggcgaagcgccgtccgcgttcatgtcggcaaacagctcgtcgaccaagaggtccattgggtggggttgatacgggaaagacgatatcgggcttttgatgcgatcgtccccgcccgcccagagagtgtgggacgcccgacggcgcgggaagagaaaacccccaaacgcgttagaggaccggacggaccttatggggggaagtgggcagcgggaaccccgtccgttcccgaggaatgacagcccgtggtcgccaccacgcatttaagcaacccgcacgggccgccccgtacctcgtgacttccccccacattggctcctgtcacgtgaaggcgaaccgagggcggctgtccaacccaccccccgccacccagtcccggtccccgtcggattgggaaacaaaggcacgcaacgccaacaccgaatgaacccctgttggtgctttattgtctgggtacggaagttttcactcgacgggccgtctggggcgagaagcggagcgggctggggctcgaggtcgctcggtggggcgcgacgccgcagaacgccctcgagtcgccgtggccgcgtcgacgtcctgcaccacgtctggattcaccaactcgttggcgcgctgaagcaggtttttgccctcgcagaccgtcacgcggatggtggtgatgccaaggagttcgttgaggtcttcgtctgtgcgcggacgcgacatgtcccagagctggaccgccgccatccgggcatgcatggccgccaggcgcccgaccgcggcgcagaagacgcgcttgttaaagccggccacccggggggtccatggcgcgtcggggtttgggggggcggtgctaaagtgcagctttctggccagcccctgcgcgggtgtcttggatcgggttggcgccgtcgacgcgggggcgtctgggagtgcggcggattctggctgggccgatttcctgccgcgggtggtctccgccgccggggccgcgggggccttagtcgccacccgctgggttcggggggcccggggggcggtggtgggtgtgcgtccggcccctccggacccagcgggtggcggaggcgcccgcgcaggccccgggccggacaaaaccgccccggaaacgggacgccgcgtccgggggacctccgggtgttcgtcgtcttcggatgacgagcccccgtagagggcataatccgactcgtcgtactggacgaaacggacctcgcccctctggcgcgagcgtgtctgtagggcgccacggcgggaggtgtcaggcggactatcgggactcgccatacctgaagacggggtgtagtacagatcctcgtactcatcgcgcggaacctcccgcggacccgacttcacggagcggcgagaggtcatggttccacgaacacgctagggtcggatgcgcggacaattaggcctgggttcggacggcgggggtggtgcaggtgtggagaggtcgagcgataggggcggcccgggagagaagagagggtccgcaaaacccactggggatgcgtgagtggccctctgtgggcggtgggggagagtcttataggaagtgcatataaccacaacccatgggtctaaccaatccccaggggccaagaaacagacacgccccaaacggtctcggtttccgcgaggaaggggaagtcctgggacaccctccacccccacccctcaccccacacagggcgggttcaggcgtgcccggcagccagtagcctctggcagatctgacagacgtgtgcgataatacacacgcccatcgaggccatgcctacataaaagggcaccagggcccccggggcagacatttggccagtgttttgggtctcgcaccgcgcgcccccgatcccatcgcgcccgccctcctcgccgggcggctccccgcgcgggcccgcgtctcccgccgctaaggcgacgagcaagacaaacaacaggcccgcccgacagacccttctgggggggcccatcgtccctaacaggaagatgagtcagtggggatccggggcgatccttgtccagccggacagcttgggtcgggggtacgatggcgactggcacacggccgtcgctactcgcgggggcggagtcgtgcaactgaacctggtcaacaggcgcgcggtggcttttatgccgaaggttagcggggactccggatgggccgtcgggcgcgtctctctggacctgcgaatggctatgccggctgacttttgcgcgattattcacgcccccgcgctagccagccccgggcaccacgtaatactgggtcttatcgactcggggtaccgcggaaccgttatggccgtggtcgtagcgcctaaaaggacgcgggaatttgcccccgggaccctgcgggtcgacgtgacgttcctggacatcctggcgacccccccggccctcaccgagccgatttccctgcggcagttcccgcaactggcgcccccccctccaaccggggccgggatacgcgaagatccttggttggagggggcgctcggggccccaagcgtgactacggccctaccggcgcgacgccgagggcggtccctcgtctatgccggcgagctgacgccggttcagacggaacacggggacggcgtacgagaagccatcgccttccttccaaaacgcgaggaggatgccggtttcgacattgtcgtccgtcgcccggtcaccgtcccggcaaacggcaccacggtcgtgcagccatccctccgcatgctccacgcggacgccgggcccgcggcctgctatgtgttggggcggtcgtcgctcaacgcccgcggcctcctggtcgttcctacgcgctggctccccgggcacgtatgtgcgtttgttgtttacaaccttacgggggttcctgtgaccctcgaggccggcgccaaggtcgcccagctcctggttgcgggggcggacgctcttccttggatccccccggacaactttcacgggaccaaagcgcttcgaaactaccccaggggtgttccggactcaaccgccgaacccaggaacccgccgctcctggtgtttacgaacgagtttgacgcggaggcccccccgagcgagcgcgggaccgggggttttggttctaccggtatttagcccacagctttgggttcgttccgggcaataaaaaacgtttgtatcgcatctttcctgtgtgtagttgtttatgttggatgcctgtgggtctatcacacccgcccctccatcccacaaacacaaaacacacgggttggatgaaaacacgcatttattgacccaaaacacacggagctgctcgagatgggccagggcgaggtgcggttggggaggctgtaggtctgggaacggacacgcggggacacgattccggtttggggtccgggagggcgtcgccgtttcgggcggcaggcgccagcgtaacctccgggggcggcgtgtgggggtgccccaaggagggcgcctcggtcaccccaatcccccccgaccgggttcccccggcaaccccgaaggcggagaggccaagggcccgttcggcgatggccacatcctccatgaccacgtcactctcggccatgctccgaatagcctgggagacgagcacatccgcggacttgtcagccgcccccacggacatgtacatctgcaggatggtggccatacacgtgtccgccaggcgccgcatcttgtcctgatgggccgccacggccccgtcgatcgtgggggcctcgaacccggggtggtggcgcgccagtcgttctaggttcaccatgcaggcgtggtacgtgcgggccaaggcgcgggccttcacgaggcgtcgggtgtcgtccagggaccccagggcgtcatcgagcgtgatgggggcgggaagtagcgcgttaacgaccgccagggcctcctgcagccgcggctccgcctccgagggcggaacggccgcgcggatcatctcatattgttcctcggggcgcgctccccagccacatatagccccgagaagagaagccatcgcgggcgggtactggcccttgggcgcgcggacgcaatggggcaggaagacgggaaccgcggggagaggcgggcggccgggactcccgtggaggtgaccgcgctttatgcgaccgacgggtgcgttattacctcttcgatcgccctcctcacaaactctctactgggggccgagccggtttatatattcagctacgacgcatacacgcacgatggccgtgccgacgggcccacggagcaagacaggttcgaagagagtcgggcgctctaccaagcgtcgggcgggctaaatggcgactccttccgagtaaccttttgtttattggggacggaagtgggtgggacccaccaggcccgcgggcgaacccgacccatgttcgtctgtcgcttcgagcgagcggacgacgtcgccgcgctacaggacgccctggcgcacgggaccccgctacaaccggaccacatcgccgccaccctggacgcggaggccacgttcgcgctgcatgcgaacatgatcctggctctcaccgtggccatcaacaacgccagcccccgcaccggacgcgacgccgccgcggcgcagtatgatcagggcgcgtccctacgctcgctcgtggggcgcacgtccctgggacaacgcggccttaccacgctatacgtccaccacgaggtgcgcgtgcttgccgcgtaccgcagggcgtattatggaagcgcgcagagtcccttctggtttcttagcaaattcgggccggacgaaaaaagcctggtgctcaccactcggtactacctgcttcaggcccagcgtctggggggcgcgggggccacgtacgacctgcaggccatcaaggacatctgcgccacctacgcgattccccacgccccccgccccgacaccgtcagcgctgcgtccctgacctcgtttgccgccatcacgcggttctgttgcacgagccagtacgcccgcggggccgcggcggccgggtttccgctttacgtggagcgccgtattgcggccgacgtccgcgagaccagtgcgctggagaagttcataacccacgatcgcagttgcctgcgcgtgtccgaccgtgaattcattacgtacatctacctggcccattttgagtgtttcagccccccgcgcctagccacgcatcttcgggccgtgacgacccacgaccccaaccccgcggccagcacggagcagccctcgcccctgggcagggaggccgtggaacaatttttttgtcacgtgcgcgcccaactgaatatcggggagtacgtcaaacacaacgtgaccccccgggagaccgtcctggatggcgatacggccaaggcctacctgcgcgctcgcacgtacgcgcccggggccctgacgcccgcccccgcgtattgcggggccgtggactccgccaccaaaatgatggggcgtttggcggacgccgaaaagctcctggtcccccgcgggtggcccgcgtttgcgcccgccagtcccggggaggacacggcgggcggcacgccgcccccacagacctgcggaattgtcaagcgcctcctgagactggccgccacggaacagcagggccccacacccccggcgatcgcggcgcttatccgtaatgcggcggtgcagactcccctgcccgtctaccggatatccatggtccccacgggacaggcatttgccgcgctggcctgggacgactgggcccgcataacgcgggacgctcgcctggccgaagcggtcgtgtccgccgaagcggcggcgcaccccgaccacggcgcgctgggcaggcggctcacggatcgcatccgcgcccagggccccgtgatgccccctggcggcctggatgccggggggcagatgtacgtgaatcgcaacgagatattcaacggcgcgctggcaatcacaaacatcatcctggatctcgacatcgccctgaaggagcccgtcccctttcgccggctccacgaggccctgggccactttaggcgcggggctctggctgcggttcagctcctgtttcccgcggcccgcgtggaccccgacgcatatccctgttattttttcaaaagcgcatgtcggcccggcccggcgtccgtgggttccggcagcggactcggcaacgacgacgacggggactggtttccctgctacgacgacgccggtgatgaggagtgggcggaggacccgggcgccatggacacatcccacgatcccccggacgacgaggttgcctactttgacctgtgccacgaagtcggccccacggcggaacctcgcgaaacggattcgcccgtgtgttcctgcaccgacaagatcggactgcgggtgtgcatgcccgtccccgccccgtacgtcgtccacggttctctaacgatgcggggggtggcacgggtcatccagcaggcggtgctgttggaccgagattttgtggaggccatcgggagctacgtaaaaaacttcctgttgatcgatacgggggtgtacgcccacggccacagcctgcgcttgccgtattttgccaaaatcgcccccgacgggcctgcgtgcggaaggctgctgccagtgtttgtgatcccccccgcctgcaaagacgttccggcgtttgtcgccgcgcacgccgacccgcggcgcttccattttcacgccccgcccacctatctcgcttccccccgggagatccgtgtcctgcacagcctgggtggggactatgtgagcttctttgaaaggaaggcgtcccgcaacgcgctggaacactttgggcgacgcgagaccctgacggaggtcctgggtcggtacaacgtacagccggatgcgggggggaccgtcgaggggttcgcatcggaactgctggggcggatagtcgcgtgcatcgaaacccactttcccgaacacgccggcgaatatcaggccgtatccgtccggcgggccgtcagtaaggacgactgggtcctcctacagctagtccccgttcgcggtaccctgcagcaaagcctgtcgtgtctgcgctttaagcacggccgggcgagtcgcgccacggcgcggacattcgtcgcgctgagcgtcggggccaacaaccgcctgtgcgtgtccttgtgtcagcagtgctttgccgccaaatgcgacagcaaccgcctgcacacgctgtttaccattgacgccggcacgccatgctcgccgtccgttccctgcagcacctctcaaccgtcgtcttgataacggcgtacggcctcgtgctcgtgtggtacaccgtcttcggtgccagtccgctgcaccgatgtatttacgcggtacgccccaccggcaccaacaacgacaccgccctcgtgtggatgaaaatgaaccagaccctattgtttctgggggccccgacgcacccccccaacgggggctggcgcaaccacgcccatatctgctacgccaatcttatcgcgggtagggtcgtgcccttccaggtcccacctgacgccatgaatcgtcggatcatgaacgtccacgaggcagttaactgtctggagaccctatggtacacacgggtgcgtctggtggtcgtagggtggttcctgtatctggcgttcgtcgccctccaccaacgccgatgtatgtttggcgtcgtgagtcccgcccacaagatggtggccccggccacctacctcttgaactacgcaggccgcatcgtatcgagcgtgttcctgcagtacccctacacgaaaattacccgcctgctctgcgagctgtcggtccagcggcaaaacctggttcagttgtttgagacggacccggtcaccttcttgtaccaccgccccgccatcggggtcatcgtaggctgcgagttgatgctacgctttgtggccgtgggtctcatcgtcggcaccgctttcatatcccggggggcatgtgcgatcacataccccctgtttctgaccatcaccacctggtgttttgtctccaccatcggcctgacagagctgtattgtattctgcggcggggcccggcccccaagaacgcagacaaggccgccgccccggggcgatccaaggggctgtcgggcgtctgcgggcgctgctgttccatcatcctctcgggcatcgcagtgcgattgtgttatatcgccgtggtggccggggtggtgctcgtggcgcttcactacgagcaggagatccagaggcgcctgtttgatgtatgacgtcacatccaggccggcggaaaccgtaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcgtatccacccccgcccgggtccccgcggaagcggaacggggtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtcatggcgactgacattgatatgctaattgacctcggcctggacctctccgacagcgatctggacgaggacccccccgagccggcggagagccgccgcgacgacctggaatcggacagcagcggggagtgttcctcgtcggacgaggacatggaagacccccacggagaggacggaccggagccgatactcgacgccgctcgcccggcggtccgcccgtctcgtccagaagaccccggcgtacccagcacccagacgcctcgtccgacggagcggcagggccccaacgatcctcaaccagcgccccacagtgtgtggtcgcgcctcggggcccggcgaccgtcttgctcccccgagcagcacgggggcaaggtggcccgcctccaacccccaccgaccaaagcccagcctgcccgcggcggacgccgtgggcgtcgcaggggtcggggtcgcggtggtcccggggctgccgatggtttgtcggacccccgccggcgtgcccccagaaccaatcgcaaccctgggggaccccgccccggggcggggtggacggacggccccggcgccccccatggcgaggcgtggcgcggcagtgagcagcccgacccacccggaggccagcggacacggggcgtgcgccaagcaccccccccgctaatgacgctggcgattgcccccccgcccgcggacccccgcgccccggccccggagcgaaaggcgcccgccgccgacaccatcgacgccaccacgcggttggtcctgcgctccatctccgagcgcgcggcggtcgaccgcatcagcgagagctttggccgcagcgcacaggtcatgcacgacccctttggggggcagccgtttcccgccgcgaatagcccctgggccccggtgctggcgggccaaggagggccctttgacgccgagaccagacgggtctcctgggaaaccttggtcgcccacggcccgagcctctatcgcacttttgccggcaatcctcgggccgcatcgaccgccaaggccatgcgcgactgcgtgctgcgccaagaaaatttcatcgaggcgctggcctccgccgacgagacgctggcgtggtgcaagatgtgcatccaccacaacctgccgctgcgcccccaggaccccattatcgggacgaccgcggctgtgctggataacctcgccacgcgcctgcggccctttctccagtgctacctgaaggcgcgaggcctgtgcggcctggacgaactgtgttcgcggcggcgtctggcggacattaaggacattgcatccttcgtgtttgtcattctggccaggctcgccaaccgcgtcgagcgtggcgtcgcggagatcgactacgcgacccttggtgtcggggtcggagagaagatgcatttctacctccccggggcctgcatggcgggcctgatcgaaatcctagacacgcaccgccaggagtgttcgagtcgtgtctgcgagttgacggccagtcacatcgtcgcccccccgtacgtgcacggcaaatatttttattgcaactccctgttttaggtacaataaaaacaaaacatttcaaacaaatcgcccctcgtgttgtccttctttgctcatggccggcggggcgtgggtcacggcagatggcgggggtgggcccggcgtacggcctgggtgggcggagggaactaacccaacgtataaatccgtccccgttccaaggccggtgtcatagtgcccttaggagcttcccgcccgggcgcatccccccttttgcactatgacagcgacccccctcaccaacctgttcttacgggccccggacataacccacgtggcccccccttactgcctcaacgccacctggcaggccgaaacggccatgcacaccagcaaaacggactccgcttgcgtggccgtgcggagttacctggtccgcgcctcctgtgagaccagcggcacaatccactgctttttctttgcggtatacaaggacacccaccacacccctccgctgattaccgagctccgcaactttgcggacctggttaaccacccgccggtcctacgcgaactggaggataagcgcggggtgcggctgcggtgtgcgcggccgtttagcgtcgggacgattaaggacgtctctgggtccggcgcgtcctcggcgggagagtacacgataaacgggatcgtgtaccactgccactgtcggtatccgttctcaaaaacatgctggatgggggcctccgcggccctacagcacctgcgctccatcagctccagcggcatggccgcccgcgcggcagagcatcgacgcgtcaagattaaaattaaggcgtgatctccaacccccccatgaatgtgtgtaaccccccccaaaaaaataaagagccgtaacccaaccaaaccaggcgtggtgtgagtttgtggacccaaagccctcagagacaacgcgacaggccagtatggaccgtgatacttttatttattaactcacaggggcgcttaccgccacaggaataccagaataatgaccaccacaatcgcgaccaccccaaatacagcatggcgccacaccacgccacaacagccctgtcgccggtatggggcatgatcagacgagccgcgcgccgcgcgttgggccctgtacagctcgcgcgaattgaccctaggaggccgccacgcgcccgagttttgcgttcgtcgctggtcgtcgggcgccaaagccccggacggctgttcggtcgaacgaacggccacgacagtggcataggttggggggtggtccgacatagcctcggcgtacgtcgggaggcccgacaagaggtcccttgtgatgtcgggtggggccacaagcctggtttccggaagaaacaggggggttgccaataacccgccagggccaaaactccggcgctgcgcacgtcgttcggcgcggcgccgggcgcgccgagcggctcgctgggcggcttggcgtgagcggccccgctccgacgcctcgccctctccggaggaggttggcggaattggcacggacaacaggggcccagcagagtacggtggaggtgggtccgtgggggtgtccagatcaataacgacaaacggcccctcgttcctaccagacaagctatcgtaggggggcgggggatcagcaaacgcgttccccgcgctccataaacccgcgtcgggttgcgccgcctccgaagccatggatgcgccccaaagccacgactcccgcgcgctaggtccttggggtaatggaaaaggccctactccccatccaagccagccaagttaacgggctacgccttcgggaatgggactggcaccccggcggattttgttgggctggcatgcgtcgcccaaccgagggccgcgtccacgggacgcgccttttataaccccgggggtcattcccaacgatcacatgcaatctaactggctcccctctccccccctctcccctctccccccctctcccctctccccccctctcccctctccccccctctcccctctccccccctctcccctctccccccctctcccctctccccccctctcccctctccccccctctcccctctccccccctctcccctctccccccctctcccctctcccctctgctctttccccgtgacacccgacgctgggggcgtggctgccgggaggggccgcggatgggcgggcctacttggtttcccgccccccccccccccccccgaaccgccccgccggctttgcccccctttgatcccctgctacccccaacccgtgctggtggtgcgggttggggggggatgtgggcgggggtgcgcgggaggtgtcggtggtggtggtggtggtggtagtaggaatggtggtgagggggggggggcgctggttggtcaaaaaagggagggacgggggccggcagaccgacggcgacaacgctccccggcggccgggtcgcggctcttacgagcggcccggcccgcgctcccaccccccgggccgtgtccttgctttccccccgtctcccccccccccgccttctcctcctcctcctcgtttttccaaaccccgcccacccggcccggcccggcccggcccggcccggccaccgccgcccacccacccacctcgggatacccagccccggtcccccgttccccgggggccgttatctccagcgccccgtccggcgcgccgccccccgccgctaaaccccatcccgcccccgggaccccacatataagcccccagccacacgcaagaacagacacgcagaacggctgtgtttatttaaataaaccaatgtcggaataaacaaacacaaacacccgcgacggggggacggaggggacggagggagggggtgacgggggacgggaacagacacaaaaacaaccacaaaaaacaaccacccaccgacacccccaccccagtctcctcgccttctcccacccaccccacgcccccactgagcccggtcgatcgacgagcacccccgcccacgcccccgcccctgccccggcgacccccggcccgcacgatcccgacaacaataacaaccccaacggaaagcggcggggtgttgggggaggcgaggaacaaccgaggggaacgggggatggaaggacgggaagtggaagtcctgatacccatcctacacccccctgccttccaccctccggccccccgcgagtccacccgccggccggctaccgagaccgaacacggcggccgccgcagccgccgcagccgccgccgacaccgcagagccggcgcgcgcactcacaagcggcagaggcagaaaggcccagagtcattgtttatgtggccgcgggccagcagacggcccgcgacaccccccccccgcccgtgtgggtatccggccccccgccccgcgccggtccattaagggcgcgcgtgcccgcgagatatcaatccgttaagtgctctgcagacaggggcaccgcgcccggaaatccattaggccgcagacgaggaaaataaaattacatcacctacccacgtggtgctgtggcctgtttttgctgcgtcatctcagcctttataaaagcgggggcgcggccgtgccgatcgcgggtggtgcgaaagactttccgggcgcgtccgggtgccgcggctctccgggcccccctgcagccggggcggccaaggggcgtcggcgacatcctccccctaagcgccggccggccgctggtctgttttttcgttttccccgtttcgggggtggtgggggttgcggtttctgtttctttaacccgtctggggtgtttttcgttccgtcgccggaatgtttcgttcgtctgtcccctcacggggcgaaggccgcgtacggcccgggacgaggggcccccgaccgcggcggtccgggccccgtccggacccgctcgccggcacgcgacgcgaaaaaggccccccggaggcttttccgggttcccggcccggggcctgagatgaacactcggggttaccgccaacggccggcccccgtggcggcccggcccggggccccggcggacccaaggggccccggcccggggccccacaacggcccggcgcatgcgctgtggtttttttttcctcggtgttctgccgggctccatcgcctttcctgttctcgcttctccccccccccttcttcacccccagtaccctcctccctcccttcctcccccgttatcccactcgtcgagggcgccccggtgtcgttcaacaaagacgccgcgtttccaggtaggttagacacctgcttctccccaatagaggggggggacccaaacgacagggggcgccccagaggctaaggtcggccacgccactcgcgggtgggctcgtgttacagcacaccagcccgttcttttccccccctcccacccttagtcagactctgttacttacccgtccgaccaccaactgcccccttatctaagggccggctggaagaccgccagggggtcggccggtgtcgctgtaaccccccacgccaatgacccacgtactccaagaaggcatgtgtcccaccccgcctgtgtttttgtgcctggctctctatgcttgggtcttactgcctggggggggggagtgcgggggagggggggtgtggaaggaaatgcacggcgcgtgtgtaccccccctaaagttgttcctaaagcgaggatacggaggagtggcgggtgccgggggaccggggtgatctctggcacgcgggggtgggaagggtcgggggagggggggatggagtaccggcccacctggccgcgcgggtgcgcgtgcctttgcacaccaaccccacgtcccccggcggtctctaagaagcaccgccccccctccttcataccaccgagcatgcctgggtgtgggttggtaaccaacacgcccatcccctcgtctcctgtgattctctggctgcaccgcattcttgttttctaactatgttcctgtttctgtctcccccccccccacccctccgccccaccccccaacacccacgtctgtggtgtggccgacccccttttgggcgccccgtcccgccccgccacccctcccatcctttgttgccctatagtgtagttaaccccccccgccctttgtggcggccagaggccaggtcagtccgggcgggcaggcgctcgcggaaacttaacacccacacccaacccactgtggttctggctccatgccagtggcaggatgctttcggggatcggtggtcaggcagcccgggccgcggctctgtggttaacaccagagcctgcccaacatggcacccccactcccacgcacccccactcccacgcacccccactcccacgcacccccactcccacgcacccccactcccacgcacccccactcccacgcacccccactcccacgcacccccactcccacgcacccccactcccacgcatccccgcgatacatccaacacagacagggaaaagatacaaaagtaaacctttatttcccaacagacagcaaaaatcccctgagtttttttttattagggccaacacaaaagacccgctggtgtgtggtgcccgtgtctttcacttttcccctccccgacacggattggctggtgtagtgggcgcggccagagaccacccagcgcccgacccccccctccccacaaacacggggggcgtcccttattgttttccctcgtcccgggtcgacgccccctgctccccggaccacgggtgccgagaccgcaggctgcggaagtccagggcgcccactagggtgccctggtcgaacagcatgttccccacgggggtcatccagaggctgttccactccgacgcgggggccgtcgggtactcggggggcatcacgtggttacccgcggtctcggggagcagggtgcggcggctccagccggggaccgcggcccgcagccgggtcgccatgtttcccgtctggtccaccaggaccacgtacgccccgatgttccccgtctccatgtccaggatgggcaggcagtcccccgtgatagtcttgttcacgtaaggcgacagggcgaccacgctagagacccccgagatgggcaggtagcgcgtgaggccgcccgcggggacggccccggaagtctccgcgtggcgcgtcttccgggcacacttcctcggcccccgcggcccagaagcagcgcgggggccgagggaggtttcctcttgtctccctcccagggcaccgacggccccgcccgaggaggcggaagcggaggaggacgcggccccggcggcggaagaggcggcccccgcgggggtcggggccgaggaggaagaggcagaggaggaagaggcggaggccgccgaggacgtcaggggggtcccgggcccaccctggccgcgcccccccggccctgagtcggagggggggtgcgtcgccgccctcttggcccctgccggcgcgagggggggacgcgtggactggggggaggggttttcctggcccgacccgcgcctcttcctcggacgcaccgccgcctcctgctcgacagaggcggcggaggggagcggggcggcgccggagggggcggcgccgcgggagggcccgtgcccaccctccacgcccggcccccccgagccgcgcgccaccgtcgcacgcgcccggcacagactctgttcttggttcgcggcctgagccagggacgagtgcgactggggcacacggcgcgcgtccgcggggcgggcggccggctccgccccgggggccggggcgcgggggccgggccccggaggcggcgctcgcacgcacggggccacggccgcgcgggggcgcgcgggtcccgacgcggccgcggacgcggggggcccggggcggggggcggagcctggcatgggcgccgcggggggcctgtggggagaggccgggggggagtcgctgatcactatggggtctctgttgtttgcaaggggggcgggtctgttgacaagggggcccgtccggcccctcggccgccccgcctccgcttcaacaaccccaaccccaaccccaacccccccggaggggccagacgccccccgcggcgccgcggctcgcgactggcgggagccgccgccgccgctgctgttggtggtggtgttggtgttactgctgccgtgtggcccgatgggcgccgaggggggcgctgtccgagccgcggccggctggggggctgcgtgagacgccccgcccgtcacggggggcgcggcggcgcctctgcgtgggggggcgcggggcgtccggcggggggcgggcggtacgtagtctgctgcaagagacaacggggggcgcgatcaggttacgccccctccccggcccgccctttcctcgcccgcccgcctattcctccctccccccccctcctcctcctcctcccccagggtccttgccgccccccgcctcaccgtcgtccaggtcgtcgtcatcctcgtccgtggtgggctccgggtgggtgggcgacagggccctcaccgtgtgcccccccagggtcaggtaccgcggggcgaaccgctgattgcccgtccagataaagtccacggccgtgcccgccctgacggcctcctcggcctccatgcgggtctgggggtcgttcacgatcgggatggtgctgaacgacccgctgggcgtcacgcccactatcaggtacaccagcttggcgttgcacagcgggcaggtgttgcgcaattgcatccaggttttcatgcacgggatgcagaagcggtgcatgcacgggaaggtgtcgcagcgcaggtggggcgcgatctcatccgtgcacacggcgcacacgtcgccctcgtcgctccccccgtcctctcgagggggggcgcccccgcaactgccggggtcttcctcgcggggggggctcccccccgagaccgcccccccatccacgccctgcggccccagcagccccgtctcgaacagttccgtgtccgtgctgtccgcctcggaggcggagtcgtcgtcatggtggtcggcgtccccccgcccccccacttcggtctccgcctcagagtcgctgctgtccggcaggtctcggtcgcagggaaacacccagacatccggggcgggctaaggggaaaaaaggggggcgggtaagaatggggggggatttcccgcgtcaatcagcacccacgagttccccctctcccccccccgcctcacaaagtcctgcccccctgctggcctcggaagaggggggagaaaggggtctgcaaccaaaggtggtctgggtccgtcctttggatcccgacccctcttcttccctcttctcccgccctccagacgcaccggagtcgggggtcccacggcgtcccccaaatatggcgggcggctcctccccacccccctagatgcgtgtgagtaaggggggcctgcgtatgagtcagtggggaccacgcccccaacacggcgaccccggtccttgtgtgtttgttgtgggggcgtgtctctgtgtatgagtcagggggtcccacggcgaccccgggccctgcgtctgagtcaaaggggccatgtgtatgtgttgggggtctgtatatataaagtcagggggtcacatggcgacccccaacagggcgaccccggtccctgtatatatagggtcagggggttccgcaccccctaacatggcgcccccggtccctgtatatatagtgtcacggggttccacgccccctaacatggcgccccaacatggcgcccggctcccgtgtatgagtgggggtcccccaacatggcggccggttccagtgtaagggtcgggggtcccccaacatggcgccccccaatatggcgccccccaatatggcgccccagacatggcgcccggcccctcacctcgcgctgggggcggccctcaggccggcgggtactcgctccggggcggggctccatgggggtcgtatgcggctggagggtcgcggacggagggtccctgggggtcgcaacgtaggcggggcttctgtggtgatgcggagagggggcggcccgagtctgcctggctgctgcgtctcgctccgagtgccgaggtgcaaatgcgaccagactgtcgggccagggctaacttataccccacgcctttcccctccccaaaggggcggcagtgacgattcccccaatggccgcgcgtcccaggggaggcaggcccaccgcggggcggccccgtccccggggaccaacccggcgcccccaaagaatatcattagcatgcacggcccggcccccgatttgggggcccaacccggtgtcccccaaagaaccccattagcatgcccctcccgccgacgcaacaggggcttggcctgcgtcggtgccccggggcttcccgccttcccgaagaaactcattaccatacccggaaccccaggggaccaatgcgggttcattgagcgacccgcgggccaatgcgcgaggggccgtgtgttccgccaaaaaagcaattagcataacccggaaccccaggggagtggttacgcgcggcgcgggaggcggggaataccggggttgcccattaagggccgcgggaattgccggaagcgggaagggcggccggggccgcccattaatgagtttctaattaccataccgggaagcggaacaaggcctcttgcaagtttttaattaccataccgggaagtgggcggcccggcccattgggcggtaactcccgcccaatgggccgggccccgaagactcggcggacgctggttggccgggccccgccgcgctggcggccgccgattggccagtcccgcccccgaggcggcccgccctgtgagggcgggctggctccaagcgtatatatgcgcggctcctgccatcgtctctccggagagcggcttggtgcggagctcccgggagctccgcggaagacccaggccgcctcgggtgtaacgttagaccgagttcgccgggccggctccgcgggccagggcccgggcacgggcctcgggccccaggcacggcccgatgaccgcctcggcctccgccacccggcgccggaaccgagcccggtcggcccgctcgcgggcccacgagccgcggcgcgccaggcgggcggccgaggcccagaccaccaggtggcgcacccggacgtggggcgagaagcgcacccgcgcgggggtcgcgggggtcgcgggggtcgcgggggtcgcgggggtcgcggggggctccggcgccccctccccgcccgcgcgtcgcaggcgcaggcgcgccaggtgctccgcggtgacgcgcaggcggagggcgaggcgcggcggaaggcggaaggggcgcgagggggggtgggaggggtcagccccgccccccgggcccacgccgggcggtgggggccggggccggggggcggcggcggtgggccgggcctctggcgccggctcgggcggggggctgtccggccagtcgtcgtcatcgtcgtcgtcggacgcggactcgggaacgtggagccactggcgcagcagcagcgaacaagaaggcgggggcccaccggcggggggcggcggcggggcggccgcgggcgcgctcctgaccgcgggttccgagttgggcgtggaggttacctgggactgtgcggttgggacggcgcccgtgggcccgggcggccgggggcggcgggggccgcgatggcggcggcggcgggccatggagacagagagcgtgccggggtggtagagtttgacaggcaagcatgtgcgtgcagaggcgagtagtgcttgcctgtctaactcgctagtctcggccgcggggggcccgggctgcccgccgccaccgctttaaagggccgcgcgcgacccccggggggtgtgttttggggggggcccgttttcggcgtctggccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcctccccccgctcccgcggccccgccccccacgcccgccgcgcgcgcgcacgccgcccggaccgccgcccgccttttttgcgcgcgcgcgcgcccgcggggggcccgggctgccacaggtgaaaccaacagagcacggcgcactccgcacgtcacacgtcacgtcatccaccacacctgcccaacaacacaactcacagcgacaactcaccgcgcaacaactcctgttcctcatccacacgtcaccgcgcacctcccgctcctccagacgtaccccggcgcaacacaccgctcctgctacacaccaccgccccctccccagccccagccctccccagccccagccctccccggccccagccctccccggccccagccctccccggccccagccctccccggccccagccctccccggccccagccctccccggccccagccctccccggcgcgtcccgcgctccctcgggggggttcgggcatctctacctcagtgccgccaatctcaggtcagagatccaaaccctccgggggcgcccgcgcaccaccaccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgccccctcccgcccctcgaataaacaacgctactgcaaaacttaatcaggttgttgccgtttattgcgtcttcgggtctcacaagcgccccgccccgtcccggcccgttacagcaccccgtccccctcgaacgcgccgccgtcgtcttcgtcccaggcgccttcccagtccacaacttcccgccgcgggggcgtggccaagcccgcctccgcccccagcacctccacggcccccgccgccgccagcacggtgccgctgcggcccgtggccgaggcccagcgaatcccgggcggcgccggcggcagggcccccgggccgtcgtcgtcgccgcgcagcaccagcgggggggcgtcgtcgtcgggctccagcagggcgcgggcgcaaaagtccctccgcggcccgcgccaccgggccgggccggcgcgcaccgcctcgcgccccagcgccacgtacacgggccgcagcggcgcgcccaggccccagcgcgcgcaggcggcgtgcgagtgggcctcctcctcgcagaagtccggcgcgccgggcgccatggcgtcggtggtccccgaggccgccgcccggccgtccagcgccggcagcacggcccggcggtactcgcgcggggacatgggcaccggcgtgtccgggccgaagcgcgtgcgcacgcggtagcgcacgttgccgccgcggcacaggcgcagcggcggcgcgtcggggtacaggcgcgcgtgcgcggcctccacgcgcgcgaagacccccgggccgaacacgcggcccgaggccagcaccgtgcggcgcaggtcccgcgccgccggccagcgcacggcgcactgcacggcgggcagcagctcgcacgccaggtaggcgtgctgccgcgacaccgcgggcccgtcggcgggccagtcgcaggcgcgcacggtgttgaccacgatgagccgccggtcgccggcgctggcgagcagccccagaaactccacggccccggcgaaggccaggtcccgcgtggacagcagcagcacgccctgtgcgcccagcgccgacacgtcgggggcgccggtccaattgcccgcccaggcggccgtgtccggcccgcacagccggttggccagggccgccagcaggcaggacagcccgccgcgctcggcggaccactccggcggcccccccgaggccccgccgccggccaggtcctcgcccggcagcggcgagtacagcaccaccacgcgcacgtcctcggggtcggggatctggcgcatccaggccgccatgcggcgcagcgggcccgaggcgcgcagggggccaaagaggcggcccccggcggccccgtgggggtgggggttatcgtcgtcgtcgccgccgccgcacgcggcctgggcggcgggggcgggcccggcgcaccgcgcggcgatcgaggccagggcccgcgggtcaaacatgagggccggtcgccaggggacggggaacagcgggtggtccgtgagctcggccacggcgcgcggggagcagtaggcctccagggcggcggccgcgggcgccgccgtgtggctgggccccgggggctgccgccgccagccgcccagggggtcggggccctcggcgggccggcgcgacacggccacggggcgcgggcgggcctgcgccgcggcggcccggggcgccgcgggctgggcgggggcgggctcgggccccgggggcgtggaggggggcgcgggcgcggggaggggggcgcgggcgtccgagccgggggcgtccgcgccgctcttcttcgtcttcgggggtcgcgggccgccgcctccgggcggccgggccgggccgggactcttgcgcttgcgcccctcccgcggcgcggcggaggcggcggcggccgccagcgcgtcggcggcgtccggtgcgctggccgccgccgccagcagggggcgcaggctctggttgtcaaacagcaggtccgcggcggcggcggccgcggagctcggcaggcgcgggtcccgcggcagcgcggggcccagggccccggcgaccaggctcacggcgcgcacggcggccacggcggcctcgctgccgccggccacgcgcaggtccccgcgcaggcgcatgagcaccagcgcgtcgcgcacgaaccgcagctcgcgcagccacgcgcgcaggcggggcgcgtcggcgtgcggcggcggcggggaagcggggcccgcgggtccctccggccgcggggggctggcgggccgggccccggccagccccgggacggccgccaggtcgccgtcgaagccctcggccagcgcctccaggatcccgcggcaggcggccaggcactcgacggccacgcggccggcctgggcgcggcgcccggcgtcgtcgtcggcgtcggcgtggcgggcggcgtcggggtcgtcgccccccgcgggggaggcgggcgcggcggacagccgccccagggcggcgaggatccccgcggcgccgtacccggcgggcaccgcgcgctcgcccggtgcggcggcggcgacggcggcgaccccctcgtcatctgcgccggcgccggggctccccgcggcccccgtcagcgccgcgttctcgcgcgccaacaggggcgcgtaggcgcggcgcaggctggtcagcaggaagcccttctgcgcgcggtcgtatcggcggctcatggccacggcggccgccgcgtgcgccaggccccagccgaagcggccggccgccatggcgtagcccaggtggggcacggcccgcgccacgctgccggtgatgaaggagctgctgttgcgcgcggcgcccgagatccggaagcaggcctggtccagcgccacgtccccggggaccacgcgcgggttctggagccaccccatggcctccgcgtccggggtgtacagcagccgcgtgatcagggcgtactgctgcgcggcgtcgcccagctcgggcgcccacacggccgccggggcgcccgaggcctcgaaccggcgtcgcgcctcctccgcctcgggcgccccccagaggcccgggcggctgtcgcccaggccgccgtacagcacccgccccgggggcgggggcccggcgccgggccacggctccccgctgacgtacccgtcgcgatagcgcgcgtagaaggcgccggaggtcgcgtcggcgtccagctcgacccgccggggctgcccggccgtgaagcggcccgtggcgtcgcggccggccaccgccgcgcgggcccggcggcgctcgatgcggcccgcggaggccgcgggggtcctcgccgccgcccggggcttgggcgcggcctcggagagggggggtggcccgggcgggggcggcgtccgcccgggggctgccggcgccgcgctcgacggaccccgcccgacggcccgcgcctcgcgtgcgtggtcggccgcgtcgttgccgtcgtcgtcctcgtcctcgtcggacgacgaggacgaagaggatgcggacgacgaggacgaggacccggagtccgacgaggtcgatgacgccgatggccgccaccggccgtgacgacgtctccgcggcggctgggccggcgggcgcggcgacaggcggtccgtggggtccggatacgcgccgcgtagcggggcctcccgttcgcggccccgggccggggcccggtcgccggcggcgtcggctgcgtcgtcgtactcgtccccgtcatcgtcgtcggctcgaaaggcgggggtccggggcggcgaggccgcggggtcgggcgtcgggatcgtccggacggcctcctctaccatggaggccagcagagccagctgtcgcggcgagacggcgtccccggcgtcctcgccggcgtcggtgcccgccgcgggggccctcccgtcccgccgggcgtcgtcgaggtcgtgggggtggtcggggtcgtggtcggggtcgtccccgccctcctccgtctccgcgccccacccgagggccccccgctcgtcgcggtctgggctcggggtgggcggcggcccgtcggtggggcccggggagccggggcgctgcttgttctccgacgccatcgccgatgcggggcgatcctccggggatacggctgcgacggcggacgtagcacggtaggtcacctacggactctcgatggggggagggggcgagacccacggaccccgacgacccccgccgtcgacgcggaactagcgcggaccggtcgatgcttgggtgggaaaaaggacagggacggccgatccccctcccgcgcttcgtccgcgtatcggcgtcccggcgcggcgagcgtctgacggtctgtctctggcggtcccgcgtcgggtcgtggatccgtgtcggcagccgcgctccgtgtggacgatcggggcgtcctcgggctcatatagtcccaggggccggcgggaaggaggagcagcggaggccgccggccccccgcccccccggcgggcccaccccgaacggaattccattatgcacgaccccgccccgacgccggcacgccgggggcccgtggccgcggcccgttggtcgaacccccggccccgcccatccgcgccatctgccatgggcggggcgcgagggcgggtgggtccgcgccccgccccgcatggcatctcattaccgcccgatccggcggtttccgcttccgttccgcatgctaacgaggaacgggcagggggcggggcccgggccccgacttcccggttcggcggtaatgagatacgagccccgcgcgcccgttggccgtccccgggccccccggtcccgcccgccggacgccgggaccaacgggacggcgggcggcccaagggccgcccgccttgccgcccccccattggccggcgggcgggaccgccccaagggggcggggccgccgggtaaaagaagtgagaacgcgaagcgttcgcacttcgtcccaatatatatatattattagggcgaagtgcgagcactggcgccgtgcccgactccgcgccggccccgggggcgggcccgggcggcggggggcgggtctctccggcgcacataaaggcccggcgcgaccgacgcccgcagacggcgccggccacgaacgacgggagcggctgcggagcacgcggaccgggagcgggagtcgcagagggccgtcggagcggacggcgtcggcatcgcgacgccccggctcgggatcgggatcgcatcggaaagggacacgcggacgcgggggggaaagacccgcccaccccacccacgaaacacaggggacgcaccccgggggcctccgacgacagaaacccaccggtccgccttttttgcacgggtaagcaccttgggtgggcggaggagggggggacgcgggggcggaggaggggggacgcgggggcggaggaggggggacgcgggggcggaggaggggggacgcgggggcggaggaggggggacgcgggggcggaggagggggctcacccgcgttcgtgccttcccgcaggaggaacgtcctcgtcgaggcgaccggcggcgaccgttgcgtggaccgcttcctgctcgtcgggcggggggaagccactgtggtcctccgggacgttttctggatggccgacatttccccaggcgcttttgcgccttgtgtaaaagcgcggcgtcccgctctccgatccccgcccctgggcacgcgcaagcgcaagcgcccttcccgccccctctcatcggagtctgaggtagaatccgatacagccttggagtctgaggtcgaatccgagacagcatcggattcgaccgagtctggggaccaggatgaagccccccgcatcggtggccgtagggccccccggaggcttggggggcggttttttctggacatgtcggcggaatccaccacggggacggaaacggatgcgtcggtgtcggacgaccccgacgacacgtccgactggtcttatgacgacattcccccacgacccaagcgggcccgggtaaacctgcggctcacgagctctcccgatcggcgggatggggttatttttcctaagatggggcgggtccggtctacccgggaaacgcagccccgggcccccaccccgtcggccccaagcccaaatgcaatgctacggcgctcggtgcgccaggcccagaggcggagcagcgcacgatggacccccgacctgggctacatgcgccagtgtatcaatcagctgtttcgggtcctgcgggtcgcccgggacccccacggcagtgccaaccgcctgcgccacctgatacgcgactgttacctgatgggatactgccgagcccgtctggccccgcgcacgtggtgccgtttgctgcaggtgtccggcggaacctggggcatgcacctgcgcaacaccatacgggaggtggaggctcgattcgacgccaccgcggaacccgtgtgcaagcttccttgtttggagaccagacggtacggcccggagtgtgatcttagtaatctcgagattcatctcagcgcgacaagcgatgatgaaatctccgatgccaccgatctggaggccgccggttcggaccacacgctcgcgtcccagtccgacacggaggatgccccctcccccgttacgctggaaaccccagaaccccgcgggtccctcgctgtgcgtctggaggatgagtttggggagtttgactggaccccccaggagggctcccagccctggctgtctgcggtcgtggccgataccagctccgtggaacgcccgggcccatccgattctggggcgggtcgcgccgcagaagaccgcaagtgtctggacggctgccggaaaatgcgcttctccaccgcctgcccctatccgtgcagcgacacgtttctccggccgtgagtccggtcgccccgacccccttgtatgtccccaaaataaaagaccaaaatcaaagcgtttgtcccagcgtcttaatggcgggaagggcggagagaaacagaccacgcggacatggggggtgtttgggggtttattggcaccgggggctaaagggtggtaaccggatagcagatgtgaggaagtcggggccgttcgccgcgaacggcgatcagagggtcagtttcttgcggaccacggcccggcgatgtgggttgctcgtctgggacctcgggcatgcccatacacgcacaacacggacgccgcaccggatgggacgtcgtaagggggcctggggtagctgggtggggtttgtgcagagcaatcagggaccgcagccagcgcatacaatcgcgctcccgtccgtttgtcccgggcagtaccacgccgtactggtattcgtaccggctgagcagggtctccagggggtggttgggggccgcggggaacggggtccacgccacggtccactcgggcaaaaaccgagtcggcacggcccacggttctcccacccacgcgtctggggtcttgatggcgataaatcttaccccgagccggattttttgggcgtattcgagaaacggcacacacagatccgccgcgcctaccacccacaagtggtagaggcgaggggggctgggttggtctcggtgcagcagtcggaagcacgccacggcgtccacgacctcggtgctctccaaggggctgtcctccgcaaacaggcccgtggtggtgtttggggggcagcgacaggacctagtgcgcacgatcgggcgggtgggtttgggtaagtccatcagcggctcggccaaccgtcgaaggttggccggacgaacgacgaccggggtacccaggggttctgatgccaaaatgcggcactgcctaagcaggaagctccacagggccgggcttgcgtcgacggaagtccggggcagggcgttgttctggtcaaggagggtcattacgttgacgacaacaacgcccatgttggtatattacaggcccgtgtccgatttggggcacttgcagatttgtaaggccacgcacggcggggagacaggccgacgcgggggctgctctaaaaatttaagggccctacggtccacagacccgccttcccgggggggcccttggagcgaccggcagcggaggcgtccgggggaggggagggtgatttacgggggggtaggtcagggggtgggtcgtcaaactgccgctccttaaaaccccggggcccgtcgttcggggtgctcgttggttggcactcacggtgcggcgaatggcctgtcgtaagttttgtcgcgtttacgggggacagggcaggaggaaggaggaggccgtcccgccggagacaaagccgtcccgggtgtttcctcatggccccttttataccccagccgaggacgcgtgcctggactccccgcccccggagacccccaaaccttcccacaccacaccacccagcgaggccgagcgcctgtgtcatctgcaggagatccttgcccagatgtacggaaaccaggactaccccatagaggacgaccccagcgcggatgccgcggacgatgtcgacgaggacgccccggacgacgtggcctatccggaggaatacgcagaggagctttttctgcccggggacgcgaccggtccccttatcggggccaacgaccacatccctcccccgtgtggcgcatctccccccggtatacgacgacgcagccgggatgagattggggccacgggatttaccgcggaagagctggacgccatggacagggaggcggctcgagccatcagccgcggcggcaagcccccctcgaccatggccaagctggtgactggcatgggctttacgatccacggagcgctcaccccaggatcggaggggtgtgtctttgacagcagccatccagattacccccaacgggtaatcgtgaaggcggggtggtacacgagcacgagccacgaggcgcgactgctgaggcgactggaccacccggcgatcctgcccctcctggacctgcatgtcgtctccggggtcacgtgtctggtcctccccaagtaccaggccgacctgtatacctatctgagtaggcgcctgaacccactgggacgcccgcagatcgcagcggtctcccggcagctcctaagcgccgttgactacattcaccgccagggcattatccaccgcgacattaagaccgaaaatatttttattaacacccccgaggacatttgcctgggggactttggcgccgcgtgcttcgtgcagggttcccgatcaagccccttcccctacggaatcgccggaaccatcgacaccaacgcccccgaggtcctggccggggatccgtataccacgaccgtcgacatttggagcgccggtctggtgatcttcgagactgccgtccacaacgcgtccttgttctcggccccccgcggccccaaaaggggcccgtgcgacagtcagatcacccgcatcatccgacaggcccaggtccacgttgacgagttttccccgcatccagaatcgcgcctcacctcgcgctaccgctcccgcgcggccgggaacaatcgcccgccgtacacccgaccggcctggacccgctactacaagatggacatagacgtcgaatatctggtttgcaaagccctcaccttcgacggcgcgcttcgccccagcgccgcagagctgctttgtttgccgctgtttcaacagaaatgaccgccccctgggggcggtgctgtttgcgggttggcacaaaaagaccccgatccgcgtctgtggtgtttttggcatcatgtcgcagggcgccatgcgtgccgttgttcccattatcccattccttttggttcttgtcggtgtatcgggggttcccaccaacgtctcctccaccacccaaccccaactccagaccaccggtcgtccctcgcatgaagcccccaacatgacccagaccggcaccaccgactctcccaccgccatcagccttaccacgcccgaccacacaccccccatgccaagtattggactggaggaggaggaagaggaggagggggccggggacggcgaacatcttgaggggggagatgggacccgtgacaccctaccccagtccccgggcccagccttcccgttggctgaggacgtcgagaaggacaaacccaaccgtcccgtagtcccatcccccgatcccaacaactcccccgcgcgccccgagaccagtcgcccgaagacaccccccaccattatcgggccgctggcaactcgccccacgacccgactcacctcaaagggacgacccttggttccgacgcctcaacataccccgctgttctcgttcctcactgcctcccccgccctggacaccctcttcgtcgtcagcaccgtcatccacaccttatcgtttttgtgtattggtgcgatggcgacacacctgtgtggcggttggtccagacgcgggcgacgcacacaccctagcgtgcgttacgtgtgcctgccgtccgaacgcgggtagggtatggggcgggggatggggagagcccacatgcggaaagcaagaacaataaaggcggtggtatctagttgatatgcatctctgggtgtttttggggtgtggcggacgcggggcggtcattggacggggtgcagttaaatacatgcccgggacccatgaagcatgcgcgacttccgggcctcagaacccacccgaaacggccaacggacgtctgagccaggcctggctatccggagaaacagcacacgacttggcgttctgtgtgtcgcgatgtctctgcgcgcagtctggcatctggggcttttgggaagcctcgtgggggctgttcttgccgccacccatcggggacctgcggccaacacaacggaccccttaacgcacgccccagtgtcccctcaccccagccccctggggggctttgccgtccccctcgtagtcggtgggctgtgcgccgtagtcctgggggcggcatgtctgcttgagctcctgcgtcgtacgtgccgcgggtgggggcgttaccatccctacatggacccagttgtcgtataatttcccccccccccccccttctccgcgtgggtgatgtcgggtccaaactcccgacaccaccagctggcatggtataaatcaccggtgcgccccccaaaccatgtccggcagggggatgggggggcaatgcggagggcacccaacaacaccgggctaaccaggaaatccgtggccccggcccccaataaagatcgcggtagcccggccgtgtgacactatcgtccataccgaccacaccgacgaatcccccaagggggaggggccattttacgaggaggaggggtataacaaagtctgtctttaaaaagcaggggttagggagttgttcggtcataagcttcagcgcgaacgaccaactaccccgatcatcagttatccttaaggtctcttttgtgtggtgcgttccggtatggggggggctgccgccaggttgggggccgtgattttgtttgtcgtcatagtgggcctccatggggtccgcagcaaatatgccttggtggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccacactcaaaaagccccaaagcgcatacgcctcccccacatccgggaagacgaccagccgtcctcgcaccagcccttgttttactagatacccccccttaatgggtgcgggggggtcaggtctgcggggttgggatgggaccttaactccatataaagcgagtctggaaggggggaaaggtggacagtcgataagtcggtagcgggggacgcgcacctgttccgcctgtcgcacccacagctttttttgcgaaccgtcccgttccgggatgccgtgccgcccgttgcagggcctggtgctcgtgggcctctgggtctgtgccaccagcctggttgtccgtggccccacggtcagtctggtatcaaactcatttgtggacgccggggccttggggcccgacggcgtagtggaggaagacctgcttattctcggggagcttcgctttgtgggggaccaggtcccccacaccacctactacgatgggggcgtagagctgtggcactaccccatgggacacaaatgcccacgggtcgtgcatgtcgtcacggtgaccgcgtgcccacgtcgccccgccgtggcattcgccctgtgtcgcgcgaccgacagcactcacagccccgcatatcccaccctggagctcaatctggcccaacagccgcttttgcgggtccagagggcaacgcgggactatgccggggtgtacgtgttacgcgtatgggtcggtgacgcgccaaacgccagcctgtttgtcctggggatggccatagccgccgaagggactctggcgtacaacggctcggcctatggctcctgcgacccgaaactgcttccgtcttcggccccgcgtctggccccggcgagcgtataccaacccgcccctaaccaggcctccaccccctcgaccaccacctccaccccctcgaccaccatccccgctccctcgaccaccatccccgctccccaagcatcgaccacgcccttccccacgggagatccaaaaccacaacctcccggggtcaaccacgaacccccatctaatgccacgcgagcgacccgcgactcgcgatacgcgctaacggtgacccagataatccagatagccatccccgcgtccatcatagccctggtgtttctggggagctgtatttgctttatacacagatgtcaacgccgctaccgacgctcccgtcgcccgatttacagcccccagatgcccacgggcatctcatgcgcggtgaacgaagcggccatggcccgcctcggagccgagctcaaatcgcatccgagcaccccccccaaatcccggcgccggtcgtcacgcacgccaatgccctccctgacggccatcgccgaagagtcggagcccgctggggcggctgggcttccgacgccccccgtggaccccacgacacccaccccaacgcctcccctgttggtataggtccacggccactggccgggagcaccacataaccgaccgcagtccctgagttgggaataaaccggtattatttacctatatccgtgtatgtcgatttctttccccccctccccggaaaccaaagaaggaagcaaagaatggatgggaggagttcaggaagccggggagagggcccgcggcgcatttaaggcgttgttgtgttgactttgcctcttctggcgggttggtgcggtgctgtttgttgggctcccattttacccgaagatcggctgctatccccgggacatggatcgcggggcggtggtggggtttcttctcggtgtttgtgttgtatcgtgcttggcgggaacgcccaaaacgtcctggagacgggtgagtgtcggcgaggacgtttcgttgcttccagctccggggcctacggggcgcggcccgacccagaaactactatgggccgtggaacccctggatgggtgcggccccttacacccgtcgtgggtctcgctgatgccccccaagcaggtgcccgagacggtcgtggatgcggcgtgcatgcgcgctccggtcccgctggcgatggcgtacgcccccccggccccatctgcgaccgggggtctacgaacggacttcgtgtggcaggagcgcgcggccgtggttaaccggagtctggttattcacggggtccgagagacggacagcggcctgtataccctgtccgtgggcgacataaaggacccggctcgccaagtggcctcggtggtcctggtggtgcaaccggccccagttccgaccccacccccgaccccagccgattacgacgaggatgacaatgacgagggcgaggacgaaagtctcgccggcactcccgccagcgggaccccccggctcccgcctccccccgcccccccgaggtcttggcccagcgcccccgaagtctcacatgtgcgtggggtgaccgtgcgtatggagactccggaagctatcctgttttcccccggggagacgttcagcacgaacgtctccatccatgccatcgcccacgacgaccagacctactccatggacgtcgtctggttgaggttcgacgtgccgacctcgtgtgccgagatgcgaatatacgaatcgtgtctgtatcacccgcagctcccagaatgtctgtccccggccgacgcgccgtgcgccgcgagtacgtggacgtctcgcctggccgtccgcagctacgcggggtgttccagaacaaaccccccaccgcgctgttcggccgaggctcacatggagcccgtcccggggctggcgtggcaggcggcctccgtcaatctggagttccgggacgcgtccccacaacactccggcctgtatctgtgtgtggtgtacgtcaacgaccatattcacgcctggggccacattaccatcagcaccgcggcgcagtaccggaacgcggtggtggaacagcccctcccacagcgcggcgcggatttggccgagcccacccacccgcacgtcggggcccctccccacgcgcccccaacccacggcgccctgcggttaggggcggtgatgggggccgccctgctgctgtctgcactggggttgtcggtgtgggcgtgtatgacctgttggcgcaggcgtgcctggcgggcggttaaaagcagggcctcgggtaaggggcccacgtacattcgcgtggccgacagcgagctgtacgcggactggagctcggacagcgagggagaacgcgaccaggtcccgtggctggcccccccggagagacccgactctccctccaccaatggatccggctttgagatcttatcaccaacggctccgtctgtatacccccgtagcgatgggcatcaatctcgccgccagctcacaacctttggatccggaaggcccgatcgccgttactcccaggcctccgattcgtccgtcttctggtaaggcgccccatcccgaggccccacgtcggtcgccgaactgggcgaccgccggcgaggtggacgtcggagacgagctaatcgcgatttccgacgaacgcggacccccccgacatgaccgcccgcccctcgccacgtcgaccgcgccctcgccacacccgcgacccccgggctacacggccgttgtctccccgatggccctccaggctgtcgacgccccctccctgtttgtcgcctggctggccgctcggtggctccggggggcttccggcctgggggccgtcctgtgtgggattgcgtggtatgtgacgtcaattgcccgaggcgcataaagggccggtggtccgcctagccgcagcaaattaaaaatcgtgagtcacagcgaccgcaacttcccacccggagctttcttccggcctcgatgacgtcccggctctccgatcccaactcctcagcgcgatccgacatgtccgtgccgctttatcccacggcctcgccagtttcggtcgaagcctactactcggaaagcgaagacgaggcggccaacgacttcctcgtacgcatgggccgccaacagtcggtattaaggcgtcgacgcagacgcacccgctgcgtcggcatggtgatcgcctgtctcctcgtggccgttctgtcgggcggatttggggcgctcctgatgtggctgctccgctaaaagaccgcatcgacacgcgcgtccttcttgtcgtctctcttcccccccatcaccccgcaatttgcacccagcctttaactacattaaattgggttcgattggcaatgttgtctcccggttgatttttgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtgggtgggtggggagtggcaaggaagaaacaagcccgaccaccagacagaaaatgtaaccatacccaaaccgactctgggggctgtttgtggggtcggaaccataggatgaacaaaccaccccgtaccacccgcacccaagggtgcggtggctcatcggcatctgtccggtatgggttgttccccacccactcgcgttcggacgtcttagaatcatggcggttttctatgccgacatcggttttctcccccgcaataagacacgatgcgataaaatctgtttgtaaaatttattaagggtacaaattgccctagcacaggggtggggttagggccgggtccccacacccaaacgcaccaaacagatgcaggcagtgggtcgagtacagccccgcgtacgaacacgtcgatgcgtgtgtcagacagcaccagaaagcacaggccatcaacaggtcgtgcatgtgtcggtgggtttggacgcggggggccatggtggtgataaagttaatggccgccgtccgccagggccacaggggcgacgtctcttggttggcccggagccactgggtgtggaccagccgcgcgtggcggcccaacatggcccctgtagccgggggcgggggatcgcgcacgtttgcagcgcacatgcgagacacctcgaccacggttcgaaagaaggcccggtggtccgcgggcaacatcaccaggtgcgcaagcgcccgggcgtccagagggtagagccctgagtcatccgaggttggctcatcgcccgggtcttgccgcaagtgcgtgtgggttgggcttccggtgggcgggacgcgaaccgcggtgtggatcccgacgcgggcccgagcgtatgctccatgttgtggggagaaggggtctgggctcgccaggggggcatacttgcccgggctatacagacccgcgagccgtacgtggttcgcggggggtgcgtggggtccggggctcccggggagaccggggctcccggggagaccggggctccctgggagaccggggttgtcgtggatccctggggtcacgcggtaccctggggtctctgggagctcgcggtactctgggttccctaggttctcggggtggtcgcggaacccggggctcccggggaacacgcggtgtcctggggattgttggcggtcggacggcttcagatggcttcgagatcgtagtgtccgcaccgactcgtagtagacccgaatctccacattgccccgccgcttgatcattatcaccccgttgcgggggtccggagatcatgcgcgggtgtcctcgaggtgcgtgaacacctctggggtgcatgccggcggacggcacgccttttaagtaaacatctgggtcgcccggcccaactggggccgggggttgggtctggctcatctcgagagccacgggggggaaccaccctccgcccagagactcgggtgatggtcgtacccgggactcaacgggttaccggattacggggactgtcggtcacggtcccgccggttcttcgatgtgccacacccaaggatgcgttgggggcgatttcgggcagcagcccgggagagcgcagcaggggacgctccgggtcgtgcacggcggttctggccgcctcccggtcctcacgcccccttttattgatctcatcgcgtacgtcggcgtacgtcctgggcccaacccgcatggtgtccaggaaggtgtccgccatttccagggcccacgacatgctcccccccgacgagcaggaagcggtccacgcaacggtcgccgccggtcgcctcgacgaggacgttcctcctgcgggaaggcacgaacgcgggtgagccccctcctccgcccccgcgtcccccctcctccgcccccgcgtcccccctcctccgcccccgcgtcccccctcctccgcccccgcgtcccccctcctccgcccccgcgtcccccctcctccgcccccgcgtcccccctcctccacccccgcgtccccccctcctccgcccacccaaggtgcttacccgtgcaaaaaaggcggaccggtgggtttctgtcgtcggaggcccccggggtgcgtcccctgtgtttcgtgggtggggtgggcgggtctttcccccccgcgtccgcgtgtccctttccgatgcgatcccgatcccgagccggggcgtcgcgatgccgacgccgtccgctccgacggccctctgcgactcccgctcccggtccgcgtgctccgcagccgctcccgtcgttcgtggccggcgccgtctgcgggcgtcggtcgcgccgggcctttatgtgcgccggagagacccgccccccgccgcccgggcccgcccccggggccggcgcggagtcgggcacggcgccagtgctcgcacttcgccctaataatatatatatattgggacgaagtgcgaacgcttcgcgttctcacttcttttacccggcggccccgcccccttggggcggtcccgcccgccggccaatgggggggcggcaaggcgggcggcccttgggccgcccgccgtcccgttggtcccggcgtccggcgggcgggaccggggggcccggggacggccaacgggcgcgcggggctcgtatctcattaccgccgaaccgggaagtcggggcccgggccccgccccctgcccgttcctcgttagcatgcggaacggaagcggaaaccgccggatcgggcggtaatgagatgccatgcggggcggggcgcggacccacccgccctcgcgccccgcccatggcagatggcgcggatgggcggggccgggggttcgaccaacgggccgcggccacgggcccccggcgtgccggcgtcggggcggggtcgtgcataatggaattccgttcggggtgggcccgccgggggggcggggggccggcggcctccgctgctcctccttcccgccggcccctgggactatatgagcccgaggacgccccgatcgtccacacggagcgcggctgccgacacggatccacgacccgacgcgggaccgccagagacagaccgtcagacgctcgccgcgccgggacgccgatacgcggacgaagcgcgggagggggatcggccgtccctgtcctttttcccacccaagcatcgaccggtccgcgctagttccgcgtcgacggcgggggtcgtcggggtccgtgggtctcgccccctccccccatcgagagtccgtaggtgacctaccgtgctacgtccgccgtcgcagccgtatccccggaggatcgccccgcatcggcgatggcgtcggagaacaagcagcgccccggctccccgggccccaccgacgggccgccgcccaccccgagcccagaccgcgacgagcggggggccctcgggtggggcgcggagacggaggagggcggggacgaccccgaccacgaccccgaccacccccacgacctcgacgacgcccggcgggacgggagggcccccgcggcgggcaccgacgccggcgaggacgccggggacgccgtctcgccgcgacagctggctctgctggcctccatggtagaggaggccgtccggacgatcccgacgcccgaccccgcggcctcgccgccccggacccccgcctttcgagccgacgacgatgacggggacgagtacgacgacgcagccgacgccgccggcgaccgggccccggcccggggccgcgaacgggaggccccgctacgcggcgcgtatccggaccccacggaccgcctgtcgccgcgcccgccggcccagccgccgcggagacgtcgtcacggccggtggcggccatcggcgtcatcgacctcgtcggactccgggtcctcgtcctcgtcgtccgcatcctcttcgtcctcgtcgtccgacgaggacgaggacgacgacggcaacgacgcggccgaccacgcacgcgaggcgcgggccgtcgggcggggtccgtcgagcgcggcgccggcagcccccgggcggacgccgcccccgcccgggccaccccccctctccgaggccgcgcccaagccccgggcggcggcgaggacccccgcggcctccgcgggccgcatcgagcgccgccgggcccgcgcggcggtggccggccgcgacgccacgggccgcttcacggccgggcagccccggcgggtcgagctggacgccgacgcgacctccggcgccttctacgcgcgctatcgcgacgggtacgtcagcggggagccgtggcccggcgccgggcccccgcccccggggcgggtgctgtacggcggcctgggcgacagccgcccgggcctctggggggcgcccgaggcggaggaggcgcgacgccggttcgaggcctcgggcgccccggcggccgtgtgggcgcccgagctgggcgacgccgcgcagcagtacgccctgatcacgcggctgctgtacaccccggacgcggaggccatggggtggctccagaacccgcgcgtggtccccggggacgtggcgctggaccaggcctgcttccggatctcgggcgccgcgcgcaacagcagctccttcatcaccggcagcgtggcgcgggccgtgccccacctgggctacgccatggcggccggccgcttcggctggggcctggcgcacgcggcggccgccgtggccatgagccgccgatacgaccgcgcgcagaagggcttcctgctgaccagcctgcgccgcgcctacgcgcccctgttggcgcgcgagaacgcggcgctgacgggggccgcggggagccccggcgccggcgcagatgacgagggggtcgccgccgtcgccgccgccgcaccgggcgagcgcgcggtgcccgccgggtacggcgccgcggggatcctcgccgccctggggcggctgtccgccgcgcccgcctcccccgcggggggcgacgaccccgacgccgcccgccacgccgacgccgacgacgacgccgggcgccgcgcccaggccggccgcgtggccgtcgagtgcctggccgcctgccgcgggatcctggaggcgctggccgagggcttcgacggcgacctggcggccgtcccggggctggccggggcccggcccgccagccccccgcggccggagggacccgcgggccccgcttccccgccgccgccgcacgccgacgcgccccgcctgcgcgcgtggctgcgcgagctgcggttcgtgcgcgacgcgctggtgctcatgcgcctgcgcggggacctgcgcgtggccggcggcagcgaggccgccgtggccgccgtgcgcgccgtgagcctggtcgccggggccctgggccccgcgctgccgcgggacccgcgcctgccgagctccgcggccgccgccgccgcggacctgctgtttgacaaccagagcctgcgccccctgctggcggcggcggccagcgcaccggacgccgccgacgcgctggcggccgccgccgcctccgccgcgccgcgggaggggcgcaagcgcaagagtcccggcccggcccggccgcccggaggcggcggcccgcgacccccgaagacgaagaagagcggcgcggacgcccccggctcggacgcccgcgcccccctccccgcgcccgcgcccccctccacgcccccggggcccgagcccgcccccgcccagcccgcggcgccccgggccgccgcggcgcaggcccgcccgcgccccgtggccgtgtcgcgccggcccgccgagggccccgaccccctgggcggctggcggcggcagcccccggggcccagccacacggcggcgcccgcggccgccgccctggaggcctactgctccccgcgcgccgtggccgagctcacggaccacccgctgttccccgtcccctggcgaccggccctcatgtttgacccgcgggccctggcctcgatcgccgcgcggtgcgccgggcccgcccccgccgcccaggccgcgtgcggcggcggcgacgacgacgataacccccacccccacggggccgccgggggccgcctctttggccccctgcgcgcctcgggcccgctgcgccgcatggcggcctggatgcgccagatccccgaccccgaggacgtgcgcgtggtggtgctgtactcgccgctgccgggcgaggacctggccggcggcggggcctcgggggggccgccggagtggtccgccgagcgcggcgggctgtcctgcctgctggcggccctggccaaccggctgtgcgggccggacacggccgcctgggcgggcaattggaccggcgcccccgacgtgtcggcgctgggcgcacagggcgtgctgctgctgtccacgcgggacctggccttcgccggggccgtggagtttctggggctgctcgccagcgccggcgaccggcggctcatcgtggtcaacaccgtgcgcgcctgcgactggcccgccgacgggcccgcggtgtcgcggcagcacgcctacctggcgtgcgagctgctgcccgccgtgcagtgcgccgtgcgctggccggcggcgcgggacctgcgccgcacggtgctggcctcgggccgcgtgttcggcccgggggtcttcgcgcgcgtggaggccgcgcacgcgcgcctgtaccccgacgcgccgccgctgcgcctgtgccgcggcggcaacgtgcgctaccgcgtgcgcacgcgcttcggcccggacacgccggtgcccatgtccccgcgcgagtaccgccgggccgtgctgccggcgctggacggccgggcggcggcctcggggaccaccgacgccatggcgcccggcgcgccggacttctgcgaggaggaggcccactcgcacgccgcctgcgcgcgctggggcctgggcgcgccgctgcggcccgtgtacgtggcgctggggcgcgaggcggtgcgcgccggcccggcccggtggcgcgggccgcggagggacttttgcgcccgcgccctgctggagcccgacgacgacgcccccccgctggtgctgcgcggcgacgacgacggcccgggggccctgccgccggcgccgcccgggattcgctgggcctcggccacgggccgcagcggcaccgtgctggcggcggcgggggccgtggaggtgctgggggcggaggcgggcttggccacgcccccgcggcgggaagttgtggactgggaaggcgcctgggacgaagacgacggcggcgcgttcgagggggacggggtgctgtaacgggccgggacggggcggggcgcttgtgagacccgaagacgcaataaacggcaacaacctgattaagttttgcagtagcgttgtttattcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcgggagggggcgaggggcggtggtggtgcgcgggcgcccccggagggtttggatctctgacctgagattggcggcactgaggtagagatgcccgaacccccccgagggagcgcgggacgcgccggggagggctggggccggggagggctggggccggggagggctggggccggggagggctggggccggggagggctggggccggggagggctggggccggggagggctggggctggggagggctggggctggggagggggcggtggtgtgtagcaggagcggtgtgttgcgccggggtacgtctggaggagcgggaggtgcgcggtgacgtgtggatgaggaacaggagttgttgcgcggtgagttgtcgctgtgagttgtgttgttgggcaggtgtggtggatgacgtgacgtgtgacgtgcggagtgcgccgtgctctgttggtttcacctgtggcagcccgggccccccgcgggcgcgcgcgcgcgcaaaaaaggcgggcggcggtccgggcggcgtgcgcgcgcgcggcgggcgtggggggcggggccgcgggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggggggaggagcggccagacgccgaaaacgggccccccccaaaacacaccccccgggggtcgcgcgcggccctttaaagcggtggcggcgggc

SEQ ID NO: 16 - DNA, 154746 bp, Herpes simplex HSV-2 (NC_001798.1)
agtccccgtcctgccgcgcgggggcgggcgcgggaaaaaagccgcgcgggggcgcccgcgggaaggcagccccgcggcgcgcggggggaggggcggcgcccgcgggggagcggccggctccgggggagggacggggaagggggcgcgcggggctgccctgccgcccgcccgccgccgccgcccgccttcgcgcccccccccaaaaaacaccccccccgggggttgactccccgggggaaaagaggcggggcgggagtccccgtcctgccgccgccccttaagagggcccgcaacacggcccgggctgcgcacgccagccgggacgggtgagttcgctaggcaagcacggactggcggttacacgtgcatgcgtgccgagtgaactctcccgccccgacgcgctccggctccgggcctacgccgagcccagccgcccgccatgtcccgccgccggggtccccgccgccggggtccccggcgccggccgcgccccggcgctccagccgtgccgcgccccggcgctccagccgtgccgcgccccggcgcgctcccaaccgcagactcccaaatggtccctgcgtacgactcgggaaccgcggtcgagagcgcgccggccgcgtcctcgctcctgcggcgctggctgctggtgccccaggcggacgacagcgacgacgcggactacgccggcaacgacgacgcagagtgggcgaacagccccccgagcgagggcggggggaaggcgccggaggccccgcacgccgcgcctgccgccgcctgccccccgccgccgccgcgcaaggagcgcgggccgcagcgcccccttccgccccacctggcgctacggctgcgcaccacgacggagtacctggcgcgcctgagcctgcgccggcggcggccccccgcgtccccgcccgcggacgcgccgcgcgggaaggtacgcctcccctccgaccccctgacgcccctccgaccccctgacgcccctccgaccccctgacgcccctccgaccccctgacgcccctccgaccccctgacgcccctccgaccccctgacgcccctccgacccccgtgtctccccgcccgcaggtgtgcttctcgccgcgcgtgcaggtgcgccatctggtggcctgggagacggccgcgcgcctggcccgacgggggtcctgggcgcgcgagcgggccgaccgcgaccggttccggcgccgcgtggcggcggccgaggcggtcatcggaccgtgcctggagcccgaggcccgagctcgggcccgagcccgagcccgggcccacgaagacggcggacccgcggaggaggaggaggcggcggcggcggcgcgcgggtcctccgccgccgcgggcccgggccgtcgggcggtctagggttgaaccggcgagggcggcctcggccggcggagccccggagctccgaaggtctgcgcgaggccgctctccgaagagacgatgggagccccgcgtatatatccgcgagggcccggcgccgccccgccgctccgcccgccccagggggcggcgccggccaaccgcgcgccgccgcgcgggcccggactccgccccggcgaccgccccgcgccggcttcccggtatggtaattagaaacttttaataggcggtcccggccgccatccccgcgcatggtaattagcaacttttaatgggccggcgttcccgctcgcggtaattagcagcttttaacgggccgccattcccgcttatggtaattaaaaacgttcggacggcccctcgctccccgcgtaattactccctcggggttccgggttatgctgattactttcttggcagaacacgcagagcctcgcgcgccgccgggtgggtgggctgatcggcccctattggtcccctgggcttcctagtatgctaatgaatttttccccgggggcgggcaccactcagggccgcgccggcggggcgccggggggactcccatctgcgtcggcggggggcggcgcatgctaatggggttcttggagtacacccggttggtccccggggacggggccgccccgagagggggggattccctccctccgcccccgccggggcgcgcggctattgggggaatcgtaaatgccgcccctttgggggagtggataggcgccgggtataaggcagccccgtgtgacggtcgggccgcattcgcaccccggcactgcgagcgacggagcggcggcccggcgggaggaggagacccggagagacagagactaaaacccggcaagagagagaccgcgggccgccgtctcgagtctaccctaccccggctcatggaaccccggcccggcacgagctcccgggcggaccccggccccgagcggccgccgcggcagacccccggcacggtgagagggcgacccccgggtctcaggcccccccttttccccggaccacccggctgcgggttgggggtggtcgcgggcggtgggctcgggggcggggacgcttgacggggccgacccccggcccgcttaagcggtcgggggacccccgtgggccgtgcgccgccccccgaccctctgggggggcgagggaggcagggaggagcccgagagcgggggacagggggggagacgaggggtcggaatccaaaggacgcagaccacctttggttacggacccctttctcccccccttccgaacaaaaagcagcgggcggggggccggggtgagggagggacacgggggacacggcgcgggggtcccgcctcacgccccgcgccctctaaatcccccccgttgctttgtcaagcagcccgccgccccgcacgcctgggggatgctcaacgacatgcagtggctcgccagcagcgactcggaggaggagaccgaggtgggaatctctgacgacgaccttcaccgcgactccacctccgaggcgggcagcacggacacggagatgttcgaggcgggcctgatggacgcggccacgcccccggcccggcccccggccgagcgccagggcagccccacgcccgccgacgcgcagggatcctgtgggggtgggcccgtgggtgaggaggaagcggaagcgggaggggggggcgacgtgtgtgccgtgtgcacggacgagatcgccccgcccctgcgctgccagagttttccctgcctgcaccccttctgcatcccgtgcatgaagacctggattccgttgcgcaacacgtgtcccctgtgcaacaccccggtggcgtacctgatagtgggcgtgaccgccagcgggtcgttcagcaccatcccgatagtgaacgacccccggacccgcgtggaggccgaggcggccgtgcgggccggcacggccgtggactttatctggacgggcaacccgcggacggccccgcgctccctgtcgctggggggacacacggtccgcgccctgtcgcccacccccccgtggcccggcacggacgacgaggacgatgacctggccgacggtgagggcgggcgggggtcgggcggggggcgggcgggggtcgggcgggggtcgggcgggggtcgggcgggggtcgggcgggggtcgggcgggggtcgggcgggggtcgggcgggggtcgggcgggggtcgggcgggggtcgggcactaaccgggggctcccgtctctgtctccctctgcagtggactacgtcccgcccgccccccgaagagcgccccggcgcgggggcggcggtgcgggggcgacccgcggaacctcccagcccgccgcgacccgaccggcgccccctggcgccccgcggagcagcagcagcggcggcgccccgttgcgggcgggggtgggatctgggtctgggggcggccctgccgtcgcggccgtcgtgccgagagtggcctctcttccccctgcggccggcggggggcgcgcgcaggcgcggcgggtgggcgaagacgccgcggcggcggagggcaggacgccccccgcgagacagccccgcgcggcccaggagccccccatagtcatcagcgactctcccccgccgtctccgcgccgccccgcgggccccgggccgctctcctttgtctcctcctcctccgcacaggtgtcctcgggccccggggggggaggtctgccacagtcgtcggggcgcgccgcgcgcccccgcgcggccgtcgccccgcgcgtccggagtccgccccgcgccgccgccgcccccgtggtgtctgcgagcgcggacgcggccgggcccgcgccgcccgccgtgccggtggacgcgcaccgcgcgccccggtcgcgcatgacccaggctcagaccgacacccaagcacagagtctgggccgggcaggcgcgaccgacgcgcgcgggtcgggagggccgggcgcggagggaggacccggggtcccccgcggcaccaacacccccggtgccgccccccacgccgcggagggggcggcggcccgcccccggaagaggcgcgggtcggactcgggccccgcggcctcgtcctccgcctcttcctccgccgccccgcgctcgcccctcgccccccagggggtgggggccaagagggcggcgccgcgccgggccccggactcggactcgggcgaccgcggccacgggccgctcgccccggcgtccgcgggcgccgcgcccccgtcggcgtctccgtcgtcccaggccgcggtcgccgccgcctcctcctcctccgcctcctcctcctccgcctcctcctcctccgcctcctcctcctccgcctcctcctcctccgcctcctcctcctccgcctcctcctcctccgcctcttcctctgcgggcggggctggtgggagcgtcgcgtccgcgtccggcgctggggagagacgagaaacctccctcggcccccgcgctgctgcgccgcgggggccgaggaagtgtgccaggaagacgcgccacgcggagggcggccccgagcccggggcccgcgacccggcgcccggcctcacgcgctacctgcccatcgcgggggtctcgagcgtcgtggccctggcgccttacgtgaacaagacggtcacgggggactgcctgcccgtcctggacatggagacgggccacataggggcctacgtggtcctcgtggaccagacggggaacgtggcggacctgctgcgggccgcggcccccgcgtggagccgccgcaccctgctccccgagcacgcgcgcaactgcgtgaggccccccgactacccgacgccccccgcgtcggagtggaacagcctctggatgaccccggtgggcaacatgctctttgaccagggcaccctggtgggcgcgctggacttccacggcctccggtcgcgccacccgtggtctcgggagcagggcgcgcccgcgccggccggcgacgcccccgcgggccacggggagtagggggagctaacactcggcttgctgcccgaaggaagccgccccccaccggaccaccggccgaggcgcctcgggggcagggggaggtggggggggggaaagacggggaggagacaggaagtgggggtgggagtgggggggggggacggacacggccccgaacagcaacacacaccagcattttgttatggactttctggccttgttgaaaacttgaggaaaaaaaaaactttatatttataaaaattttacaataaagttttgtgatgcttttgacacactttgttgttggcctttgatgcagctcccccgcgcaggggggccggggatgggggggaagggaggaggaggagggggggcgggcacgagaagccgcccccacccccgaggcctgttggtctttatcatagaacagagccggggcccggcctcgttctggctccctgtcttggtgggtgggcgggctggctggcgggtaaaaaaagagtgtgtccgtgttgacagggaggggggcccgatcgtgcagagcacgcacgtctggccggccagaccctgggggtggtgggcaggagtgggagggcgcctggctcggggagggaggagggggggggtcagccgcaccaccggcgcgaagccaggggccagggaactttgatagagaggggggaaagtggggcgggggcgagggcggttgaatcacaacgcatgcacgccctctgcccccggggacgggtgggaggaaggaggagggagaagagaagacccgaggcatgcacccgcacttacgcccgtgcccacccccgccccggcgcccaccccgcccgcacacctgcccgccacgcccgcccctcctcaccctggctgggagaaaggaggaggagcaggaagaggagacccgaggcatgcaaccgcactcaccccaccccgcccgcacacctgcccgccacgcccgcccctccttaccctggctgcggggagactcccatcggggcgagggggctcgcgcgttcgcaacaccacaccacaccacacggcccaccacaacacggcccaccacgacacaacacgacacgacgcgttttgcggggcatgcaagtcgacacaccgcgcgcgtgcctacctttccctagcggccccggcccccggcccgtttccttccgccaccactaccaccacccccccgcccgcgcccacgcggtagaggaaggggacgggcgccacacccacggctgtggccgggcacgcgcctttggggttgttgggggggggtgaccggcgcgtgggggcggtgggcgtacgggcccgacccgcgcctgcccccccgggaacgacgacgggggggggggaaacgggggtgggtggaagggaagaggaaggagaaagggggggtggatccgaacacgccggatccgcgaaaataataacaaaacaaacaaaaacagaaacaaaaacaaaaacacctagaaaaaaaggatacgggttggctcgcgggcggtgcggctgacctgcctgccctttctgggacccccgcctcgtgtttcttgaaagggggaggaagaacagttctcccccaacccctgctctcttctctcttccgcccgccccccccccctctccccgccgcctcagcagaagctcacctgtacgaccctaaacctacctgcgagaacgcgcggcgttcgaggggcgcgctctctcacacgagacacacgcaggcgccccccccccccggagcctgggtcccccggcggacggctcacgcggcgcggcgtctcggtgggacgcgggcaaagggcggcggcggcgggggggggggggggaaatgtgaggagagcgagacagagagagagaaggaagagggaaggggcgcggcgggacgggggaagacgaggagaagggaaggggcgagggtcgggcccgggagcggggcggcccgggagggagaagaaacggaacgcggaaacgccgccggcgcggcccggggccccggggcccccgcgctccgccgggggcccgggccggaccgccgggcgggggacgccttccgcccggcgccgggcggctacccgggacccccggccgggaatcgaaaaaagcctccgggggcccctttcgcgcctttcgcgaacgcgcggcgccggagggggcggccgccgaggtgcgggggcccctccggccggggcgcacctcggcggccaagccccggcccgcccgggggtccccgaggcaagaggcggaccctcggaggcgcggaagaagacgggaggcgggggaaaaaagggggaagagagggggaggtagggaggggagaggagaagggcgcgccggtgcgcggagcagccttccttctccggagtccctctcgatcggcggcgggcccctgcgttcgttgctgccgcgcccccggttttataaagacagggatgacgcagcagaaatgcccacagcaacacgcgggcggggctcgggctctccggcggcttaatggatctccgggcacggcgcccgcaaccgcagagcactcagctggcgcgcccccccccaacgtgggagtgtttaatggaagggcgtggggccggccgccggatgcccgcgggggcctaatgcggcgggaggcgtgggccgctggcgccgcggcccgtctgctggcccgcggcccgtctgctggcccgcggccacgtaaacaatgacacaggggttctctccgccgcggccggcgcggggcgttgccggcccggcccggccccggagcccgcggcgctgctcggctgcggccgcgggctccgggggctccgcactctgcccggctcgccccgtcccccctcttgctgcttttccgcgcgcctctctttcccgttgctttccctctccccccccccccctctctctctctctctctctctctccgccatcctcccgcccggccgcccactccccgctcggcctctccggctgcggtgcttgggtctccttcgtcgggcggcggggggggggcgtcgggactcgcggagggccggagaatggaaggcgaggggatgcaggaggaggatcgggactccccatcttctgcccttccatcctccgtttttccgctttccaccgccgccgccaccacccccccttccttcgcccgcccgcctcgccccggacccctcccccccgtgttccccccatcgttcaccaccacgccccccaccgcgccttggctgtttggggggtggcggcggtggtcggcgtgctgccggaggctgcgggcgcggggtaggtgggtgggcgggtggtgggggggggcccggctgcgtctcgccgcgatcccgccggtggggcgcggcggcggtcggggtggggggagagtgtcgtgggtgtgttttcgtgtcccccaccaccactcccaccccgaccgccgccgcgcccgcgtttctgccgcccgcgcgctcctgtgtggaccccggggtgggcggcgggggggggtgccgtgggtgtggcggcggggcgcgggccggggccggggctcgctggtccgccgaagtaaagaaaagatcgccaccgtgtgttcgtctgtgtgttctgcgcggcgccggggcccccctgccgggcggggcggtggggcggggtcggggtcgcggcggggaaggaaggaaagaccccggaagcgccgggagggggcgccggcgcgacgcgggcggccgggcgggggcgcgcggcggccgggcgggggcgcgcggcggccgggcgggggcgcgcggcggccgggcgggggcgcgcggcggccgggcgggggcgcgcggcggccgggcgggggcgcgcggcggccgggcgggggcgcgctttccccgcgtcgcccctcgggttcccaagacctatcacgtgtgcgcaggggaggggaggacgcgggggaggggaggacgcgggggaggggaggacgcgggggatatataaagcggtagaaagcgcgggaatgggcatattggacccgcgtgattcggttgctcgcggttgtcttgtttggacgttttttatgcgggaacaagggggcttaccggttacactgtccgctcgctatggggttcgtctgtctgtttgggcttgtcgttatgggagcctggggggcgtggggtgggtcacaggcaaccgaatatgttcttcgtagtgttattgccaaagaggtgggggacatactaagagtgccttgcatgcggacccccgcggacgatgtttcttggcgctacgaggccccgtccgttattgactatgcccgcatagacggaatatttcttcgctatcactgcccggggttggacacgtttttgtgggataggcacgcccagagggcgtatctggttaacccctttctctttgcggcgggatttttggaggacttgagtcactctgtgtttccggccgacacccaggaaacaacgacgcgccgggccctttataaagagatacgcgatgcgttgggcagtcgaaaacaggccgtcagccacgcacccgtcagggccgggtgtgtaaactttgactactcacgcactcgccgctgcgtcgggcgacgcgatttacggcctgccaacaccacgtcaacgtgggaaccgcctgtgtcgtcggacgatgaagcgagctcgcagtcgaagcccctcgccacccagccgcccgtcctcgccctttcgaacgcccccccacggcgggtctccccgacgcgaggtcggcgccggcatactcgcctccgacgcaactagccacgtctgcatcgcaagccaccctgggtcgggagcaggacagccgacccgtctagcggccgggtcggctgtccagcgtcgtcgccctagaggctgtccgccgggcgtgatgttttccgcatctacgacccccgaacagcccctggggctgtcgggcgatgcgacgccgcccctgccgacttccgtgcccctggactgggccgcgtttcggcgcgcgtttctgatcgacgacgcctggcggcccctgttggagccggagctcgcgaaccccctaaccgcgcgcctcctcgcggagtatgaccgtcggtgccagaccgaagaggtgctgccgccgcgggaggatgtgttctcctggacgcggtattgtacccccgacgacgtgcgcgtggttatcatcgggcaggacccgtaccaccatcccggccaggcgcacggcctggcgtttagcgtgcgtgcggatgtgccggtgcctccgagtctacggaacgtgctggcggcggttaaaaattgttaccccgacgcgcgcatgagcggccgcggctgcctggaaaagtgggctcgcgacggcgtgctgttgttgaacacgaccctgaccgtcaagcgcggggcggcggcgtcccactccaagcttggatgggaccgttttgtgggcggggtggtccaacggctggccgcgcgccgcccgggcctggtctttatgctctggggcgcccatgcccagaacgcgatcaggcccgaccctcgccaacactacgtcctcaagttttctcacccgtcgcccctctccaaggtcccgtttgggacgtgccagcatttcctcgccgcgaatcgctacctcgaaacccgggacattatgccgatcgactggtcggtataagatgccgacatccggggtcttgatttacgagggggcaattaataaagactgttgatggttaaatctcgggtctcataccggtccgtgatgtcgggcgtgggggaagagagggtcccctctgcgtttactatccttgcctcgtggggctggacgtttgcaccccagaaccatgatcctggcgcgtcgccgaatacgacgcccatagagtcgattgcggggaccgcaccggacgcgcacgtggggcctctcgacggagagccggaccgggatgcgatctccccgcttacgtcgagcgtggccggcgacccgccgggggcggacggcccctacgtcacctttgatactctgtttatggtatcttcgatcgacgaactggggcgccgccagctcacggatacgatccgtaaggacctgcggctgtcgctggccaagttcagcatcgcgtgtaccaagacctcgtcgttttcggggacggccgcgcgccagcgcaagcgcggagcaccgccgcaacgcacatgcgtaccacgcagcaacaagagcctccagatgttcgttttgtgcaagcgcgccaacgccgcgcaggtgcgcgagcagctgcgggcggttattcggtcgcgcaagccgcgcaagtattacacgcggtcctcggatgggcggctctgcccggccgtccccgtgtttgtacacgagtttgtttcgtccgaacccatgcgcctccatcgagataacgtcatgctgtctacggaaccagactaagcacccccgccgtcccctttcttttccccctacccttcccccgttactgatgtgttgtacgtttcaataaataacacgtagcttattttgttggatgatggattgattgattttattgaccgttcgttcgcccggcggtgccgtcgccgcgcgcagagggaatatgcaagcgggcggggtggggaggaaagaaggtttcaggttccgggggttgggtctgcgtcgtccagggtggggctgatctgaatttcccgcagaacctcgaccagtaggtctgttgtgtttgctgggaactcgcccgccgttggggatacgggggcggggggtgtggtcgggcggacgtccaggggtgcgttatcgcacccccgcgccgcctcgggggccgtcccgtagatcgttgcggtgatgtagatggtgtccggggtccacaccaccgtcaggatgccggccgtcgcactccggacgctttcgccgtgcgatgagctgacccaggagtcaaaggggtacgcgtacatatgggcgtcccaccagcgctccagcctctgggtactagcgcgtcctataaagcggtatgcgcaaaattcggcacgacagtcgataatcaccagcagcccgatgggggtgtgttgtatcaccacgcctccgcggggcaggcggtcctggcgcgctcgaccccgcgtcagaaccgcgcgcgtccctgactcaaacacgtgcaccacctgtgccgcgtccggcagcgcgctcgttagcgacgccctggggtgatgtaggctgtacgcgatggtcgtctgggggttccccatgtctcgggggggtgggggtgaatgtcacccggcccgggtgcggtgggaacgcgagggaatggagggttaatagacaatgaccacattcggatcgcgtagagcagatagtatgtgctcgctaatgacgtcatcgcgttcgtggcgctcccggagcgggtttagattcatgtgcaggaactcggatgaggtggtgcgggacatggctacgtacgcgctgtttaggcgcaggtttccgggcgtgaagcatatggcgaccttgtccagactgagcccctgggagcgcgtgatggtcatcgcgagtttggagctgatgccgtagtcggcgttgatggccatggccagctccgtggagtcgatcgactcgacaaactcactgatgttggtattgacgacagacatgaagccgtgctggtcccgcaggacgatgtagggcaggggggactcctccaagaactcggccacgccggccgtcgcgtgccgccgccgcagctcctccgcgaacgcgaacacccgggtgtacgtgtaccccatcagcgtgtagttgtccgtctgcagggccacggacatcagccccccgcgcggcgagccggtcagcagctcgcagccccggaaaatgacattgtccacgtaggtgctgaagggggcgctctcaaacacctccccgaagagctcccgtaggataaggtatcgccccagaaaggccctcttcaggagcccaaactgggcgtggacggccgcggtggtctcaggctcttcgagggcgtagtggcagtagaacacgtccagctgctgttcgtccagcccggcgaagataacgtcaaggtcgtcgtcggggaagtcgtccgggcccccgtcccgcgggcccaggtgcttaaaattgaacgcacgctcccccggagagcggtcgctggtgtcggcggccctggttgccgatgcgccggcggcgtcccggcgtagcgacaggagttctgccgtcagctcccctaggcggccgtaggccagggtcctctgggtcgcgtccaggccggggcgctggagaaagttgtaaaagtgaatcagcccgccgaacatgagccgcgacaggaaccggtaggcgaactccaccgaggtctccccctgggtcttcacgaagctgtcgtcgcgcagcacagcctcgaaggtccgaaacgtcccgtcgaacccaaacaccatctttcggaggcgcgcggtcaccgcgacctggctgttgaggacgtacgtgatgtcgttccgggccacgactagctgttgcttgctgtgcacctcacagcgcacgtgccccgcgtcctggtcctgactctgggagtagttggtgatgcgactggcgttggccgtgatccacttttccatggtcagcgtgggttgctgcgtgagccgtcgatactcgtcaaactctttgaccgacacaaacgtgagcacggggagggtaaacacaacaaactccccctcgcgagtcacctttaggtaggcgtggagcttggccatgtacgcgctgacctccttgtgggacgagaacagccgcgtccaccccggaaggttggccgggttggtgatgtaactttccgggacgacaaagcggtccacaaactgcatgtgctcctcggtgatgggaaggccgtactccagcaccttcatgaggttcccgaactcgtgctccacacatcgcttgttgttaatgaaaatggcccagctgtgcgagaggcgcgtgtactcgcgtagggtgcggttgcagatgaggtacgtgagcacgttttcgctctgccggacggagcatcgcagtttttggtgttcgaaggtggactccagcgaggccgtctgggtcggcgaccccacgcacaccagcaccggccgcaggcggcccgcgtactggggggtgtggtacagggcgttaatcatccaccagcaatacaccacggtcgtgagtaggtgccgccccaggagcccggcctcgtcgatgacgataatgttgctgcgggtgaaagccggcagcgccccgtgtgtgaccgaggccaggcgcgtgagggcaccctggcccagccccaaagtctgctctagggcggtgagggcgtggaactcgtttcgcgcgtcttcgcccccgtgcgccgccagggcccgcttggtgatgtcgaggatcacctcccagtagtacgtcaggtctcgccgctgcaggtcttccagcgaggcggggctgctggccagggtgtacgggtgctgccccagctgggcctggacgtgattcccgcgaaacccgaactcgtgaaagatggtgttgatgggtcgactcagaaacgcccccgagagcttaacgtacatgttctgcgccgcgattcgcgtggcgcccgtgaccacgcagtccaggacctcgttgagggtctgcacgcacgtactctttccggatccggcgttgccggtgatgagatacgccgcgaacggaaactcccggagcggcaggccggtcgggacctccaaggccgccacgtcccggaaccactgcaggcgcggcacctgcgtgacgtcgagctgctgctgcgagagctctcggatgcgtgcgatgattggttggaccccgtgcatggacgtaaaatttaaaaacgcctcgtccctgaaccgcacggcgggtctggccccgggctgctgtgggggcggacctggtgcccggacgtcccgcgagccctccccgccggacgccgccatggccgcacagcgcgcgcgggcgccggcgatgcggacgcggggcggcgacgcggcgctatgcgcccccgaggacggctgggtgaaggttcaccccacccccgggacgatgttgttccgcgagattctcctcgggcagatggggtacaccgagggtcagggggtgtacaacgtcgtccggtccagcgaggccgccacccgacagctgcaggcggcgatcttccacgcgctcctcaacgccacgacgtaccgggacctggaggaggactggcgccgccacgtggtggcccgcggcctccagccgcagcggctggttcgcaggtaccggaacgcccgggagggcgatatcgccggggtggccgagcgggtgttcgacacgtggcgatgcacgctcaggacgacgctgctggactttgcccacggggtggtagactgctttgcgccgggcggcccaagcggaccgaccagcttccccaaatatatcgactggctgacgtgtctggggctggttcccatattgcgcaagacgcgcgagggggaggcgacgcagcgcctgggggcgtttctcaggcagcacacgctgccccggcagctggccacggtcgccggggccgcggagcgcgccggcccggggcttctggatctggccgtcgcgttcgactccacgcgcatggcggaatacgaccgcgtgcacatctactacaaccatcgccggggggagtggctggtgcgcgacccggtcagcgggcagcgcggcgagtgcctggtgctgtgcccccccctgtggaccggcgaccgcctggtcttcgattcgcccgttcagcggctgtgccccgagatcgtcgcgtgccacgccctccgggaacacgcgcacatctgccgtctgcgcaacaccgcgtccgtcaaggtgctgttggggcgcaagagcgacagcgagcgcggggtggctggcgccgcgcgggtcgtcaataaggcgctgggggaggatgacgagacgaaggccggctcggccgcctcgcgtctcgtgcggctcatcatcaacatgaagggcatgcgccacgtgggcgacatcaacgacacggtacgcgcctacttggacgaggcgggggggcacctgatcgacacccccgccgtcgaccacaccctccctgggttcggcaagggcggcaccggccgcgggtcgcgcccccaggacccgggggcgcgaccgcagcagcttcgccaggcgtttcagacggccgtggtcaacaacatcaacggcatgctggagggctatatcaataatctctttggaaccatagaacgcctgcgagagacgaacgcgggtctggcgacccagctgcaggcgcgcgaccgcgagctgcggcgcgcccaggcgggggcgctggagcgggagcagcgcgcggcggaccgggcggccgggggaggcgcgggccgcccggcggaggcggatcttctccgggccgactacgacattatcgacgtcagcaagtccatggacgacgacacgtacgtggccaacagtttccagcaccagtacatccccgcgtacggccaggacctcgagcgcctgtcgcgcctctgggagcacgagctggtgcgctgcttcaagattctgcgccaccgcaacaagcagggccaggaaacgtcgatctcgtactctagcggggcgatcgcctccttcgtggccccgtatttcgagtacgtgcttcgcgccccccgagcgggcgcgctcatcaccggctccgatgtcatcctaggggaggaggagttatgggaggcggtctttaagaaaacccgcctgcagacgtacctgacagacgtcgcggccctgttcgtggcggacgtacagcacgcggctctgccccggcccccctccccaacccccgccgatttccgggcgagcgcgtccccgcggggcgggtcccggtcccggacccggacccgatcccggtcgcccgggagaacgccgaggggtgcgccggaccagggctggggcgtcgaacgcagggatggccgaccccacgcccgccgatgagggaacggccgccgccatcctcaaacaggccatcgccggggaccgcagtctggtcgaggtggcggaggggatcagcaaccaggcgctgctgcgcatggcctgcgaggtgcgccaggtcagcgatcgccagccgcggtttaccgcgaccagcgtcctgcgcgttgacgtcacccccagggggcggttgcggttcgttctggacgggagttccgacgacgcgtacgtggcgtcggaggattactttaagcgctgcggggaccagccgacgtatcgcggttttgcggtcgtcgtcctcacggccaacgaggaccacgtgcacagcctggccgtgccccccctcgttctgctgcaccggctctccttgtttcgccccacggacctccgggacttcgagctcgtctgcctgctgatgtacctggagaactgtccccggagccacgccacgccctcgctgttcgtcaaggtgtcggcgtggttgggggtcgtggcccgccacgcgtctcccttcgagcgcgtccgctgccttctcctccgcagctgccactggatcctgaacacgctaatgtgcatggcgggcgtgaagcccttcgacgacgagctagtcctgccccactggtacatggcccactacctgctggccaacaatccgccccccgtcctctcggccctgttttgcgccaccccgcagagctctgcgttgcagttgcccgggcccgtcccccgcacggactgtgtggcctataacccggccggcgtcatgggaagctgctggaattccaaggacctgcgttcggctctggtgtattggtggctttcggggagccccaaacgacggacctcgtcgcttttctatcggttttgctaactccggaaaataaacgtgttttttatggaacgttccccacctgtcgtgtcatctctcgggggatggtggtgggcctgtgtgtgtgtcttgtgcaccgaaggaggaaagtgggggggtggtggtgctggtggtggaaagacatgatagagggaacaaagaaatagaagaaaaccacaaccggcgcgtgccagtaaatacggacgcgcgcacacgcggggggtaagttggagcacggggccccggtttattgaccaaattcagggaaacagaaaccgaatcttttcatcgaaagggtacacaaagctcccgccctcgccccacacgccttccagaacccccgtaaacaccagttgaatctcgcgcaggatctcgcgcaggtgatgggcgcagtccacgggggggagcaccaagggccgcgggtacagatccacggggacgccgaccgactccccgcccccgggacatacgcgcacgacgcgtctccagtattgctccgcgtccagcagggcgcctccgcggaaggccgtttggggcagggggtcgtcggcctcgcctgggggggtcagaacgctccagtactccgcgtccagacgcctcccgaaggcatccaggacaaagcggtcacaggcgtcctccatgacgccccgggccgcgcacacggcctcctccggcgggccggcggccggccgccggaggattcgtctcagcgcgtcgcgcataacctcggccgccgcggcgtacgcggccccgcggagaggaaatccctgcaggaagtcggtgtcatcgcgggagttccagaaccacgccccggtctggctccaggtgacgacgtgggtgtagacgccctctggcgccagggagggggcgaggcgcgggcgtatgccgttggccgaaagtacggcgcgcacggacgcctcgagggcccggcgggcgtcctggatcgcgccgtgcgcggcgtccgcgtccccggggtccacgttgaacagcccccagaacgcagccccggtgccgccgcagaccgcaaacttcaccgagctggccgtctgctcgatctgcaggcagacggcggccatgaccccgccgagcagctgccggagcgcggggcaggcgtcgcacgcgtccggcaccaggcgctccagcacggcccgggcccagggctccgagggggcggccgccaccagcgcgtccagcctttccaggcccgcccgcccccgggcttccggcagcccggcctccccgaggcccgcgagggcggccaggagctgggcctggagcccggagaaacaaaaccgcgccgtccagaccggcccgacggccgccggggggtcgagtagttggatggtggtggccgtggggtgccaccgcgcgaccgcttcccgaaaggcgggcaggaggcggccggccgcctccgaggccacggccggccatgcccgcgggggcaggacgaccctggcgcccaccgcgggccaggcccccaggcacgcggcatgggtggccgcggcgccccgcaccaggtcacgcgccgactcggcggcggcggcggccggcacggtaaacgtgggccagcccggaaatcccagcacggcaaagtattggacgggccctccccggacctcaaacccgggccccagaaaagcgaagacgggggccagggctccgggggcggcgtggaccgtggtatgccactgccggaagagggcgaccagcgccggggcggagaacccgtcgccggcgctcacgaagtagtcgtagccgcgcggcagcagcacccgcgccgtgacccgctgcgggtgtccgcggggccgcaggccgacctcgcacacctcgaccaggtccgcgaaggcgccctccttcctggtcggcggaaacgccagggtggtgtattcgcgcgcaaaacgcgcggtcctcgtcgtgatggtgacggcgagcgaggcggaggacgcgcactgggggctgtcgcgaatggcggccaggcgcgcccacgccaaccgcgcgccggggtgctcggcgacgcgcgcggacagggccagcgggtcgacgtcgaccttggcctccacgtccaggagggcggcgcgaggagcggccggcgggccccacgacgccctttcgaccctcacgaccagacccgtctgcgggtcccagcccaggcgcagcgggacgaagagggcccaccggcccgtctggcgctccagggccgccagaacgcacgcatacagcgcccgccacagggtcgggtcccccaggggctccagcggggaggcggccggggccgtcgcggcgcgggcggccgcgacggccccgggggccgagacgtcgggggagccgtagaagtcctgcaggtcggacgaaccaacggacacctccgcgaagcgcgcgcgcgcctcccccgcggcgtcgcgacagaccagatacagcagggcgtggaggcagtcgcgcgtgcgcgggggcagccataccgcgtatagggtaatggcgctgacgctctcctccacccaaacgatgccgggggcttccatgccacgacgcccgggggttgccgtgtatcgaacgagcgcggccccagacttatagggtgctaaagttcaccgccccctgcatcatgggccaggcctcggtgggaagctccgacagagccgcctcgagaatgatgtcagtgttgggctgggcgccggaggcgtgcgtgcgcaagcagcgcccccacgcgggcgcgcgcagcttgaagcgcgcgcccgcaaactcccgcttatgggccatcagcagcgcgtacagctgtctgtgcgtccggcaggcgctgtggtcgatgcggtgggcgtccagcagctccacgatggctcgcttggtgaggtttttaacgcgccccgccccgggaaacgtctgcgtgctcttggccagctgcaccccgaacagttcgccccagatgatcttgaacagcgacagcgcgtgctccgtctcgctcacggacccgcgcggggggcagccgctcagggcgtcggccacgcgcttaaccgcgtcctccgacagcaaggggccgtcggtcacgttacagtggcccagttcgaacaccagctgcatgtagcggtcgtagtgggggttcagcagctccagcacgtcctcggggctaaaggttcgccccgaccccccggccatcgagtcccactgcaggcacgcggccatggtgctgcacagacggaacagctcccagacgggggcgacgtttagggtggggtgtagggccacaagctccagctctccggcggcgttgatcgtggggatgacgcccgtggcgtagtggtcgtaaagccgccggaagatggcgctgctatgggcggccatggggacgcgaagacaggcctccagcagcaccaggtagatgaaccgcgtgcggccgaccaggctgttgaggccgcgcatgagcgcgaccacctcggccggcgcgacgtccggccggaggtacttttcgacgaaaaggcccacctcctccgtctcggcggcctgggccgacagggacgtgtcggggtcctggcagcgcagctcccgcagatcccgctgggccctcagggcatcaaaatgtatcccccgcaaaaacagacaaaagttcctcggggtcagcgcggcgtcgtggccccagaaccgcacgtgcatgcagttgagggtcagaagcatgtggaggatgttaagactgtccgcgaggcacgccagcgtgcacctctcgaagtagtgcttgtaccggaatttgctgtagatgcgcgacccccgcgcctgcgccgcgtcggcgtgcgacgcgtcgcagcgccctttgaaccggcggcacaacaggttcgtcacctgggaaaactgtgccggccactgcccgctggcgctcaccacgtggttgagcagcatgggcgtaaagacgggctccgagcgcgccccggacccgtccatgtagatcagcagctcccccttgcggagagtccgtacccgccccagcgactggtacacggacaccatgtccggcccgtagttcatgggtttcacgtaggcgaacatgctgtcaaagtgcggcggatcgaagctaaggcccaccgtcacgaccgttgtgtagatgaccacccggtaccggccccatgtggtcacgtcgccgggcggggtgagcgagtggagcagcagcacgcggtccgtaaactgccggcagaacctggcaacgacctccgcgaaggagaccgtcgacgagaagatgcagacgttatctccgccggccaggcgcgcctccagctccccgaagaaggtggcgtccgggggggcgtccggggggggcgccccgcccgccggcccccggcggcgcagggccgcctgcaggacctcgggccccaggcgcgggagaaacagacaacggcgcgccgaaaatccgggcatggcgtactccccgatgaccacgtgaacgttcttttcgccccggaggctgcacagaaagtccaccagctgcgcgttggcggtggcgtccatggcgatgatccgcgggcacgtgcgcagcaggcgcagcatcaacgcgtcgacgcggcccagctgctgcatcgtcggcgagtacagttggcccaacgtcgacatgacttcgtccaggacgagcacgtcgtagttgttcaacaggttcgggcccacgcgatgaagactttccacctgcacgatgagacggtggaaggggcggtcgttcatgatgtaattggtggatgagaagtaggtgacgaagtcgggcaaccctgactcagcgaaccgcgtcgccagggtctgagtaaaactccgacgacaggagacgaccagcacactcgtgtccggagagtggatcgcttcccccaaccagcggatcagcgcggtagtttttcccgagcccattggcgcgcggaccacagttacgcaccgggccgtcggggcgctcgcgtccgggaaggtgacgggtccgtgttgctgccgctcgatcgttgttttcgggtggacccggggaacccactcggccaaatcccccccgtaaagcatccgcgccagcgatacactcgacgtgtactgctcgcactcgtcatccccgatgggacgccgggcccccaggggatcccccgaggccgcgccgggcgccgacgtcgcgcccggggcgcgggcggcgtggtgggtctggtgtgtgcaggtggcgacgttcatcgtctcggccatctgcgtcgtggggctcctggtgctggcctctgtgttccgggacaggtttccctgcctttacgcccccgcgacctcttatgcgaaggcgaacgccacggtcgaggtgcgcgggggtgtagccgtccccctccggttggacacgcagagcctgctggccacgtacgcaattacgtctacgctgttgctggcggcggccgtgtacgccgcggtgggcgcggtgacctcgcgctacgagcgcgcgctggatgcggcccgtcgcctggcggcggcccgtatggcgatgccacacgccacgctaatcgccggaaacgtctgcgcgtggctgttgcagatcacagtcctgctgctggcccaccgcatcagccagctggcccaccttatctacgtcctgcactttgcgtgcctcgtgtatctcgcggcccatttttgcaccaggggggtcctgagcgggacgtacctgcgtcaggttcacggcctgattgacccggcgccgacgcaccatcgtatcgtcggtccggtgcgggcagtaatgacaaacgccttattactgggcaccctcctgtgcacggccgccgccgcggtctcgttgaacacgatcgccgccctgaacttcaacttttccgccccgagcatgctcatctgcctgacgacgctgttcgccctgcttgtcgtgtcgctgttgttggtggtcgagggggtgctgtgtcactacgtgcgcgtgttggtgggcccccacctcggggccatcgccgccaccggcatcgtcggcctggcctgcgagcactaccacaccggtggttactacgtggtggagcagcagtggccgggggcccagacgggagtccgcgtcgccctggcgctcgtcgccgcctttgccctcgccatggccgtgcttcggtgcacgcgcgcctacctgtatcaccggcgacaccacactaaatttttcgtgcgcatgcgcgacacccggcaccgcgcccattcggcgcttcgacgcgtacgcagctccatgcgcggttctaggcgtggcgggccgcccggagacccgggctacgcggaaaccccctacgcgagcgtgtcccaccacgccgagatcgaccggtatggggattccgacggggacccgatctacgacgaagtggcccccgaccacgaggccgagctctacgcccgagtgcaacgccccgggcctgtgcccgacgccgagcccatttacgacaccgtggaggggtatgcgccaaggtccgcgggggagccggtgtacagcaccgttcggcgatggtagccgtttcgttcgttttaataaaccgacgttgtgcgtttcaccatacttcggcgcgcgtgtgtgtgtgtttttttttttgtggtgtttattttccccccaccccttccttttctttcggccaccacccccctcctcccccgtactatacaacaaaaaataccacacatacgaccaaatacggacaatcatttctgtctttattcgctatcagagagtgggggcgtgagcgtggcaggagggcgggccacgtcggggtcccgccgtctggtgtgacgcgatggggggtccgatgcgcgccggtactggggccccggcgcccgggtgaccacgcgcacgtcggggggcacgtagaagttaccctcttcttcggactcgatgtccacgacgtcaaattcgtgggcggtcagcgagacgacctccccgccgtcggtggtgatgacgttgtgtcggcagcagcagggccgcgccccggagaacgcgaggcccataacttggcgagcgtatcgtcgaaggccaggcggctgtttcgccggatgtcccggtagatccccggctcgacgcggacgggggtgatgatcagggcgatcggaacggcctggtccgggaggatcgatgccttggcgggtccgggggccccgccaggcccggcgggcgctccgcggccgtcctccaggcggaacgtcacgccctcctccgcgcccgcgcggtgcctgccgaggaacgtcaccaggtgcggttgcagggggcagtcgggaaagtggctgtcgaggacgtatccctgcaccaagatctgtttgaagttcgggtggcgggggttggcgaagatgggctcgcggcgaaccagctccccggagctccaggccacgggagagatggtgcgacgctcaaggtcggggacgccaaacagaagcacctccgagacaacgccgctatttaactccaccagcgcccgatccggggcggagcatcgccttttttcgccggcggcgcgggaatcgagccagtcccggtcttgggtgacgagcgcctcctccgggcccggaacgcgcccgggcgcgaagtagcgcacgccggggttggggatggaccggatgaacgcccggaacgcctccggcgatcgccgcgccatcaggtcctcgtacgcggaggccgcgggggcgccggggtccgcggggtcgaacgcgtacttggctcggcacttaacctcgtagaaggccaggggggtctggggggcgggggccaggtagccgtgagggtccctggggcacacgaggatgtccagggacgcccccaccatgcccgtgtggccgtccatgaggaccccgcacgcgtgcacgttctcctcggcgaggtccccgggttggtgaaagacgaagcgcccggcgtcggcgtcgtcgttgacgcccgcgtccgcgcggcccacgcagtagcgaaacagcaggtttcgggccgtcggctcgttcacccgcccgaacatcaccgccgacgactgggcgtccagccgcaggctggcgttgtgggtgagccactgggacgagaagcacggaccctgcgcgccccaccgcagcgtggaggcggtcgtcaggccccgccgaagcagggcccagagctggcagtcggcctggttttgcgtcgccgcctcgtaaaatcccataagcgggcggggggcgacggcttcggcggcggacgggggggcgcggcgcgtcaggcgccagaggtgccggccgagcccgcggtccaccatgccggccgcctccagcgacacgacgagggagcacagatagtccaggcgagcccacaggggcccgatggccagaggggagcggacgccgcgcagcaggccgcgcaggtggcgctcgaacgtttccgccaagatatgggggggcagtgcgttggggatcgccgacgccgaccacatcgggtcggggtccgggggaccggggctgcagtccgggtcgatggcgtgtgcgccccccggcgagaggggaatgtcgggggttggcgggccggatgaggcctcagagagggccggggacgcgggccgggccttttcgcccggggccccgccgtcgggttgcccacgtggggggctctggggccaatgggaacccggggcccccggtgacgtggggcggggtggggcggggcggggcccaaagacggtcgccagatctaggctgttgggtcggggccgcttcgggggactatcggggtcgcgggcggggtccgcggggcgcttggcgccgggtgttgcggcggccgccatttttacgagcagccgaagagctcgagggcggaagggatcctcacgacagagagtggcgcgcggccgggttggcgtgacagaggcgggagaccagcaccagcagcggcctcagctcgggcggcagcgacaccgacgacaggacggccttgtgcgtgcgctggtaatttatacactgctccgtgaacgcgcgccgaatcttgggattgcgaaggtggcgccggatgccctccggcacgtcatacgccaggccgtgggtgttggtctcggccgagttgacaaagagggcggggtgcagaacgcagcgataggcgaggagggccacggcaaagtccggcgagagctggttgttaaagtactggtagcccgggacgcgggtcacggggacgcccaggctcggggccacgtacacgctaaccagcagctccagcagcgtctgccccagggcgtagagatcgaccgccagcccgacgtcgtgcttcagggggcggttgttaaactcggcccgctcgttgttgaggtactttaccgagagctccggtggctggttgtacccgtgccccaccagagtgtgaaagttggccgtggtcagggcggcgggcatcccaaacccccggggggactcgaggtccggctcctggaggcaaaactggccccgggatatcgtggagttggagttcagggtcaccaggctaaagtcggccaggacggccggccggagcgacaccgcgtccgatcgcagcatcacgaggacgttggcgcacttgatgtccaggtggctgatcccgcacctggtgttcaggaacaccacggcgcgcgccaggtctgtgaagcagtggtggagggccgtcgcgacggagggggtggtcgcgcgcagggacgccagctggccgatgtacttgccgaggtccatgtcgtacgcggggaacacgatctggcgctgctgcagcgagaacccgagcggggtgataaagccgcggatgtcgtgggtgcggccgccgcgaagagcgcactcccccacgagcagggtcgcgacgagctccacggcaaaccactctttttcccggatggtcttcacggcgagcttgtgttcgcgaatcaactgcacctcgccgtacccccccgagcccccgaagctgcgggccccggggatctccagggtcgtgtagcggagggcggggttgacggcgaatacggggatgcatagcttgtggatgcgcgcgagggacaggatgtgcgaggggggcgacgggggcgaggtcatggccgtctcggacctgcgcaggggcgggcgccttagcttggccgcagggccgggggcctcgggggacgagcggcgacgagacgagcggctcactcgccatcgggacagtcccgcgcgaagccgctcccggaagctggatcggcggcgggacccggggcgggctccggagacggcgccgtctcggggggaggggccgcttgggcgtccggacgcccggcggctgagggagtgtatgtaggacgcgagccaggccttgaaggagcgtcggtgtgcaccttgggggctgatgtcagctgccacatgactagcaggtcgctgtcgcccggactcatccatccgtccgccaggtcgccgtccccccacagagacgcgttcgccgcggcctcttcgagctgctcctcctggtccgcaagacgatcgtccgccgcgtccaggcgctcgctaagcgcgggatcgaggtaccgtcggtgtgcggttagaaaatcacgtcgcgccgcttgctcttccacgcgaattttaacacaggtcgctcgctgtcgcatcatctctaagcgcgcgcgggactttagccgcgcctccaattccaagtgggccgccttggcggccataaaggcgccaacaaacctaggatcttgtgtactcacgccctcccggtgtagctgcagggtctggtccctgtacacctcggcccggaggtgcgtctcggccaaacgtcggcgcagggccgcgtggctggcgtctcggctcatctcgccgcccccgcgcgcgcccgacgtcggactccttcgccccgacccccctgacctcagccgcccccgcctcgcccgcgatgtttggccagcagctggcgtccgacgtgcagcagtacctggagcgcctggagaaacagaggcaacagaaggtgggcgtcgacgaggcgtcggcgggcctgacgctcggcggcgatgcgctgcgcgtcccttttttggattttgccaccgcgacgcccaagcgccaccagaccgtggtcccgggcgtcgggacgctccacgactgctgcgagcactcgccgctcttctcggccgtcgcgcggcggttgctgtttaatagcctggtgccggcgcaactcagggggcgtgactttgggggcgaccacacggccaagctggagttcctggcccccgagctggtgcgggcggtggcgcgcctgcggtttcgggagtgcgcgccggaggacgccgtgccccaacgcaacgcctactacagcgtcctgaacacgtttcaggccctgcaccgctccgaagcctttcggcagttggttcacttcgtgcgggacttcgcccagttgttgaaaacctcgttccgggcctctagtctcgcggagactacgggccccccgaagaaacgggccaaggtggacgtggccacccacgggcagacgtacggcaccttggagctcttccagaaaatgatactaatgcacgcgacctactttctggccgccgtgctgctcggggaccacgcggagcaggtcaacacgttcctgcggctcgtgttcgagatccccctgtttagcgacacggccgtgcggcacttccgccagcgcgccaccgtgtttctagtccccaggcgccacggaaagacctggtttttggtgcccctcatcgcgctgtcgctcgcgtccttccgggggatcaagataggctacacggcccacatccgcaaggcgaccgagcccgtgtttgatgagatcgacgcctgcctgcggggctggtttggctcgtcccgggtggaccacgtcaagggggaaaccatctcgttctcgttcccggacggctcgcgcagcacgatcgtgtttgcctccagccacaacacgaacgtaagtacgccttcctcccgcggtgcctgtttccccggtgccgccctccccgagatcgaccgacagacaaacacagccagacgcgagtgtgggacgacacgcccgcagccccccccccgccatggcgggggggaagccttactgtttatttgtaatcggacgatgaggctctggccacggcccgcgcgaccgcggggcagctcgttgcaaacaggcggctggtatacgatgacagaacgcagaggcgccacccggcgctggtcgggcggatgacgctttccgcgccgtcccggcccacgacgacctcgtgcaggtgggccgtgatgcgcgggcggcgggtcgcctgccgcaggataaccgcgtccacggggtgcccgaagaggagctgacacaggctcgcgtccccccggacggccagggtgcgctgggccatattggaccacatgcacggggcgacgcagggacaggcctccgccacggcgggggcgcgccacagcgcgttggcggaatcgatgtgggccgtcggggcgcaggcgccgcctcctcccggggggtcggtaatcctggatagcagccatcctaaatggcgggcccggctgcccgggggacagagcgaccccaggtcatcatccatggcccagcagtatatgcggccgccggggaggtgccaccaggcccccggacccagggcacagcacgccccggattcgggggccgtgtccgtgggtaccaggtaggcgccgtcgagctcgtgggccacgggctcgtccgcgagctgttcggcggcggggtcgggggtttcctccgggggggaggcagcttccaggtggccgaaggctagggtgcacagcagcggggtccgggggtgcgttacgctgcggaggtggacggtggcgcagtagcggcgctcgcggttaaagaagaaaatggcaaagaacgtgttcgaaggcaggcgcagcgccttgggccgcgtcaggtacaggaagatctcgcagaaaagggcacgctcggggtcggggtccggaagggccacctggcacagcggctcggtgaggaccgtgaggcaccgaaaaatcttaagccgctcgtccccccgaacgacgcgccacacgaagacagagttggcgatgcgcgcgacgaggtcggcttcgggccccgggtcgggggcgcgcgcgtcggggggggcgccccggtgacccggcggggccgcggctcccggggggcctggcgtcgcctggggacgccagagtgcccgctgtgccaggttggtggtggggaagggaccggagacgcaccaaaagcagaggggccagcgcgtgtatgagttggggggggggtgggtgagcggtggaacaaaagcacgcgtcagcggacaaggccgggtcccgtagccgccccgcgacagaaccggagtccgacggcacgcgcgacggggtctgcgaggctgaggtacgccgcggtgttaatggtaaacgcaaagcctcccggaaagaccactagcccgcagaggcggcgattgaacccaaggcagaggtacgcgtagctctctcccggaaggtattgctcgcagaccctgtgtggggcagtggaggggctgccctccatgaagcgacatttactctgctcgcgtccattgacgtcaccgtcaatcaccactgcgattggacggttggtgaggcgcagcgtgtctccgctggtgctgtagtagtcaaacgcgtagtgggcgtcggagtcggcgaagcgggcggggatgtcgtcgctgagagggacgagccgccgccgccgcccccgaccgccctggccgcccagatgcgccagcacggccagggcgtacgcggtgtgaaagaacgcgtcgggggcggtcccctcgagggcgcgcatcaggttctccaggagcacggggaagcgccgcgtcacctcccctagccactcgctctggtgggggccaaagtcgtagcgcaggcgctggaagatgcgcgggccgccttggagcgcggcccggatagagtggcccagggcccgcagacacgcgatctggatgcgcgcgacgaaggccacctcggccgcgatgtcaaagggctgcagcacggggcgcgggtggcgcaggggtccctcgagcgcgggaaagcgacgcagcagcgccgtctgggccgcgggggacagctggtgggggcgcacgacgcgctcggcggcacaggcctccgtcagggccgtggccagctcggaggacagccgcggggggcgggcgcgtcgcccgccccacgccaccgaattctcgtaggagacgacgacgaagcgctgcttggtcccgtagtgatggcgcaggaccacggagatggagcgacggctccacagccagtcgggccggtcgccgccggccagagcttcccacccgcggtccagccactcgaccagcgatcgcggcttggcggtccccggcacgagggtgagcacgtcgttgaggacgtcctcgcccgcggcccgggggcccccccggctggcaaagcgccccccgccgggcggctccaggcccgccagcaccgcctccgcgtccgacgcgcccagggctcccccgctgacggcctggtggaccagggcgccctggcggagccccgaggcgacgccggaggccgcgtgcttggggcgcgcgcggaccgggtggcggcgggtgacgtcctgcacggcccgctggaccagcgcgaggatctcctcgttctcttgcgtgatggacacgtcctccgcggtggccgtgtcgcctcccggggccgtgagctgctcctccggggagatgggggggtctggggtgccgacaacggccggcccggccccgcccgagaccgaggacgcctggggagtgggggtgccgctttcccccatccccagggacaggtgggccgccgcctccgtcgcggcggcgggagccgcggcccccagccgcgcgacgtagcgacaaaagtggcgacagaggcgcatgaggcgcgcgccgtcggccgcgtatcgcgtgtttggcgggacgagctcgtcgtaactgaacaggagcacgcgggcacaggtcgcccacgggccccacgccaggcgcagcgccgcgaccgtgtacgggtcgtacacgccttgggcgtcgcacgcgaccggcagggagacgaacagcccgcccgcgctggggacgcgcggcaggaggtccgggtgcgccgggatgacgggggctaggatcgcccccaccgcatccgccggcacgtaggcggcaaacgccgaacgccacggggtgcagtcgccggtcgcgtgggcccgggtctgggtttcgacccggaagttcgcggccgccccaccgtcggggcggccgcgcacgagggcggacagcgggacccccgccgccgccaggcactcgctggagatgatgacgtgaatcagcgaggcggggctgctcgggtcccgggtgagatcgtattggacctcgttggcaaagtgcgcgttcatggcccggccggcggtgcgagcccttcccggtgccggaaggggcgtgggtggggggtgcgtgtgcgcgtcctcggggcccgcgggcgcacgtgcgcttatacgctgtgtgtttcgtctgtccccagggaatccggggccaggactttaacctgcttttcgtcgacgaggccaactttattcgcccggatgcggtccagacgattatgggctttctcaatcaggccaactgcaagatcatcttcgtctcgtcgaccaacaccgggaaggccagcacgagctttttgtacaacctccgcggggccgccgacgagctgctcaacgtggtcacctatatatgcgacgaccacatgccgcgggtggtgacgcacaccaacgccacggcctgttcctgctatatcctgaacaaacccgtgtttatcacgatggacggcgccgttcgccggacggccgatctgtttctgcccgactccttcatgcaggagatcatcggggggcaggcccgcgagaccggcgacgaccggcccgtcctaacaaagtcggcgggggagcggtttctgctgtaccgcccctccaccaccaccaacagcggcctgatggcccccgagctgtacgtgtacgtggacccggcgttcacggccaacacgcgcgcctccggcaccggcatcgcggtcgtcgggaggtaccgcgacgatttcattatcttcgccctggagcactttttcctccgcgcgctcacgggatcggcccccgcggacatcgcccgctgcgtcgtgcacagcctcgcccaggtgctggcgctgcaccccggggcgtttcgcagcgttcgcgtggcggtcgagggcaacagcagccaggactcggccgtggccatcgccacacacgtgcataccgagatgcaccgcatcctggcctcggcgggggccaacggcccggggcccgagctcctcttctatcactgcgagccgcccggcggcgcggtattgtaccccttctttctgctcaacaaacagaagacgcccgccttcgaatactttatcaaaaagttcaactccgggggcgtcatggcgtcccaggagctcgtctccgtgacggtgcgcctgcagaccgacccggtcgagtatctgtccgagcagctcaacaacctcatcgaaaccgtctctcccaacaccgacgtccgcatgtactccggaaaacgcaacggtgccgcggacgacctcatggtcgcggtcatcatggccatttacctggcggccccgaccgggatccccccggccttttttccgatcacgcgcacgtcttgagtctttcttgccgtttcttttgtttctctttctttccccccctctctccgcaataaacgccttcccggaactgtgtttccccccctacaacagtgttgtccgttggttgggtggttggggtgcgggggtgggcgggggaagcaagaaaacggtcggcgaacacaacatcgggaaaacggattcccgcacgtgcgtcttcccagattcgacacacacaccccccttctccttaaataaacacaaaccacacgctcgttggttggttaatgccagcgctttatttacgtcttgttttttttgcgtttcctccgcgggtcccttcccaacacgcctgcccccgcctcaggggtagcggataaccggggccatgtcgccggattgcacaacggcggcgccgtcgaacgtacacacccgaaccgccggggccagggccaggatgtccccgagttggcccgcgtgcgccagccaggcgaccagcgcctcgtaaagcggcagcctgcgttcgccgtcctgcatcagcatgggggcttcggggtggatgagctgggcggcttctcgcgtgacgctctgcatctgcaggagcgcgttcacgtatccgtcctgggcgctcagcgcgagcagccgggggatgagcgtgaggatgagggtggttccttcggttatggagtagaccatgttgaggacgagcgaccgcagctcggtgtttacggaggcgagttgctggacgtcggccacgagcgagagacgggccccgttgtaatacagcacgttgaggtcggggagctccccgggcgtccgggggtcggggttgaggtcccggatgccccgggcgaccagccgcgcgactatctcgcgggccaggggcgttgggagcgggaccggaaaccgcagcgtgaggtccagcgactccaggcgcacgtccgtcgcctggccctcgaagacgggcgggacgaggctgacgggatccccgttgcagaggtcgacgggggaggtgttgcggagattgacggtgccggcgtgcgtgagccccaggtccacggggcaggcgacgattcgcgtgggcagcacccgcgtgattaccgcggggaagcgcctgcggtacgccagcaacaaccccaacgtgtcgggactaactcctccggagacgaacgattcgtgcgccacgtccgcgagcgccagctggcggcggatggtcggcagaaagaccactcgaccctcgcaccgctgcagcgccgcggcatcggggcgcgagatacccgaggggatcgcgatgtctgcttcgaaacaatccgtgatcatggcgccgggccgcgagacaccggaacgcgggggtgcgggagggccggaaagcgcaacgcaaccgggacgatgatgaaacagagatggggggcaccgaccgtgtgggagagggggcggggcagggctcagcagcacgcacggggaggtctgtcgtgcgcaggagccccaggtgagaatcagtcccccggagctcgggtctgggttttattgggacctgccctcggaatcgcggctcccagtccaagcccccctggggggggcggggacagggggtgtgtgtgggtaaaagcaacgtcggaaaatcaaacccaatgccccaaacaggaaaaaaaaagacgggcgggtggagggaaagctggggaagaagaagccaattttacagagacaggccctttagcggggaggcgtcgtagatgagatactgcgtaaagtgggtctctcgcgcgtgggcctccccatcgcgggcgctgcgtagcagggcggggtcgctggcgcaggtgatcgggtaggcttcctgaaacaggccgcacgggtcttccacgagctcgcggcaccccggcgggcgcttaaactgcacgtcgctggcagcggtggccgtggataccgccgatcccgtttccacgatgagacgctccaggcagcgatgtttggccgtgatgtcggccgcggtgaagaacttgaagcaggggctgaggacgggcgaggccccgttgaggtgataggccccgttgtacagcaggtccccgtacgagaaccgctgcgacgcccacgggttggccgtggccgcgaagggccgcgccgggtcgctctggccgtggtcgtacatgagggctatgacgtccccctccttgtcccccgcgtacacgccgccggccgcgcgtccccgcgggttgcagggccggcgaaagtagttgatgtccgtggccacgggggtggcgatgaactcacacacggcatcctgcccgtggtccatgccggcgcgccgcggcacctgggcgcagccaaagaccgggaggggctgggccggccccagccggtttcccgccacgaccgcgttgcgcaggtacacggcggccgcgttgtctagcagcgggggggccccgcggccgaggtaaaagttttgggggaggttgcccatgtccgtaacggggttgcggacggtgcccgtggccgcgacggcggtgtagcccacacccaggtccacgtttccgcgcggctgggtgagcgtgaagttgacccccccgcccgtttcgtggcgggccacctggagctggcccagaaagtacgcctccgacgcgcgctcggaaaacagcacgttctcggtcacgaagcggtcctgccgcacgacggtgaacccgaacccggggtggaggcccgtcttgagctggtgatacagggccacggggctcatcttgaagtaccccgccatgagcgcgtaggtcagcgcgttctcccccgccgcgctctcgcgggcgtgctgcaccacgggctggcggatggaggagaagtagttggcccccagggccggggggaccagggggacgtcgcgcgccaggtcgcgcagggccggggggaagttgggcgcgttggccacgtggtcggcgcccgcaaacagcgcgtggacgggcaggacgtagaagtattcgccattttggatggtgtggtccaggtgctggggggccatgagcagcacgccggcgtgcagcgccccgtcgaagatgcgcatgttggccgtcgacgcggtgttggcgcccgcgtcgggcgccgcggagcacagcagcgccgtcgtgcgctcggccatgttgtgcgccagcacctgcagcgtgagcatggcgggcccgtcgacgacgacgcgcccgttgtggaacatgcgcttgaccgtgttggccaccagattggcgggatgcagcgggtgggcggggtcggtcacgggatcgctcgggcactcctcaccgggggcgatctccgggaccaccatgttctgcagcgtggcgtacacgcggtcgaagcggacccccgcggtgcagcagcgcccccgcgagaaggccggcaccagcacgtaatagtagattttgtggtggacggtccagtcggccggccggtgcggccggtcgtcggcggcgtcggccgcgcgggcctgggtgttgtgcagcagccggccgtcgttgcggttaaagtcggccgtcgccacgttgcacgccgccgcgtagacgggctcgtgcccccccgcgtcaatccggcagtctcggtggcggtccagggccgcgtgtcgcataaggccgtcgcagtcccacacgaggggcggcagcagcgccgggtcgcgcatcaggtgattcagctcggcctgagcctgcccgcccagctccgggcccggcagggtaaagtcgtccaccagctgggccagggcctcgacgtgggccaccaggtcccgatacacggccatgcactcctcggggaggtcgcccccgaggtaggtcacgatgtacgagaccagcgagtagtcgttcacgaacgccgcgcatcgcgtgttgttccagtagctggtgatgcactgagtcacgagccgcgccagggcgcagaacacgtgctcgttgccgtgaatcgcggcttgcagcaggtaaaacaccgccgggtagctgcggtcctcgaacgccccgcggacggcggctatggtagccggcgccatggcgtggcggccaacgccgagctccaggccccgggcgtcacgaaacgccaccggacacagcgccaggggcaggttgccgttgaccacgcgccaggtggcctggatcgcccccggaccggccggggggacttcgccgccgggaagctcgacgtcggccacgcccgcgaagaagtcgaacgcggggtgcagctccagagccaggttggcgttgtcgggctgcatgaactgctccgcggtcatctggcactcggcgacccaccggacccggccgtgggcgaggcgctgccgccaggcgttcagaaaacgctgctgcatgtccgcgccggggccggccggggccgcgacgtacgccccgtacggattcgcggcctcgacggggtcgtggttcacgcccccgacggccgcgtcgatgttcatgagcgaaggatgacacacggtcccgaccgcgttctccatggacagccgcagaacctggtggtcctttccccaaaaaaacagctgccggggagggaacgcgcggggctccgggtggccgggggcgggcaccaggtccccggcgtgcgcggcgaagcgctccatggccgggttgaacagccccaggggcaggacgaacgtcaggtccatggcgcccaccagggggtagggcacgttggtggcggcgtagatgcgtctctccagggcctccaggaagaccagcctgtcgcctatggccaccagatccgcgcgcacgcgcgttgtctggggggcgctttcgagttcatccagcgtctcccggttcgcctcgagttgctcctcctgcatatccagcaggtggcggcccacgtcgtccaggctccgcacggccttgcccatcaccagcgccgtgacgaggttggccccgttcaagaccatctcgccgtaggtcaccggcacgtcggcctcggtgtcctccaccttcaggaaggactgcaggaggcgctgtttgatggcggcggtggtgaccagcaccccgtcgaccggccgcccgcgcgtgtcggcgtgcgtcaggcggggcacggccacggacggctgcgtcgccgtggtcaggtccacgagccaggcctcgatggcctcgcggcgatggcccgccttgcccaggaagaagctcgtgtcgcaaaagctccgcttcagctcggcgaccagggtcgcccgggcaaccctggtcgccaggcgcccgttgtcgagatatcgttgcatgggcaacagcagggccaggggaggcgccttctccaacagcacgtgcagcatctggtcggccgtgccgcgctcaaacgcccccaggacggcctggacgttgcgcgcgagctgctggatggcgcgcagctggcgatgcaggctaatgcccgtcccgtccagggcctcccccgtgagcagggcaatggcctcggtggccaggctgaaggcggcgttcagggcccggcggtcgatgaccttcgtcatgtaattatgcacgggctgctcgacggggtgcgggccgtcgcgggcgatgaggggctggtggacctcgaactgcacacgcccttcgttcatgtaagccagctccgggaacttggtgcacacgcacgccacggacaggccgagctccagaaagcgcacgagcgacagggtgttgcagtaggaccccagcagggcgtcaaactctacgtcatacaggctgttttcgtcggagcgcacggcggcgaaaaaatcaaagagtctgcggtgggacgccacctcgatcgtactcaggatggagccggtgggcaggatggccgcggcgtaccggtaacccggggggtcgcgggcaggagcggccattgggttccttgggggattcgcaggctccatcaagccgagctcgggaaggccaagcccctcccgcacaacgcctcaccgccggcggacgcgactaacaacccacgggccgccaaaaccccaaggggcaacccgaccaacaacaggcgaggggaggaaaggcgtaaagggggcgttgggaggcaaaaagaaagaaaacacccagacgtaggcccgaggaccggccggcgtcctctgtccccgagcacccactgtgcccaacaggcacgggggcgagctgcccctgccttatatacccccccgccacacccccgttagaacgcgacgggtgccttcaagatggccctggtccaaaagcgtgctagaaaaaagttggtaaaggcggcaaagcagtccgccgccgccacccacatggcggcgccggccgcgcaggcgattcccagagaacgggcgcggaggggatccgtgcggggcagcagctggctggcggtgatccaatggaaaagcccgtcgggactgaacgtctcatgggcggccgccaccagggcgcacagggccgcgccgcccatgatcacgcacaacccccaaaacacgggtggcgacaacggcaggcgatcccgtttgatgttcacgtacaggaggagcgcccgtgccagccacgtgacatagtaggcgaggacggcggctataatacatgccggcgccaccgcccgtccggtccacccgtaatacatgcccgcggccaccagctccagcggcttgaggaccaggaacgaccaagcaaacatcaccacccgcttggaaaagaccggctgggtgtggggcggaagacgcgagtaggccgaactgacaaaaaaatcagacgtgccgtacgaggacagcgaaaactgttcatcgagcggcagttctccgtcctccccgccacacgcggcctcgtctaccagctcgcgatccaacaaaggaacatcatcccgcattgtcatggtcggtgcggggagccggcgaggcagcaaaaccgaaagtagtgctggcggcgcgggcccgggtccggacccaagcttcagggatggggggcggaggccaaaatcaaacaagcaccgcgcgggttctacacacaacccccacccgggtagtatccgcggatgcgagtgcctggcgaagtcacgtcccagcaggatataaacctcggccgttgggcccggaacccccgaaattcacacccacgccctgacgcccaaatcatgggtggatgtggttcgcgagccgcacatccgtgcgtccgccctcccccgcgggctgatgacgtggcggttagtcagtgggaaggcagggggaaagatgggttgggggaggaaacgaagaaaacacccagagggccacgtcgggaatgcgcccggagttgtccttaaaaggccggccgtgcgtgacggaagccgtcgtttgcccaagcaccgacgccgcgatccacagtggggggagttcctccgtccggccacaaccctacgcgcgggcggcacgcgcgagagcaacccacgggtcccgttcgcgccaccgccagcccttgctcccaccaccctcctcccaccaccccactattcccccccccaagtccgccccgtggctcgccggccatggagctcagctatgccaccaccctgcaccaccgggacgttgtgttttacgtcacggcagacagaaaccgcgcctactttgtgtgcggggggtccgtttattccgtagggcggcctcgggattctcagccgggggaaattgccaagtttggcctggtggtccgggggacaggccccaaagaccgcatggtcgccaactacgtacgaagcgagctccgccagcgcggcctgcgggacgtgcggcccgtgggggaggacgaggtgttcctggacagcgtgtgtctgctaaacccgaacgtgagctccgagcgagacgtgattaataccaacgacgttgaagtgctggacgaatgcctggccgaatactgcacctcgctgcgaaccagcccgggggtgctggtgaccggggtgcgcgtgcgcgcgcgagacagggtcatcgagctatttgagcacccggcgatcgtcaacatttcctcgcgcttcgcgtacaccccctccccctacgtattcgccctggcccaggcgcacctcccccggctcccgagctcgctggagcccctggtgagcggcctgtttgacggcattcccgccccgcgccagcccctggacgcccgcgaccggcgcacggatgtcgtgatcacgggcacccgcgcccccagaccgatggccgggaccggggccgggggcgcgggggccaagcgggccaccgtcagcgagttcgtgcaagtgaagcacatcgaccgtgttgtgtccccgagcgtctcttccgcccccccgccgagcgcccccgacgcgagtctgccgcccccggggctccaggaggccgccccgccgggccccccgctcagggagctgtggtgggtgttctacgccggcgaccgggcgctggaggagccccacgccgagtcgggattgacgcgcgaggaggtccgcgccgtgcatgggttccgggagcaggcgtggaagctgtttgggtcggtgggggctccgcgggcgtttctcggggccgcgctggccctgagcccgacccaaaagctcgccgtctactactatctcatccaccgggagcggcgcatgtcccccttccccgcgctcgtgcggctcgtcggtcggtacatccagcgccacggcctgtacgttcccgcgcccgacgaaccgacgttggccgatgccatgaacgggctgttccgcgacgcgctggcggccgggaccgtggccgagcagctcctcatgttcgacctcctcccgcccaaggacgtgccggtggggagcgacgcgcgggccgacagcgccgccctgctgcgctttgtggactcgcaacgcctgaccccgggggggtccgtctcgcccgagcacgtcatgtacctcggcgcgttcctgggcgtgttgtacgccggccacggacgcctggccgcggccacgcataccgcgcgcctgacgggcgtgacgtccctggtcctgaccgtgggggacgtcgaccggatgtccgcgtttgaccgcgggccggcgggggcggctggccgcacgcgaaccgccgggtacctggacgcgctgcttaccgtttgcctggctcgcgcccagcacggccagtctgtgtgagatatcccaataaagtgcagtcgttttctaacccacggatgccgttgtatgcctatacgggggactatggggggggaaaggaaaggaaacaggaatggagaagggaaaggaacagaggcggtagcggacgcacggcggacacaataacaaacagaccgcggacacggagggagtcggttgggttgggcgtggacgccgctgcgtccacacacccgtttattcgcgtctccacaaaaatgggacgcacgttcggaccaccctaaggatgcccgccagggccgcggtaatcataacgacccccagcgcggacgcggccagaaacccgggggcgatggtggcgatgggcagcgtgtcaaaggccagcagatgaatcacagttccgttggggaacaacaacagggccacggacggcacgtcgctggaaaacacgttcggggtgcccgccaccggcccctgggccagctgctgttgggtggcatccgtgtccaccagcagcaccgacatgacctccccggccggggtgtagcgcagaaacacggcccccacgaggccgaggtcgcgccggttttcggtgcgcaccagccgcttcggctcaatctcccgcgcgtgcccttcgcaggtggcggtgagataggtgataaacagcgggcggcggacgtcaacgcccgtaagcttgtatccgatcccgcggggcaagggggtgtgggtgacgacgtagctggcgttgtgggtgatgggcacgaggatccggggctccgcgttgtgcgacgggccgctacactggtgggtggcctccgggacgaaggcgcggatcagggcgttgtagtgcgcccagcgcgtgagaacggaggccacgccgcgggtctgttgtgccatgacgtccgccgggatgtcggatcgggtggccatggccagcgcgtccaggatgaacccgccctcggcgagatcgaagcgcagggaagctgcgcatggggaaaagtggtccgggagccagaagaggtttttctggtggtcggtcctggctagcgcggcccggagatcggcgtgggtcgccgcggcgacgtcggacgtacacagggccgtggttatgaggaggccccggcgggcgcgttcccgctgctcggccgagggcgcgcccgccaggaacggcgcccggaggacggccgtggcgtaaaacagcgctcggcggaccatcggggcggttagcgcgcggccgccgagaaactcggcgtacagggcgtcgatcaggcgggccgcgctcggggccaccgcgccataggccgcggggctgtccaacacgaacgccagctgatagcccagcgcgtgcgccaccaggctctgctctcgctcgaggatcgcggccaccagatgcccgaggcgcgcctccagccgcaggcgggccgccgggtccaacacggacacgttcaggaacaccgagtcggccgcgcagcccgctgctccccgggcggccaggccggccagcacgcgcgagtgggccaaaaagcccagcaggtcggagaggcgaatcgcgtcgtgggcgtgggccgcgttgacgaacgcaaaccccgacgaggcgagcagccccgcgaggcgccagaacagggacggacgcgcgtccgtgccggagcccgggtcctcccccaaaaactccgcataggcccgcgacatatactgggcgtagttcgtgctctcctcggggtagccggccacccgccggagggcgtccagcgccgagccgttgtcggcgggcgtcggggcccccaggacaaagacgcgatacctggggccggccggaggcccggggagcaccgcgggggcgttttcgtcggtcggatttccgacccgagcgagggtcttgtccgcaggcaccactatgatctcggccggagggctgtcccgcatcgatatcacgagccccatgaagcccttcccgtatcgcgcgcgcacgagcgcggcgtcgcacccgaacgccagcccgcccgtcgtccagacgcccacgggccacgtcgaggccgacggggagaggtacacgtaccgacccggagtccgtagcaggcccctggcggccagccaggtcacggatgcgttgtgcagatgcgcgatgctcaggttcgtcgtcggatgcctcggtgtccccgcgggcggccccgggggcggcgcgttgcgtcggccgtccgggtgcctctcggtcgccccgtcgtctccccgcgggaacgtaagcccctcgcggtccggcgcggccgcgaatgttacccaggcccgggaccgcaacagcgcggaggcgccggggttgtgcgacagtcccttgagctgggtcacctcggcggggggacgggacgtgggccccgcctcggggagctcgggcaggctcgcgttccgaggccggccgagcagataggtctttgggatgtaaagcagctgcccggggtcccgaggaaactcggccgtggtgaccaacacgaaacaaaagcgctcggcgtaccaccgaagcatgggcacggatgccgtagtcaggttgagttcgcccgggggcgccaagcgtccgcgctgggggtcgctggcgtcgggggtgttgggcaaccacagacgcccggtgtttgtgtcgcgccagtacgtgcgggccaaccccagaccgtgcaaaaaccacgggtcgatttgctccgtccagtacgtgtcatggcccccggcaacgcccaccaggacccccatcaccacccacagaccggggcccatggtcgtcgtcccggctgccagtccgcagatgggggggggtgtccgtacccacggcccaaagaggctccgcacctcggaggctatcggaggccctttgttgccgtaagcgcgggccaaaggatggggtggggtgagggtaaaagcacaaagggagtaccagaccgaaaacaaggacggatcggcccgctccgtttttcggtggggtgctgatacggtgccagccctggccccgaacccccgcgcttatggacacaccacacgacaacaatgccttttattctgttcttttattgccgtcatcgccgggaggccttccgttcgggcttccgtgtttgaactaaactccccccacctcgcgggcaaacgtgcgcgccaggtcgcgtatctcggcgatggacccggcggttgtgacgcgggttgggatcatcccggcggtgaggcgcaacagggcgtctcgacacccgacgggcgactgatcgtaatccaggacaaatagatgcatcggaaggaggcggtcggccaagacgtccaagacccaggcaaaaatgtggtacaagtccccgttgggggccagcagctcgggaacgcggaacagggcaaacagcgtgtcctcgatgcggggcagagaccccgcgccgtcctcggggtcggggcgcggggtcgccgcggcgacccccgtcagccggccccagtcctcccgccacctcccgccgcgctgcaggtaccgcaccgtgttggcgagtagatcgtagacacggcgaatggcggacagcatggccaggtcaagccgctcgcccgggcgttggcgtctggccaggcggtcggcgtgttcggcctccggaaggacacccaggaccaggttcgtgccgggcgcggtcgggggcatgagggccacgaacgccaacacggcctggggggtcatgcttcccatgaggtaccgcgcggccgggtagcacagcagggaggcgatagggtgccggtcgaaaacaagggtgagggccgggggcggggcttgcgggcccacagcctcccccccgatatgaggagccaaaacggcgtccgtcgccgcataaggcgtgctcattgttatctgggcgctggtcattaccaccgccgcctccccggccgatatctcgccgcggtccagacggtgctgcgtgttgtagatgttcgtcagggtctcggaggcccccagcacctgccagtaagtcatcggctcggggacgtagacgatattgtcgcgcggccccagggcctccatcagctgcgcggaggtggtggtcttccccaccccgtggggtccgtctatataaacccgcagcagcgtgggcagctccggatccccgcgggctccggaggccccctggcgatggctaggacgggacgccgcgcggccgtcggtaggcccgctcgcacgagcagcctgaccgaacgcaggcgcgtgctgttggccggcgtgagaagccatacccgcttctacaaggcgttcgcccgagaggtgcgggagttcaacgccaccaggatttgtggaacgctgctgacgctgatgagcgggtcgctgcagggtcgctcgctgttcgaggccacgcgcgtcaccttaatatgcgaagtggacctcgggccgcgccgcccagactgcatctgcgtgttcgaattcgccaatgacaaaacgttgggaggtgtgtgcgtcatcctggagctaaagacatgcaaatcgatttcttccggggacacggccagcaaacgcgaacagcggaccacgggcatgaagcagctgcgccactccctgaagctgctgcagtcgctcgcgcctccgggggacaaggtcgtctacctgtgtcctattttggtgtttgtcgcgcagcgtacgctgcgcgtcagccgcgtgacccggctcgtcccgcaaaagatctccggcaacatcaccgcggccgtgcggatgctccaaagcctgtccacgtatgccgtgccgccggaaccgcagacccggcggtcgcggcgccgggtcgccgcgaccgccagaccgcaaaggcccccctccccgacacgtgacccggaaggcacggcgggtcatccggccccaccagagagcgaccccccctccccaggggtcgtaggcgtcgctgcggagggtgggggtgtgcttcagaaaatcgcggcgcttttttgcgtgccggtggccgccaagagcagaccccggaccaaaaccgagtgaggttctgtgtgttgtttttttttttttttttcctcgttttgttttctcttctttcccccccccctcccccgcttctggccaagcatcctcacctgcttaagcggaacccgcgggcgcgcggggactcatttgtcgccggcgacacccacccgacaacagcccctgggtgtcgaccgctgtcgcccccgtctgtcgcctctcccttttttccccccctcaaagaacgtggtgttgggcgccggccaattcttcccggagcgccgtcgtcgcccgcccgccgccctcgaacatggacccgtactaccctttcgacgcgctggacgtttgggaacacaggcgcttcatcgtcgccgactccaggagcttcatcacccccgagttcccccgggacttctggatgttgcccgtgttcaacatcccccgggagacggcggcggagcgggcggcagtgctgcaggcccagcgcaccgcggccgcggcggccctggagaacgccgccctccaggccgccgagctgcccgtcgacatcgagcgccggatacgcccgatcgagcagcaggtgcatcacatcgccgacgccctggaggcgctggagaccgcggcggccgcggccgaagaggcggatgccgcgcgggacgccgaggcgaggggggagggcgctgcggacggggcagcgccgtcgcccaccgcgggccccgccgccgcggagatggaggttcagatcgtacgcaacgacccgccgctacgatacgataccaacctccccgtggatctgctacacatggtgtacgcgggccgcggggccgcgggttcgtcgggagtcgtctttggtacctggtaccgcacgatccaggaacgcaccatcgcggacttccccctgaccacccgcagcgccgactttcgagacgggcgcatgtccaagaccttcatgaccgcgctggtcctgtctctgcagtcgtgcggccggctgtacgtgggccagcgccactattccgccttcgagtgcgccgtgctgtgtctgtatctgctgtaccgaaccacccacgagtcctcccccgatcgcgatcgcgctcccgttgcgttcggggacctgctggcccgcctgccgcgctacctggcgcgtctggccgcggtaatcggcgacgagagcggacgcccgcagtaccgctaccgcgacgacaagctgcccaaagcgcagttcgcggcggccggcggccgctacgagcacggggccctggccacccacgtcgtgatcgccacgttggtgcgccacggggtgctaccggcggccccgggcgacgttccccgagacaccagcacccgcgtgaaccccgacgacgtggcccaccgcgacgacgtcaaccgcgccgccgccgcgtttttggcacgcggccacaacctcttcctgtgggaggaccagacgctgctgcgggcgaccgccaacaccattacggccctggccgtgcttcggcggctcctcgcgaacggcaacgtgtacgcggaccgcctcgacaaccgcctgcagctgggcatgctgatcccgggagccgtcccggcggaggccatcgctcggggggcgtccggattggactcgggcgccataaaaagcggcgacaacaacctggaggcgctgtgcgttaactatgtacttccgctgtatcaggcagaccccacggtcgagctgacccagttgtttccggggctggccgccctgtgcctggacgcccaggcggggcggccactggcgtcgacgaggcgcgtggtggatatgtcgtcgggcgcccgccaggcggcgctcgtgcgcctcaccgcgctggagctcatcaaccgcacccgcacaaacaccacccctgtgggggagattattaacgcccacgatgccttggggatacaatacgaacaggggcctgggctgctcgcccagcaggcacgcatcggcttggcgtcaaacaccaagcgattcgccacgttcaacgtgggcagcgactacgacctgttgtactttttgtgtctcgggttcattccccagtacctgtccgtggcctagggaagggtgggggtggtggtggtggggtgtttttctgttgttgtttctggtccgcctggtcacaaaaggcacggcgccccgaaacgcgggctttagtcccggcccggacgtcggcggacacgcaacaacggcgggccccgtgggtgggtaagttggttcgggggcatcgctgtattcccttgcccgcttccaccccccccccccttcccgttttgtttgtttgtgcgggtgcccatggcgtcggcggaaatgcgcgagcggttggaggcgcctctgcccgaccgggcggtgcccatctacgtggccgggtttttggccctgtacgacagcggggacccgggcgagctggccctggacccagacacggtgcgtgcggccctgcctccggagaaccccctgccgatcaacgtagaccaccgcgctcggtgcgaggtgggccgggtgctcgccgtggtcaacgaccctcgggggccgttttttgtggggctgatcgcgtgcgtgcagctggagcgcgtcctcgagacggccgccagcgccgctatttttgagcgccgcggacccgcgctctcccgggaggagcgtctgctgtacctgatcaccaactacctgccatcggtctcgctgtccacaaaacgccggggggacgaggttccgcccgaccgcaccctgtttgcgcacgtggccctgtgcgccatcgggcggcgccttggaaccatcgtcacctacgacaccagcctagacgcggccatcgctccgtttcgccacctggacccggcgacgcgcgagggggtgcgacgcgaggccgccgaggccgagctcgcgctggccgggcgcacctgggcccccggcgtggaggcgctcacacacacgctgctctccaccgccgtcaacaacatgatgctgcgtgaccgctggagccttgtggccgagcggcggcggcaggccgggatcgccggacacacgtaccttcaggcgagcgaaaaatttaaaatatggggggcggagtctgcccctgcgccggagcgcgggtataaaaccggcgccccgggtgccatggacacatcccccgccgcgagcgttcccgcgccgcaggtcgccgtccgtgcgcgtcaagtcgcgtcgtcgtcttcttcttcttcttttccggcaccggccgatatgaaccccgtttcggcatcgggcgccccggcccctccgccgcccggcgacgggagttatttgtggatccccgcctctcattacaatcagctcgtcaccgggcaatccgcgccccgccacccgccgctgaccgcgtgcggcctgccggccgcggggacggtggcctacggacaccccggcgccggcccgtccccgcactacccgcctcctcccgcccacccgtacccgggtatgctgttcgcgggccccagtcccctggaggcccagatcgccgcgctggtgggggccatcgccgccgaccgccaggcgggtgggcttccggcggccgccggagaccacgggatccgggggtcggcgaagcgccgccgacacgaggtggagcagccggagtacgactgcggccgtgacgagccggaccgggacttcccgtattacccgggcgaggcccgccccgagccgcgcccggtcgactcccggcgcgccgcgcgccaggcttccgggccccacgaaaccatcacggcgctggtgggggcggtgacgtccctgcagcaggaactggcgcacatgcgcgcgcgtacccacgccccctacgggccgtatccgccggtggggccctaccaccacccccacgcagacacggagacccccgcccaaccaccccgctaccccgccaaggccgtctatctgccgccgccgcacatcgcccccccggggcctcctctatccggggcggtccccccaccctcgtatcccccagttgcggttacccccggtcccgctcccccgctacatcagccctcccccgcacacgcccacccccctccgccgccgccgggacccacgcctccccccgccgcgagcttaccccaacccgaggcgcccggcgcggaggccggcgccttagttaacgccagcagcgcggcccacgtgaacgtggacacggcccgggccgccgatctgtttgtgtcacagatgatggggtcccgctaactcgcctccaggatccggacttggggggggtgtgtgttttcatatattttaaataaacaaacaaccggacaaaagtatacccacttcgtgtgcttgtgtttttgtttgagaggggggggtggagtgggggggaaagtgggccgaatgacacaaaaattaggtcggaggggtgaggggggggggctaggagccgaaccgatggcccccacacgcgacggaaggcccggaagactaccacggggagggggtgtggaaagcgaccggtcgcagggagacggggttggtttggggttggtttggggttggttttcccgttagcacatgtctgcatttgtttttctagtcacacgcccccccccccccaaataaaaaccaaggcaaaacaataccagaagtcatgtgtatttttgaacatcggtgtctttttatttatacacaagcccagctcccctcccctcccttagagctcgtcttcgtctccggcctcgtcctcgttgtggagcggagagtacctggctttgttgcgcttgcgcagaaccatgttggtgaccttggagctgagcagggcgctcgtgcccttctttctggccttgtgttccgtgcgctccatggccgacaccaaagccatatatcggatcatttctcgggcctcggccaacttggcctcgtcaaacccgcccccctccgcgccttcctccccctccccgcccacgcccccggggtcggaagtcttgagttccttggtggtgagcggatacagggccttcatgggattgcgttgcagttgcaggacgtagcggaaggcgaagaaggccgcgaccaggccggccaggaccagcagccccacggcaagcgccccgaaggggttggacataaaggaggacacgcccgagacggccgacaccacgccccccactactcccatgactaccttgccgaccgcgcgccccaagtcccccatcccctcgaagaacgcgcacagccccgcgaacatggcggcgttggcgtcggcgcggatgaccgtgtcgatgtcggcaaagcgcaggtcgtgcagctggttgcggcgctggacctccgtgtagtccagcaggccgctgtccttgatctcgtggcgcgtgtagacctccaggggcacaaactcgtggtcctccagcatggtgatgttcaggtcgatgaaggtgctgacggtggtgacgtcggcgcgactcagctggtgagagtacgcgtactcctcgaagtacacgtagcccccgccgaagatgaagtagcgccggtggcccacggtgcacggctcgagcgcgtcgcgggtgaggcgcagctcgttgttctcgcccagctgcccctcgatcagcgggccctggtcttcgtaccgaaagctgaccagggggcggctgtagcacgtccccggccgcgagctgacgcgcatcgagttctgcacgatcacgttgtccggggcgacgggcacgcacgtggagacggccatgacgtctccgagcatgcgcgcgctcacccgccggccgacggtggcggaggcgatggcgttggggttgagcttgcgggcctcgttccagagagtcagctcgtggttctgcagctcgcaccacgcgacggcgatgcgccccagcatgtcattcacgtggcgctgtatgtggttatacgtaaactgcagccgggcgaactcgatcgaggaggtggtcttgatgcgctccacggacgcgttggcgctgggcgcctcccgcagtggcgcgggcgtggcattccggggcttgcggtcctgctcccgcatgtactcccgcacgtacagctcggcgagcgtgttgctgaggaggggctggtacgcgatgaggaagccccccgtggccaggtagtactgcggctggcccaccttgatgtgcgtggcgttgtacttgcgcgcaaacatgcggtcgatggcctcgcgggcatcccggccgatgcagtcgcccaggtcgacgcgcgagagcgagtactcggtcaggttggtggtgaaggtggtcgagatggcgtcggaggagaagcggaaggagccgccgtactcggcgcggagcatctcgtccacctcctgccacttggtcatggtgcagaccgccggtcgcttcggcacccagtcccaggccacggtaaacttgggggtcgtcagcaagttgcgggtcgtcggcgacgtggcccgggccttcgtggtgaggtcgcgcgcgtagaagccgtcgacctgcttgaagcggtcggcggcgtagctggtgtgctcggtgtgcgacccctcccggtagccgtaaaacggggacatgtacacaaagtcgcccgtcgccagcacaaactcatcgtacgggtacaccgaccgcgcgtccacctcctcgacgatgcagttgaccgtcgtgccgtaccgatggaacgcctccacccgcgaggggttgtacttgaggtcggtggtgtgccacccccggctcgtgcgcgtggcgaccttcgccggcttgagctccatgtcggtctcgtggtcgtcccggtgaaacgcggtggtctccatgttgttccgcacgtacttggccgtggagcggcagacccccttggtgttaatcttgtcgatcacctcctcgaagggaacgggggcgcggtcctcgaatatccccataaactgggagtagcggtggccgaaccacacctgcgacacggtcacgtctttgtagtacatggtggccttgaatttgtacggggcgatgttctccttgaagaccaccgcgatgccctccgtgtagttctgcccctccgggcgcgtcgggcagcggcgcggctgctcaaactgcaccaccgtggcgcccgtcgggggcgggcacacgtaaaactgggcatcggcgttctcgaccttgatttcccgcaggtgcgcgcgcagcgtggcgtggccggcggcgacggtcgcgttggcgtcggggggcggggtcgcctcgggccgcttgggcggctttttggttttccgcttccgggccttggtggtcgcggggctcgggacggggggcggccgggaggcgggacccccgttcgccgcgacggtcgcggccacgccgcccgaggcgcggggggccgccggggccgccggggccgccgacgccaccgcggccaccagcgcccccacgaccagcgcgcaaatcaagccccccccgcgcatggcgggcctacgggggcgcgtcgctcccgccgcccgctagtctgggggcgaggtgctgcaggaccgagtagaggatggaaaaaacgtctcggtcgtaaaccacgaccgagcggggtccgatgcagccgtcggggccgctctcgacgatggccaccagcggacagtcggagttgtacgtgaggtacacgcccggcgggtagcggtacagaccttcggaggtcgggcggctgcagtcggggcggcgcaactcaagctccccgcaccggtagaccgacgcaaagagtgtggtggcgataatgagctcgcgaatatatcgccaggcggcgcgctgggtgggcgtgattccggaaacaccgtcaaaacagtagaacttttgaaactcgctgacggcccaatcagcgcccgaaccccccgcgcccatgatgaagcgggcgagttcctccttgaggtgcggcaggagccccacgttctcgacgctgtagtacagcgcggtgttggggggctgggcgaagctgtgggtggagtggtcgaacaggggcccgttgacgagctcgaagaagcgatgggtgatgctggggagcagggccgggtccacctggtggcgcagcagcgacgctcgcatgaaccggtgcgcgtcaaacacgcccggggcggcgcggttgtcgatgaccgtgcccgcgcccgccgtcagggcgcagaagcgcgcgcgcgccgcgaagccgttggcgaccgcggcgaaggtcgcgggcagcacctcgccgtggacgctgacccgcagcatcttctcgagctccccgcgctgctcgcgcacgcagcgcccgaggctggccagcgaccgcttggtcaggcggtccgcgtacagccgccggcgctcccgcacgtccgcggcggcccgcgtcgcgatgtcgccccagctctccggcccctgcgcccctggctcggggccgcgctccccgtcctcgctcgcgggcgtccccgcgccacgcctccgccccccctcctccgcggcggcccggggctcttcctcctcggcccccccggtcgcgccgccggcccccagccgcgccagcacgcggcgcagcgcctcctcgtcgcactgctcggggctgacgagccgccgcagcagcggcgtcgtcaggtggtggtcgtagcacgcgcgtatcagcgcctcgatctgatcgtcgggcgacgtcgcctggccgccgatgatcagggcgtccaccatgtccagcgccgccaggtggcccccgaacgcgcgatcgaagtgctccgcccgccgcccgaacagcgccagctccacggccaccgcggcggtctcctgctgcagctcgcgctgcgccagcgcgttcaggttgtcggcgaaggcgtccatggtggagtggcgggcgcgatcgccggacgccagccagaagcgcagctcgctgatggcgtacaggccgggcgtagtggcctgaaacacgtcatgcgcctccagcagggcgtcggcctcctcgcggacagaagagctatcggcgggcggcgggccggccctggccccgccgcccgccgcggtccgcgccagcgcctggtccagcacacagagcgctcgcgcgcgggcggcgtccgacagcccggcggcgtggggcaggtaccgtcgcagctcgttggcgtccagccgcacctgggcctgttgggtgacgtggttacagatgcggtccgccaggcggcgggcgatggtcgccccttggttcgcggtgacgcacagctcctcgaaacagaccgcgcacgggtgggacgggtcgctcagctccgggggcacgatgaggcccgaccccaccgccgccaccataaactcccggacgcgctccagcgcggccgtggcgccgctcgggggggtgatgaggtggcagtagttcagctgcttgagaaaattctcgacatcatgcaggaagcacagctccatgcggacgtccccgccgtacgtctgcagccggatctgctggtggtacggacagggtcgggccagacccatggtctcggtgaaaaaggcagagacgtcacccgtggtcgcgaacgtttccaggtggcccaggagccgctccccctcgcgccacgcgtactccaggagcaactccagggtgaccgacagcggggtgagaaaggcggcggcctgagcctccagccccggccgcaggtgccgccgcagcacgcgcacctggagcgcgttgagttttagctgggcgagcttccccaggccgatctgggggtcgcatcgtcgaagcagctctagctgaaaaacgtacgtctgtacctgcccgagcagggccaacagtttctgtcgggccgcagtgggctcggaaaccgcggccgggggcgcggccgccatggcgagtcacccggccgtgctgtggtttagttaaggtttgggggggggtgggtcagaggcgcgccccgcgcggactgatgcggcggcgggcccctgacatcccctctttatgcccgtcgcccgcccgcccgccccgccggtgtgccgtgattcgcggagtcggggccttgtgtttctttctttcccccccgaatccgttctttcttcctcacccccccctccccacacacccacccaggactcgccaccacaaggaggcgagagcccgtcgctaacccaaagacacagtcacgagacacgatatcgactgtagttgcgatcgtttattttatacacaacaccaacctttccttcgaccccccccacccccgcccctagagcatatccaacgtcaggtcctttttctccggtggtccctccccaaacggatcgtcgccgtgaaacgcccgctttcgggcgacgccggccgcccccgccgccgccgccaaaccgccgaacgacgccgcgtggtcatcctcgtcgccgaaatccccaaagttaaacacctccccggcggcgccgagctggctgaccagggcctccgcctcgtgggccacctccagggccgcgtcggtcgaccactcgccatgcccgcgctccagggcgcgggtggtaaactccatcatttcctcgctcaggtactcgtcctccagcagcgccagccagtcctcgatctgcagctgctgggtgcgggggcccaggctcttgacggtcgccacaaacacgctgctggcgaccgccgccccgccctccgcaatgatgccccggagctgctcgcacagcgaatgctcgtgggccccgcccccgagactcgacgccgcgcacacaaacccggccctggggcaggccaggacaaacttgcgggtgcggtcaaagatcagcagcgggcacgcgtttttgccgcccagcaggctggcccagttcccggcctgaaacacgcggtcgttgccggccatgccgtagtatttgctgatgctgaggcccagcacgaccatcgggcgcgcggccatcacgggccgcagcaggttgcagctcgcgaacatggacgtccaggcgccggggtgcgcgtcgagggagtccatcagcgcgcgggccccggcctccaggcccgcgccgccctgcggggcccaggcggccgccgcctgcacgctggggggacggcgggacccggcgatgacggccgtgagggtgtttatgaagtacgtcgagtggtcgcagtacctcaagatctggttggccatgtagtacatggccagttcgctcacgttattgggggccaggttgataaagttaatcgcgccgtagtccagggagaacctcttaatgaacgcgatggtctctatgtcctcgcgcgacaagagccgggcggggagctggttgcgctggagggcggtccagaaccactgcgggttcggctggttcgaccccgggggcttgccgttgggaaagatgaccgcgtggaactgcttcagcaggaagcccagcggtccgaggaggatgtccacgcgcttgtcgggcttctggtaggcgctctggaggctggcgacccgcgccttggcggcctcggacgcgttggcgctcgcgcccgcgaacaacacgcggctcttgacgcgcagttccttgggaaacccaagggtcacgcgggcaacgtcgccctcgaagctgctctcggcgggggccgtctggccggccgttaggctgggggcgcagatagccgccccctccgagagcgcgaccgtcagcgtcttcgccgacaggaacccgttgttgaacaggtccatgacgcgccgccgcagcaccggttggaattgattgcgaaagttgcgcccctcgaccgactgcccggcgaacaccccgtggcactggctcagggccaggtcctggtacacggcgaggttggaccgccgcgcgaggagctgcagcagggggcacggcccgcaggtgtacgggtccagcgacagcgacatggcgtggttggcctcggccagaccgtcgcggaacttaaagttgcgcccctcgatcaggttgcgcatcagctgttccacctcgcgatccaccagctgcttgatgttgttcaccaccgtgtgcagggcctcgcggttgccgataatcgtctccagcctccccagggccgtgggcaccgcctggtccacgtactgcagggcctcgagctcggccatgacgcgctcggtggccgcgcggtacgtctcctgcatgatggtccgggtgttctcggacccgtccgcgcgcttcagggccgagaaggcggcgtagttccccagcacgtcgcagtcgctgtacgcgctgttcatcgttccgaagaccccaatggccccccgggcggcgctcgcgaacttggggtggcgggcccgcagccgcatcagcgtcgtgtgcgcgcaggcgtggcgggtctcgaaggtacacaggttgcagggcacgtcggtctggcccgagtccgcgacgtagcgaaacacgtccatctcctggcgcccgacgatgactccgccgtcgcagcgctccaggtaaaacagcatcttggccagcagggccggagagaacccgcacagcatggccaggtgctcgccggcgaactcctgggttccgccgacgaggggcgccgtggggcgcccctcgtacccgggcaccacgtggccctcgcggtccagctgcgggttggccgccacgtgcgtgccgggcacgagaaagaagcggtaaaaggagggcttgctgtggtccttggggtccgccggcccggcgtcgtccacctcggtcaggtggagggccgaattggtgctgaacaccatggcgcccacgaggcccgcggcgcgcgccaggtacgccccgacggcgccggcgcgggccgcgggcgtttcctggccctcaagcaggggccacgtggtgatgtcggggggcggctcgtcaaagaccgccatcgacacgatggactccagggccagggcggcgtcgcccgccatcaccgaggccaggcgctgctcaaacccgcccgccgggcccttgttcccggcgtcgcgcgcgccccgctggggcttaccctggctggcctcgaaggccgtgaacgtaatgtcggcggggagggccgcgccctcgtggttttcgtcgaacgccaggtgggcggccgcgcgggccacggcgtccacgttccgggcacgcagggccacggcggcgggcccgacgaccgcctcgaacagcaggcgggcgagggggcggttgaaaaacggaagggggtagttgaaattctccccgatcgatcggtggttgcagttaaacggatcggcgatgacccggctaaaatccggcataaacatctgcagcggatacacggggatgcggtgaacctccgcgtccccgatggttaccttgtccatcccgcccagatgcaggaaggtgttgctgatgcacacggcctcccggaagccctccgtgatcaccagatacagcaaggcccggtccgggtccagtccgagccgctcgcacagcgcgtcccccgtcgtctcgtgctttaggtcgcagggccggggcgcgtagtccgcgaagccaaaatgcgggcgcgcccgctcgcagagccgcgtcaggttgggggcctgggtgctgggggccaggtggcggccgccgtgaaagacgtaaacggacgggctgtagtgcgagggcataagcttgagggacaccgcggtccccccaaggcccgtcgtgcgggacccgacgaccgcggccacgttggcctcaaacccgctctccacggtcaggccgacgatgaggggcgcgacggcgacgtccgcgtcgccgctgcgcgccgacagtagcgacagcagctccaggccttcggccggacaggcgcggccatacacgtaccccatcggccccggaggaaccttgacggtggtcgtcgttttgggcttggtgtccatggctttcgggagatcggcgaccggcaggaacgggggcccggcaagacgaccgggggcagacgggggaggccgcgcgtggtcgacggctgctgcccgccgtcgtctctccgatggggtcgaatgccggcgctgggggtggggtctacacccgcccgttcgccgagcggcccctggtgggggtgggatgggtgggatggggtgggcgagaatggcccgccaccggatcgcgccggacgggggggcccggggttgggcaaggtttgggcgcaaggctccagcggcgattcgagaggcctgcggatggcggcccagagctgggtatgctcggccggggcggccggtatatgtacggcgtgctgggaggggcggcgtcgggccccgcccacggtccgccacgccccgcgcgtcatcggcagggggcgtggccgcccttctaaaaaaagtgagaacgcgaagcgttcgcactttgtcctaataatatatatactattaggacaaagtgcgaacgcttcgcgttctcactttttttagaagggcggccacgccccctttgacgtcacgctcacccgggcggccggccgcccataagcgcggcctgccgggccgataaaaagaaaccgcggcgcccccgcggacaccacacactggctctcgaaccccggacgcgcagaagggacccgggcgcgggtccgccggtaagagccggggggaacatcggcaccgccatcccaccccgagctgttgggtgggcgggtgggggggctggtgaggcggtggtgggagggggcggcgtatagcaggacaacgaccggcggcgatgttttgtgccgcgggcggcccggcttcccccggggggaagccggcggctcgggcggcgtctgggttttttgccccccacaacccccggggagccacccagacggcaccgccgccttgccgccggcagaacttctacaacccccacctcgctcagaccggaacgcagccaaaggccctcgggccggctcagcgccatacgtactacagcgagtgcgacgaatttcgatttatcgccccgcgttcgctggacgaggacgcccccgcggagcagcgcaccggggtccacgacggccgcctccggcgcgcccctaaggtgtactgcgggggggacgagcgcgacgtcctccgcgtgggcccggagggcttctggccgcgtcgcttgcgcctgtggggcggtgcggaccatgcccccgaggggttcgaccccaccgtcaccgtcttccacgtgtacgacatcctggagcacgtggaacacgcgtacagcatgcgcgccgcccagctccacgagcgatttatggacgccatcacgcccgccgggaccgtcatcacgcttctgggtctgacccccgaaggccatcgcgtcgccgttcacgtctacggcacgcggcagtacttttacatgaacaaggcggaggtggatcggcacctgcagtgccgtgccccgcgcgatctctgcgagcgcctggcggcggccctgcgcgagtcgccgggggcgtcgttccgcggcatctccgcggaccacttcgaggcggaggtggtggagcgcgccgacgtgtactattacgaaacgcgcccgaccctgtactaccgcgtcttcgtgcgaagcgggcgcgcgctggcctacctgtgcgacaacttttgccccgcgatcaggaagtacgaggggggcgtcgacgccaccacccggtttatcctggacaacccggggtttgtcaccttcggctggtaccgcctcaagcccggccgcgggaacgcgccggcccaaccgcgccccccgacggcgttcggaacctcgagcgacgtcgagtttaactgcacggcggacaacctggccgtcgagggggccatgtgtgacctgccggcctacaagctcatgtgcttcgatatcgaatgcaaggccgggggggaggacgagctggcctttccggtcgcggaacgcccggaagacctcgtcatccagatctcctgtctgctctacgacctgtccaccaccgccctcgagcacatcctcctgttttcgctcggatcctgcgacctccccgagtcccacctcagcgatctcgcctccaggggcctgccggcccccgtcgtcctggagtttgacagcgaattcgagatgctgctggccttcatgaccttcgtcaagcagtacggccccgagttcgtgaccgggtacaacatcatcaacttcgactggcccttcgtcctgaccaagctgacggagatctacaaggtcccgctcgacgggtacgggcgcatgaacggccggggtgtgttccgcgtgtgggacatcggccagagccactttcagaagcgcagcaagatcaaggtgaacgggatggtgaacatcgacatgtacggcatcatcaccgacaaggtcaaactctccagctacaagctgaacgccgtcgccgaggccgtcttgaaggacaagaagaaggatctgagctaccgcgacatccccgcctactacgcctccgggcccgcgcagcgcggggtgatcggcgagtattgtgtgcaggactcgctgctggtcgggcagctgttcttcaagtttctgccgcacctggagctttccgccgtcgcgcgcctggcgggcatcaacatcacccgcaccatctacgacggccagcagatccgcgtcttcacgtgcctcctgcgccttgcgggccagaagggcttcatcctgccggacacccaggggcggtttcggggcctcgacaaggaggcgcccaagcgcccggccgtgcctcggggggaaggggagcggccgggggacgggaacggggacgaggataaggacgacgacgaggacggggacgaggacggggacgagcgcgaggaggtcgcgcgcgagaccgggggccggcacgttgggtaccagggggcccgggtcctcgaccccacctccgggtttcacgtcgaccccgtggtggtgtttgactttgccagcctgtaccccagcatcatccaggcccacaacctgtgcttcagtacgctctccctgcggcccgaggccgtcgcgcacctggaggcggaccgggactacctggagatcgaggtggggggccgacggctgttcttcgtgaaggcccacgtacgcgagagcctgctgagcatcctgctgcgcgactggctggccatgcgaaagcagatccgctcgcggatcccccagagcacccccgaggaggccgtcctcctcgacaagcaacaggccgccatcaaggtggtgtgcaactcggtgtacgggttcaccggggtgcagcacggtcttctgccctgcctgcacgtggccgccaccgtgacgaccatcggccgcgagatgctcctcgcgacgcgcgcgtacgtgcacgcgcgctgggcggagttcgatcagctgctggccgactttccggaggcggccggcatgcgcgcccccggtccgtactccatgcgcatcatctacggggacacggactccattttcgttttgtgccgcggcctcacggccgcgggcctggtggccatgggcgacaagatggcgagccacatctcgcgcgcgctgttcctccccccgatcaagctcgagtgcgaaaaaacgttcaccaagctgctgctcatcgccaagaaaaagtacatcggcgtcatctgcgggggcaagatgctcatcaagggcgtggatctggtgcgcaaaaacaactgcgcgtttatcaaccgcacctccagggccctggtcgacctgctgttttacgacgataccgtatccggagcggccgccgcgttagccgagcgccccgcagaggagtggctggcgcgacccctgcccgagggactgcaggcgttcggggccgtcctcgtagacgcccatcggcgcatcaccgacccggagagggacatccaggactttgtcctcaccgccgaactgagcagacacccgcgcgcgtacaccaacaagcgcctggcccacctgacggtgtattacaagctcatggcccgccgcgcgcaggtcccgtccatcaaggaccggatcccgtacgtgatcgtggcccagacccgcgaggtagaggagacggtcgcgcggctggccgccctccgcgagctagacgccgccgccccaggggacgagcccgcccccccagcggccctgccctccccggccaagcgcccccgggagacgccgtcgcatgccgaccccccgggaggcgcgtccaagccccgcaagctgctggtgtccgagctggcggaggatcccgggtacgccatcgcccggggcgttccgctcaacacggactattacttctcgcacctgctgggggcggcctgcgtgacgttcaaggccctgtttggaaataacgccaagatcaccgagagtctgttaaagaggtttattcccgagacgtggcaccccccggacgacgtggccgcgcggctcagggccgcggggttcgggccggcgggggccggcgctacggcggaggaaactcgtcgaatgttgcatagagcctttgatactctagcatgagccccccgtcgaagctgatgtcccgcatcttgcaataaatgtctgcggccgacacggtcggaatttccgcgtccgctggtttctctgcgttgcgtctgaccacgagcacaaacgtgctctgccacacgtgggcggcgaaccggtagccggggcacgcggtcagcatccgatcgatgagccggtagtgcaggtgggccgacgtgccggggaagatgacgtacagcatgtggcccccgtacgtggggtccgggtaaaaaagaaaccgggggtcgcacgccccccctccgcgcaggatcgtgtgcacgaaaaagagctcgggctggccgagcgtatcggccaggaggtcctggaggggggtgctgtggcggtcggccagcacgaccagggaggccagaaaggtgcggtgctcaaagatcgtattgatctgctgcacgaaggccaggatgagggcctcgcggctgacggtggccagccgcccgtcgcccgcgctgcacgcggggcagcagcccccgatccccaggtagtagcccatgcccgagagggtcaggcagttgtcggccacggtctggtccaggctgaaggggagcgacacgggggtcgtcttcaccaggggcacggagagcgagcgcacgatggcgatctcctcggagggcgtctgggcgagggcggcgaagaagccgcggtagcgacggcgctcgtgcaggcagagctccagcctgcgcgcgtgcgacggcaggctcttgcgggaggcccggcgctccacgccggggttcccggcggcggaaaagcgcgaccgccgccgggtcttgtcgcggccgggcccgggccgggagccggagcgacggggggcgatgtcatacataggtacagagggtgtgctccagggacaggagagagatcgagtgtcgtctgagcagcgcgccggcctcgcggacaaatgtggccagcgcggtgggcttcggcacaaatacctggtacgtcttgaaggtgtagatgagggcccgcagggctatacagacccgcccctcgaactcgttgccgcaggccaacttggccttgtgaagctgcagctcgtcgcgatggtcggcgcgggggtggccaaacaggacccaggggtcgacttccatctccgtgatggcgcacatcggatcgcagaacatgtgcttgaagatggcctcggggcccgcggcccgaagcaggctcacgaaccggcccccgtccccgggctgcgcctcggggtccgcctcgagctggtccacgaccggcactatgcagtcgaagaggctggtgttgttctccgagtagcggacgacggacgccctcaggcgtcgcatggccagccagtaggcccgcaccagcaacagattgcacagcaggcattccccgccggtgcgcccgcgcccccggccgtgcttcagcacggtggccatcagcgggcccaggtccaggtcgggctggggctggggctcggcgaactgcgcaaaacgcggggccgcgtcgcgcatgcgcgccccgcggtgcgcttcccaggactcgctgaccgcggcgcggcgggcgtccgcggcggcgcgcagccggggccccgactcccagacggcgggggtgccggcgagcagcagcaggatcaggtcggcgtacgcccacgtctccggctcacccccctgcgccagcgccccggcggcggcctcgaactccccgttgcgggcggcggcgcgcgtgcagcagctgtctccgcccccgcgcttgccctcggtgcagtcgagcaggcgggcgcagtccttccagttcatcagggcggtggtgagggagggttgcgttcccgagcccccgcccgcccccgcccccgccccgtcatcgcccccggaggccagggtcccgatgagggcccgggttgcggactgcgcgaggaaggaatagttggagtactgcaccttggcggcgcccggggagggcgtcggcctgggttgcttctgggcgtggcgcccgggcaccccgccgtcggtccggaagcagcagtggagaaagaaatgccggtggatgtcgttgatggtcagggcgaagcgcgcgaaggagccgacaagggtcgccttcttggtgcgcaggaagtggtggtccatgacgtagacgaactcgaaggcggccacgaagatgctcgcggcgcagtggggcgcgcccaggcacttggcgcagaggaacgcgtaatcggccacccactggggcgagaggcggtaggcctgcttgtacagctcgatggtgcggcagaccagacaggggcggtccagcgcgaaggtgtcgacggacgccgcggcgaagggccccgtgtccaagagtccctctgccgtggggtctgcgggcgggccgcgggcggaccccggcccccgcccccccgaagcctcgcgcgcggccccgcgcggccgcgggggggcgggcgcgacgtcgctctccacgtcctcgtcgagcgcgctcgcgggcggcacgcctaccacgtgacaggccgccaggagctcggcgcacagggcctcgttaagagccagaaggtcgggatcgaaggccacatacggacgctcgaacgcgccctccttccagctgctgcccggcgactcttcgcgcacggcggcgctcgacggcacccccggggcggacgtcgccatggccggtcgagcggggcgcacgcgtccgcgaacgttacgggacgcgatccccgactgcgcgctgcggtcccagaccctggaaagtctagacgcgcgctacgtctcgcgagacggcgcgggggacgcggccgtctggttcgaggacatgacccccgccgaactagaggttatattcccgaccacggacgccaagctgaactacctctcgcggacgcagcggctggcctccctcctgacgtacgccgggcctataaaagcgcccgacggccccgccgccccacatacgcaggacaccgcgtgcgtgcacggcgagctgctcgcccgaaagcgcgaacggttcgcggcggtcattaaccggttcctggacctgcaccagatcctgcggggctgacgcgcgcttcggcggggcaccggcaccgggaccgacttgttttacataacagtagggggtgggggaacgcgcacccttgcccggtcgcgatggcggggatggggaagccctacggcggccgcccgggggacgcgttcgagggtctcgttcagcgcatcaggctcattgttcccgccacgctgcgcggcgggggtggggagtcgggcccctactcgccatccaacccgccctcgagatgtgccttccagttccacggccaggatgggtccgacgaggccttcccgatcgagtacgtcctgcggctcatgaacgactgggccgatgtgccctgcaacccctacctgcgcgtgcagaacaccggcgtttcggtgctgtttcaggggttttttaaccggccccacggcgccccggggggcgcgatcacggcggagcagaccaacgtgattctgcactccaccgagacgacgggactgtccctcggagacctggacgacgtcaaggggcgcctcggcctggacgcccggccgatgatggccagcatgtggatcagctgctttgtgcgcatgccccgggtgcagctcgcgtttcggttcatgggccccgaggacgccgttcgcacgcggcggatcctgtgtcgcgccgccgagcaggccctcgcccgtcgccgccggtccaggcggtcccaggatgactacggggcggtggtggtggcggcggcgcaccactcttccggagcgcccgggccgggggtcgccgcctcgggcccgccagcgccgcccggacggggaccggcccgtccgtggcatcaggccgtgcagttgttccgggccccgcgtccgggccccccggcgcttctgttgctggcggcggggctgtttctgggggccgctatctggtgggcggttggcgcgcgcctatgaaagggggcgagccaccgtcccgcccgccagtgcatcccagacgcccgcgagccgcacatcccctccgctcccgcctccggcccgattcttacggcgcgacccaaggtcccgatggccgccccgcagtttcaccgccccagcaccattaccgccgacaacgtccgggcgctcggcatgcgcgggctcgtgttggccaccaacaacgctcagttcatcatggataacagctacccgcatccgcacggaacgcagggtgcggtgcgagagtttcttcgcgggcaggccgcggcgctgacggacctcggggtgacccacgccaacaacacgttcgccccgcagcctatgttcgcgggcgacgccgcggccgaatggctgcggccctcgttcggtcttaagcgcacgtattccccctttgtcgttcgcgaccccaagacccccagcaccccgtgagtcctcggcgggtccctccgcggccgtctctcgttgcccccctttcccccttcccgggtggttcaataaaaaacaccaacatacgatattcgcgtttgatacgtttattggggggggtgtagggcccaacgatcggcgattaacaacaccaaacaatcgagcgcgtctaacccagtaacatgcgcacgtgatgtaggctggtcagcacggcgttgctgcgctgaaacagcgccctgcgggtccgctgcagctgttgttgtatgcggcggcatgcgcggatcaaaaccgccagggcgctacgaccggtgcttcgtacgtagcgtcgcgacaagacggcatttgcctgtacgggcaaggggccaaattgcgagtgtggtgactggaggtggtcggcggccaatgggccgggtggttcgtcggcggggggcaagtgcggttccggtgggagggggtcgagcgcctcggtatcatccgagtccgagaaacgcagggagtctgcgtcggagtgttcatcatcggaggagatgtgcagcgtctgaagcagcgatgcgggtgggggcgcggagtcgacgtgaagcgcgagagaggaagcccacgaagtcacagcggacactgggaggtgggtgtttgtatgtgtgggagactcgggcgtcgggaccgagtctcggctctggggtgtaagcgtccgagttacgggcggcaggggcggctggggcaggggcggctggggcaggggcggctggggcaggggcggctggggcaggggcggctggggcaggggcggctggggcaggggcggctggggcaggggcggctggggcaggggcggctggggcaggggcggctggggcaggggcggctggggcaccgagcgcgcgcggatgcgcgtccgcgcggcgggtttggtcgcgggtgactggggtggggggcggcgggcaaccgggcctccgggcacgacccaaccgcacaaaggctcgctcggggcaaccgggcctggggccaaaggcggggggctggtctggacggcggaggtcgggggggcaaggcccggagaaggcggcactgccgccgctgcggcggaaaccgcggccgcgtggtcggctgggtcccggggagaggggagggagttcaacgaggccgagagcgaggcgaccgcggggcgcgtgaggcgccggggtgggccggccgcggggccccgggggggtgtcggcgagggacccgctgttgtctggcggcggccgcggcggcggtcgcccccggggacgaccgctccttcggcgggcggaggcgggatgggcgcgagcgtgggggcgggaaaggccccgcgagccgaggcggggccgggcggaaggggcaaagcagaaacccaagccgggggcgcggactccggggtgggcggctggtcgggaggacgcgcggaagcggcgaccggggcgaccggggcggggagtgccggcggacgccacccctcggggggggcggaggcccggggcgcgcgcgatttggcacgcgtccggcgggacctgcgcacgcgcggcacggcggcggagaaagcggcggcagagccggaaaaggccgggggaggaagcgcggcatccgcggggggactcggtgtgggtggcgagggccgtgggtcgtcgcgaggggccacgggcacgcgccccgtgttttgttgaggcgggacactcggtcgtgtttcgcgagccgtagctgccggcccgatgggccgcggtgcgtactgggacgtggggacggactgatcggtggcgggggggggaagaagggccggggccggattgggcgtggggccgccggcgtcgtcggacgccagctcctccaggccgtggatccaggcccacatgcgaggggggacgggctcgccggtggtggcgtcggtgaggagagtgggggcgaggacccccgggtccgcctgccgtgcggggggggcagcggggtcctcgggacccgatccgccatccccccccgcaaggtcccgcgggtcgcgggcggcggtcggggcagagggacctgcctcgtcggcgagggggcgctggtaaaccgggtgtcccgggaacagctcccccgtcaggagggaggcgtcgaagggccgcccgaggatggcccgcgcgaagaaggggtccgcgtcggcggcgctcgccgcgagaacgtcccccgcggtagccacaaacggaagctcctcggtggcctcgctgcccacaaaccgcacgtcaggggggccggggggctccggggcttcccacaagaccgcgaccggggtcatggagatgtccacgaggaccaggcacgggggcccgtcggcgagagggcgctcggcgatgagcgccgacaggcgcgggagctgcgccgccagacacgcgttttcgatcgggttgagatcggtgtggaggaggccgacggcccacgtctcgatgtcggacgacacgacgtcgcgcagggcggcgtccggcccgccggggcgcgagtcgaagagcgtcaggcacagttccagttccgactcgcgggagaaggccgtggtgttgcggagcgccaccacgacgggcgcgccgaggagcaccgcggccagaaccaggtccatggccgtaacgcgcgcggcgggggtgcggtgggtcgcggcggccagcacggccacgtgctggcccgtgggtcggtagagggcgtggggggcctcggggagggacgcctcgcgcccccccgccgggccgagcgtctggccagactccaggcgtgcggccaggagggcgtcgaagctgtcgtactcggtgtagtcgtcgggaaacatgcaggtccacagcgcggccaaagcggcgctcggcagacacatgcgcccgaggacgctcaccgccgccagggcctgggccggactgagcttcccgagcgccgggacgtcccggcgctgggtcccgagctccaaggccgagcgccagggcgccagcgggtcggtttcggacagcttgccccggcgccagtcggccagccgcgtgccgaacaggaggccccgggtcggggggcctccgtccaaaaacgtcggcaacacgcggatgcgggcgtcgggatgcggggtcaggcgctggacgaacagcatggactccgctgcgtcctcgaacgcgcgttcgagggtgaggtgcatgtactcgtgctggcgaacgaggtccaggcgccagaagttgtagatgtgttccggaacgccggccaccagcgcgaccagcacgtcgttctcgttgaaggcgacgcagtggcgctgggacccccgggggcccggcggcggacgcggcgccgccgctccggacgcccagcccagctgggcccagcgacacccaaactcgcgcgtgagggtggtggcgacgagggcgacgtacagctcggccgccgcgtccatcgaggcgccccacgtcgcctggcgatggcgcacgaagcgaccgaacagctgaaagttggcggcctgggcgtcgctgagggccagctggagccggttcacgacggtcagcacgtacatggccgtgaccgtcggggccgattcgaggacgtccgtcggaagcgggggccgcacgcaggccgcctcgggacgcatcagcagcgcgccgagtttgtcggtgacggccgggaagcatagcgcgtactgcagcggcgttccgtccggggccaaaaagctggtggcgaacggcagatccagagcgctgacggcctcacgcagcaccaggggccccgggtctccgccggcgcgcagatacgcctcgccccggcggcgcagcagctgcgggtcgacctcgtggccctcgggggaagaagaggcccgggcgcgggcgtcgagggcgcgaagatcaacgagcaggggcgcgggcgcggactccgcgcccgcgcccgtctggccgccggccctggcgtacgcgctatataagcccatgcggtattggatgagttcccgcgcgccccggaactcctccaccgcccacggggccaggtccgcggccgccgcgtcgaactccgccagcaggccccccagggcgtcaaagttcatctcccagggcaccctgcgcaccacctcatcccgcagccgggcgcacagggcggtgtgcttggtgacgcgcgcgcccagctcctccacggcctccgcgcgctcggcgcccttggcgcccaggacgccctggtacctggcggaaaggcgctcgtaggccggctgggcccgcagccccgacaccgtgttggtggtgtcctgcagggcgcgcagctgctcgtgcatggcgcggaacccctcgggggacttccaggcgcccccccggacgcggccaaagcgaccccagacctcgtcccactccgcctcggcctcctccagggacctccgcagggcgtcgacgcggcgccgagtatcaaagagcgcccccaggcggccggcgtgccgcgccagggggccggggccgtcgccgcgggcggcgcttagcgggtgcgtctcgaaggtgcgctgggcgtgctctagccagataaccgcgggcacgtcgagctcgcgcgttttctcggtctgatccaacagaacctcgacctggtcggcgatctccgccaccgagcgcgcctggtcgagcgtcttggccacggtcgccgggacggcgaccaccttcagcatggtcttgaggttggccaggccctcggcctcgatctgggcccggcgctcgcgcgcggccagcgcctcccgcaggcccgccatgacccgctcggtggcctccgcgcgctgctgtttggcgcgcaccactgcgtccttggtctcggccgtgtcctgccgggtcacgaaggcgacatactcggcgtacgccgtgttcttcacggggctctggtccacgcgctccaacgccgccgcgcacgcgaccagcgcgtcctcgctgggacacggcagggtgaccccggtccggaccagctccgcggtggcctccgggtcattccgggccgcggatatctgctccgcggcggccgccaggtccaggggcacgccgccgagcgcccggtgcacgtcggcccggatggcgtccaggcgatcgcggagctccacgtagtcggcgtagccatgttggaagaacggcacgtaccggcgcaggccgggcacgctcgtcatgtcgtccgccaggcgccccacggcctcgtggtagtcgataaacccgtcgcccgcctgggccatttccaggagcccctccgcgatgcgcagcagccgcgccaggggctcggcgtcgacccgaaacatgtcggcgtaggtttcggcggcggcgtggaacgccgcgctccagccgaggcggtggatggcggcgagcggggggagcatggggtggcgctggttctcgggggtgtaggggttaaacgcgaaggccgtatccagggcgagggtgaccgcctcggcgttggccgcgagcgcctgctcggcgcgcttgcggaagtcccgggggttgtagccgtgcgtgcccgccagcgcctgcaggcggcgcagctcgaccacgtcgaactcggcgcggttctcgacgcggtccagcgccgcctcgacgccggcggcccagcgctcgctgctgccccgggcgcgctgggccgccatcttcgccgtcaggtcggcgacggcggcctcaagttcgtcggcgcggcgtcgcgtggcgccgatgaccttgcccagctcctgcagggcgcgcccgctgggggaatggtccccggccgtcccttcggcgtgcagcaggcccccgaacccagcctcgtgccccgcgaggctttcccgagcagcggtcgtcgcgcgggccgcggcatcgatgagggcggcatggtccccctccggctgggcgcaggcccggcgcgcctggactaccaggtcggcggccgccgaccccagggtcgtgagctcgtcgatggccccccgcgcctccagggccagccgagtcgcctttacataccccgcggcgctatcggccagcaccgcgaggaaggacaggggcgaggccgggtcgcgggcggccgcgcccagggccgacaccgcgtccgccagggcgccatgcgcccgcacggccgcgtccaccgtcgccgcgggacttgccgtcgcgacggcggcgctcccggcgttgatggcgtttgacacggctttggcgattgtgggggcgtgatcggaaaagaactgcacgaggaccggcgtctcgggggcgtcggcgaacagggtcttcagcaccaccacgaaggcgggatgcaggccggccagagccgtcgcggtatccggggtcgggtgttccagggcctcccggtactgccccagcagcccccacaggtccgcccgcagcgccgccgtgacttccgggggggggccccggacggcatcggccaggtcggtccaccccgcgggcagggaggcccgcagggtcgccagcacggccggacacgcctttagccccacaaagtccgggaggggccgcaggaccccttggagtttgtgcaggaacttctcccgggcgtcgtgggccaccttggcgcgctcccgcgcgtcgttgagcatcgcctccagggcgtgggcgcgctcccgaagccgggagcgcgcctccggagcgagctccgccgtcatcttggccgcctccatggccctcgcctgccgcagcgcgtcttcggccatgcgcgtggcctcgggggacagcccgcccccgtcgacgtacggcgcggggccggtcgccgggacgaaggccgcgtcgctgtccagctgctgcgcgagcgccgcgtcgagggcgtcgaagcgctgcagttcggccagccccgagctgcgccgcgcctgctggtcgttgatgccgtggatgctgcgcgccagctcttccaggggcttgcgttcgatgagcccctgggtcgcggcgtcggtcaggaccgagagccaggccgccaggtcctcgggggcatctaggggctggccccgctggagcaggtcccgcagcaggatggcctgggggctggtggcgagggggggcgggggggggagcgcggcgcgctgagcgacgtcccgcgtgtgttggtcaaaggccggtagcgattccagcaactggaccatgggcacgaccgcggccgaggccacgtgaaaccgacagtcgtggctgtcgctggcctgcagggccttcgcgctgtatacggctccccggtggaagtactccttgaccgcgctctcgatcgcccggcgggcctggatccgcacgtcctccagccgcgcctggatggcctcggggcccagggcgggcgggcacggggccctgccgccggcgcccggggcggcgggcacgggcatcacggtcaggggcccggcgcgctgcgagaccgagtcgaccccgcgggcgagggcgtctaaggcctcgcgcatctcgcgggcctccgcctcgacccgcatctcttcgccccgggcaaactgggccagcgcctggatccgatggagaagcggctccgggtgcgtcggggtggcgggggcgaacagggtgttcgggtgggcgcgcgagcgctccaggagccactctccgaggcgtgcgtacagattggccggcggggcggcgcgcagctgcagatccaggtccgcgaggtccccgtaaaaggcgtccgtctcccgaataacgtccctggcgaccaggaccagcttagcgagggccaggcgcccgatctgcgaattttcgtccagcacgtgctggatgaggggccggtgggcggccacgtccgccaggctcatgcgcgtggacgccaggaagtccccgacggccgttttgcggggcagcatgcgcagggtgaagtccagcagggccgcggccgggccggccaccccggcctgcgtatgcgtgcgggccccgttctcgatcaaaaaggcgaggacgcgctcaaagaagaagatgacgcagagctccaacagccccgggtgcgccgggtacggcgaccgcagggcgttgatggtgagctgcgaacacgcggccacctcgcgggccagggcggcatcgcgcgccgcgagccggaccgccgtggcggccacattggggtggacctcgaacagctgcgccaggtcggcgccggggggctccggggggcggcgggcccccagcgtctcgagcacggacggcgacgacgggctcgcgggcccgtcgtcgccgccgccctgcccggactgcgggggggtatccggtgcgggagggaccgtggcggctatgggcgtcggggaggaggcggggacctcggcggcgacgggggccttcttcttgggcgcggacttcttcttggccttggcgggcggggccttgggggcgggcctctcgcccgaggtcagatcctccacgctggacggtggggtccaggtgggccggcggcgcttgggcaagccggtagaatagcgcgcccggtggcgacccaccggcactgcccccacctccaggacccgcaggtcctcggcttcttcggccgcgtccccggcgggtgtctgcgggggcggggcggcgtgcggtggacccgaggccgcggcgtccggggccgagggcttcgcgggcggggtcccctccagggctgctgcccacacatcatcgggggggcggtttgggtgccccgcctgcggtgtgtcgggtgggcccgaggccccccggggggcctcggggggccggtcggccccaggggtctggacgtgggtgggcgcggggagcgcggggacgaccgggcccgagccttctccgtcccccctggggaccacaccgacaaagagcgccccgagccccccgatctcgccccgcagggggtgggtgatggccacgcgccgctcgacgaacggttcgtcctgcaggtaagtctcgctggccccgtagaggtgcagggccgcggcggtcaggtccgccggcgccacggcccccgggccggagggcacaaaaaacaccatggcgcccgcccaccgcaccttggggcggtcgtgggcgtaatacgtcaggtacgggtacacgtcgcccgcccgcaccttggcgataaacgcgggcgttcccgcgggcaggccgtgcgggtcaaacagataggccgtgtcgccgtcccggtagagccccatgcccagggggccgatggtcaggagcgtgtaggacagcggccgcatggcccaggggccggcgaagaacgtgtgcgcggggcattgcgtctccagcagccccgccgtgggctccccgaagaagcccacctcgccgtacacccgcgaaaacacgcaacgcaggccgccgcgcgccgccgggtactccaggaagttggggagctcgataatggaacacatgcgcggcggcccggagcccgcggccgcgcgcgtccactcgcccccctccaccagacatccctcgatggcctccgcggacagcacgtcgcggggccccacgtcgaaaagaagactgagaaacgacagggacgagcgcatgcacgataccgacccccccggctccagatcggtcgcgaactggttccgaacaccggtgaccacgatatcgcgatccccctggcgcttcatcgtggggtgaggtagcgcggccggaatcatgtgtgccgcgcccgccacgagcggggcctgtttatgggccgggcgtcccgatgagtactgttgtttccgccgcccgaacccccccgcccatcaaccgcctgttcgtccccctaaccacacacccggtatcgcgtgtgtgtggtttcccgggaagacacatcccaccccatgaagttttgccctttttttccgtcccgcactacgccacctttccacccccccccaaaaaaacaacaaccaactcccagatggatgggtgcgataataaagctttattattgtttaaccaaaggcgagtcctacgggtgtaccggtggtgtctcctgcggcgtcatctcgtcgtcctccacgggggtgttgggccaagggaccgtctcgcggcccgccgggcgcgtcgacggcgcgcgggcctgcgtgtcctgtgggccgggtgtcgtgggttcgggggtgctaccgccggcatcttgggcctccaggtccccgggggcccccgggccggcggaaggccgaaacgccgaggcgcgaaacacgccgtcggtgacctgcaggagctcgtttattaatagccagtccatgctcagcgtagcggccagcccctggggagacaggtccacggagtccggaaccaccgtcggctgacccaggggccccaggctgtagtccccccaggcccccaggtcatgacggttcgtgagcacgacgaggtctgcggccgggctggggggcgcgtcctcggtcgcgtgggccatcacctcctgaatggctgcggtgcgctgatcggccgagctggcgaagcgcgccacgaccagcgcgcgctccgtctgcaggcccttccacgtgtcgtggagttcctgaacgaactcggccacccgctcggggcccgtggccgcgcgtgcggcctgatagccggccgagaggcgccgccagcgcgccaggaactgactcatgtaacagaacccggggacctggtcccccgacatcaactttgacgccctggcgtggatgcccgacacgatggccaggaacccgtggatttcccgccgcacgacggccagcacgttaccctcgtgcgagacctgggccgccagctcgtcgcataccccgaggtgcgccgtcgtctcggtgacgacggaccgcagccccgcgagggacgcgaccagcgcgcgcttggcgtcgtgatacatgccgcagtactggctcaccgcgtcgcccatggcctcggggcgccagggccccaggcgctcgtgggcgtctgcgaccacggcgtacaggcggtgcccgtcgctctcgaaccggcactcaaagaaggcggcgagcgtgcgcatgtgcagccgcagcagcacgatcgcgtcctccagctggcggaccagggggtcggcgcgctcggcgagctcctgcagcaccccccgggccgccagggcgtacatgctgatcagcagcaggctgctgcccacctcgggaggctgggggggaggcagctggaccgcgggccgcagctgctcgacggcccccctggcgatcacgtacagctcgcgcagcagctgctcgatgttgtcggccatctgcatcgtgggcccgacgccggcccgggtggccggttcgaggagggtgatcagcgcgcccaattttgtgcggtgcccctcgacggtggggagatagcccaggccgaagtcgcgcgcccaggccagcacccgcagggcaaactcgatggggcggggcaggtaggcagcgttgcacgtggccctcagcgcgtccccgaccaccagggccagcacgtaagggacgaaccccgggtcggcgaggacgttggggtggatgccctccagggccgggaagcggatcttggtggccgcggccaggtgaaccgagggggcgtggctaggcggcccgacggggagcagcgcggacagcggcgtggccggggtggtgggggtcaggtcccagtgggtctggccgtacacgtcgagccagatgagcgccgtctcgcgcaggaggctgggctggccggcgctgaagcggcgctcggccgtctcaaactcccccacgagcgtgcgccgcaggctcgccaggtgttccgtcggcacggccgggcccatgatgcgcgccagcgtctggctgaggacgccgcccgacaggccgaccgcctcacagagccgcccgtgcgtgtgctcgctggcgccctggatccgccggaacgttttcacgtagccggcgtagtgcccgtactcccgcgcgagcccgaacacgttcgcccccgcaagggcaatgcacccaaagagctgctggatctcgctgagcccgtggccggggggcgtccgcgcgggcacccccgccaccaaaaacccctccagggccgatatgtactgggtgcagtgcgcgggcgtgaaccccgcgtcggtaagcgtgttgatcaccacggagggcgagttgctgttctggaccaaagcccacgtctgctgcagcagcgcgaggagccgttgctgggccccggcggagggcggctcccctagctgcagcaggccggtgacggccggacggaagatggccagcgccgacgcactcagaaacggcacgtcggggtcgaagacggccgcgtccgtccgcacgcgcgccatcagcgtccccgggggcgcgcacgccgaccgcgggctgacgcggcttagggcggtcgacacgcgcacctcctcgcgactgcgaaccattttggtggcctcgaggggcgggatcatgatagccgggtcgatctcccgcaccgtgtgctgaaactgggccagcagcggcggcgggaccaccgcgccccgatcgggggtcgtcaggtagtcgtccaccagcgccagcgtaaacagggcccgcgtgaggggggtcagggcggcgtcgtcgatgcgctgtaggtgcgccgagaacagcgtcacccaattgctgaccagggccaagaaccggagaccctcttgcacgatcggggacgggaagagcaggctgtacgccggggtggtcaggttggcgccgggttgccccaggggaaccggggacatcttaagcgacatctccccgagggcctccagggaggtccgcgggttcatggccaggcagctctgggtgacggtccgccagcggtcgatccactccacggcacactggcggacgcgcaccggccccagggccgccgtggtgcgcagcccggcggcctccagcgcgtgggtcgtgtcggagccggtgatcgccaggaccgtgtccttgatgacgtccatctcccggaaggccgcctcgggggtctcggggagcgccaccgccatgcggtgcaccagcagcccggggaggttctcggccaagagcgccgtctccggaagcccgtgggcccggtgcaaggcgcacagttgctccaggagcgggtgccagcacgcccgcgcctccgccgggccgaccgccgcgcccgacaacagaaacgccgccgtggcggcgtgcagtttggccgcggacagaaacgccggctcgtccgcgctgcccgccggctcgctcgagggggagggcggccggcggaggttggtcaggctccccaacaggacctgcaacggtccgtttgggggtggagcggacgggggggtcatgccggcgggcgccgggacctggagcgcgctgtccgacatggcgaccggcgtgcgcgctcggcgacgcggcgcggagaccgcgggcccaaacgggaatgactgccgccgccctatacggaggggctaagtatcgcccggggacccttcgaaaccccgggcgtgtcgcaagtacgccgcgaaggcgcggcgtgttatacggcgcgttatgtcccggcattccgttcgtgggttcgggcccgggtgctgtcgggtgggagtgtgtgtgggggggggcggcgcgacggcggcccggaccaagtgtatcgcggccgttccgtggggcggcccaacaggccctttaaacatttgcgtatgcaccggcccagccagtcggacaccggaacccaccagaggcggaagccgccttcgcccgtgagggtgcgtgtgttttctggtggcgtgtttttcctttccgccctcctccctccccacctccaccacccccccccacaactcgcccgttggcgatcggcgggaaaaccatgaaaaccaagccactcccgacagccccgatggcgtgggccgagagtgccgtggaaaccaccaccagcccgcgcgagctcgcgggccacgccccgctccggcgcgtcctgcgcccgcccatcgctcgccgcgacggcccggtgcttttgggggacagggcccccaggaggacggccagtacgatgtggctgctggggatcgaccccgcggagtcgtctccgggaacgcgcgctacccgagacgataccgagcaggccgtggacaagatcctcaggggagcccggcgcgcgggagggctgaccgtccccggcgccccccgctatcacctgacccgccaggtaaccctgacggatctctgccaaccaaacgcggagccggccggggcgctccttttggccctgcggcaccccaccgacctcccccacctggcccgccatcgggctccgcccggccggcagaccgagcgactggccgaggcctggggccagctcctggaggcctccgccctggggtccgggcgggccgagagcggctgcgcgcgcgcgggccttgtgtcgtttaactttctggtggccgcgtgcgccgccgcctacgatgcgcgcgacgccgccgaggcggtccgggcccacatcacgaccaactacggcgggacgcgggccggggcgcggctggaccggttttccgaatgcctgcgcgccatggtccacacgcacgtgtttccccacgaggtcatgcggtttttcggggggctagtgtcgtgggtcacacaggacgagctggctagcgtcaccgccgtctgcagcggaccccaggaggccacacacaccggccacccgggcaggccccgttcggccgttaccatcccggcctgcgccttcgtggacctggacgccgagctgtgcctggggggcccctggggggcgttcctgtacttggtcttcacctaccgacagtgccgggaccaagagctctgttgcgtgtacgtggtcaagagccagctccccccgcgcggactggaggcggccctcgagcggctgttcgggcgcctccggataaccaacacgattcacggggccgaggacatgacgccccctcccccgaaccgaaacgttgactttccgctcgccgtcccggccgcgagctcgcaatccccgcggtgctcggcgagccaagtcacgaacccccagtttgtcgacaggctgtaccgctggcagccggatctgcgggggcgccctaccgcacgcacctgcacatacgccgccttcgcagagctgggtgtcatgccagacgacagcccccgctgtctgcaccgcaccgagcggtttggggcggtcggcgttccggttgtcatcctggagggcgtggtgtggcgcgcggcggggtggcgggcctgcgcgtgatcgtctattgacgacggccgcccaacccgagcgaccttcccctcccacttccccccccctacacaccaactccgccctcgccgtcttggccgtgcgcggccccgtgcgtccgtctcaataaagccaggttaaatccgtgacgtggtgtgtttggcgtgtgtctctgaaatggcggaaaccgacatgcaaatgggattcatggacatgttacacccccctgactcaggagataggcatatcctccttagattgactcagcacacgatcgcaccccacccctgtgtgccggggataaaagccaacgcgggcggtctgggttaccacaacaggtgggtgcttcggggacttgacggtcgccactctcctgcgagccctcacgtcttcgcccaccgattcctgttgcgttcctgtcggccggtgctgtcctgtcgacagattgttggcgactgcccgggtgattcgtcggccggtgcgtcctttcggtcgtaccgcccaccccgcctcccacgggcccgccgctgtttccgttcatcgcgtccgagccaccgtcaccttggttccaatggccaaccgccctgccgcatccgccctcgccggagcgcggtctccgtccgaacgacaggaaccccgggagcccgaggtcgccccccctggcggcgaccacgtgttttgcaggaaagtcagcggcgtgatggtgctttccagcgatccccccggccccgcggcctaccgcattagcgacagcagctttgttcaatgcggctccaactgcagtatgataatcgacggagacgtggcgcgcggtcatttgcgtgacctcgagggcgctacgtccaccggcgccttcgtcgcgatctcaaacgtcgcagccggcggggatggccgaaccgccgtcgtggcgctcggcggaacctcgggcccgtccgcgactacatccgtggggacccagacgtccggggagttcctccacgggaacccaaggacccccgaaccccaaggaccccaggctgtccccccgccccctcctcccccctttccatggggccacgagtgctgcgcccgtcgcgatgccaggggcggcgccgagaaggacgtcggggccgcggagtcatggtcagacggcccgtcgtccgactccgaaacggaggactcggactcctcggacgaggatacgggttcggagacgctgtctcgatcctcttcgatctgggccgcaggggcgactgacgacgatgacagcgactccgactcgcggtcggacgactccgtgcagcccgacgttgtcgttcgtcgcagatggagcgacggccccgcccccgtggcctttcccaagccccggcgccccggcgactcccccggaaaccccggcctgggcgccggcaccgggccgggctccgcgacggacccgcgcgcgtcggccgactccgattccgcggcccacgccgccgcaccccaggcggacgtggcgccggttctggacagccagcccactgtgggaacggaccccggctacccagtccccctagaactcacgcccgagaacgcggaggcggtggcgcggtttctgggggacgccgtcgaccgcgagcccgcgctcatgctggagtacttctgtcggtgcgcccgcgaggagagcaagcgcgtgcccccacgaaccttcggcagcgccccccgcctcacggaggacgactttgggctcctgaactacgcgctcgctgagatgcgacgcctgtgcctggaccttcccccggtcccccccaacgcatacacgccctatcatctgagggagtatgcgacgcggctggttaacgggttcaaacccctggtgcggcggtccgcccgcctgtatcgcatcctgggggttctggtccacctgcgcatccgtacccgggaggcctcctttgaggaatggatgcgctccaaggaggtggacctggacttcgggctgacggaaaggcttcgcgaacacgaggcccagctaatgatcctggcccaggccctgaacccctacgactgtctgatccacagcaccccgaacacgctcgtcgagcgggggctgcagtcggcgctgaagtacgaagagttttacctcaagcgcttcggcgggcactacatggagtccgtcttccagatgtacacccgcatcgccgggtttctggcgtgccgggcgacccgcggcatgcgccacatcgccctggggcgacaggggtcgtggtgggaaatgttcaagttctttttccaccgcctctacgaccaccagatcgtgccgtccacccccgccatgctgaacctcggaacccgcaactactacacgtccagctgctacctggtaaacccccaggccaccactaaccaggccaccctccgggccatcaccggcaacgtgagcgccatcctcgcccgcaacgggggcatcgggctgtgcatgcaggcgttcaacgacgccagccccggcaccgccagcatcatgccggccctgaaggtcctcgactccctggtggcggcgcacaacaaacagagcacgcgccccaccggggcgtgcgtgtacctggaaccctggcacagcgacgttcgggccgtgctcagaatgaagggcgtcctcgccggcgaggaggcccagcgctgcgacaacatcttcagcgccctctggatgccggacctgttcttcaagcgcctgatccgccacctcgacggcgagaaaaacgtcacctggtccctgttcgaccgggacaccagcatgtcgctcgccgactttcacggcgaggagttcgagaagctgtacgagcacctcgaggccatggggttcggcgaaacgatccccatccaggacctggcgtacgccatcgtgcgcagcgcggccaccaccggaagccccttcatcatgtttaaggacgcggtaaaccgccactacatctacgacacgcaaggggcggccatcgccggctccaacctctgcaccgagatcgtccacccggcctccaagcgatccagtggggtctgcaacctgggaagcgtgaatctggcccgatgcgtctccaggcagacgtttgactttgggcggctccgcgacgccgtgcaggcgtgcgtgctgatggtgaacatcatgatcgacagcacgctacaacccacgccccagtgcacccgcggcaacgacaacctgcggtccatgggcattggcatgcagggcctgcacacggcgtgcctcaagatgggcctggatctggagtcggccgagttccgggacctgaacacacacatcgccgaggtgatgctgctcgcggccatgaagaccagtaacgcgctgtgcgttcgcggggcgcgtcccttcagccactttaagcgcagcatgtaccgggccggccgctttcactgggagcgcttttcgaacgccagcccgcggtacgagggcgagtgggagatgctacgccagagcatgatgaaacacggcctgcgcaacagccagttcatcgcgctcatgcccaccgccgcctcggcccagatctcggacgtcagcgagggctttgcccccctgttcaccaacctgttcagcaaggtgaccagggacggcgagacgctgcgccccaacacgctcttgctgaaggaactcgagcgcacgttcggcgggaagcggctcctggacgcgatggacgggctcgaggccaagcagtggtctgtggcccaggccctgccttgcctggaccccgcccaccccctccggcggttcaagacggccttcgactacgaccaggaactgctgatcgacctgtgtgcagaccgcgccccctatgttgatcacagccaatccatgactctgtatgtcacagagaaggcggacgggacgctccccgcctccaccctggtccgccttctcgtccacgcatataagcgcggcctgaagacggggatgtactactgcaaggttcgcaaggcgaccaacagcggggtgttcgccggcgacgacaacatcgtctgcacaagctgcgcgctgtaagcaacagcgctccgatcggggtcaggcgtcgctctcggtcccgcatatcgccatggatcccgccgtctcccccgcgagcaccgaccccctagatacccacgcgtcgggggccggggcggccccgattccggtgtgccccacccccgagcggtacttctacacctcccagtgccccgacatcaaccaccttcgctccctcagcatcctgaaccgctggctggagaccgagctcgtgttcgtgggggacgaggaggacgtctccaagctctccgagggcgagctcggcttctaccgctttctgtttgccttcctgtcggccgcggacgacctggtgacggaaaacctgggcggcctctccggcctcttcgaacagaaggacattcttcactactacgtggagcaggaatgcatcgaggtcgtccactcgcgcgtctacaacatcatccagctggtgctctttcacaacaacgaccaggcgcgccgcgcctatgtggcccgcaccatcaaccacccggccattcgcgtcaaggtggactggctggaggcgcgggtgcgggaatgcgactcgatcccggagaagttcatcctcatgatcctcatcgagggcgtcttttttgccgcctcgttcgccgccatcgcgtacctgcgcaccaacaacctcctgcgggtcacctgccagtcgaacgacctcatcagccgcgacgaggccgtgcatacgacagcctcgtgctacatctacaacaactacctcgggggccacgccaagcccgaggcggcgcgcgtgtaccggctgtttcgggaggcggtggatatcgagatcgggttcatccgatcccaggccccgacggacagctctatcctgagtccgggggccctggcggccatcgagaactacgtgcgattcagcgcggatcgcctgctgggcctgatccatatgcagcccctgtattccgcccccgcccccgacgccagctttcccctcagcctcatgtccaccgacaaacacaccaacttcttcgagtgccgcagcacctcgtacgccggggccgtcgtcaacgatctgtgagggtctgggcgcccttgtagcgatgtctaaccgaaataaaggggtcgaaacggactgttgggtctccggtgtgattattacgcaggggaggggggtggcggctggggaaagggaaggaacgcccgaaaccagagaaaaggaccaaaagggaaacgcgtccaaccgataaatcaagcgccgaccagaaccccgagatgcataataacgattttattactcttattattaacaggtcgggcatcgggaggggatgggggcgcgcgtttcctccgttccggctactcgtcccagaatttagccaggacgtccttgtaaaacgcgggcgggggcgcgtgggcccacagctgcgccagaaaccggtcggcgatgtccggggcggtgatatgccgagtcacgatggagcgcgctaaatcttcgtcgcggaggtcctgatagatgggcagtctttttagaagagtccagggtccccgctccttggggctgataagcgatatgacgtacttgacgtatctgtgctccaccagctcggcgatggtcatcggatcgggcagccagtccagggcctccggggcgtcgtggatgacgtggcggcgacgtccggcgacatagccgcggtgttccgcgacccgctgcgcgttggggacctgcacgagctcgggcggggtgagtatctccgaggaggacgaccgggcgccgtcgcgcggcccaccggcgacgtccgggggctggaggggggggtcttcttcgtagtcgtcctcgcccgcgatctgttgggccagaatttcggtccacgagatgcgcgtctcgaggccgaccggggccgcggtcagcgtaggcatgctctccagggagcgcgagttggcgcgctcccgccgggccgcccggcgggcctgggatcggctcggggcggtccagtgacactcgcgcagcacgtcctcgacggacgcgtaggtgttattggggtgcaggtctgtgtggcagcggacgaacagcgccaggaactgcgggtaactcatcttgaagtactgcagcaggtcgcggcagtgaatcgtcggaatgtagccggtgctgatgtccaacacgatatcgcagcccatcagcaggagatcggtatccgtggtatgcacgtacgcgaccgtgttggtatgatagaggttcgcgcaggcgtcgtcggcctccagctgacccgagttgatgtaggcgtaccccagcgcccgcagaacgcggatacagaacaggtgagccaggcgcagggccggcttcgagggcgcgcccgagggggccgccgggcctgggccggcggcccgcgttccccggtcccccggggcgaaggcgtgcccgcggcggcgcatgttggaaaaggcgaaactgggcctggagtcggtgatgggggaaggcggcggcgaggcgtctacgtcactggcctcctcgtccgtgcggcactgggccgtcgtgcgggccaggatcgccttggccccgaacacaaccggctcggtacactcgaccccgcgatcggtcacgaagatggggaacagggacttttgggtaaacacccgtaacatactacagagacagtgtagcgtgattgcctcgcggtcgtaacttgggtagcggcgctgatatttaaccaccagggtatacatgacattccacaggtccacggcgatgggggtaaagtagccctccggggcccggaggccccggcgcttcaccagatggtgagtctgggcaaacttcatcatgccaaacagacccattccggcacgattgtaggtgcggataggtctctctacagagctgtataggtgtgacggtccgggacacccaagcccgccgcccctgtgtacagtggctgcggcgacgaccccgctccaacaagacgctatcccgggaaaggcacgctctttataattcttttttatttcccatctacgtgcggattggtgcaaccgccggcgcgcgccggtgcaggccgaccatctctctcttccccccctccccctcccccgagccctcaaagagggtgtggcctaactagcggaaggcgtatttaaccagactagggcggcgggtccgccgtagtccttggctcgggtagccactgctctgtggctcgggtcccccggcccccctaacccccatccggtccgcgtcatccgccccctccgcctgcgacacaaacggccgcgcctccgggcccggtgacacgacgcgcctcgtctctgcggattgtcccgggagcgtcgcggcatggctcatcttcccggcggtgcggccgccgcccccctttcggaggacgcgatcccgtcgccgcgcgagcggacggaagactggccgccctgccagatagtgctgcagggcgccgagctgaacgggatcctgcaggcctttgcgccgcttcgcacgagccttttggactcgctcctggtcgtgggcgaccgaggcatccttgtacataacgcgattttcggcgagcaggtgtttctgcccctcgaccattcgcagttcagtcgctatcgatggggcggacccaccgcggcgttcctgtctctcgtggaccagaagcgatccctgctgagcgtgtttcgcgccaaccagtaccctgacctgcggcgggtggagctgacggtcacgggccaggccccgtttcgcacgctggtgcagcgcatatggacgaccgcgtccgacggagaggccgtggagcttgccagcgagacgctcatgaaacgcgagttgacgagcttcgcggtactactcccccagggcgaccccgacgtccagctgcgcctcacgaagccccagctcacgaaggtggtgaacgccgtcggggacgagaccgccaaacccaccacgttcgagctcggccccaacggcaagttttccgtgtttaacgcgcgcacctgcgtcacctttgccgcccgcgaggagggcgcgtcgtccagcaccagcgcccaggtccagattctgaccagcgcgctgaagaaggcgggccaagcggccgccaacgccaagacggtctacggggaaaacacacaccgcacattctcggtggtcgtcgacgactgcagcatgcgggcggtcctccggcggctccaggtcggcggggggaccctcaagttcttcctcacggccgacgtccccagcgtgtgtgtcaccgccaccggccccaacgcggtgtcggcggtgtttcttttaaaaccccagcgggtctgcctgaactggctcggccggagcccgggttcctcgaccgggagcttggcgtcccaggactctcgggccggcccgaccgacagccaggactcctcctccgagccggacgcgggcgaccgcggcgccccagaagaagaaggcctcgagggccaggcccgggtaccgcccgcgttcccggaaccgccgggaaccaagcggaggcaccccggggccgaagttgtccccgcggacgacgccaccaagcgcccgaagacgggcgtgcccgccgcccccacgcgagccgagtcgccccccctctccgcgagatacggacccgaggcggcggagggtggtggggacggcggccgctacgcgtgctactttcgcgacctccagaccggcgacgcgagccccagccccctctccgccttccggggtccccaaagacccccatacggctttgggttgccctgacggcaacgggtggtggccgaacgcctcaccgcgcccgggcacgcggggtgcgttgtgttaaaaaaataaataaatggggtagtgtgtcccccccccctccaaccaatatggctgtcgtgtgtggttccgggttgcgcctccgtcctttccaccccccttccccctccttttttgttttgcgtgcgcttataagagcgggcccggggcccttcgcagcttcaccgagagcgccgtcgggccccgggtgcgggatgtgtcgcggggacagccccggggtcgcgggcgggagcggcgaacactgcctcggaggggatgatggggacgacgggcgcccccgcctcgcctgcgtgggtgccatcgctcgggggttcgcgcatctctggctccaggccgccacgctgggcttcgtggggtctgtcgttctgtcgcgcggcccgtatgcggacgccatgtcgggggcgttcgtgatcgggagcaccggcctggggttcctccgcgccccccccgcgttcgcccggccgccgacgcgtgtgtgcgcgtggctgaggctggtcggcgggggagcggccgtggccctgtggagcctcggggaggccggcgcgcctccgggggttccgggcccggcgacccagtgcctggcgctcggggccgcctacgcggcgctgctggtgctggccgacgacgtccatccccttttcctcctcgccccgcggcccctgtttgtcggcaccctgggggttgtcgtcggcgggctgacgataggcggcagtgcgcgctactggtggatcgacccccgcgccgccgcggccctgacggcggcggtggtggcgggcctcgggacaaccgccgccggggacagcttttccaaggcctgtccccgccaccgccgcttttgcgtcgtctccgcggtcgagtctcccccgccccgatacgccccggaggacgccgagcggccaacagaccacggacccctgttaccgtcgacgcaccaccagcgatctccgcgggtctgcggcgacggggccgcacggcccgaaaacatctgggttcccgtggtgacctttgcgggcgcgctcgcgctggccgcctgcgccgcgcgagggtctgacgcggctccgtcaggcccggtcctgccgctgtggccccaggtgtttgtcgggggccacgcggcggcgggcctgacggagctgtgtcagaccctcgcgccccgggacctcacggacccgctgctgtttgcgtacgtcggattccaggtcgtgaaccacgggctgatgtttgtggtccccgacatcgccgtatacgcgatgctggggggcgccgtgtggatctcgctgacgcaggtgcttgggctccggcgccgccttcacaaggacccagacgccgggccctgggcggccgcgaccctgcggggcctctttttctccgtctacgcattggggtttgcggcgggggtgctggtgcggccgcggatggcggcgagccggcggtcggggtgatcgccatttcaaataaaaggcacgagttccccgaataccaccggcgtgtgatgatttcgccctaccgctccgatccccggggggaggggggaaggaaatgggggcgggggtgccgtggacgggtataaaggccaggggggcaggcgggcccatcactgttagggtgttaggttgggaggtggcacaaaaagcgacactcccgtgttgtagttgtccgcgggaggcggtggtttccggcaaccctcctcgctgcgccgggcgcgcccaccggtccttcgcgggggccggggctcttctggtcatggcccttggacgggtgggcctagccgtgggcctgtggggcctgctgtgggtgggtgtggtcgtggtgctggccaatgcctcccccggacgcacgataacggtgggcccgcgggggaacgcgagcaatgccgccccctccgcgtccccgcggaacgcatccgccccccgaaccacacccacgcccccccaaccccgcaaggcgacgaaaagtaaggcctccaccgccaaaccggccccgccccccaagaccgggcccccgaagacatcctcggagcccgtgcgatgcaaccgccacgacccgctggcccggtacggctcgcgggtgcaaatccgatgccggtttcccaactccacccgcacggagttccgcctccagatctggcgttatgccacggcgacggacgccgagatcggaacggcgcctagcttagaggaggtgatggtaaacgtgtcggccccgcccgggggccaactggtgtatgacagcgcccccaaccgaacggacccgcacgtgatctgggcggagggcgccggcccgggcgccagcccgcggctgtactcggtcgtcgggccgctgggtcggcagcggctcatcatcgaagagctgaccctggagacccagggcatgtactactgggtgtggggccggacggaccgcccgtccgcgtacgggacctgggtgcgcgttcgcgtgttccgccctccgtcgctgaccatccacccccacgcggtgctggagggccagccgtttaaggcgacgtgcacggccgccacctactacccgggcaaccgcgcggagttcgtctggttcgaggacggtcgccgggtgttcgatccggcccagatacacacgcagacgcaggagaaccccgacggcttttccaccgtctccaccgtgacctccgcggccgtcggcggccagggccccccgcgcaccttcacctgccagctgacgtggcaccgcgactccgtgtcgttctctcggcgcaacgccagcggcacggcatcggtgctgccgcggccaaccatcaccatggagtttacgggcgaccatgcggtctgcacggccggctgtgtgcccgagggggtgacgtttgcctggttcctgggggacgactcctcgccggcggagaaggtggccgtcgcgtcccagacatcgtgcgggcgccccggcaccgccacgatccgctccaccctgccggtctcgtacgagcagaccgagtacatctgccggctggcgggatacccggacggaattccggtcctagagcaccacggcagccaccagcccccgccgcgggaccccaccgagcggcaggtgatccgggcggtggagggggcggggatcggagtggctgtccttgtcgcggtggttctggccgggaccgcggtagtgtacctcacccacgcctcctcggtgcgctatcgtcggctgcggtaactccggggccgggcccggccgccggttgtcttcttttccaccccttccgtcccccgtacccaccacaccccaccccacccccccgccgtcccccgggcgttataagccgccgcactcgcttttcccaccggaaaatcctcggcccgatccgaacggcgcacgccgcgtgggctccaaacgcctccggaagagagcgccccgccccgatattcaagcccgcggtggtgctatggctttccgtgcttcgggacccgcctaccagcccctcgcccccgcggcctccccggcgcgggctcgtgttccggccgtggcctggatcggcgtcggagcgatcgtcggggcctttgcgctcgtcgccgcgttggttctcgtaccccctcggtcctcgtggggactctcgccgtgcgacagcggctggcaggaattcaacgcgggatgcgtcgcgtgggaccccacccccgtcgagcacgagcaggcggtcggcggctgcagcgcgccggccacccttatcccccgtgcggccgccaagcacctggccgctctgacacgcgtccaggcggagagatcgtcgggttactggtgggtgaacggagacggcatccggacctgtctgagactcgtcgacagcgtcagtggcatcgacgagtttttcgaggagctcgcgatccgcatatgctactacccacgaagccccggcgggtttgtccgcttcgtaacttcgatacgtaacgccctggggttgccgtgaggcgcgcgtccgacggtcccgcttctcgcctctcttcttcccccaccccacccaccgaccaacgacggcgtttggccaataccctccttttttctttttctcttcccccccccccaaaaaaaacaataaacagctaattgcgtacgacaaaccatgcggaactcgctgttttttttctctgtttgttactttttattgaaacagacatacggggaaaggggccggaaaccgagacggtggggccggcggtcgcatttttttaatggctctggtgtcggccgcgtttgagcttcgtcaacagggcgctgagggcggcgacgttcgtcgggccgtcgttggccagcgcgttggtccgggggcgggcgggcatgggcgacaggcttagtcccgggtccggggcgcgtgtggcccgccgaggggagaagagggcagacccgccccagtcgtacaggggattttccgcctcgatgtacggggagtccggggcgtctcccggcagggcggccccgccggcaagacgccggcgagggcagatgttttcgtatacccgaacccaggggatctcctcgtagacgcgccccccatcctcgcccaccgactcgtaaatggaatctgcgtcctcggagggggcgcggggggcgtggctttcggccggccaggcggcggcggcggtggtgtcggcggcgggggtggcgccaagcccgacgcccgcgggcatggcggcgtcatcgtcgggcagcagatacgtgttttccatctggtccggttcggcctccgcgtccggcccccaggtccgcaccgcgtcgtagaccccggcggcctcgcgctgagccgcgagcgggcgcgccgcggctgccggccgctgctcggggggcgcggggttgcggggcgggaggcgcgggggcgccccggccatatgcgtgtaatacgtggccggccggccggcgcagggctcgggaccccggtcggccgcgtcgacgtgcgggggctcggggaggtcctcgcggtggcgcctgcacctccgaggggccgcgggggtcgagtgggggcgagcccgggggagcggcgggggtgcgttgtcgcgccgggtccgttgtatcttgtcccggcagctcccgccgaccgcgccgcggccccccggtgggccggacgccgcgaggcgcaggatggactcgtagtggggcgacggggttccgctccgaagcaggtccggggccagggcggccccgaaccaggacttgatgctgagttccatccgggcccagctcggggcggtcatcgtggggaacaggggggcggcggtcctgcagaagcgctcctggctgtccaccgccgccgtaaggtactcgttgttcaggctgtcggaggcccagacgacatacccggtaagcgtcgcgttaattatatactgggcgtggtggtggactatggatagaacctcgacggtcgagacgatggcgtccacgatcccgtacgtgccgccgctgcgcttgccggtctcccacaggtgggccaggcgcgtcaggtggcccaggacgtcgctgaccgccgcccgcagggccatgcactgcatcgagcccgtggtgccgctgggcccgcggtccaggtggcgcgcaaacgtctccgcgggcgcctccagactcccgctgagcgccacgaaccggcgatcggcggggcccaggcggcgacacacgtacttgtccgccgtccacagcatccacgaggcccaatggtacaacacggagacgtaggccaggagctcgctcagccgcagtgcggtgtccgtgctcggccggctcgggtctgcggggcgcataaagaacatgtactgctggagcctgtgggccgcgtcgcgcaaccccgccaccgcggcggcgtacttggccgcggcggccccgctcttgaacggggcgcgcaccagcagcttcgggagcagggtgggccgcagcagcacgtgcaggctggggtcgcagtcgcccgccgggtcgtcggggatgtccaggccgctgggcacgaccgtctggaggtacttccagtactgcgctaggatggcgcggctcagctggccgcccgacagctccacctcgccgagcgcctgcttggcggccgacgcgtagtgccggatgtagtcgtagtgcgggtcgctggcgagcccgtctacgatcaggctctcggggacggtgttatggtgccgcgccgccagccggacgctgcgatcggcgccggtcagaaacgccggctgcaggtcgtcggcgcgctgccgcaggacgcccacggccgcgctgaggagcccctccggggtggggagcagacacccggcgaagatgcgccgctcggggacgcccgcgttggcgccgcggatgaggttggccggcgtcaggcaccgcgccagccgcagggagctcgcgccgcgcgcccggcgttgcatggcggagaccgttcggtcgggggccccgccggtcggaggtatgccgcgtcccgggatatagggttgctttttatggggaggcgcctatgggcgtggcgggccgcccagcccggtcgcgcgcctcccggacacgtgcgcccggagggcggcggtctcctcgtcgcccatgagcagtttccgaaactgcgccatgatgtccacgacgcggacccgcggccccagcacggactcgctattcaggggggcgggggggaaggccgccaggtcttcgagcaggaaggcggggtctgccgtcccgctcacgggcgcccggggcgccgaggacgcggggcgaaggtccacgtgttccgcggcggcgcgcacgtccgcccaaaatttggcgggggtggtccgcgcgtacaggggctgggtcgcgcggaggacgcacgcgtagcgcaggggggtgtacgtgcccacctcgggggccgtcgacccgccgtcaaacgcggccagggccacgcacgcgaccaccgtgtcggccaggcccagcagccgctgcaggatgagccccgtcgccagcacggcgcgcgcggccgccgcgtcgtccctgcgccggcgcgcgtccccgcaggccagggcgtatttcagggtaacggtcgccagggccgtgtgcagcgcgtacacggccgcgcccagcacggcgttcagcccgctggtggcgagcaggcggcgcgccgcggtgtcgcccagcgcctcgtgctcggccgccacgaccccggggctgcccaggggcagggcgcgaaacagcgcctcctgctccacgtccgcaaacgcggggtgggcggagtgcgggtgcaggcgcgcccccacgaccaccgagagccactggaccgtctgctccgccaggaccgccagcacgtccaggacgcgccccgcaaacgcggcctcccgcgggagcacgcatttgacggcgccggggttgaagcgggcgagcagagccccggtggcgatgtacgtcatgcgccccgcgtagcgggcggccacgcgacagtcgcgccccaggagcgcgcgcaccccgggccagtacagcagggaccccagcgaactgcgaaagaccgcggcgtcggggccggggtgggggggcgcggcccctcccgcgctgagcagcggcacggcggcggcccccacgggccgcaacgccgtgaggctcgcgaactgccgtcggagctcggccgccctgtcgtcgagctccgagccgcgcccctccgtgtgcaggcgcgtcccgcagacccacccgttgatcgccacccgcacgatggcgtccaccagaaaacccatcgcgcgggaggggctggtttttgcccgccgatccgtcaggtcgaggatcgcgtcgcccgtgacgtaccaggccagcgcctcgccctgctgcagcgtctggcggaaaaacacctttgggtcggccggggaggcaaagtgcatgacccccacgcgcgacagcccgaacgcgctatccggacacgggtagaacccggccggatgtcccagggccagggccgagcgcacggactcgtcccacgcggcgactcggggggtcaggcggtccagggggaatgccgcctgcagctccgggcccgacacgcggcccgcgagaatctcgaccgtcgcggaaggccgcgccccggggccgtcatcgtgcgcgacggcggcggggtagtcgtcctcctcgtagttgagctcgtccaggaacagcggcgagggcaccacccgcgaaccgcccacccgccccaaaacgtcgcgtgggtccatcgggcccaggtagcctccccgcggggcccgcgtgatggcgctgtcccggcgtccgcgaacggactggctcctggccgtaacggacctggggcgcggaaaggacgcccggcgggggggcgccgccgcccgggcctcggacgcgcgtcgggacccggggtgaccgcgggcctcccggcgacggcgcgggggcggctcttcgctcgccatctcccccgcggcctcgacctcgctgtcgtcgtccacgttaaacaccgcccgcaggtaccccattaacccgactccaccgccctcgggctcgtcctccacgggcgagtcggcgcgatgcgcggacggggcatgggaccgggtggaggcgcgcctccggcgtacggcatgcccgcgcacggacatggtggccggaggcccgattttttacacacgccctccccgcagacggacgaggaaaggggtggtgcgaggggggaggcccaaacggggaggtggggggtagggggcggtcccagggagcggggggtaggaaccggcacgacgggaacagagaaaacgcgaccgctccaacaagggtgggggggtgggcctcgtccccacgcagacccgcgggcaaatgcgagaacgggacccgcgcgcctgcctttatacgcggaccccagcaccacgagccgttctgtgacgcgaatctacacgaccgcgggctcgtaggcgcgactaacgcccaacccaacggcacacaccccccaccccgcgcgtaaccccatttctttcatggtcccgtaataaacagccaacgcacgccgcgtatgatgagttgcttgccaatgtttattgctgtggttgcgaaccctctatcgcgatacagacggaggtgaggcggggcggtggtggggggggggcgcgccgcccggtcgcacatcctaccccccaaagtcgtcaatgcccatggcatcggtaaacatctgttcaaactcaaaatcgtccacgtccaaagccccatacgagacggggtcgtgggtcattcccggggagggggactccacgtcccccagcatctccaagtcgaagtcgtccagggcgtcggcgggcgtcatatccacctcctcgccgtccaggcggagttcgtctcccaggctgacgtcggtaatgggggcggtggtggacagtctgcgggggcgttgtcccgcggagagaaacgacatgcgcggcgccaccagcccggcctccgcaggagcgtcatcgtcgtccgggaggtcgagcaggccctcgattgtcgatccgtaattgtttctggtccgcccgcggctatacgcgtgctcccgcatgacggactcgccctccgaggtcgcgacgctggagtacgagtccaacttggcccggatcagcagcataaagtacccagaggagcgggcctggttgccctgcaggacgggcggggtcgtgaggggcgccccgggttcctccgccgccgcacttcgcaccagcgggaggttcaggtgctcgcgaatgtggtttagctcccgcagtcgccgggcctccacgggaactccccgcacggtgagcgatccgttgataaacatcaggggctgaaacagacacgccaactggcgccagctctccaggtcgcagcagaggccgtcgaacagatcgggccgcatcatctgctcggcgtacgcggcccataggatctcgcggctcagaaagaggtatagatgcagaaacaggacgcgcgccaggcgcgcggtctcgcggtagtacctgtccgcgatcgtggtgcgcagcatctcccgcaggtcgcggttgcggccccgcatgtgtgcctggcggtgtagctgccgaacgctggcgcgcaggtaccggtacagggccgagcaaaaatttgccaacacggtccggtagctctcctcccgcgcccgcagctcaccgcggaaaaactgcgccatggcctcgtagtacgaaggcagctcgtcgcgggtggcgggcagggtggggaacgccacgtcgccgtgggcgcgaatgtcgatcggggagcgctcggggacgtgcgcatccccccagtcgatcacgtcgctgggcagcgtcgacagaaacttgcactcccggtacatgtcggcgttggtcgggaacccagagaacaggtcctcgttccaggtatctagcatggtacacagcgcgggacccgcgctgaagcccagatcgtcgaggagacggttaaacagggccgcgggggggacgggcatgggcggcgagggcatcagctgggcctgactcagccgaccggtggcgtacagcggaggggcggctggggtgttcttgggacccccggctggcctggggggcggtggcgaaaccccgtccgcgtccgcaaacagatcgtcgaccaacaggtccatgggggcggttgggtccgggaataacgatctcgagaggcgaatgagacgtgcccgagcgcccggcggcggagagggggggagggatccgggacccgcgacagaaaaaggccggggccctcgcgaagggaatcgccgggggtgccgtgcgtccccgaggactgacatctcgcgtccaccaccccgcatttaagtatcaccccagtgccgccccaaacctcgtgacttccccaccgctccgggcggcccgtcccccgcgctcggaagggaggcgtgtccttcctcccgcccctcccgcccctcccgcccctcccgcccctcccgcccctcccgcccctcccgcccctcccgcccctcccgcccctcccgcccctcccgcccctcccgcccctcccgcccctcgccacaaacgcgtgctgacagcgaagtggttaaatcgaccgtgatgctttattgtctgtcgtctgaacgcggtcggggtcgctactcgagggggcggcggggacgggaagccgagcgggcgggggcccgtgcggtcgcggcggcacgccccgcggggcggccccgggcggccgcggtcgcgtcgacgtcctgcgccgcgtcgggattcaccaactcgttcgcgcgctgcaggaggttcttgccctcgcagaccgtcacgcgaatggtggtgaggtcgaggagctcgttgaggtcttcgtcggtgtgcggccgcgacatgtcccacagctgtaccgccgccagccgggcgtgcgtggccgccaggcgcccgaccgcggcgcagaagacgcgcttgttgaacccggccacccggggggtccacggcgccgtggggctcggtggggcggtgctgaagtgcagcttcttggccagtccctgggcgggtgtcttggttcttcccgaggccgtgggagcgggggcgtctaggagcacggcggagtcggcctgggcgggtcgcctgccgcgggcggggtcggtcgccggggtcgcggaggccttaggcgccccgcgcgtcattttgggggtccgcgcgggaggggcgtgcgagcgcccgccggcgcccacggggcccccggggggtggaggagcgcgcgcggggccggggccgtgagagcccgcgacggacgccgaacgacgcggtcgcgcggtatcccgggactcgtcgtcgtccgaagacgagtcccggtagagggcatacccagcctcgtcataatggagaaagcgaacctcgcccctcgggcgcgcgcgcatcgggccagcgccgcggcggaagtcgtcgcgcggactctctgggtccgccggggagaccgggccatagtacagctcctcgtgggtcccgcgcggcgcttcccgcggacacgacttgacggagcggcgagaggtcatggtctatcggagacaccggggacgcccgtgcggatcacagggaaggcgtcggcgaaggaggcagagagcgtcggaaggcggcgagggagggaaagagggagaccggcggggtacgggagagcagcgagggcctgcgtaacccacgggggccgcgggagtggctccctgcgggttgcgggggagagtttataggaagtggatataaccgcaggcgacgggactaaccaatccccgggggggcaacggacagacacgccccgaacaggcccgacttccgcgaggaagcaaaggccgggggccgcccaacgacacgcccaccccttcccaacagggcgggctcaggctgacccggcggccagtgcccgctgacatatctgatacacgtgcgcgatcatacatacgcccatcgaggtcatgcctagataaaagggcaccaggacccccgggacggacaccacaccggcgctgtcgccccggcattgcgcgtccccgataacgccgcgtgcgcctgccgcgttcggcggctccccgggcacgcccgcgacgagcgcgacgaacaacagcaccacccagcggcccagtcttgcgggtttccccgtcatcgcggcgatgagtcagtgggggcccagggcgatccttgtccagacggacagcaccaaccggaatgccgatggggactggcaagcggccgtagctattcgcgggggcggagtcgttcaactgaacatggtcaacaaacgcgccgtggattttaccccggcagaatgcggggactccgaatgggccgtgggccgcgtctctctgggcctgcgaatggcaatgccgcgggacttctgcgcgattattcacgcccccgcggtatccggccccgggccccacgtgatgctcggtctcgtcgactcgggctaccgcggaaccgtcctggccgtggtcgtagccccgaacgggacgcgcgggtttgcccccggggccctccgggtcgacgtgacgtttctggacatccgggccacccccccgaccctcaccgagccgagctccctgcaccggtttccgcagttggcgccgtccccgctggcagggttacgagaagatccttggttggacggggcgctcgcgaccgccgggggggcggtggccctgccggccagacggcgcgggggatcgctggtctacgcgggcgagctaacgcaggtgaccaccgagcacggcgactgcgtgcacgaggcgcccgcctttctgccaaagcgcgaggaggacgcaggctttgacattctcatccaccgagccgtgaccgtcccggccaacggcgccacggtcatacagccgtccctccgcgtattgcgcgcggccgacggaccagaggcctgctatgtgctggggcggtcgtcgctcaatgccaggggcctcctggtcatgcctacgcgctggccctccgggcacgcctgtgcgtttgttgtatgtaacctgaccggagtcccggtgaccctacaagccgggtccaaggtcgcccagctgctcgtcgcggggacccacgccctcccctggatcccccccgacaacatccacgaggacggcgcattccgggcctaccccagaggggttccggacgcgaccgccaccccccgagacccgccgattttggtgtttacgaacgagtttgacgcggacgcccccccaagcaagcggggggccggggggtttggctccactggcatctagaccgcgcctcgcgtcgggccagatggggccccggtcaataaagagctctgtttcgcatatgccctggtgttggcggtttttttttgttgtctgtctgcccggcgctcggttgtccgttctgtcgtcgctatcacatacgcacaaacacacgggtagagtggaaccgaaaccggtcgacgtttattcaccacacagaaacacaagctaagcgagaaggaggggggcctcggtcgacgaggcctggcgtttgggggcggacgtgcgatgacgtgggtccggtgtagggtccgcggggggcacgggcccggggcgaacgggggatctgtcgccggcgtgggtgactgggaccgacgcaacctccggggcttgtgccctcgtaggcccggggggggcctcggtcgctccaagccccgcggtgcgggtccctccggccagagccgaggtggagagaccaagggcccgctccgcgatcgccacgtcctccatgaccacgtcgctctcggccatgctccgaatggcctgggagacgagcacgtccgccgacttgtccgcggcccccaccgacatgtacatctgcaggatggtggccatgcacgtgtccgccaggcggcgcatcttgtcccgatgcgccgcaacggccccgtcgatggtggagccctcgagtcccgggtggtggcgcgccagcctctcgaggttgaccatgcaggcgtggtatgtgcgggccagggcgcgcgccttcacgaggcgccgggtgtcgtccagcgactctagggcgtcgtcgagcgtgatgggggcgggcaaaagcgcattgaccaccgccagggcctcctgcagccgcggctccgcctccgagggcggagccgcggcccgaatcatctcatattgttgttcctcggggcgcgttccccaaccgcacagcaccccgagcagggacgccatcccggaacacgcgcgcggctctgcgccggctttcccccaccccaccccctccgggttcgcaggggcgatggggacggaagactgcgatcacgaagggcggtcggttgcggctcccgtggaggttacggcgctgtatgcgaccgacgggtgcgttatcacctcctcgctcgccctcctcacaaactgcctgctgggggccgagccgttgtatatattcagctacgacgcgtaccggtccgatgcgcccaatggccccacgggcgcgcccaccgaacaggagaggttcgaggggagccgggcgctctaccgggatgcgggggggctaaatggcgattcatttcgggtgaccttttgtttattggggacggaagtgggcgtgacccaccacccgaaagggcgcacccggcccatgtttgtgtgccgcttcgagcgagcggacgacgtcgccgtgctccaagacgccctgggccgcgggaccccattgctcccggcccacgtcacagcaactctggacttggaggcgacgtttgcgctccacgctaacatcatcatggctctcaccgtggccatcgtccacaacgcccccgcccgcatcggcagcggcagcaccgcccccctgtatgagcccggcgaatcgatgcgctcggtcgtcgggcgcatgtccctggggcagcgcggcctcaccacgctgttcgtgcaccacgaggcgcgcgtgctgggggcgtaccgccgggcgtattatgggagcgcccaaagccccttttggtttctgagcaaattcggcccggacgaaaagagcctggtgctggccgctaggtactacctactccaggctccgcgcttggggggcgccggagccacgtacgatctgcaggccgtgaaagacatctgcgcgacctacgcaatcccccacgacccacgccccgacaccctcagtgccgcgtccttgacctcgttcgccgccatcactcggttctgttgcacgagccagtactcccgcggggccgcggccgctgggtttccgctgtatgtggagcgccgcatcgccgccgacgtacgcgagaccggcgcgctggagaagttcatcgcccacgatcgcagctgcctgcgcgtgtccgaccgggaattcattacgtacatctacctggcccactttgagtgcttcagccccccgcgcctggccacgcatctccgggccgtgaccacccacgaccccagccccgcggccagcacggagcagccctcgcccctgggtcgggaggcggtggaacagttcttccggcacgtgcgcgcccagctgaacatccgcgagtacgtaaagcaaaacgtcacccccagggaaaccgccctggcgggagacgcggccgccgcctacctgcgcgcgcgcacgtatgccccggcggccctcacgcccgcccccgcgtactgcggggtcgcagactcgtccaccaaaatgatgggacgtctggcggaagcagaaaggctcctagtcccccacggctggcccgcgttcgcaccaacaacccccggggacgacgcggggggcggcactgccgccccccagacctgcggaatcgtcaagcgcctcctcaagctggccgccacggagcagcagggcacgacgcccccggcgatcgcggctctcatgcaggacgcgtcggtccaaacccccctgcccgtgtacaggattaccatgtccccgaccggccaggcgtttgccgcggcggcgcgggacgactgggcccgcgtgacgcgggacgcgcgcccgccggaagcgaccgtggtcgcggacgcggcggcggcgcccgagcccggcgcgctcggccggcggctcacgcgccgcatttgcgcccggggccccgcgctccccccgggcggcctggccgtcgggggccagatgtacgtgaaccgcaacgagatcttcaacgccgcgctggccgttacgaacatcatcctggatctggacatcgccctgaaggagcccgtcccctttccccggctccacgaggccctgggtcactttaggcgcggggcgctggcggcggttcagctgttgtttcccgcggcccgcgtagaccccgacgcctatccctgttattttttcaaaagcgcctgtcggccccgcgcgccgcccgtctgtgcgggcgacgggccctcggccggtggcgacgacggcgacggggactggttccccgacgccggtggtcccggcgacgaggagtgggaggaggacacggaccccatggacacgacccacggccccctcccggacgacgaggccgcgtacctcgacctgctacacgaacagataccagcggcgacgcccagcgaaccggactccgtcgtgtgttcctgcgccgacaagatcgggctgcgcgtgtgcctaccggtccccgccccgtacgttgtgcacggctccctgacgatgcgtggggtggcgagggtgatccagcaggcggtgctgttggaccgcgacttcgtggaggccgtagggagccacgtaaagaactttttgctgatcgatacgggcgtgtacgcccacggccacagcctgcgcttgccgtatttcgccaagatcggccccgacggctccgcgtgcggccggttattgcccgtcttcgtgatcccccccgcgtgcgaggacgttccggcgttcgtcgccgcgcacgccgacccgcggcgcttccactttcacgccccgcccatgttttccgcggccccgcgggagatccgcgtcctccacagcctgggcggggactatgtcagctttttcgagaagaaggcgtcgcgcaacgccctggagcactttgggcgacgcgagaccctgacggaggttctgggccgctacgatgtgcggcccgacgccggggagaccgtggaggggttcgcgtcagaactgctggggcgaatagtcgcgtgcatcgaggctcactttcccgagcacgcgcgggaatatcaggccgtgtccgttcgccgggccgtcattaaggacgactgggtcctgctgcagctgatccccggccgcggcgccctgaaccaaagcctctcgtgtctgcgcttcaagcacggcagggcaagtcgcgcgacggcccggacctttctcgcgctgagcgtcgggaccaacaaccgcctatgcgcgtccctgtgtcagcagtgctttgccactaaatgcgataacaaccgcctgcacacgctgtttaccgtcgatgcgggcacgccatgctcgcggtccgctccctccagcacctcacgaccgtcatcttcataacggcctacggcctcgtgctcgcgtggtacatcgtctttggtgccagtccgctccaccgatgtatttacgcggtgcgccccgccggggcgcacaacgataccgccctcgtgtggatgaagataaaccagacgctgttgtttctgggcccgccgaccgccccccccggcggggcatggaccccccacgcccgcgtctgctacgccaatatcatcgaaggtcgggccgtgtccctcccggccatccccggcgccatgagccgccgggtcatgaacgtgcacgaggccgtaaactgcttggaggccctctgggacacccagatgcgcctggtggtcgtcggttggtttctgtatctagcgttcgtcgcccttcaccaacgacgatgcatgttcggcgtcgtgagtcccgcgcacagcatggtggccccggcgacctatcttttgaactacgccggccgcatagtgtcgagcgtgttcttgcaatacccctacacgaaaatcacccgcctcctctgcgagctatccgttcaacgccagaccctggtgcagctgttcgaggcggatccggtcaccttcttgtaccaccgcccggccattggcgtcatcgtgggctgcgagctgctgctccgcttcgtggccctcggtctcatcgtcggcaccgctctcatctcccggggcgcctgcgcgatcacacaccccctgtttctaacaatcaccacctggtgtttcgtgtccatcatcgccctgacggagctgtatttcatcctgcggcggggctcggcccccaaaaacgcggaaccagcggcccccagggggcgctccaaagggtggtcgggcgtctgcgggcgctgctgttccatcatcctctccggtatcgccgtgcgcctgtgctatatcgccgtcgtggccggggtggtgctcgtggcgcttcgctacgaacaggagattcagcggcgcctgtttgatctgtgacgtaacgcctcttccgttggaagaggcggacccagtcgcccatacaaattaaatacacgacccgcctcgggcctacgcaccctcgcacgtcgcatgcaaattaaaatcgtgcacagagccgatccggcctcgggtctgcttgcccctcccccggcccagcacaggcaggctcgtccgacttccgcatacaccccaccctaccgcgtgcttccgcacccccgcctacgcgtgtacgcgaaggcggacccagacctgccgtatgctaattaaatacataaaacccaccctcggtgtccgattggtttctggggacggcgggggcgggggcggtgacgcccgacggggagggacaaggaggagtttcggaaagccggccccggtcgtgcgggtataagggcagccaccggcccactgggcgctgtgtgctgccgtgtgccgaccccggttgcgcgtcggtgccgctcctcgattcggacccggccactctcttccgacacgcgccccctcggaggacacccgccatcccagccccggcgacctacaacatggctaccgacattgatatgctaatcgacctaggattggacctgtccgacagcgagctcgaggaggacgctctggagcgggacgaggagggccgccgcgacgaccccgagtccgacagcagcggggagtgttcctcgtcggacgaggacatggaagacccctgcggagacggaggggcggaggccatcgacgcggcgattcccaaaggtcccccggcccgccccgaggacgccggcacccccgaagcctcgacgcctcgcccggcagcgcggcggggagccgacgatccgccacccgcgaccaccggcgtgtggtcgcgcctcgggaccaggcggtcggcttccccccgggaaccgcacggggggaaggtggcccgcatccaacccccgtcgaccaaggcaccgcatccccgaggcgggcggcgaggtcgccgccggggccggggtcgatacggccccggcggcgccgactccacaccaaaaccccgccggcgcgtctccagaaacgcccacaaccaagggggtcgccaccccgcgtcggcgcggacggacggccccggcgccacccacggcgaggcgcggcgcggaggggagcagctcgacgtctccgggggcccgcggccacgaggcacgcgccaggccccccctccgctgatggcgctgtccctgacccccccgcacgcggacggccgcgccccggtcccggagcgaaaggcgccctctgccgacaccatcgaccccgccgttcgggcggttctgcgatccatatccgagcgcgcggcggtcgagcgcatcagcgaaagctttggacgcagtgccctggtcatgcaagacccctttggcgggatgccgtttcccgccgcgaacagcccctgggctcccgtgctggccacccaagcgggggggtttgacgccgagacccgtcgggtttcctgggaaaccctggtcgctcacggcccgagcctctaccgcacattcgcagccaacccgcgggccgcgtcgacagccaaggccatgcgcgactgcgtgctgcgccaggaaaatctcatcgaggccctggcgtccgcggatgagacgctggcgtggtgcaagatgtgcattcaccacaatctgccgctccgcccccaggaccctatcatcggaacggcggccgccgtgctggaaaacctcgccacgcgcctgcgcccctttctgcagtgctacctgaaggcccgaggcctgtgcgggctggacgacctgtgctcgcggcgacgcctgtcggacattaaggatattgcctcctttgtgttggtcatcctggcccgcctcgccaaccgcgtcgagcgcggcgtgtcggagatcgactacacgaccgtgggggttggggccggcgagacgatgcacttttacatcccgggggcctgcatggcgggtctcattgaaatactggacacgcaccgccaggagtgttccagtcgcgtgtgcgagctgacggccagtcacactatcgcccccttatatgtgcacggcaaatacttctactgcaactccctattttaggcaagaataaacatattgacgtcaacccaagtggttccgtgtgatgttcttggcgcgcgcggcgggtggggcggagactccggggcgatgccggcgtgcgcgtgggaggagggcgatgacccaccggataaatgtggggccccggcccggcccgcttcatagcgcgtccaggaactcacggcagacgcgtattcaccgacccccccctcgcaacatgacaacgacgcccctctcgaacctgtttttacgggccccggacatcacccacgtcgcccccccgtactgtctgaatgccacgtggcaggccgaaaacgccctgcacacgaccaaaacggaccccgcgtgcctggccgcgcggagttatttagtccgcgcctcctgctcgaccagcggccccatccactgttttttctttgcggtgtacaaggactcgcagcactcccttccgctggttaccgagctccgcaacttcgcggacctggtcaaccacccgcccgtcttgcgcgaactagaggataagcgtggggggcggctgcggtgcacgggcccattcagctgcggaaccatcaaggacgtctccggtgcatcccccgcgggggaatacacgataaacggtatcgtgtaccactgtcactgtcggtatccgttctccaaaacctgctggctcggggcatccgcggccctacaacaccttcgctctataagctcaagcggcacggccgctcgcgcggcagaacagcgacgccacaaaatcaaaatcaaaatcaaggtataacccacccccttccctccgagtccgtatgcaacctcattaataaagagtgagaaccaaccaaaacagacgcggtgtgagtttgtgggttataggaacccggtaaataccacgcgacgaaccagcatgtgtgttaacgcaacttttattcgttgtatcgcgggaggggggaagcttaccgccaaaggaaggccaagatgataacgacgaccaccgcgaccaccccaaaaaccgcatgacgacacgtcccgccacaccaccctggggcttggggcgtgtcggagctcgacgcacagcgggccgcgcgttgggcccggtacagctctcgcgaattgacaagcgggggtcgccacgtgcgcgagctttgcacgcggggttggtcggccggccccacggacccgcccggtggctcggtcggacatgcggccatgaccatggcgtaggtgggggggcgatccgaggtcgcctctgcgtaagtagggaggcccgacgggaggtcgcctcccacgccagggtgggccccaatcatagtttccggtagaaacaggggggtctccacaaacaacccccctgggccaaagctccggcgccgcgcccgtcgttcggcgcggcgcctggcgcgccgagcggcccgccaggcggcgcggcgcgagcggccacgctcacacacctcgccgtcaccggaagaagccggtgaaacaagcccaaccggcgacgtccctgcagagtacggtggaggcgagtccgtgggggtgtcgatatcaataacgacaaactggcccgcgctcgcgccggccacactctcgtatgggggcggggcgtcaatcacgctatcatctccgtcatccctgcatgcgtgggcatgcccagcccccaacgccatggtggggattcgcggctcagaagcctgcatgtcgtgtggtcggtcgtagtccaacgtgcctcccccacccaccacacagccggtccccacgccgaccactagaccgcagacgtcgcccaaccgaggtccccgtgcacagaccgcgccttttatagccccaggggttgctaattaacgcacgcatgcagacgcaatttattttgctcccccgcgtcctcccctcccctgcgcacacgtgataggtcttgggaacccgaggggcgacgcggggaaagcgcgcccccgcccggccgccgcgcgcccccgcccggccgccgcgcgcccccgcccggccgccgcgcgcccccgcccggccgccgcgcgcccccgcccggccgccgcgcgcccccgcccggccgccgcgcgcccccgcccggccgcccgcgtcgcgccggcgccccctcccggcgcttccggggtctttccttccttccccgccgcgaccccgaccccgccccaccgccccgcccggcaggggggccccggcgccgcgcagaacacacagacgaacacacggtggcgatcttttctttacttcggcggaccagcgagccccggccccggcccgcgccccgccgccacacccacggcacccccccccgccgcccaccccggggtccacacaggagcgcgcgggcggcagaaacgcgggcgcggcggcggtcggggtgggagtggtggtgggggacacgaaaacacacccacgacactctccccccaccccgaccgccgccgcgccccaccggcgggatcgcggcgagacgcagccgggcccccccccaccacccgcccacccacctaccccgcgcccgcagcctccggcagcacgccgaccaccgccgccaccccccaaacagccaaggcgcggtggggggcgtggtggtgaacgatggggggaacacgggggggaggggtccggggcgaggcgggcgggcgaaggaagggggggtggtggcggcggcggtggaaagcggaaaaacggaggatggaagggcagaagatggggagtcccgatcctcctcctgcatcccctcgccttccattctccggccctccgcgagtcccgacgccccccccccgccgcccgacgaaggagacccaagcaccgcagccggagaggccgagcggggagtgggcggccgggcgggaggatggcggagagagagagagagagagagagagggggggggggggagagggaaagcaacgggaaagagaggcgcgcggaaaagcagcaagaggggggacggggcgagccgggcagagtgcggagcccccggagcccgcggccgcagccgagcagcgccgcgggctccggggccgggccgggccggcaacgccccgcgccggccgcggcggagagaacccctgtgtcattgtttacgtggccgcgggccagcagacgggccgcgggccagcagacgggccgcggcgccagcggcccacgcctcccgccgcattaggcccccgcgggcatccggcggccggccccacgcccttccattaaacactcccacgttgggggggggcgcgccagctgagtgctctgcggttgcgggcgccgtgcccggagatccattaagccgccggagagcccgagccccgcccgcgtgttgctgtgggcatttctgctgcgtcatccctgtctttataaaaccgggggcgcggcagcaacgaacgcaggggcccgccgccgatcgagagggactccggagaaggaaggctgctccgcgcaccggcgcgcccttctcctctcccctccctacctccccctctcttcccccttttttcccccgcctcccgtcttcttccgcgcctccgagggtccgcctcttgcctcggggacccccgggcgggccggggcttggccgccgaggtgcgccccggccggaggggcccccgcacctcggcggccgccccctccggcgccgcgcgttcgcgaaaggcgcgaaaggggcccccggaggcttttttcgattcccggccgggggtcccgggtagccgcccggcgccgggcggaaggcgtcccccgcccggcggtccggcccgggcccccggcggagcgcgggggccccggggccccgggccgcgccggcggcgtttccgcgttccgtttcttctccctcccgggccgccccgctcccgggcccgaccctcgccccttcccttctcctcgtcttcccccgtcccgccgcgccccttccctcttccttctctctctctgtctcgctctcctcacatttcccccccccccccccgccgccgccgccctttgcccgcgtcccaccgagacgccgcgccgcgtgagccgtccgccgggggacccaggctccggggggggggggcgcctgcgtgtgtctcgtgtgagagagcgcgcccctcgaacgccgcgcgttctcgcaggtaggtttagggtcgtacaggtgagcttctgctgaggcggcggggagagggggggggggcgggcggaagagagaagagagcaggggttgggggagaactgttcttcctccccctttcaagaaacacgaggcgggggtcccagaaagggcaggcaggtcagccgcaccgcccgcgagccaacccgtatcctttttttctaggtgtttttgtttttgtttctgtttttgtttgttttgttattattttcgcggatccggcgtgttcggatccaccccccctttctccttcctcttcccttccacccacccccgtttcccccccccccgtcgtcgttcccgggggggcaggcgcgggtcgggcccgtacgcccaccgcccccacgcgccggtcacccccccccaacaaccccaaaggcgcgtgcccggccacagccgtgggtgtggcgcccgtccccttcctctaccgcgtgggcgcgggcgggggggtggtggtagtggtggcggaaggaaacgggccgggggccggggccgctagggaaaggtaggcacgcgcgcggtgtgtcgacttgcatgccccgcaaaacgcgtcgtgtcgtgttgtgtcgtggtgggccgtgttgtggtgggccgtgtggtgtggtgtggtgttgcgaacgcgcgagccccctcgccccgatgggagtctccccgcagccagggtaaggaggggcgggcgtggcgggcaggtgtgcgggcggggtggggtgagtgcggttgcatgcctcgggtctcctcttcctgctcctcctcctttctcccagccagggtgaggaggggcgggcgtggcgggcaggtgtgcgggcggggtgggcgccggggcgggggtgggcacgggcgtaagtgcgggtgcatgcctcgggtcttctcttctccctcctccttcctcccacccgtccccgggggcagagggcgtgcatgcgttgtgattcaaccgccctcgcccccgccccactttcccccctctctatcaaagttccctggcccctggcttcgcgccggtggtgcggctgaccccccccctcctccctccccgagccaggcgccctcccactcctgcccaccacccccagggtctggccggccagacgtgcgtgctctgcacgatcgggcccccctccctgtcaacacggacacactctttttttacccgccagccagcccgcccacccaccaagacagggagccagaacgaggccgggccccggctctgttctatgataaagaccaacaggcctcgggggtgggggcggcttctcgtgcccgccccccctcctcctcctcccttcccccccatccccggcccccctgcgcgggggagctgcatcaaaggccaacaacaaagtgtgtcaaaagcatcacaaaactttattgtaaaatttttataaatataaagttttttttttcctcaagttttcaacaaggccagaaagtccataacaaaatgctggtgtgtgttgctgttcggggccgtgtccgtcccccccccccactcccacccccacttcctgtctcctccccgtctttccccccccccacctccccctgcccccgaggcgcctcggccggtggtccggtggggggcggcttccttcgggcagcaagccgagtgttagctccccctactccccgtggcccgcgggggcgtcgccggccggcgcgggcgcgccctgctcccgagaccacgggtggcgcgaccggaggccgtggaagtccagcgcgcccaccagggtgccctggtcaaagagcatgttgcccaccggggtcatccagaggctgttccactccgacgcggggggcgtcgggtagtcggggggcctcacgcagttgcgcgcgtgctcggggagcagggtgcggcggctccacgcgggggccgcggcccgcagcaggtccgccacgttccccgtctggtccacgaggaccacgtaggcccctatgtggcccgtctccatgtccaggacgggcaggcagtcccccgtgaccgtcttgttcacgtaaggcgccagggccacgacgctcgagacccccgcgatgggcaggtagcgcgtgaggccgggcgccgggtcgcgggccccgggctcggggccgccctccgcgtggcgcgtcttcctggcacacttcctcggcccccgcggcgcagcagcgcgggggccgagggaggtttctcgtctctccccagcgccggacgcggacgcgacgctcccaccagccccgcccgcagaggaagaggcggaggaggaggaggcggaggaggaggaggcggaggaggaggaggcggaggaggaggaggcggaggaggaggaggcggaggaggaggaggcggaggaggaggaggcggcggcgaccgcggcctgggacgacggagacgccgacgggggcgcggcgcccgcggacgccggggcgagcggcccgtggccgcggtcgcccgagtccgagtccggggcccggcgcggcgccgccctcttggcccccaccccctggggggcgaggggcgagcgcggggcggcggaggaagaggcggaggacgaggccgcggggcccgagtccgacccgcgcctcttccgggggcgggccgccgccccctccgcggcgtggggggcggcaccgggggtgttggtgccgcgggggaccccgggtcctccctccgcgcccggccctcccgacccgcgcgcgtcggtcgcgcctgcccggcccagactctgtgcttgggtgtcggtctgagcctgggtcatgcgcgaccggggcgcgcggtgcgcgtccaccggcacggcgggcggcgcgggcccggccgcgtccgcgctcgcagacaccacgggggcggcggcggcgcggggcggactccggacgcgcggggcgacggccgcgcgggggcgcgcggcgcgccccgacgactgtggcagacctccccccccggggcccgaggacacctgtgcggaggaggaggagacaaaggagagcggcccggggcccgcggggcggcgcggagacggcgggggagagtcgctgatgactatggggggctcctgggccgcgcggggctgtctcgcggggggcgtcctgccctccgccgccgcggcgtcttcgcccacccgccgcgcctgcgcgcgccccccgccggccgcagggggaagagaggccactctcggcacgacggccgcgacggcagggccgcccccagacccagatcccacccccgcccgcaacggggcgccgccgctgctgctgctccgcggggcgccagggggcgccggtcgggtcgcggcgggctgggaggttccgcgggtcgcccccgcaccgccgcccccgcgccggggcgctcttcggggggcgggcgggacgtagtccactgcagagggagacagagacgggagcccccggttagtgcccgacccccgcccgacccccgcccgacccccgcccgacccccgcccgacccccgcccgacccccgcccgacccccgcccgacccccgcccgacccccgcccgacccccgcccgccccccgcccgacccccgcccgccctcaccgtcggccaggtcatcgtcctcgtcgtccgtgccgggccacgggggggtgggcgacagggcgcggaccgtgtgtccccccagcgacagggagcgcggggccgtccgcgggttgcccgtccagataaagtccacggccgtgccggcccgcacggccgcctcggcctccacgcgggtccgggggtcgttcactatcgggatggtgctgaacgacccgctggcggtcacgcccactatcaggtacgccaccggggtgttgcacaggggacacgtgttgcgcaacggaatccaggtcttcatgcacgggatgcagaaggggtgcaggcagggaaaactctggcagcgcaggggcggggcgatctcgtccgtgcacacggcacacacgtcgcccccccctcccgcttccgcttcctcctcacccacgggcccacccccacaggatccctgcgcgtcggcgggcgtggggctgccctggcgctcggccgggggccgggccgggggcgtggccgcgtccatcaggcccgcctcgaacatctccgtgtccgtgctgcccgcctcggaggtggagtcgcggtgaaggtcgtcgtcagagattcccacctcggtctcctcctccgagtcgctgctggcgagccactgcatgtcgttgagcatcccccaggcgtgcggggcggcgggctgcttgacaaagcaacgggggggatttagagggcgcggggcgtgaggcgggacccccgcgccgtgtcccccgtgtccctccctcaccccggccccccgcccgctgctttttgttcggaagggggggagaaaggggtccgtaaccaaaggtggtctgcgtcctttggattccgacccctcgtctccccccctgtcccccgctctcgggctcctccctgcctccctcgcccccccagagggtcggggggcggcgcacggcccacgggggtcccccgaccgcttaagcgggccgggggtcggccccgtcaagcgtccccgcccccgagcccaccgcccgcgaccacccccaacccgcagccgggtggtccggggaaaagggggggcctgagacccgggggtcgccctctcaccgtgccgggggtctgccgcggcggccgctcggggccggggtccgcccgggagctcgtgccgggccggggttccatgagccggggtagggtagactcgagacggcggcccgcggtctctctcttgccgggttttagtctctgtctctccgggtctcctcctcccgccgggccgccgctccgtcgctcgcagtgccggggtgcgaatgcggcccgaccgtcacacggggctgccttatacccggcgcctatccactcccccaaaggggcggcatttacgattcccccaatagccgcgcgccccggcgggggcggagggagggaatccccccctctcggggcggccccgtccccggggaccaaccgggtgtactccaagaaccccattagcatgcgccgccccccgccgacgcagatgggagtccccccggcgccccgccggcgcggccctgagtggtgcccgcccccggggaaaaattcattagcatactaggaagcccaggggaccaataggggccgatcagcccacccacccggcggcgcgcgaggctctgcgtgttctgccaagaaagtaatcagcataacccggaaccccgagggagtaattacgcggggagcgaggggccgtccgaacgtttttaattaccataagcgggaatggcggcccgttaaaagctgctaattaccgcgagcgggaacgccggcccattaaaagttgctaattaccatgcgcggggatggcggccgggaccgcctattaaaagtttctaattaccataccgggaagccggcgcggggcggtcgccggggcggagtccgggcccgcgcggcggcgcgcggttggccggcgccgccccctggggcgggcggagcggcggggcggcgccgggccctcgcggatatatacgcggggctcccatcgtctcttcggagagcggcctcgcgcagaccttcggagctccggggctccgccggccgaggccgccctcgccggttcaaccctagaccgcccgacggcccgggcccgcggcggcggaggacccgcgcgccgccgccgccgcctcctcctcctccgcgggtccgccgtcttcgtgggcccgggctcgggctcgggcccgagctcgggcctcgggctccaggcacggtccgatgaccgcctcggccgccgccacgcggcgccggaaccggtcgcggtcggcccgctcgcgcgcccaggacccccgtcgggccaggcgcgcggccgtctcccaggccaccagatggcgcacctgcacgcgcggcgagaagcacacctgcgggcggggagacacgggggtcggaggggcgtcagggggtcggaggggcgtcagggggtcggaggggcgtcagggggtcggaggggcgtcagggggtcggaggggcgtcagggggtcggaggggcgtcagggggtcggaggggaggcgtaccttcccgcgcggcgcgtccgcgggcggggacgcggggggccgccgccggcgcaggctcaggcgcgccaggtactccgtcgtggtgcgcagccgtagcgccaggtggggcggaagggggcgctgcggcccgcgctccttgcgcggcggcggcggggggcaggcggcggcaggcgcggcgtgcggggcctccggcgccttccccccgccctcgctcggggggctgttcgcccactctgcgtcgtcgttgccggcgtagtccgcgtcgtcgctgtcgtccgcctggggcaccagcagccagcgccgcaggagcgaggacgcggccggcgcgctctcgaccgcggttcccgagtcgtacgcagggaccatttgggagtctgcggttgggagcgcgccggggcgcggcacggctggagcgccggggcgcggcacggctggagcgccggggcgcggccggcgccggggaccccggcggcggggaccccggcggcgggacatggcgggcggctgggctcggcgtaggcccggagccggagcgcgtcggggcgggagagttcactcggcacgcatgcacgtgtaaccgccagtccgtgcttgcctagcgaactcacccgtcccggctggcgtgcgcagcccgggccgtgttgcgggccctcttaaggggcggcggcaggacggggactcccgccccgcctcttttcccccggggagtcaacccccggggggggtgttttttgggggggggcgcgaaggcgggcggcggcggcgggcgggcggcagggcagccccgcgcgcccccttccccgtccctcccccggagccggccgctcccccgcgggcgccgcccctccccccgcgcgccgcggggctgccttcccgcgggcgcccccgcgcggcttttttcccgcgcccgcccccgcgcggcaggacggggactagcaggctgtgccgcagaccaccacacactcccaagctccccgcccccccgaagacgccagtcgcaccaccgctcgccctcgcagaccagacagttgcaccaagcacccgcccgcccgcacacggttccccgccaccccctccctcccctccatcccgccgagctcgcggcagcccctcccccccgcgcgccacggggctgcggtcccgcggccgcctcccccgcggccgcctcccccgcgccccgccccgggggcttcccccgcccctccccccgcgcccgcggccccgagctcgcagcagcccctccctcccgcgccccgtgccttccctcccgctcctgcgggggggctcgggccacctgaccttcgtaacctgcactcaggtcagagccccagaccccccgcgggcgcgggagacgtgccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgcccgcccgacccccgaataaaccacacaaggcggtacgttttcgtctgtctcgttctttatttctcacacacgcgcgcggccatcgccgcgtctgtcttaaaggcgcacagacgcccgattccttccccctctccccatctcccccctcccccgctcccggaagtttccccccccgtcactccccaaacagtccgtcgtcgtcgtcctccagctccgcgtccatgtccacgggctcgcgcctcggcggcgtggccagccccgcggcggtccccaccacctccacgccgccgcccgccgcggccagcaccgtccccgcgcggcccgcggccgacgcccagcgtatctgcgggggcgggcccgcgtccgcgtcgtcgcgcagcaccagcgggggcgcgtcgccgtcgggctcgagcagcgcccgcgcgcagaactcccgccgcggcccgcgcagctccgccgggccgccgcgcacggcgtcgcgccccagcgccacgtagacgggccgcagcggcgcgcccaggccccagcgcgcgcaggcgcggtgcgagtgcgcctcgtcctcgcagaagtccggcgcgccgggcgccatggcgtcgcccgcgcccgaggcggcggcccggccgtccagcgccgggagcacggcgcggcggtactcgcgcggggacatgggcaccagcgtgtcggggccgaagcgcgtgcgcacgcggtaccgcacgttggccccgcggcagaggcgcagcggcggcgcgtcggggtacaggcgcgcgtgcgcggcctccacgcgcgcgaagacccccggcccgaacacgcggccggaggccagcacggtgcggcgcaggtcccgcgccgccggccagcgcacggcgcactgcacggcgggcagcacctcgcaggccaggtaggcgtgctgccgcgagaccacgggcccgtcggcgggccagtccgcggcgcgcacggcgttgacgacgatgaggcggcggtcgcaggcgccggccagcagccccaggaactccacggcgccggcgaaggccaggtcccgcgtggacagcagcagcacgccctgcgcgcccagcgccgagacgtcgggggcgccggtccagttgcccgcccaggcggccgtggcgggcccgcagagccggttgcccagggccgccagcaggcaggacagcccgccgcgctcggcggaccactccggggggggcccgcccccggcgcggcccgcggccaggtcctcgcccggcagcggcgagtagaggatcaccacgcgcacgtcctccgggtcgggcacctggcgcatccaggccgccgcgcggcgcagcgggcccgaggcgcgcagcgggccgaaggcggcgggcgcgccgccggggggcggggcggcgcagcgcgcggccagcgaggccagcgcgcgcgggtcgaacatgagggccgggcgccacggcgcggggaagagcgggtggtccgtgagctcggccacggcccgcggggcgcagtaggcctccagggcggcggccgagggcgccggcgtgtggctgggccccggcggctggcggcgccagccgccctgcgggtcggggccctcggcgggccggcgggtcagcgccgcggggcgcggcggccgcggcggcggcgtcggcggggcggggggcgcggcccccgcgggaggggcggccgcggggcggggggcgtccgcgcggctcttcttcggggggcgcggggcgccgcccggcggcgccctggccggggcggggctcttgcgcttgcgcgcctcccgcggcgcggaggcgggcgcggcgagcgagtcggccgcggcgacggtgtcggccagcagggggcgcaggctctggttctggaagagcaggtccgcggcggcggcggcggcggagctcagcaggcgcgggctccgcggcagcgccgggcccagggccccggcgaccaggctcacggcgcgcacggcggccacggcggcctcgctgccgccggccacgcgcaggtccccgcgcaggcgcatcagcaccagcgcgtcgcgcacgaaccgcagctcgcgcagccaggcgcgcaggcggggcgcgtcggcgtgcggcggggcggccgcgcccgcgggccccgggcgcgggggcgcggcgggccgggctccggccagccccggcacggccgccaggtcgccgtcgaagccctccgccagcgcctccaggatcccgcggcaggcggccaggcactccacggccacgcggcccgcctccgcgcgccggccgccgccaccaccgccggcgccgtcgtcgtcgtcgtcgtcgtcggccccggccggcgcggaggcgggcgcggcgctcaggcgccccagggcggcgagcacccccgcggcgccgtagccggcgggcaccgcgcgctcgtcggccggcgacgccgccgccgacggcaacggggcggcggcggcggcgggcttcccgcgggcgtcgtcgccgtcgtggcggttggcgtcgccgccgtcgtcgggggttcgcgccccggtcagcgccgcgttctcgcgcgccagcaggggcgcgtaggcgcggcgcaggctggtcagcaggaagcccttctgcgcgcggtcgtagcggcggctcatggccacggcggccgccacgtgcgccaggccccagccgaagcggcccgccgccatggcgtaccccaggtggggcacggcccgcgccacgctgccggagatgaaggagctgctgttgcgcgccgcgcccgagatccggaagcaggcctggtccagcgccacgtccccgggcgccacgcgcgggttctggagccaccccatcgcctccgcgtccggcgtgtacagcagccgcgtgatcagggcgtactgctgcgccgcgtcgcccagctcgggcgcccacacgggcgccggggcgcccgaggcctcgaaccgggcccgcgcctcctccgcctcgggcgccccccagaggccggggcggctgtcgcccagcccgccgtacagcacgcgccccgggggcggggggccggccccgggccacggctccccgctgacgtacccgtcgcggtagcgcgcgtagaaggcgccggaggccgcgtcggcgtccagctcgacccgccggggccgcccggccgtgaagcggcccgtggcgtcgcggccggccaccgccgcgcgggcccggcggcgctccaggcggcccgcggtcgccgcgggggtccgggccggggcgggctcggccctgggcgggctcggccggggcgccgcccccggggccctcgcgggcacccccgcctcctcgtcgtccgcgccgagggtcccgcccgcggcgtggtctgcggcgctggcgggggcgcgggcggcgtcgtcgtcgtcgtcgtcgtcagacgaggaggcggatgcagacgaggaggaggaggcggaggaggaggcggaggacgccgacgacgaggatccggattttgatgagtcagaggcggccgagcgccggcggggggcgcgccggcggcggtggtggtggtggtggtggtgtcggcggggcgccgggggtcgcggcgacaggctggccatggggtccgggtacgccccgcggaccgcggacgtcgtctccggtccgcggacccagcggcccgcgtcgcggtcgtcgtcatcgtcgtcgtcgtcgtcgtcgtcgttctcctcgccataatcggcgcgcatggagggggtccgcggcggagaaggcgagcgggccgcttcttcttgcgcgccgtcgcgctccggggggggcgacgggatcgtgcgaacggcctcgtccaccatcgaggccagcagggccagctgccgcggcgagacgacgccgtccgcggcaggctcgtcgacggcctccccggacgccggggccgcctcgtcggcatcggcatcggcggcggcgtcgtcggcctcgtcttcgttctcctccggcccaccgtgccacccgaacccgggccgcgcggcggggcgacggtccgggttcggggtgggcggcggtccgtcggctggatccggagatccggggccgccggtcgtctccgccgcggcccggagacgtcccccgtcctcgtccgccatcgcgacctcggccccgcggccctgcgtcgtcgtcgtcgtcttcttcttcttccgctgctccgccgacatcgcctccgaccggggtgtgcggggggggggtcttcttcttcttcttcaggggcggcagtgggggggggtggttggcagtctctctcccccccgtgcggtgcgtgcgtgtgcctgtgtcttttcgcctctccgcgccgatcgggtagatcctggcggccgcgtcggtagccgcgctccgtgtggacgatcgccccgtcgcctggctgatatagtcctcggggcgcgcggggcggggggaaaggaggaggacgcggaggaggagcgatcgacgccgccgcgccccggctcgccggggttccgcccccaggtggaaccgcattatgcgcggccccgccccgacgcccgcgcgtccgcgtccgtggcggcggcccgttggtcgcgccgccgccgctccgcccgcgcggcatctcattagcgcccggcgcgggcggcttccgcttccgcccgcgatgctaatgagaccctcgtcgcgggcgggctcgctcccctgcccttccgggttcgtggtaatgagatgccggccccgcgctcccgttggcccccgccggccccaaaggggccggcgaggtcgccccgttggtccgcgggcggctccgccccaaagggggcggggccgcagggtaaaagaagtgagaacgcgaagcgttcgcacttcgtcctaatagtatatatattattagggcaaagtgcgagcgctggcgccctgcccggggcccgcgtcatcccgcgctccgccccaaagggggcggggccgcagggtaaaagaagtgagaacgcgaagcgttcgcacttcgtcctaatagtatatatattattagggcaaagtgcgagcactggcgccctgcccggggcccgcgtcatcccgcgggctccgccccgaggcgggcccggacggggggcgggccgttcctcgcgcacataaagggccggcgtcccggtcgccgccgcaccaggggcacaccggctgcgcggcggagaccgggacggcagcggcggcatcgcgaagggggccacagcgagacagagacgccggcggcgagcggggcaccgacgcacccggatcggatcggatacagagacgcgggcgcatcggttccttttcgttctgcctttccctcccccccccccccccccaccctgtacgtaccgcgaggacccatccacccactgcagccttatcgcaggtacggtgacccggggggccggccggggggacgggcgggggacggggggacgggccggggggacgggccggggggacgggccggggggacgggccggggggacgggccggggggacgggccggggggacgggccggggggacgggccggggggacgggccggggggccggggggccggggggccggggggccggggggacggggggacggggggacggggggacggggggacggggggacggggggacggggggacggggggacggggggacggggggacggggggacggggggacggggggacggggggacggggggacggggggacgggccggggggacggggggacgggccggggggacggggggacgggccggggggacggggggacgggccggggggacggggggacgggccggggggacggggccccgatcccaacatccgcgctttctcgcaggccgggcgccgccttcgtggacgggacaccggtgtggtaactggcgacaaggcgttgccactatggcagacatccccccggacccgcccgcgctcaacacgacgcctgcgaatcatgctcccccatccccacccccgggttcacggaagcgcagacgccccgtcctccccagctcgtcggaatctgagggtaagcccgacacagaatcggaatcctcctcgaccgagtcgtccgaggatgaggcgggagacctacgcggcgggcgccgtcgctccccgcgggagctcggggggaggtattttttggatctgtcggcagaatcgaccacggggacggaatcggagggaacggggccgtcggacgacgatgatgatgatgcgtcagacggctggttggttgacacacccccccgcaaatccaagcgaccccgaatcaacctgcgattaacgagctcccccgaccggcgtgcgggtgtggttttccccgaggtgtggagaagcgacagacctatccgcgcggcgcaaccccaggccccggccagtcttccggggatcgcgcacgcgcaccggcgctctgctcgccaggcccagatgcggagcggagccgcctggacgcttgatctgcattacatacgccagtgcgtcaaccagctctttcggatcctgcgtgccgccccgaacccgcccggcagcgccaaccgcctgcgccacctggtgcgagactgctacctcatgggctactgccggacccgcctggggccgcgcacgtggggccgcctgctgcagatctcgggcggaacctgggacgtgcgcctgcgaaacgcaatccgggaggtcgaggcgcattttgaacccgccgccgagcccgtgtgcgagctgccctgtctgaacgccaggcgttacggccccgagtgtgatgttggcaatctcgagaccaacggcggctcgacgagcgatgatgagatatcggatgcgacggactcggacgataccctcgcgtcccattccgacacggagggggggccctccccggccggccgggagaacccggaatccgcgtccggcggggctatcgcggctcggctggagtgtgagtttgggacgtttgactggacgtccgaggagggctcccagccctggctgtccgcggtggtcgccgataccagctccgccgaacgctctggcctacccgccccgggcgcgtgtcgcgcaacggaagccccagaacgcgaggacgggtgccgaaaaatgcgcttccccgccgcctgcccctatccctgcggccacacatttctccggccatgagcgcgggacccccagcccggtgtgtttgccaaacgaaaaataaacgccctacaagaaagcttttgtgtctgagtgtctggtttttctgggggtggaggaaggaacgacaaaaaaaagaaacaaacgcgacaccgctcgtacgtgtaatggggccgcagtgttttttattagcatcggggggggttagaggttggtgattggatagcaaacgtgggatgacggaggccactcgtcgccaacggccagcgggggcccggggttctgggggtcatcgtcccccgtctgccaggagggctcatcgggaatctcgggtcgccccatgcacgtaaaacacgggcgctgcgtggggtgggtcgccggatgcgggcgggatgatgcggggcggggtttgttgtgaggagccacgagggaccgtagccagcgaagacagctgcgttcccggtcgccgggcaccaccacgccgtattggtattcgtatcggctaaggagattttccagggggtgattaggcgctgcggggaacggggtccacgacacggtccgctcgggcaaaaaccgatcgggcaggggccacggttcccccacccacgcgtcgttggtcttcgtggcgatgaagcgaaaccccagccgggttttttgtgcgtactcgaaaaacggcacacacaggtccgccgccccgaccacccacaggtggtatagccggtgggggccggggcgctcttgatgcaggagccgaaaacacgcaggggcatccagaatctcgatgctttccagggggtcgtcctccgcaaacaggcccgtcgtggtgtttgggggacagcgacaggagcgggttcgcacgatcggtcgggtgaatttgggcaagtccatcagaggctcggccagcctgcgaaggttcgccgggcgaaccaccaccggggttcccagaggctcggaggccaggatccggcattgccgaagcagaaaactccacagagccgggcttgcgtcagcggaagtccgcggcagggcgtttcgttggtctaggagggtaaccacacttacaacaacaacgcccatgtcggtatattaggcccgtggtccgatcttcactcactcgcctgtctgcggacctatgcacggcgggacggcgcgcggacccgggggggctgcttgctatcacacggcccgttcgcacgttcgattttttcagccttgtttggttggctaggtatcccggataatctgacgttccggatatagggggcggggggtagtggggggtgtgtcgacaaactgccgcttcttaaaacaccggggcccgtcgctcggggtgctcgttggttggcacgcgcgacgcggcaaatggcctgtcgtaagttctgtggggtctaccgtagacccgacaagagacaggaggcgtccgtcccgccggagacaaacacggccccggccttcccggcgagcaccttttatacccccgcggaggatgcgtacctggcccccgggcccccggaaaccatccacccttcccgcccaccgtcccccggcgaggctgcgcgcctgtgtcagctgcaggagatcttggcccagatgcacagcgacgaggactaccccatcgtggacgccgcgggtgcggaggaggaagacgaggccgacgatgacgccccggatgacgtggcctacccggaggactacgcggaggggcgttttctgtccatggtttcggccgcccccctgcccggagccagcggccatcctcctgttccgggccgcgcagccccccccgacgtccggacctgcgacacgggtaaggtgggggccacggggttcaccccggaagagctcgacaccatggaccgggaggcacttcgggccatcagccgcgggtgcaagcccccttcgaccctggcaaaactggtgaccgggctgggattcgcgatccacggagcgctcatcccggggtcggaggggtgtgtctttgatagcagccacccgaactaccctcatcgggtaatcgtcaaggcggggtggtacgccagcacgagccacgaggcgcggctgctgagacgcctgaaccaccccgcgatcctacccctcctggacctgcacgtcgtttctggggtcacgtgtctggtcctccccaagtatcactgcgacctgtatacctatctgagcaagcgcccgtctccgttgggccacctacagataaccgcggtctcccggcagctcttgagcgccatcgactacgtccactgcaaaggcatcatccaccgcgatattaagaccgagaacatcttcatcaacacccccgagaacatctgtctgggggactttggggcggcgtgctttgtgcgcgggtgtcgatcgagccccttccattacgggatcgcaggcaccatcgatacaaacgcccccgaggtcctggccggggatccgtacacccaggtaatcgacatctggagcgccggcctggtgatctttgagaccgccgtccacaccgcgtccttgttctcggccccgcgcgaccccgaaaggcggccgtgcgacaaccagatcgcgcgcatcatccgacaggcccaggtacacgtcgacgagtttccgacgcacgcggaatcgcgcctcaccgcgcactaccgctcgcgggcggccgggaacaatcgtccggcgtggacccgaccggcgtggacccgctactacaagatccacacagacgtcgaatatctcatatgcaaagcccttacctttgacgcggcgctccgcccaagcgccgcggagttgctgcgcctgccgctatttcaccctaagtgaccccgctccccccggggggcgtggaggggggggctggttggatgtttttgcacaaaaagacgcggccctcgggctttggtgtttttggcaccttgccgcccggcgtcatgcacgccatcgctcccaggttgcttcttctttttgttctttctggtcttccggggacacgcggcgggtcgggtgtccccggaccaattaatccccccaacagcgatgttgttttcccgggaggttcccccgtggctcaatattgttatgcctatccccggttggacgatcccgggcccttgggttccgcggacgccgggcggcaagacctgccccggcgcgtcgtccgtcacgagcccctgggccgctcgttcctcacgggggggctggttttgctggcgccgccggtacgcggatttggcgcacccaacgcaacgtatgcggcccgtgtgacgtactaccggctcacccgcgcctgccgtcagcccatcctccttcggcagtatggagggtgtcgcggcggcgagccgccgtccccaaagacgtgcgggtcgtacacgtacacgtaccagggcggcgggcctccgacccggtacgctctcgtaaatgcttccctgctggtgccgatctgggaccgcgccgcggagacattcgagtaccagatcgaactcggcggcgagctgcacgtgggtctgttgtgggtagaggtgggcggggagggccccggccccaccgcccccccacaggcggcgcgtgcggagggcggcccgtgcgtccccccggtccccgcgggccgcccgtggcgctcggtgcccccggtatggtattccgcccccaaccccgggtttcgtggcctgcgtttccgggagcgctgtctgcccccacagacgcccgccgcccccagcgacctaccacgcgtcgcttttgctccccagagcctgctggtggggattacgggccgcacgtttattcggatggcacgacccacggaagacgtcggggtcctgccgccccattgggcccccggggccctagatgacggtccgtacgcccccttcccaccccgcccgcggtttcgacgcgccctgcggacagaccccgagggggtcgaccccgacgttcgggccccccgaaccgggcggcgcctcatggccttgaccgaggacacgtcctccgattcgcctacgtccgctccggagaagacgcccctccctgtgtcggccaccgccatggcaccctcagtcgacccaagcgcggaaccgaccgcccccgcaaccactactccccccgacgagatggccacacaagccgcaacggtcgccgttacgccggaggaaacggcagtcgcctccccgcccgcgactgcatccgtggagtcgtcgccactccccgccgcggcggcggcaacgcccggggccgggcacacgaacaccagcagcgcctccgcagcgaaaacgccccccaccacaccagcccccacgacccccccgcccacgtctacccacgcgaccccccgccccacgactccggggccccaaacaacccctcccggacccgcaaccccgggtccggtgggcgcctccgccgcgcccacggccgattcccccctcaccgcctcgccccccgctaccgcgccggggccctcggccgccaacgtttcggtcgccgcgaccaccgccacgcccggaacccggggcaccgcccgtacccccccaacggacccaaagacgcacccacacggacccgcggacgctccccccggctcgccagcccccccaccccccgaacatcgcggcggacccgaggagtttgagggcgccggggacggcgaaccccccgaggacgacgacagcgccaccggcctcgccttccgaactccgaaccccaacaaaccaccccccgcgcgccccgggcccatccgccccacgctcccgccaggaattcttgggccgctcgcccccaacacgcctcgcccccccgcccaagctcccgctaaggacatgccctcgggccccacaccccaacacatccccctgttctggttcctaacggcctcccctgctctagatatcctctttatcatcagcaccaccatccacacggcggcgttcgtttgtctggtcgccttggcagcacaactttggcgcggccgggcggggcgcaggcgatacgcgcacccgagcgtgcgttacgtatgtctgccacccgagcgggattagggggtgggggtggggggcgagaaacgatgaaggacgggaaagggaacagcgaccaaatgtcacgataagaacaataaacctgtgacgtcaatcagatatgtgagtttggttgtgttttgtgggactgggggcggggggtgggaggtatcagtgggtgacagagtcttttaaaagacgtgtcccggggccctcgagatgcgcaacttttggccacacagagaaaggcccccagacgaagtcacccgggtccccgaacaaaaacaaaaaccttgaccgccgccggggggcgtgcctgttgttttggtctcaatggatcggtatgccgttcggacctgggggattgtgggaatcctcgggtgtgctgctgttggggccgcacccaccggccccgcgtccgatacaacaaacgcgaccgcacgcctccccacgcaccccccactcatccgttccgggggctttgccgtccccctcatcgtgggggggctgtgtctcatgattctggggatggcgtgtctactcgaggtcctgcgtcgcctgggtcgcgagttggcgaggtgctgcccccacgcgggccaatttgccccatgatttttcgcctttctggccttgcccccaccccatcgccccgattgtgtgtcgggtgcccggggtacagcagctatggagcggtcggtaatataactttggttgtcgccacacgccccgtgccgggcatgggttgtgcgggaaggacgaaataatccggcgatccccaagcgtaccaactgggggggggggggggggggaaaagaaactaaaaacacatcaagcccacaacccatcccacaatgggggttatggcggacccaccgcaccaccatactccgattcgaccacatatgcaaccaaatcacccccagaggggaggttccatttttacgaggaggaggagtataatagagtctttgtgtttaaaacccggggtcggtgtggtgttcggtcataagctgcattgcgaacgactagtcgccgtttttcgtgtgcatcgcgtatcacggcatggggcgtttgacctccggcgtcgggacggcggccctgctagttgtcgcggtgggactccgcgtcgtctgcgccaaatacgccttagcagacccctcgcttaagatggccgatcccaatcgatttcgcgggaagaaccttccggttttggaccagctgaccgacccccccggggtgaagcgtgtttaccacattcagccgagcctggaggacccgttccagccccccagcatcccgatcactgtgtactacgcagtgctggaacgtgcctgccgcagcgtgctcctacatgccccatcggaggccccccagatcgtgcgcggggcttcggacgaggcccgaaagcacacgtacaacctgaccatcgcctggtatcgcatgggagacaattgcgctatccccatcacggttatggaatacaccgagtgcccctacaacaagtcgttgggggtctgccccatccgaacgcagccccgctggagctactatgacagctttagcgccgtcagcgaggataacctgggattcctgatgcacgcccccgccttcgagaccgcgggtacgtacctgcggctagtgaagataaacgactggacggagatcacacaatttatcctggagcaccgggcccgcgcctcctgcaagtacgctctccccctgcgcatccccccggcagcgtgcctcacctcgaaggcctaccaacagggcgtgacggtcgacagcatcgggatgctaccccgctttatccccgaaaaccagcgcaccgtcgccctatacagcttaaaaatcgccgggtggcacggccccaagcccccgtacaccagcaccctgctgccgccggagctgtccgacaccaccaacgccacgcaacccgaactcgttccggaagaccccgaggactcggccctcttagaggatcccgccgggacggtgtcttcgcagatccccccaaactggcacatcccgtcgatccaggacgtcgcgccgcaccacgcccccgccgcccccagcaacccgggcctgatcatcggcgcgctggccggcagtaccctggcggtgctggtcatcggcggtattgcgttttgggtacgccgccgcgctcagatggcccccaagcgcctacgtctcccccacatccgggatgacgacgcgcccccctcgcaccagccattgttttactagaggagtttccccgctcccgtgtacctctgggcccgtgtgggagggtggctggggtatttgggtgggacttggactccgcataaagggagtctcgaaggagggaaactaggacagttcataggccgggagcgtggggcgcgcaccgctgtcccgacgattagccaccgcgcccacagccacctcgacccgtccgatcccggtatgcccggccgctcgctgcagggcctggcgatcctgggcctgtgggtctgcgccaccggcctggtcgtccgcggccccacggtcagtctggtctcagactcactcgtggatgccggggccgtggggccccagggcttcgtggaagaggacctgcgtgttttcggggagcttcattttgtgggggcccaggtcccccatacaaactactacgacggcatcatcgagctgtttcactaccccctggggaaccactgcccccgcgttgtacacgtggtcacactgaccgcatgcccccgccgccccgccgtggcgttcaccttgtgtcgctcgacgcaccacgcccacagccccgcctatccgaccctggagctgggtctggcgcggcagccgcttctgcgggttcgaacggcaacgcgcgactatgccggtctgtatgtcctgcgcgtatgggtcggcagcgcgacgaacgccagccggtttgttttgggggtggcgctctctgccaacgggacgtttgtgtataacggctcggactacggctcctgcgatccggcgcagcttcccttttcggccccgcgcctgggaccctcgagcgtatacacccccggagcctcccgacccacccctccacggacaacgacacccccgtcctccccccgagacccgacccccgcccccggggacacagggacgcccgcgcccgcgagcggcgagatagccccgcccaattccacgcgatcggccagcgaatcgagacacaggctaaccgtagcccaggtaatccagatcgccataccggcgtccatcatcgcctttgtgtttctgggcagctgtatctgcttcatccatagatgccagcgccgatacaggcgcccccgcggccagatttacaaccccgggggcgtttcctgcgcggtcaacgaggcggccatggcccgcctcggagccgagctgcgatcccacccaaacaccccccccaaaccccgacgccgttcgtcgtcgtccacgaccatgccttccctaacgtcgatagctgaggaatcggagccaggtccagtcgtgctgctgtccgtcagtcctcggccccgcagtggcccgacggccccccaagaggtctaggtccaagcgggccgttcggcaggcccgccccaccgcccccatcgtggttatttccccccccccccccccaataaaccgatgttatttgcctatatgcgtgtgttggatccctttgtgatcgttcgtcattccccggatggcatgggaggcgggtaatggatgggcggggcccggggggaggaaaaagaataaagggggtagtgtcggagaggcccgccgcgcatttaaggagtcgccgccccgactctgtgtcttcgggtgacttggtgcgccgccgtcagctagtctccgatctgccccgaccgacggctcctgccacccgaacatggctcgcggggccgggttggtgttttttgttggagtttgggtcgtatcgtgcctggcggcagcacccagaacgtcctggaaacgggtaacctcgggcgaggacgtggtgttgcttccggcgcccgcggaacgcacccgggcccacaaactactgtgggccgcggaacccctggatgcctgcggtcccctgcgcccgtcgtgggtggcgctgtggcccccccgacgggtgctcgagacggtcgtggatgcggcgtgcatgcgcgccccggaaccgctcgccatagcatacagtcccccgttccccgcgggcgacgagggactgtattcggagttggcgtggcgcgatcgcgtagccgtggtcaacgagagtctggtcatctacggggccctggagacggacagcggtctgtacaccctgtccgtggtcggcctaagcgacgaggcgcgccaagtggcgtcggtggttctggtcgtggagcccgcccctgtgccgaccccgacccccgacgactacgacgaagaagacgacgcgggcgtgacgaacgcacgccggtcagcgttccccccccaaccccccccccgtcgtccccccgtcgcccccccgacgcaccctcgtgttatccccgaggtgtcccacgtgcgcggggtaacggtccatatggagaccctggaggccattctgtttgcccccggggagacgtttgggacgaacgtctccatccacgccattgcccacgacgacggtccgtacgccatggacgtcgtctggatgcggtttgacgtgccgtcctcgtgcgccgatatgcggatctacgaagcttgtctgtatcacccgcagcttccagagtgtctatctccggccgacgcgccgtgcgccgtaagttcctgggcgtaccgcctggcggtccgcagctacgccggctgttccaggactacgcccccgccgcgatgttttgccgaggctcgcatggaaccggtcccggggttggcgtggctggcctccaccgtcaatctggaattccagcacgcctccccccagcacgccggcctctacctgtgcgtggtgtacgtggacgatcatatccacgcctggggccacatgaccatcagcaccgcggcgcagtaccggaacgcggtggtggaacagcacctcccccagcgccagcccgagcccgtcgagcccacccgcccgcacgtgagagccccccatcccgcgccctccgcgcgcggcccgctgcgcctcggggcggtgctgggggcggccctgttgctggccgccctcgggctgtccgcgtgggcgtgcatgacctgctggcgcaggcgctcctggcgggcggttaaaagccgggcctcggcgacgggccccacttacattcgcgtggcggacagcgagctgtacgcggactggagttcggacagcgagggggagcgcgacgggtccctgtggcaggaccctccggagagacccgactctccctccacaaatggatccggctttgagatcttatcaccaacggctccgtctgtatacccccatagcgaggggcgtaaatctcgccgcccgctcaccacctttggttcgggaagcccgggccgtcgtcactcccaggcctcctatccgtccgtcctctggtaaggcgtcttccgacgacgcggacgtcggcgatgaactgattgccatcgcggacgcacgcggggacccgccagagaccctgccccccggcgcgggcggcgccgcgcccgcgtgccgcagaccacctcgcggcggctcccccgcggcctttcccgtggccctccacgccgtggacgccccctcccaattcgtcacctggctcgccgtgcgctggctgcggggggcggtgggtctcggggccgtcctgtgcgggattgcgttttacgtgacgtcaatcgcccgaggcgcataaaggtccggcggccaccccgccgcagctcataaaaatcgtgagtcacggcaaccccaccttcgcctccgccctccgccagcgcccttccgcgtccgcgatgacctcccggcccgccgaccaagactcggtgcgttccagcgcgtcggtgccgctttaccccgcggcctcgcccgtcccggcagaagcctactactcggaaagcgaagacgaggccgccaacgacttcctcgtgcgcatgggccgccagcagtcggtcctaaggcgccgacggcggcgcacgcggtgcgtcgggctggttatcgcctgtctcgtcgtggccctcctatctggagggttcggggcacttttggtgtggctgctccgctaaatgacgcctcgatgtatggcgccttcttcgcccccacccctcgccgcgacccacgtccgtatgttaattgcaataaagtggttgattgtcattacggtctactaggttgtcttttttttttgggggggggggaggaaatgcagaaaagggtaagaaattctcggaatttcacccccgggggggggcaagtgcagtaacccagttcctcagtgtttgggaaatctattgaactctcccggctcctccgtgttagggaagtctcttggggaaatctattgacctctcgcccccccccccccaggaggggggcagtgcagtaccccagttcctccgtgctggggaaatctctctgccgggtacgggctccagacgaaggacccatacatttccccatccgcaccccacatctggcgttctagagtcacgacgcatttgcccccgtccccgcagcaacacacaaagcgatttcaattttcacgattttattattaattacaccaaccaccctgtccccgggacgtggtcaggaccgggggtccgcacccaaacgcacgaaacaaatgctggcagtgtgccgaatataaccccgcgtaggaacacgtcgacgcgtgcgccaaacagcaccagaaggcgcatgccatcagcaggtcgtgcatatggcgatgtgtttggacgcagggcgcagccgcggcgataaaattcatggcggccgtccgccagggccacagcggcgaggactccctgttggcccgaagccattgggtatgaaccagctgcgcctcctgtccgaccctggctcccgccagcgggggcggtgggtcgtgggtgttgagagcacacaggcgggacacctcgatcaccgtccgaaaaaaggcccggtggtccgcgggcagcatctgcaggtgcgccagggcctgggcgttgagagggtacaactcggagccgggggactccgggggccggtccgcgcggtgccgcgagttggcacgctttggggcccgggtgtcggacgcgggcgcgttatggatcccgacgcggggcagaacgtacgtgcgttggcgcggcgatgaggggtccgggctgccgaggggggcgtaggggaccgggctaggcaagcccgcgggttgcgcggggttcccgtgggggtctaggctccctgggcacccgtgggggtcgtgggggtcgcgggtccctgggtatgcgcgggaccctgggttctctgggagatcgtggaactcgcggttccctgggctctcggggaacccggggctccctggggacacgtggtgccctgggaattcttgatggtcggacggcttcagatggcttcgggatcgagagggccgcacagactcgtagtagacccgaatctccacgtttccccgccgccggatcatggtcgccgccccggtgcgggggcccgtcggtcggaagcgagtgcccttcaagcgtgtccgctcctctgggctgcatgccgtcggatggggtgccttttaaggaaaggtctcggctgcccgccccaaccggggtttgggggtgggccggggaaaccccggatgccatgggggggtcacaccctaagcgccggcgcgctggttgggtgggggtagaggggagtccccggtcgacgagatcgtatcaaggggccagcacgcgatcctgccgctcgttcgatctagcacacccacgggtctgctgtgtgggatttcgactcgcgggatccgatcgcacgtccggaggacacagcagcgggagctccgggtcggtcaccgcagttctggccgcctctcggtcctcccgttcccttttatggatctccgcgcagacatcgccatacgtccggtgtgtgcaccgcgaagaatccagaaacatgtccgtcgttttcagggcccaagacatggtgtcccgtccacgaaggcggcgcccggcctgcgagaaagcgcggatgttgggatcggggccccgtccccccggcccgtccccccgtccccccggcccgtccccccgtccccccggcccgtccccccgtccccccggcccgtccccccgtccccccggcccgtccccccgtccccccgtccccccgtccccccgtccccccgtccccccgtccccccgtccccccgtccccccgtccccccgtccccccgtccccccgtccccccgtccccccgtccccccgtccccccgtccccccggccccccggccccccggccccccggccccccggcccgtccccccggcccgtccccccggcccgtccccccggcccgtccccccggcccgtccccccggcccgtccccccggcccgtccccccggcccgtccccccggcccgtccccccgtcccccgcccgtccccccggccggccccccgggtcaccgtacctgcgataaggctgcagtgggtggatgggtcctcgcggtacgtacagggtgggggggggggggggggagggaaaggcagaacgaaaaggaaccgatgcgcccgcgtctctgtatccgatccgatccgggtgcgtcggtgccccgctcgccgccggcgtctctgtctcgctgtggcccccttcgcgatgccgccgctgccgtcccggtctccgccgcgcagccggtgtgcccctggtgcggcggcgaccgggacgccggccctttatgtgcgcgaggaacggcccgccccccgtccgggcccgcctcggggcggagcccgcgggatgacgcgggccccgggcagggcgccagtgctcgcactttgccctaataatatatatactattaggacgaagtgcgaacgcttcgcgttctcacttcttttaccctgcggccccgccccctttggggcggagcgcgggatgacgcgggccccgggcagggcgccagcgctcgcactttgccctaataatatatatactattaggacgaagtgcgaacgcttcgcgttctcacttcttttaccctgcggccccgccccctttggggcggagccgcccgcggaccaacggggcgacctcgccggcccctttggggccggcgggggccaacgggagcgcggggccggcatctcattaccacgaacccggaagggcaggggagcgagcccgcccgcgacgagggtctcattagcatcgcgggcggaagcggaagccgcccgcgccgggcgctaatgagatgccgcgcgggcggagcggcggcggcgcgaccaacgggccgccgccacggacgcggacgcgcgggcgtcggggcggggccgcgcataatgcggttccacctgggggcggaaccccggcgagccggggcgcggcggcgtcgatcgctcctcctccgcgtcctcctcctttccccccgccccgcgcgccccgaggactatatcagccaggcgacggggcgatcgtccacacggagcgcggctaccgacgcggccgccaggatctacccgatcggcgcggagaggcgaaaagacacaggcacacgcacgcaccgcacgggggggagagagactgccaaccacccccccccactgccgcccctgaagaagaagaagaagaccccccccccgcacaccccggtcggaggcgatgtcggcggagcagcggaagaagaagaagacgacgacgacgacgcagggccgcggggccgaggtcgcgatggcggacgaggacgggggacgtctccgggccgcggcggagacgaccggcggccccggatctccggatccagccgacggaccgccgcccaccccgaacccggaccgtcgccccgccgcgcggcccgggttcgggtggcacggtgggccggaggagaacgaagacgaggccgacgacgccgccgccgatgccgatgccgacgaggcggccccggcgtccggggaggccgtcgacgagcctgccgcggacggcgtcgtctcgccgcggcagctggccctgctggcctcgatggtggacgaggccgttcgcacgatcccgtcgccccccccggagcgcgacggcgcgcaagaagaagcggcccgctcgccttctccgccgcggaccccctccatgcgcgccgattatggcgaggagaacgacgacgacgacgacgacgacgatgacgacgaccgcgacgcgggccgctgggtccgcggaccggagacgacgtccgcggtccgcggggcgtacccggaccccatggccagcctgtcgccgcgacccccggcgccccgccgacaccaccaccaccaccaccaccgccgccggcgcgccccccgccggcgctcggccgcctctgactcatcaaaatccggatcctcgtcgtcggcgtcctccgcctcctcctccgcctcctcctcctcgtctgcatccgcctcctcgtctgacgacgacgacgacgacgacgccgcccgcgcccccgccagcgccgcagaccacgccgcgggcgggaccctcggcgcggacgacgaggaggcgggggtgcccgcgagggccccgggggcggcgccccggccgagcccgcccagggccgagcccgccccggcccggacccccgcggcgaccgcgggccgcctggagcgccgccgggcccgcgcggcggtggccggccgcgacgccacgggccgcttcacggccgggcggccccggcgggtcgagctggacgccgacgcggcctccggcgccttctacgcgcgctaccgcgacgggtacgtcagcggggagccgtggcccggggccggccccccgcccccggggcgcgtgctgtacggcgggctgggcgacagccgccccggcctctggggggcgcccgaggcggaggaggcgcgggcccggttcgaggcctcgggcgccccggcgcccgtgtgggcgcccgagctgggcgacgcggcgcagcagtacgccctgatcacgcggctgctgtacacgccggacgcggaggcgatggggtggctccagaacccgcgcgtggcgcccggggacgtggcgctggaccaggcctgcttccggatctcgggcgcggcgcgcaacagcagctccttcatctccggcagcgtggcgcgggccgtgccccacctggggtacgccatggcggcgggccgcttcggctggggcctggcgcacgtggcggccgccgtggccatgagccgccgctacgaccgcgcgcagaagggcttcctgctgaccagcctgcgccgcgcctacgcgcccctgctggcgcgcgagaacgcggcgctgaccggggcgcgaacccccgacgacggcggcgacgccaaccgccacgacggcgacgacgcccgcgggaagcccgccgccgccgccgccccgttgccgtcggcggcggcgtcgccggccgacgagcgcgcggtgcccgccggctacggcgccgcgggggtgctcgccgccctggggcgcctgagcgccgcgcccgcctccgcgccggccggggccgacgacgacgacgacgacgacggcgccggcggtggtggcggcggccggcgcgcggaggcgggccgcgtggccgtggagtgcctggccgcctgccgcgggatcctggaggcgctggcggagggcttcgacggcgacctggcggccgtgccggggctggccggagcccggcccgccgcgcccccgcgcccggggcccgcgggcgcggccgccccgccgcacgccgacgcgccccgcctgcgcgcctggctgcgcgagctgcggttcgtgcgcgacgcgctggtgctgatgcgcctgcgcggggacctgcgcgtggccggcggcagcgaggccgccgtggccgccgtgcgcgccgtgagcctggtcgccggggccctgggcccggcgctgccgcggagcccgcgcctgctgagctccgccgccgccgccgccgcggacctgctcttccagaaccagagcctgcgccccctgctggccgacaccgtcgccgcggccgactcgctcgccgcgcccgcctccgcgccgcgggaggcgcgcaagcgcaagagccccgccccggccagggcgccgccgggcggcgccccgcgccccccgaagaagagccgcgcggacgccccccgccccgcggccgcccctcccgcgggggccgcgccccccgccccgccgacgccgccgccgcggccgccgcgccccgcggcgctgacccgccggcccgccgagggccccgacccgcagggcggctggcgccgccagccgccggggcccagccacacgccggcgccctcggccgccgccctggaggcctactgcgccccgcgggccgtggccgagctcacggaccacccgctcttccccgcgccgtggcgcccggccctcatgttcgacccgcgcgcgctggcctcgctggccgcgcgctgcgccgccccgccccccggcggcgcgcccgccgccttcggcccgctgcgcgcctcgggcccgctgcgccgcgcggcggcctggatgcgccaggtgcccgacccggaggacgtgcgcgtggtgatcctctactcgccgctgccgggcgaggacctggccgcgggccgcgccgggggcgggccccccccggagtggtccgccgagcgcggcgggctgtcctgcctgctggcggccctgggcaaccggctctgcgggcccgccacggccgcctgggcgggcaactggaccggcgcccccgacgtctcggcgctgggcgcgcagggcgtgctgctgctgtccacgcgggacctggccttcgccggcgccgtggagttcctggggctgctggccggcgcctgcgaccgccgcctcatcgtcgtcaacgccgtgcgcgccgcggactggcccgccgacgggcccgtggtctcgcggcagcacgcctacctggcctgcgaggtgctgcccgccgtgcagtgcgccgtgcgctggccggcggcgcgggacctgcgccgcaccgtgctggcctccggccgcgtgttcgggccgggggtcttcgcgcgcgtggaggccgcgcacgcgcgcctgtaccccgacgcgccgccgctgcgcctctgccgcggggccaacgtgcggtaccgcgtgcgcacgcgcttcggccccgacacgctggtgcccatgtccccgcgcgagtaccgccgcgccgtgctcccggcgctggacggccgggccgccgcctcgggcgcgggcgacgccatggcgcccggcgcgccggacttctgcgaggacgaggcgcactcgcaccgcgcctgcgcgcgctggggcctgggcgcgccgctgcggcccgtctacgtggcgctggggcgcgacgccgtgcgcggcggcccggcggagctgcgcgggccgcggcgggagttctgcgcgcgggcgctgctcgagcccgacggcgacgcgcccccgctggtgctgcgcgacgacgcggacgcgggcccgcccccgcagatacgctgggcgtcggccgcgggccgcgcggggacggtgctggccgcggcgggcggcggcgtggaggtggtggggaccgccgcggggctggccacgccgccgaggcgcgagcccgtggacatggacgcggagctggaggacgacgacgacggactgtttggggagtgacggggggggaaacttccgggagcgggggaggggggagatggggagagggggaaggaatcgggcgtctgtgcgcctttaagacagacgcggcgatggccgcgcgcgtgtgtgagaaataaagaacgagacagacgaaaacgtaccgccttgtgtggtttattcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcgggggtcgggcgggcggcacgtctcccgcgcccgcggggggtctggggctctgacctgagtgcaggttacgaaggtcaggtggcccgagcccccccgcaggagcgggagggaaggcacggggcgcgggagggaggggctgctgcgagctcggggccgcgggcgcggggggaggggcgggggaagcccccggggcggggcgcgggggaggcggccgcgggggaggcggccgcgggaccgcagccccgtggcgcgcgggggggaggggctgccgcgagctcggcgggatggaggggagggagggggtggcggggaaccgtgtgcgggcgggcgggtgcttggtgcaactgtctggtctgcgagggcgagcggtggtgcgactggcgtcttcgggggggcggggagcttgggagtgtgtggtggtctgcggcacagcctgctagtccccgtcctgccgcgcgggggcgggcgcgggaaaaaagccgcgcgggggcgcccgcgggaaggcagccccgcggcgcgcggggggaggggcggcgcccgcgggggagcggccggctccgggggagggacggggaagggggcgcgcggggctgccctgccgcccgcccgccgccgccgcccgccttcgcgcccccccccaaaaaacaccccccccgggggttgactccccgggggaaaagaggcggggcggg

SEQ ID NO: 17 - DNA, 9181 bp, Human Immunodeficiency Virus HIV1 (NC_001802.1)
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccggccataaggcaagagttttggctgaagcaatgagccaagtaacaaattcagctaccataatgatgcagagaggcaattttaggaaccaaagaaagattgttaagtgtttcaattgtggcaaagaagggcacacagccagaaattgcagggcccctaggaaaaagggctgttggaaatgtggaaaggaaggacaccaaatgaaagattgtactgagagacaggctaattttttagggaagatctggccttcctacaagggaaggccagggaattttcttcagagcagaccagagccaacagccccaccagaagagagcttcaggtctggggtagagacaacaactccccctcagaagcaggagccgatagacaaggaactgtatcctttaacttccctcaggtcactctttggcaacgacccctcgtcacaataaagataggggggcaactaaaggaagctctattagatacaggagcagatgatacagtattagaagaaatgagtttgccaggaagatggaaaccaaaaatgatagggggaattggaggttttatcaaagtaagacagtatgatcagatactcatagaaatctgtggacataaagctataggtacagtattagtaggacctacacctgtcaacataattggaagaaatctgttgactcagattggttgcactttaaattttcccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtactatttttagatggaatagataaggcccaagatgaacatgagaaatatcacagtaattggagagcaatggctagtgattttaacctgccacctgtagtagcaaaagaaatagtagccagctgtgataaatgtcagctaaaaggagaagccatgcatggacaagtagactgtagtccaggaatatggcaactagattgtacacatttagaaggaaaagttatcctggtagcagttcatgtagccagtggatatatagaagcagaagttattccagcagaaacagggcaggaaacagcatattttcttttaaaattagcaggaagatggccagtaaaaacaatacatactgacaatggcagcaatttcaccggtgctacggttagggccgcctgttggtgggcgggaatcaagcaggaatttggaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccatatgtatgtttcagggaaagctaggggatggttttatagacatcactatgaaagccctcatccaagaataagttcagaagtacacatcccactaggggatgctagattggtaataacaacatattggggtctgcatacaggagaaagagactggcatttgggtcagggagtctccatagaatggaggaaaaagagatatagcacacaagtagaccctgaactagcagaccaactaattcatctgtattactttgactgtttttcagactctgctataagaaaggccttattaggacacatagttagccctaggtgtgaatatcaagcaggacataacaaggtaggatctctacaatacttggcactagcagcattaataacaccaaaaaagataaagccacctttgcctagtgttacgaaactgacagaggatagatggaacaagccccagaagaccaagggccacagagggagccacacaatgaatggacactagagcttttagaggagcttaagaatgaagctgttagacattttcctaggatttggctccatggcttagggcaacatatctatgaaacttatggggatacttgggcaggagtggaagccataataagaattctgcaacaactgctgtttatccattttcagaattgggtgtcgacatagcagaataggcgttactcgacagaggagagcaagaaatggagccagtagatcctagactagagccctggaagcatccaggaagtcagcctaaaactgcttgtaccaattgctattgtaaaaagtgttgctttcattgccaagtttgtttcataacaaaagccttaggcatctcctatggcaggaagaagcggagacagcgacgaagagctcatcagaacagtcagactcatcaagcttctctatcaaagcagtaagtagtacatgtaatgcaacctataccaatagtagcaatagtagcattagtagtagcaataataatagcaatagttgtgtggtccatagtaatcatagaatataggaaaatattaagacaaagaaaaatagacaggttaattgatagactaatagaaagagcagaagacagtggcaatgagagtgaaggagaaatatcagcacttgtggagatgggggtggagatggggcaccatgctccttgggatgttgatgatctgtagtgctacagaaaaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacagtattggagtcaggaactaaagaatagtgctgttagcttgctcaatgccacagccatagcagtagctgaggggacagatagggttatagaagtagtacaaggagcttgtagagctattcgccacatacctagaagaataagacagggcttggaaaggattttgctataagatgggtggcaagtggtcaaaaagtagtgtgattggatggcctactgtaagggaaagaatgagacgagctgagccagcagcagatagggtgggagcagcatctcgagacctggaaaaacatggagcaatcacaagtagcaatacagcagctaccaatgctgcttgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttc

SEQ ID NO: 18 - DNA, 10359 bp, Human Immunodeficiency Virus HIV2 (NC_001722.1)
tgcaagggatgttttacagtaggaggagacatagaatcctagacatatacctagaaaaagaggaagggataataccagattggcagaattatactcatgggccaggagtaaggtacccaatgtacttcgggtggctgtggaagctagtatcagtagaactctcacaagaggcagaggaagatgaggccaactgcttagtacacccagcacaaacaagcagacatgatgatgagcatggggagacattagtgtggcagtttgactccatgctggcctataactacaaggccttcactctgtacccagaagagtttgggcacaagtcaggattgccagagaaagaatggaaggcaaaactgaaagcaagagggataccatatagtgaataacaggaacaaccatacttggtcaaggcaggaagtagctactaagaaacagctgaggctgcagggactttccagaaggggctgtaaccaagggagggacatgggaggagctggtggggaacgccctcatacttactgtataaatgtacccgcttcttgcattgtattcagtcgctctgcggagaggctggcagatcgagccctgagaggttctctccagcactagcaggtagagcctgggtgttccctgctggactctcaccagtacttggccggtactgggcagacggctccacgcttgcttgcttaaagacctcttcaataaagctgccagttagaagcaagttaagtgtgtgttcccatctctcctagtcgccgcctggtcattcggtgttcatctgagtaacaagaccctggtctgttaggacccttctcgctttgggaatccaaggcaggaaaatccctagcaggttggcgcccgaacagggacttgaagaggactgagaagccctggaactcggctgagtgaaggcagtaagggcggcaggaacaaaccacgacggagtgctcctagaaaggcgcgggccgaggtaccaaaggcggcgtgtggagcgggagtgaaagaggcctccgggtgaaggtaagtacctacaccaaaaactgtagccagaaaaggcttgttatcctacctttagacaggtagaagattgtgggagatgggcgcgagaaactccgtcttgagagggaaaaaagcagacgaattagaaaaagttaggttacggcccggcggaaagaaaaagtacaggttaaaacatattgtgtgggcagcgaatgaattggataaattcggattggcagagagcctgttggagtcaaaagaaggttgccaaaagattctcagagttttagatccattagtaccaacagggtcagaaaatttaaaaagcctttttaataccgtctgcgtcatttggtgcttgcacgcagaagagaaagtgaaagatactgaggaagcaaagaaactagcacagagacatctagtggcagaaactggaactgcagagaaaatgccaaatacaagtagaccaacagcaccacctagtgggaaaagaggaaactaccccgtgcaacaagcgggtggcaactatgtccatgtgccactgagcccccgaactctaaatgcatgggtaaaattagtggaggaaaagaagttcggggcagaagtagtgccaggatttcaggcactctcagaaggctgcacgccctatgatattaatcaaatgcttaattgtgtgggcgatcaccaagcagctatgcaaataatcagagagattattaatgaagaagcagcagactgggattcgcagcacccaataccaggccccttaccagcaggacagctcagagacccaagagggtctgacatagcaggaacaacaagcacagtagatgaacagatccagtggatgtataggccacaaaatcccgtaccggtagggaacatctacagaagatggatccaaatagggctgcaaaagtgtgtcagaaagtacaacccaactaacatcttagacataaaacagggaccaaaagaaccgttccaaagctatgtagacaggttctacaaaagcttgagggcagaacaaacagacccagcagtaaaaaattggatgacccaaacgctgctaatacagaatgccaacccagactgcaagttagtactaaaaggactggggatgaatcccaccctagaagagatgctaaccgcctgccagggggtaggcggaccaggccagaaagccaggctaatggctgaagccctaaaagaggctatgggaccaagccctatcccatttgcagcagcccaacaaagaaaggcaattaggtattggaactgtggaaaggagggacactcggcaagacagtgccgagcacctagaagacagggctgctggaagtgtggcaagccaggacacatcatggcaaactgcccggaaagacaggcaggttttttagggttgggcccacggggaaagaagcctcgcaacttccccgtgacccaagcccctcaggggctgataccaacagcacctccggcagatccagcagcggaactgttggagagatatatgcagcaagggagaaagcagagggagcagagggagagaccatacaaagaggtgacggaggacttgctgcacctcgagcagagagagacacctcacagagaggagacagaggacttgctgcacctcaattctctctttggaaaagaccagtagtcacagcgtacatcgaggatcagccggtagaagtcttactagacacaggggctgatgactcaatagtagcaggaatagaattaggggacaattacactccaaaaatagtagggggaatagggggatttataaacaccaaagaatacaaaaatgtagaaataaaagtactaaataaaagagtaagagccaccataatgacaggagataccccaatcaacatctttggcagaaatattctgacagccttaggcatgtcattaaatttaccagttgccaagatagagccaataaaagtaacattgaagccagggaaagatggaccaaggctgaaacaatggcccctaacaaaagagaaaatagaagcactaaaagagatctgtgaaaaaatggaaaaagagggccagctagaagaggcacctccaactaatccttataatacccccacatttgcaattaagaaaaaggacaagaacaaatggaggatgctgatagattttagagaactaaataaggtgactcaagatttcacagaaattcagctaggaattccacacccggcaggactagccaaaaagaaaaggatctctatattagatgtaggggatgcctatttttccataccactacatgaagattttaggcagtatactgcatttaccctaccagcagtaaacaatatggaaccaggaaaaagatatatatataaagtcttgccacaaggatggaagggatcaccagcaatttttcaatacacaatgaggcaagtcttagaacctttcagaaaagcaaacccagatgtcattctcatccagtacatggatgatatcttaatagctagtgacaggacaggtttagagcatgacaaagtggtcctgcagctaaaagaacttctaaatggcctagggttttctactccagatgagaagttccaaaaagaccctccatttcaatggatgggctgtgaactatggccaactaaatggaagctgcagaaactacaactgccccagaaagacatatggacagtcaatgacatccaaaagctagtgggagtcttaaattgggcggcacaaatctattcaggaataaaaaccaaacacttatgtagactaattagaggaaaaatgacactcacagaagaagtgcagtggacagaactagcagaagcagagctagaagaaaacaaaattatcttgagccaggaacaagaaggatattattaccaagaagaaaaagaattagaggcaacaatccaaaaaagccaaggacatcaatggacatacaaaatacaccaggaagagaaaatcctaaaagtaggaaagtatgcaaagataaaaaatacccataccaatggggtcagattactagcacaggtagttcagaaaataggaaaagaggcactagtcatttggggacggataccaaaatttcacctgccagtggagagagagacctgggagcagtggtgggataactactggcaagtgacatggatcccagagtgggactttgtatctaccccaccactggtcaggttaacatttaacctagtaggagatcctataccaggcgcagagaccttctacacagatggatcatgcaatagacagtcaaaagagggaaaagcaggatatgtaacagatagaggaaaagacaaagtaaaagtattagaacaaactaccaatcagcaggcagaattagaagtctttcggatggcactggcagactcaggcccaaaggttaatatcatagtagattcacagtatgtaatggggatagtagcaggccagccaacagagtcagaaaatagaatagtgaaccagatcatagaagaaatgataaagaaggaagcagtctatgttgcatgggtcccagcccataaaggcataggaggaaaccaggaagtagaccatttagtaagtcaaggcatcagacaagtattattcctggaaaagatagagcccgctcaagaggaacatgaaaaatatcatagcattataaaagaactaacccataaatttggaataccccttctagtagcaagacagatagtaaactcatgtgcccaatgccaacagaaaggagaagccatacatgggcaagtaaatgcagaaataggcgtttggcaaatggactacacacacttagaaggaaaaatcattatagtagcagtacatgttgcaagtggattcatagaagcagaagtcatcccacaggaatcaggaaggcagacagcactcttcctattaaaactggccagtaggtggccaataacgcacttgcacacagacaatggccccaacttcacttcacaggaagtgaagatggtggcatggtgggtaggtatagaacaatcctttggagtaccttacaacccacaaagccagggagtagtagaagcaatgaatcaccacctaaagaatcagataagtagaattagagaacaggcaaatacaatagaaacaatagtactgatggcagttcattgcatgaattttaaaagaaggggaggaataggggatatgaccccagcagaaagactaatcaacatgattaccacagaacaagaaatacaattcctccaaagaaaaaattcaaattttaaaaatttccaggtctattacagagaaggcagagatcagctgtggaaaggacctggtgaactactgtggaagggagaaggagcagtcatagtcaaggtagggacagacataaaagtagtaccaagaaggaaggccaagattatcagggactatggaggaagacaggaactggatagtagtccccacctggagggtgccagggaggatggagaaatggcatgcccttgtcaagtacctgaaatacagaacaaaagacctagaggaggtgcgctatgttccccaccacaaggtgggatgggcatggtggacttgcagcagggtaatattcccactacaaggaaaaagtcatctagaaatacaggcatattggaacctaacaccagaaaaaggatggctctcctctcatgcagtaagattaacctggtatacagaaaagttctggacagatgttaccccagactgtgcagacatcctaatacatagcacttatttctcttgctttacggcaggtgaagtaagaagagccatcagaggggaaaagttattgtcctgctgcaactatccccaagctcataaagcacaggtaccatcacttcaatacctagccctagtagtagtacaacaaaatgacagaccccagagaaagggtaccgccaggaaacagtggagaagagaccattggagaggccttcgagtggctagagaggaccatagaagccttaaacagggaggcagtgaaccatctgccccgagagctcattttccaggtgtggcaaaggtcctggagatattggcatgatgaacaagggatgtcagcaagctacacaaagtatagatatttgtgcctaatgcaaaaagctatatttacacatttcaagagagggtgcacttgctggggggaggacatgggccgggaaggattggaagaccaaggacctccccctcctccccctccaggtctagtctaatgactgaagcaccaacagagtttcccccagaagatgggaccccacggagggacttagggagtgactgggtaatagaaactctgagggaaataaaggaagaagccttaagacattttgatccccgcttgctaattgctcttggctactatatccataatagacatggagacacccttgaaggcgccagagagctcattaaaaccctacaacgagccctcttcgtgcacttcagagcgggatgtaaccgctcaagaattggctaaacaaggagaagaactccttgcccagctgcaccgacccctagagccatgcactaacaaatgctattgtaagcgatgcagtttccattgccagctgtgtttctcgaaaaaggggctcggaatatcatatgagcgaaagggcagacgaagaaggactccaaggaaaactaagactccttcgccttctgcaccagacaagtgagtatggagcctggtaggaatcagctgtttgttgtcattttactaacaagtgcttgcttagtatattgtagccagtatgtgactgttttctatggcatacccgcgtggaaaaatgcatctattcccttattttgtgcaactaaaaatagagacacttgggggaccatacagtgcttgccagacaatgatgattatcaggaaataattttaaatgtgacagaggcttttgatgcatggaataatacagtgacagaacaagcagtagaagatgtctggcatctatttgagacatcaataaaaccatgtgtcaagctaacacctctatgtgtggcaatgaattgtagcagggttcaagggaataccacgaccccgaatcccaggacctcgagttccacaacctcgagaccacccacatccgcagcctccataataaatgaaacttctaactgcatagaaaacaacacatgcgcaggattagggtatgaggagatgatgcaatgtgagttcaatatgaaggggttagaacaagataagaaaaggaggtataaggacacatggtatttagaagatgtggtttgtgacaacacaacagctggcacatgttacatgagacattgcaacacatcaatcatcaaagagtcatgtgataagcactattgggatgctatgaggtttagatactgtgcaccaccgggctttgccctattaagatgtaatgataccaactattcaggctttgaacctaagtgcactaaagtagtagctgcttcatgcacaaggatgatggaaacgcaaacttctacttggtttggctttaatggcactagagcagaaaatagaacatatatctattggcatggcagagataataggactatcattagcttaaacaagtattataatctcacaatgcgttgtaagagaccaggaaataagacagttttaccaataacacttatgtcaggattagtgtttcactctcagccaatcaacacaaggcctaggcaggcatggtgccggtttggaggcagatggagggaagccatgcaggaggtgaagcaaacccttgtacaacatcccagatacaaaggaatcaatgatacagggaaaattaactttacgaaaccgggagcaggctcagacccggaagtggcatttatgtggactaactgcagaggagaatttctctactgtaacatgacttggttcctcaattgggtagaagacaagaaccaaacacggcgcaactattgccatataaagcagataattaatacctggcataaagtagggaaaaatgtatatttgcctcctagggaaggggagttggcctgtgaatcaacagtaaccagcataattgctaacattgacatagataaaaatcggactcataccaacattacctttagtgcagaagtggcagaactgtaccgattagaactgggagactacaaattaatagaaataacaccaattggcttcgcacctacagatcagagaaggtactcctcaactccagtgaggaacaaaagaggtgtgttcgtgctagggttcttgggttttctcgcgacagcaggttctgcaatgggcgcgcggtccctgacgctgtcagcccagtcccggactttactggccgggatagtgcagcaacagcaacagctgttggacgtagtcaagagacaacaagaaatgttgcgactgaccgtctggggaacgaaaaacctccaggcaagagtcactgctatcgagaagtacctaaagcatcaggcacagctaaattcatggggatgtgcgtttagacaggtctgccacactactgtaccgtgggtaaatgactctttatcgcctgactggaaaaatatgacatggcaggagtgggagaaacaagtccgctacctagaggcaaatatcagtcaaagtttagaagaagcccaaattcaacaagaaaagaatatgtatgaattacaaaaattaaatagctgggatattcttggcaactggtttgacttaacctcctgggtcaagtatattcaatatggagtgcatatagtagtgggaataatagctttaagaatagcaatctatgtagtgcaattgttaagtagatttagaaagggctataggcctgttttctcttccccccccggttatctccaacagatccatatccacaaggaccggggacagccagccaacgaaggaacagaagaagacgtcggaggcgacagtggttacgacttgtggccttggccaataaactatgtgcagttcctgatccacctactgactcgcctcttgatcgggctatacaacatctgcagagacttactatccaagaactccccgacccgccgactgatctcccagagtctaacagcaatcagggactggctgagacttaaggcggcccaactgcaatatgggtgcgagtggatccaagaagctttccaagcattcgcgaggactacgagagagactcttgcgggcgcgtggggatggttatgggaagcagcgcgacgcatcgggaggggaatactcgcagttccaagaagaatcaggcagggagcagaactcgccctcctgtgagggacagcagtatcagcagggagagtacatgaacagcccatggagaaacccagcaacagaaagacagaaagatttgtataggcagcaaaatatggatgatgtagattctgatgatgatgacctaataggagttcctgttacaccaagagtaccacggagagaaatgacctataaattggcaatagatatgtcacattttataaaagaaaaagggggactgcaagggatgttttacagtaggaggagacatagaatcctagacatatacctagaaaaagaggaagggataataccagattggcagaattatactcatgggccaggagtaaggtacccaatgtacttcgggtggctgtggaagctagtatcagtagaactctcacaagaggcagaggaagatgaggccaactgcttagtacacccagcacaaacaagcagacatgatgatgagcatggggagacattagtgtggcagtttgactccatgctggcctataactacaaggccttcactctgtacccagaagagtttgggcacaagtcaggattgccagagaaagaatggaaggcaaaactgaaagcaagagggataccatatagtgaataacaggaacaaccatacttggtcaaggcaggaagtagctactaagaaacagctgaggctgcagggactttccagaaggggctgtaaccaagggagggacatgggaggagctggtggggaacgccctcatacttactgtataaatgtacccgcttcttgcattgtattcagtcgctctgcggagaggctggcagatcgagccctgagaggttctctccagcactagcaggtagagcctgggtgttccctgctggactctcaccagtacttggccggtactgggcagacggctccacgcttgcttgcttaaagacctcttcaataaagctgccagttagaagcaagttaagtgtgtgttcccatctctcctagtcgccgcctggtcattcggtgttcatctgagtaacaagaccctggtctgttaggacccttctcgctttgggaatccaaggcaggaaaatccctagca

SEQ ID NO: 19 - DNA, 3215 bp, Hepatitis B Virus HBV (NC_003977.1)
ctccacaacattccaccaagctctgctagatcccagagtgaggggcctatattttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaacttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcagaacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagctatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccgcaggaacatattgtacaaaaactcaagcaatgttttcgaaaattgcctgtaaatagacctattgattggaaagtatgtcaaagaattgtgggtcttttgggctttgctgccccttttacacaatgtggctatcctgccttgatgcctttatatgcatgtatacaatctaagcaggctttcactttctcgccaacttacaaggcctttctgtgtaaacaatatctaaacctttaccccgttgcccggcaacggtcaggtctctgccaagtgtttgctgacgcaacccccacgggttggggcttggccataggccatcggcgcatgcgtggaacctttgtggctcctctgccgatccatactgcggaactcctagcagcttgttttgctcgcagccggtctggagcgaaacttatcggaaccgacaactcagttgtcctctctcggaaatacacctcctttccatggctgctaggctgtgctgccaactggatcctgcgcgggacgtcctttgtctacgtcccgtcggcgctgaatcccgcggacgacccgtctcggggccgtttgggcctctaccgtccccttcttcatctgccgttccggccgaccacggggcgcacctctctttacgcggtctccccgtctgtgccttctcatctgccggaccgtgtgcacttcgcttcacctctgcacgtagcatggagaccaccgtgaacgcccaccaggtcttgcccaaggtcttacacaagaggactcttggactctcagcaatgtcaacgaccgaccttgaggcatacttcaaagactgtttgtttaaagactgggaggagttgggggaggagattaggttaaaggtctttgtactaggaggctgtaggcataaattggtctgttcaccagcaccatgcaactttttcccctctgcctaatcatctcatgttcatgtcctactgttcaagcctccaagctgtgccttgggtggctttggggcatggacattgacccgtataaagaatttggagcttctgtggagttactctcttttttgccttctgacttctttccttctattcgagatctcctcgacaccgcctctgctctgtatcgggaggccttagagtctccggaacattgttcacctcaccatacagcactcaggcaagctattctgtgttggggtgagttgatgaatctggccacctgggtgggaagtaatttggaagacccagcatccagggaattagtagtcagctatgtcaatgttaatatgggcctaaaaattagacaactattgtggtttcacatttcctgccttacttttggaagagaaactgtccttgagtatttggtgtcttttggagtgtggattcgcactcctcccgcttacagaccaccaaatgcccctatcttatcaacacttccggaaactactgttgttagacgacgaggcaggtcccctagaagaagaactccctcgcctcgcagacgaaggtctcaatcgccgcgtcgcagaagatctcaatctcgggaatctcaatgttagtatcccttggactcataaggtgggaaactttactgggctttattcttctactgtacctgtctttaatcctgattggaaaactccctcctttcctcacattcatttacaggaggacattattaatagatgtcaacaatatgtgggccctctgacagttaatgaaaaaaggagattaaaattaattatgcctgctaggttctatcctaaccttaccaaatatttgcccttggacaaaggcattaaaccgtattatcctgaatatgcagttaatcattacttcaaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcatttggtccagggttcaccccaccacacggaggccttttggggtggagccctcaggctcagggcatattgacaacactgccagcagcacctcctcctgcctccaccaatcggcagtcaggaagacagcctactcccatctctccacctctaagagacagtcatcctcaggccatgcagtggaa

SEQ ID NO: 20 - DNA, 7905 bp, Human Papilloma Virus HPV16 (NC_001526.2)
actacaataattcatgtataaaactaagggcgtaaccgaaatcggttgaaccgaaaccggttagtataaaagcagacattttatgcaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgtaatcatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataatctaccatggctgatcctgcaggtaccaatggggaagagggtacgggatgtaatggatggttttatgtagaggctgtagtggaaaaaaaaacaggggatgctatatcagatgacgagaacgaaaatgacagtgatacaggtgaagatttggtagattttatagtaaatgataatgattatttaacacaggcagaaacagagacagcacatgcgttgtttactgcacaggaagcaaaacaacatagagatgcagtacaggttctaaaacgaaagtatttggtagtccacttagtgatattagtggatgtgtagacaataatattagtcctagattaaaagctatatgtatagaaaaacaaagtagagctgcaaaaaggagattatttgaaagcgaagacagcgggtatggcaatactgaagtggaaactcagcagatgttacaggtagaagggcgccatgagactgaaacaccatgtagtcagtatagtggtggaagtgggggtggttgcagtcagtacagtagtggaagtgggggagagggtgttagtgaaagacacactatatgccaaacaccacttacaaatattttaaatgtactaaaaactagtaatgcaaaggcagcaatgttagcaaaatttaaagagttatacggggtgagtttttcagaattagtaagaccatttaaaagtaataaatcaacgtgttgcgattggtgtattgctgcatttggacttacacccagtatagctgacagtataaaaacactattacaacaatattgtttatatttacacattcaaagtttagcatgttcatggggaatggttgtgttactattagtaagatataaatgtggaaaaaatagagaaacaattgaaaaattgctgtctaaactattatgtgtgtctccaatgtgtatgatgatagagcctccaaaattgcgtagtacagcagcagcattatattggtataaaacaggtatatcaaatattagtgaagtgtatggagacacgccagaatggatacaaagacaaacagtattacaacatagttttaatgattgtacatttgaattatcacagatggtacaatgggcctacgataatgacatagtagacgatagtgaaattgcatataaatatgcacaattggcagacactaatagtaatgcaagtgcctttctaaaaagtaattcacaggcaaaaattgtaaaggattgtgcaacaatgtgtagacattataaacgagcagaaaaaaaacaaatgagtatgagtcaatggataaaatatagatgtgatagggtagatgatggaggtgattggaagcaaattgttatgtttttaaggtatcaaggtgtagagtttatgtcatttttaactgcattaaaaagatttttgcaaggcatacctaaaaaaaattgcatattactatatggtgcagctaacacaggtaaatcattatttggtatgagtttaatgaaatttctgcaagggtctgtaatatgttttgtaaattctaaaagccatttttggttacaaccattagcagatgccaaaataggtatgttagatgatgctacagtgccctgttggaactacatagatgacaatttaagaaatgcattggatggaaatttagtttctatggatgtaaagcatagaccattggtacaactaaaatgccctccattattaattacatctaacattaatgctggtacagattctaggtggccttatttacataatagattggtggtgtttacatttcctaatgagtttccatttgacgaaaacggaaatccagtgtatgagcttaatgataagaactggaaatcctttttctcaaggacgtggtccagattaagtttgcacgaggacgaggacaaggaaaacgatggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaagtggtgccaacactggctgtatcaaagaataaagcattacaagcaattgaactgcaactaacgttagaaacaatatataactcacaatatagtaatgaaaagtggacattacaagacgttagccttgaagtgtatttaactgcaccaacaggatgtataaaaaaacatggatatacagtggaagtgcagtttgatggagacatatgcaatacaatgcattatacaaactggacacatatatatatttgtgaagaagcatcagtaactgtggtagagggtcaagttgactattatggtttatattatgttcatgaaggaatacgaacatattttgtgcagtttaaagatgatgcagaaaaatatagtaaaaataaagtatgggaagttcatgcgggtggtcaggtaatattatgtcctacatctgtgtttagcagcaacgaagtatcctctcctgaaattattaggcagcacttggccaaccaccccgccgcgacccataccaaagccgtcgccttgggcaccgaagaaacacagacgactatccagcgaccaagatcagagccagacaccggaaacccctgccacaccactaagttgttgcacagagactcagtggacagtgctccaatcctcactgcatttaacagctcacacaaaggacggattaactgtaatagtaacactacacccatagtacatttaaaaggtgatgctaatactttaaaatgtttaagatatagatttaaaaagcattgtacattgtatactgcagtgtcgtctacatggcattggacaggacataatgtaaaacataaaagtgcaattgttacacttacatatgatagtgaatggcaacgtgaccaatttttgtctcaagttaaaataccaaaaactattacagtgtctactggatttatgtctatatgacaaatcttgatactgcatccacaacattactggcgtgctttttgctttgcttttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacataatgtatatgtacataatgtaattgttacatataattgttgtataccataacttactattttttcttttttattttcatatataatttttttttttgtttgtttgtttgttttttaataaactgttattacttaacaatgcgacacaaacgttctgcaaaacgcacaaaacgtgcatcggctacccaactttataaaacatgcaaacaggcaggtacatgtccacctgacattatacctaaggttgaaggcaaaactattgctgaacaaatattacaatatggaagtatgggtgtattttttggtgggttaggaattggaacagggtcgggtacaggcggacgcactgggtatattccattgggaacaaggcctcccacagctacagatacacttgctcctgtaagaccccctttaacagtagatcctgtgggcccttctgatccttctatagtttctttagtggaagaaactagttttattgatgctggtgcaccaacatctgtaccttccattcccccagatgtatcaggatttagtattactacttcaactgataccacacctgctatattagatattaataatactgttactactgttactacacataataatcccactttcactgacccatctgtattgcagcctccaacacctgcagaaactggagggcattttacactttcatcatccactattagtacacataattatgaagaaattcctatggatacatttattgttagcacaaaccctaacacagtaactagtagcacacccataccagggtctcgcccagtggcacgcctaggattatatagtcgcacaacacaacaggttaaagttgtagaccctgcttttgtaaccactcccactaaacttattacatatgataatcctgcatatgaaggtatagatgtggataatacattatatttttctagtaatgataatagtattaatatagctccagatcctgactttttggatatagttgctttacataggccagcattaacctctaggcgtactggcattaggtacagtagaattggtaataaacaaacactacgtactcgtagtggaaaatctataggtgctaaggtacattattattatgatttaagtactattgatcctgcagaagaaatagaattacaaactataacaccttctacatatactaccacttcacatgcagcctcacctacttctattaataatggattatatgatatttatgcagatgactttattacagatacttctacaaccccggtaccatctgtaccctctacatctttatcaggttatattcctgcaaatacaacaattccttttggtggtgcatacaatattcctttagtatcaggtcctgatatacccattaatataactgaccaagctccttcattaattcctatagttccagggtctccacaatatacaattattgctgatgcaggtgacttttatttacatcctagttattacatgttacgaaaacgacgtaaacgtttaccatattttttttcagatgtctctttggctgcctagtgaggccactgtctacttgcctcctgtcccagtatctaaggttgtaagcacggatgaatatgttgcacgcacaaacatatattatcatgcaggaacatccagactacttgcagttggacatccctattttcctattaaaaaacctaacaataacaaaatattagttcctaaagtatcaggattacaatacagggtatttagaatacatttacctgaccccaataagtttggttttcctgacacctcattttataatccagatacacagcggctggtttgggcctgtgtaggtgttgaggtaggtcgtggtcagccattaggtgtgggcattagtggccatcctttattaaataaattggatgacacagaaaatgctagtgcttatgcagcaaatgcaggtgtggataatagagaatgtatatctatggattacaaacaaacacaattgtgtttaattggttgcaaaccacctataggggaacactggggcaaaggatccccatgtaccaatgttgcagtaaatccaggtgattgtccaccattagagttaataaacacagttattcaggatggtgatatggttcatactggctttggtgctatggactttactacattacaggctaacaaaagtgaagttccactggatatttgtacatctatttgcaaatatccagattatattaaaatggtgtcagaaccatatggcgacagcttatttttttatttacgaagggaacaaatgtttgttagacatttatttaatagggctggtactgttggtgaaaatgtaccagacgatttatacattaaaggctctgggtctactgcaaatttagccagttcaaattattttcctacacctagtggttctatggttacctctgatgcccaaatattcaataaaccttattggttacaacgagcacagggccacaataatggcatttgttggggtaaccaactatttgttactgttgttgatactacacgcagtacaaatatgtcattatgtgctgccatatctacttcagaaactacatataaaaatactaactttaaggagtacctacgacatggggaggaatatgatttacagtttatttttcaactgtgcaaaataaccttaactgcagacgttatgacatacatacattctatgaattccactattttggaggactggaattttggtctacaacctcccccaggaggcacactagaagatacttataggtttgtaacccaggcaattgcttgtcaaaaacatacacctccagcacctaaagaagatgatccccttaaaaaatacactttttgggaagtaaatttaaaggaaaagttttctgcagacctagatcagtttcctttaggacgcaaatttttactacaagcaggattgaaggccaaaccaaaatttacattaggaaaacgaaaagctacacccaccacctcatctacctctacaactgctaaacgcaaaaaacgtaagctgtaagtattgtatgtatgttgaattagtgttgtttgttgtgtatatgtttgtatgtgcttgtatgtgcttgtaaatattaagttgtatgtgtgtttgtatgtatggtataataaacacgtgtgtatgtgtttttaaatgcttgtgtaactattgtgtcatgcaacataaataaacttattgtttcaacacctactaattgtgttgtggttattcattgtatataaactatatttgctacatcctgtttttgttttatatatactatattttgtagcgccaggcccattttgtagcttcaaccgaattcggttgcatgctttttggcacaaaatgtgtttttttaaatagttctatgtcagcaactatggtttaaacttgtacgtttcctgcttgccatgcgtgccaaatccctgttttcctgacctgcactgcttgccaaccattccattgttttttacactgcactatgtgcaactactgaatcactatgtacattgtgtcatataaaataaatcactatgcgccaacgccttacataccgctgttaggcacatatttttggcttgttttaactaacctaattgcatatttggcataaggtttaaacttctaaggccaactaaatgtcaccctagttcatacatgaactgtgtaaaggttagtcatacattgttcatttgtaaaactgcacatgggtgtgtgcaaaccgattttgggttacacatttacaagcaacttatataataatactaa

SEQ ID NO: 21 - DNA, 7857 bp, Human Papilloma Virus HPV18 (X05015.1)
attaatacttttaacaattgtagtatataaaaaagggagtaaccgaaaacggtcgggaccgaaaacggtgtatataaaagatgtgagaaacacaccacaatactatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccccatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtataatattaagtatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagtaagcaacaatggctgatccagaaggtacagacggggagggcacgggttgtaacggctggttttatgtacaagctattgtagacaaaaaaacaggagatgtaatatcagatgacgaggacgaaaatgcaacagacacagggtcggatatggtagattttattgatacacaaggaacattttgtgaacaggcagagctagagacagcacaggcattgttccatgcgcaggaggtccacaatgatgcacaagtgttgcatgttttaaaacgaaagtttgcaggaggcagcacagaaaacagtccattaggggagcggctggaggtggatacagagttaagtccacggttacaagaaatatctttaaatagtgggcagaaaaaggcaaaaaggcggctgtttacaatatcagatagtggctatggctgttctgaagtggaagcaacacagattcaggtaactacaaatggcgaacatggcggcaatgtatgtagtggcggcagtacggaggctatagacaacgggggcacagagggcaacaacagcagtgtagacggtacaagtgacaatagcaatatagaaaatgtaaatccacaatgtaccatagcacaattaaaagacttgttaaaagtaaacaataaacaaggagctatgttagcagtatttaaagacacatatgggctatcatttacagatttagttagaaattttaaaagtgataaaaccacgtgtacagattgggttacagctatatttggagtaaacccaacaatagcagaaggatttaaaacactaatacagccatttatattatatgcccatattcaatgtctagactgtaaatggggagtattaatattagccctgttgcgttacaaatgtggtaagagtagactaacagttgctaaaggtttaagtacgttgttacacgtacctgaaacttgtatgttaattcaaccaccaaaattgcgaagtagtgttgcagcactatattggtatagaacaggaatatcaaatattagtgaagtaatgggagacacacctgagtggatacaaagacttactattatacaacatggaatagatgatagcaattttgatttgtcagaaatggtacaatgggcatttgataatgagctgacagatgaaagcgatatggcatttgaatatgccttattagcagacagcaacagcaatgcagctgcctttttaaaaagcaattgccaagctaaatatttaaaagattgtgccacaatgtgcaaacattataggcgagcccaaaaacgacaaatgaatatgtcacagtggatacgatttagatgttcaaaaatagatgaagggggagattggagaccaatagtgcaattcctgcgataccaacaaatagagtttataacatttttaggagccttaaaatcatttttaaaaggaacccccaaaaaaaattgtttagtattttgtggaccagcaaatacaggaaaatcatattttggaatgagttttatacactttatacaaggagcagtaatatcatttgtgaattccactagtcatttttggttggaaccgttaacagatactaaggtggccatgttagatgatgcaacgaccacgtgttggacatactttgatacctatatgagaaatgcgttagatggcaatccaataagtattgatagaaagcacaaaccattaatacaactaaaatgtcctccaatactactaaccacaaatatacatccagcaaaggataatagatggccatatttagaaagtagaataacagtatttgaatttccaaatgcatttccatttgataaaaatggcaatccagtatatgaaataaatgacaaaaattggaaatgtttttttgaaaggacatggtccagattagatttgcacgaggaagaggaagatgcagacaccgaaggaaaccctttcggaacgtttaagttgcgtgcaggacaaaatcatagaccactatgaaaatgacagtaaagacatagacagccaaatacagtattggcaactaatacgttgggaaaatgcaatattctttgcagcaagggaacatggcatacagacattaaaccaccaggtggtgccagcctataacatttcaaaaagtaaagcacataaagctattgaactgcaaatggccctacaaggccttgcacaaagtcgatacaaaaccgaggattggacactgcaagacacatgcgaggaactatggaatacagaacctactcactgctttaaaaaaggtggccaaacagtacaagtatattttgatggcaacaaagacaattgtatgacctatgtagcatgggacagtgtgtattatatgactgatgcaggaacatgggacaaaaccgctacctgtgtaagtcacaggggattgtattatgtaaaggaagggtacaacacgttttatatagaatttaaaagtgaatgtgaaaaatatgggaacacaggtacgtgggaagtacattttgggaataatgtaattgattgtaatgactctatgtgcagtaccagtgacgacacggtatccgctactcagcttgttaaacagctacagcacaccccctcaccgtattccagcaccgtgtccgtgggcaccgcaaagacctacggccagacgtcggctgctacacgacctggacactgtggactcgcggagaagcagcattgtggacctgtcaacccacttctcggtgcagctacacctacaggcaacaacaaaagacggaaactctgtagtggtaacactacgcctataatacatttaaaaggtgacagaaacagtttaaaatgtttacggtacagattgcgaaaacatagcgaccactatagagatatatcatccacctggcattggacaggtgcaggcaatgaaaaaacaggaatactgactgtaacataccatagtgaaacacaaagaacaaaatttttaaatactgttgcaattccagatagtgtacaaatattggtgggatacatgacaatgtaatacatatgctgtagtaccaatatgttatcacttatttttttattttgcttttgtgtatgcatgtatgtgtgctgccatgtcccgcttttgccatctgtctgtatgtgtgcgtatgcatgggtattggtatttgtgtatattgtggtaataacgtcccctgccacagcattcacagtatatgtattttgttttttattgcccatgttactattgcatatacatgctatattgtctttacagtaattgtataggttgttttatacagtgtattgtacattgtatattttgttttataccttttatgctttttgtatttttgtaataaaagtatggtatcccaccgtgccgcacgacgcaaacgggcttcggtaactgacttatataaaacatgtaaacaatctggtacatgtccacctgatgttgttcctaaggtggagggcaccacgttagcagataaaatattgcaatggtcaagccttggtatatttttgggtggacttggcataggtactggcagtggtacagggggtcgtacagggtacattccattgggtgggcgttccaatacagtggtggatgttggtcctacacgtcccccagtggttattgaacctgtgggccccacagacccatctattgttacattaatagaggactccagtgtggttacatcaggtgcacctaggcctacgtttactggcacgtctgggtttgatataacatctgcgggtacaactacacctgcggttttggatatcacaccttcgtctacctctgtgtctatttccacaaccaattttaccaatcctgcattttctgatccgtccattattgaagttccacaaactggggaggtggcaggtaatgtatttgttggtacccctacatctggaacacatgggtatgaggaaatacctttacaaacatttgcttcttctggtacgggggaggaacccattagtagtaccccattgcctactgtgcggcgtgtagcaggtccccgcctttacagtagggcctaccaacaagtgtcagtggctaaccctgagtttcttacacgtccatcctctttaattacatatgacaacccggcctttgagcctgtggacactacattaacatttgatcctcgtagtgatgttcctgattcagattttatggatattatccgtctacataggcctgctttaacatccaggcgtgggactgttcgctttagtagattaggtcaacgggcaactatgtttacccgcagcggtacacaaataggtgctagggttcacttttatcatgatataagtcctattgcaccttccccagaatatattgaactgcagcctttagtatctgccacggaggacaatgacttgtttgatatatatgcagatgacatggaccctgcagtgcctgtaccatcgcgttctactacctcctttgcattttttaaatattcgcccactatatcttctgcctcttcctatagtaatgtaacggtccctttaacctcctcttgggatgtgcctgtatacacgggtcctgatattacattaccatctactacctctgtatggcccattgtatcacccacggcccctgcctctacacagtatattggtatacatggtacacattattatttgtggccattatattattttattcctaagaaacgtaaacgtgttccctatttttttgcagatggctttgtggcggcctagtgacaataccgtatatcttccacctccttctgtggcaagagttgtaaataccgatgattatgtgactcccacaagcatattttatcatgctggcagctctagattattaactgttggtaatccatattttagggttcctgcaggtggtggcaataagcaggatattcctaaggtttctgcataccaatatagagtatttagggtgcagttacctgacccaaataaatttggtttacctgatactagtatttataatcctgaaacacaacgtttagtgtgggcctgtgctggagtggaaattggccgtggtcagcctttaggtgttggccttagtgggcatccattttataataaattagatgacactgaaagttcccatgccgccacgtctaatgtttctgaggacgttagggacaatgtgtctgtagattataagcagacacagttatgtattttgggctgtgcccctgctattggggaacactgggctaaaggcactgcttgtaaatcgcgtcctttatcacagggcgattgcccccctttagaacttaaaaacacagttttggaagatggtgatatggtagatactggatatggtgccatggactttagtacattgcaagatactaaatgtgaggtaccattggatatttgtcagtctatttgtaaatatcctgattatttacaaatgtctgcagatccttatggggattccatgtttttttgcttacggcgtgagcagctttttgctaggcatttttggaatagagcaggtactatgggtgacactgtgcctcaatccttatatattaaaggcacaggtatgcctgcttcacctggcagctgtgtgtattctccctctccaagtggctctattgttacctctgactcccagttgtttaataaaccatattggttacataaggcacagggtcataacaatggtgtttgctggcataatcaattatttgttactgtggtagataccactcccagtaccaatttaacaatatgtgcttctacacagtctcctgtacctgggcaatatgatgctaccaaatttaagcagtatagcagacatgttgaggaatatgatttgcagtttatttttcagttgtgtactattactttaactgcagatgttatgtcctatattcatagtatgaatagcagtattttagaggattggaactttggtgttcccccccccccaactactagtttggtggatacatatcgttttgtacaatctgttgctattacctgtcaaaaggatgctgcaccggctgaaaataaggatccctatgataagttaaagttttggaatgtggatttaaaggaaaagttttctttagacttagatcaatatccccttggacgtaaatttttggttcaggctggattgcgtcgcaagcccaccataggccctcgcaaacgttctgctccatctgccactacgtcttctaaacctgccaagcgtgtgcgtgtacgtgccaggaagtaatatgtgtgtgtgtatatatatatacatctattgttgtgtttgtatgtcctgtgtttgtgtttgttgtatgattgcattgtatggtatgtatggttgttgttgtatgttgtatgttactatatttgttggtatgtggcattaaataaaatatgttttgtggttctgtgtgttatgtggttgcgccctagtgagtaacaactgtatttgtgtttgtggtatgggtgttgcttgttgggctatatattgtcctgtatttcaagttataaaactgcacaccttacagcatccattttatcctacaatcctccattttgctgtgcaaccgatttcggttgcctttggcttatgtctgtggttttctgcacaatacagtacgctggcactattgcaaactttaatcttttgggcactgctcctacatattttgaacaattggcgcgcctctttggcgcatataaggcgcacctggtattagtcattttcctgtccaggtgcgctacaacaattgcttgcataactatatccactccctaagtaataaaactgcttttaggcacatattttagtttgtttttacttaagctaattgcatacttggcttgtacaactactttcatgtccaacattctgtctacccttaacatgaactataatatgactaagctgtgcatacatagtttatgcaaccgaaataggttgggcagcacatactatacttttc

SEQ ID NO: 22 - DNA, 7912 bp, Human Papilloma Virus HPV31 (J04353.1)
taataataataatcttagtataaaaaagtagggagtgaccgaaagtggtgaaccgaaaacggttggtatataaagcacatagtattttgtgcaaacctacagacgccatgttcaaaaatcctgcagaaagacctcggaaattgcatgaactaagctcggcattggaaataccctacgatgaactaagattgaattgtgtctactgcaaaggtcagttaacagaaacagaggtattagattttgcatttacagatttaacaatagtatatagggacgacacaccacacggagtgtgtacaaaatgtttaagattttattcaaaagtaagtgaatttagatggtatagatatagtgtgtatggaacaacattagaaaaattgacaaacaaaggtatatgtgatttgttaattaggtgtataacgtgtcaaagaccgttgtgtccagaagaaaaacaaagacatttggataaaaagaaacgattccacaacataggaggaaggtggacaggacgttgcatagcatgttggagaagacctcgtactgaaacccaagtgtaaacatgcgtggagaaacacctacgttgcaagactatgtgttagatttgcaacctgaggcaactgacctccactgttatgagcaattacccgacagctcagatgaggaggatgtcatagacagtccagctggacaagcagaaccggacacatccaattacaatatcgttaccttttgttgtcagtgtaagtctacacttcgtttgtgtgtacagagcacacaagtagatattcgcatattgcaagagctgttaatgggctcatttggaatcgtgtgccccaactgttctactagactgtaactacaatggctgatccagcaggtacagatggggaggggacgggatgcaatggttggttttatgtagaagcagtaattgacagacagacaggggacaacatttcagaggacgaaaatgaagacagtagtgatactggggaggatatggttgactttattgacaattgtaatgtatacaacaatcaggcagaagcagagacagcacaggcattgtttcatgcacaggaagcggaggaacatgcagaggctgtgcaggttctaaaacgaaagtatgtaggtagtcctttaagtgatattagtagttgtgtggattataatattagtccacggttaaaagctatatgcatagaaaataacagtaaaacagcaaaacgaagactctttgaacttccagacagcgggtatggcaatactgaagtggaaacgcagcagatggtacaggtagaggagcaacaaacaacattaagttgtaatggtagtgacgggacacatagtgaacgagagaatgaaactccaacacgtaatatattgcaagtgttaaaaactagcaatggtaaagctgctatgttaggtaaatttaaagaattatatggtgtaagttttatggaactaattaggccatttcaaagcaataaaagcacatgtactgattggtgtgtagctgcgtttggagttacaggtacagttgcagaaggatttaaaaccctattgcaaccatattgtttgtattgccatttacaaagtttagcatgttcctggggcatggttatgttaatgcttgtgagatttaaatgtgcaaaaaatagaataacaattgaaaaattattagaaaaattattgtgtatatctacaaattgtatgttaattcagccacccaaattacgtagcacagctgcagcattatattggtacagaacaggaatgtcaaacattagcgatgtatatggtgaaacaccagaatggatagaaagacaaacagtattacagcatagttttaatgacacaacatttgatttgtcccaaatggtacaatgggcatatgacaatgatgttatggatgatagtgaaattgcctataaatatgcacaattagctgacagtgatagtaatgcatgtgcatttttaaaaagtaattcgcaggcaaaaatagttaaagattgtggaacaatgtgtagacattataaacgagcagaaaaacgacaaatgtccatgggacagtggattaaaagtagatgtgacaaagttagtgacgaaggtgactggagggacatagtaaagtttttaagatatcaacaaatagaatttgtgtcatttttatctgcattaaagctgtttttaaaaggagtgccaaagaaaaactgtattttaatacatggtgcacctaatacaggtaaatcatattttggaatgagccttattagctttttacaaggatgtataatatcatatgcaaattcaaaaagtcatttttggttacaaccactggctgatgctaaaataggcatgttagatgatgctacaacgccatgttggcattatatagacaattacctacgaaatgcactagatggcaaccctgtatctatagatgtaaagcataaagctttaatgcagttaaaatgtcctcctttattgattacatctaatataaatgcaggtaaggatgacagatggccatacctacatagcagactggtggtttttacatttccaaatccatttccatttgacaaaaacggaaatccagtatatgaattaagtgataaaaactggaaatcctttttctcaaggacgtggtgcagattaaatttgcacgaggaagaggacaaagaaaacgatggagactctttctcaacgtttaaatgtgtgtcaggacaaaatattagaacattatgaaaatgatagtaaacgactttgtgatcatatagactattggaaacatattcgacttgaatgtgtattaatgtataaagcaagagaaatgggaatacacagtattaaccaccaggtggtgccagcgttgtcagtatcaaaggccaaagccttacaagctattgaactacaaatgatgttggaaacattaaataacactgaatacaaaaatgaggactggacaatgcagcaaacaagtcttgaactgtatttaactgcacctacagggtgtttaaaaaaacatggatatactgtagaggtgcaatttgatggtgatgtacacaacaccatgcattatactaactggaaatttatatacctatgtatagatggccaatgtactgttgtggaagggcaagttaattgtaagggcatttattatgtacatgaaggacatataacatattttgtaaattttacagaagaggcaaaaaaatatgggactggtaaaaaatgggaagtgcatgcgggtggtcaggtaattgtttttcctgaatctgtatttagcagtgacgaaatatcctttgctgggattgttacaaagctaccaacagccaacaacaccaccacatcgaattccaaaacctgcgccttgggcaccagtgaaggtgtgcggcgggcgacgacgtctactaagcgaccaagaacagagccagagcacagaaacacccaccaccccaacaagttgttgcgaggcgactccgtggacagtgtcaactgtggggttatcagtgcagctgcatgcacaaaccaaacaagggctgtcagttgtcctgcaactacacctataatacacttaaaaggtgatgcaaatatattaaaatgtttaagatataggctgtcaaaatataaacaattgtatgaacaagtgtcatctacatggcattggacatgtacagatggaaaacataaaaatgctattgtaaccttaacatatataagtacatcacaaagagacgattttttaaatactgtaaaaatacctaacacagtatcagtgtcaacaggatatatgactatttagcctaatgattgaactaaatatttctacagtaagcattgtgctatgctttttgctttgcttttgtgtgctactatttgtgtgtcttgtcatacgtccacttgtgctgtctgtgtcggtatatgcaacactactattattaattgtgattttatgggttattgcaacctctccattacgttgtttttgtatatatgttgtgtttatatatattccattatttgtaattcatacacatgcatcttttttaagtcaacagtaacttttttacttgtgtatactgttgtttgtattggtattggtattggtattggtattggtattggtataataaacttttttactttttttttattattaccatgcggtccaaacgctctacaaaacgcactaaacgtgcgtctgctacacaattatatcaaacatgtaaagcagcaggtacttgtccatcagacgttatacctaaaatagaacatactaccattgcagaccaaatattaaggtatggtagtatgggtgttttttttggtgggttgggtattgggtccggctctggtactgggggtcgcactggatatgtccctcttagtacacgtccttctacagtatctgaggcaagtatacctattagaccaccagttagcattgaccctgtaggtcccttggacccctctatagtaagtcttgttgaagaatctggaattgttgatgttggtgcccctgctcctataccacaccctcctacaacatctgggtttgacattgctacaactgcagacacaacacctgcaattttagatgtaacaagtgttagcacacatgaaaatcctacttttactgatccatctgtattgcagcctcctacacctgcagaaacatcaggtcatttactactttcatcatcatctattagcacacataattatgaggaaatacctatggatacatttattgtttctactaataatgaaaacataacaagtagcacacccattccaggggtgcgccgtcctgcacgtttagggttatatagtaaggctacacaacaagtaaaagttattgatccaacgtttcttagtgctccaaaacagctaattacatatgaaaaccctgcctatgaaactgtaaatgctgaagaatctttatacttttccaatacatcgcataatatagcccctgatcccgactttctagatattatagcattacataggcctgcccttacctcacgtaggaacactgttagatatagtagactaggtaataaacaaactttgcgcactcgtagtggtgctactattggtgcaagggtgcattattattatgatattagtagtattaatcctgcaggtgaaagtattgaaatgcaacctttaggggcgtctgcaactactacttctactttaaatgatggcttatatgacatttatgcagacactgattttactgtggatacacctgccacacataatgtttccccttctactgctgtacagtccacatctgctgtgtctgcctatgtacctacaaataccactgtgccactaagtacaggttttgacattcccatattttctgggcctgatgtacctatagagcatgcacctacacaggttttcccatttcctttggcccctacaacgccacaagtgtctatttttgttgatgggggtgatttttatttgcaccctagttattatatgttaaaacgtcgacgtaaacgtgtatcatatttttttacagatgtctctgtggcggcctagcgaggctactgtctacttaccacctgtcccagtgtctaaagttgtaagcacggatgaatatgtaacacgaaccaacatatattatcacgcaggcagtgctaggctgcttacagtaggccatccatattattccatacctaaatctgacaatcctaaaaaaatagttgtaccaaaggtgtcaggattacaatatagggtatttagggttcgtttaccagatccaaacaaatttggatttcctgatacatctttttataatcctgaaactcaacgcttagtttgggcctgtgttggtttagaggtaggtcgcgggcagccattaggtgtaggtattagtggtcatccattattaaataaatttgatgacactgaaaactctaatagatatgccggtggtcctggcactgataatagggaatgtatatcaatggattataaacaaacacaactgtgtttacttggttgcaaaccacctattggagagcattggggtaaaggtagtccttgtagtaacaatgctattacccctggtgattgtcctccattagaattaaaaaattcagttatacaagatggggatatggttgatacaggctttggagctatggattttactgctttacaagacactaaaagtaatgttcctttggacatttgtaattctatttgtaaatatccagattatcttaaaatggttgctgagccatatggcgatacattatttttttatttacgtagggaacaaatgtttgtaaggcatttttttaatagatcaggcacggttggtgaatcggtccctactgacttatatattaaaggctccggttcaacagctactttagctaacagtacatactttcctacacctagcggctccatggttacttcagatgcacaaatttttaataaaccatattggatgcaacgtgctcagggacacaataatggtatttgttggggcaatcagttatttgttactgtggtagataccacacgtagtaccaatatgtctgtttgtgctgcaattgcaaacagtgatactacatttaaaagtagtaattttaaagagtatttaagacatggtgaggaatttgatttacaatttatatttcagttatgcaaaataacattatctgcagacataatgacatatattcacagtatgaatcctgctattttggaagattggaattttggattgaccacacctccctcaggttctttggaggatacctataggtttgtcacctcacaggccattacatgtcaaaaaactgccccccaaaagcccaaggaagatccatttaaagattatgtattttgggaggttaatttaaaagaaaagttttctgcagatttagatcagtttccactgggtcgcaaatttttattacaggcaggatatagggcacgtcctaaatttaaagcaggtaaacgtagtgcaccctcagcatctaccactacaccagcaaaacgtaaaaaaactaaaaagtaatggatgtgtatgtaatacatgtgtctgtatgtgtatgtgcttgtgctgtattgtatatgtgtgtgtttgtgtgttatatatggtatatgtatgtttatgtatgcgtgtgtacttgtatatatgtatagtatgttatgtgtgtatgtatgctatgtatgttaataaatatgtgtatacctgtgtgtgttgtgtatgttgtccttatatacaccctattagtaacatactattactattttataaactattgttcctacttgttcctacttgttcctgctcctcccaatagtcatgtacttatttctgcctataatttaggtgtcacgccatagtaaaagttgtacacccggtccgttttttgcaactaaagctactccattttgattttatgcagccattttaaatccctaaccgttttcggttgcattgtttaaacatgctagtacaactatgctgatgcagtagttctgcggtttttggtttcctgaatactagtttttgccaacattctggcttgtagtttcctgcctaacacaccttgccaacatataatccagtccaactttgcaattatactatgaatcatgtttgtttaaatacaactgtagttcaactatgtgtcatgcacatatattatattatcctacacaccttaaactgcttttaggcacatattttgtagattatctatatccttgattgcagtgctggcttttgcacatgtttaaactgccaaggttgtgtcatgcattataaataagttgtatgttactcatataattaattgcatataggtattacaccgttttcggttacagttttacaagcaattgttctttttatact

SEQ ID NO: 23 - DNA, 7909 bp, Human Papillomavirus  HPV33 (M12732.1)
gtaaactataatgccaagttttaaaaaagtagggtgtaaccgaaagcggttcaaccgaaaacggtgcatatataaagcaaacattttgcagtaaggtactgcacgactatgtttcaagacactgaggaaaaaccacgaacattgcatgatttgtgccaagcattggagacaactatacacaacattgaactacagtgcgtggaatgcaaaaaacctttgcaacgatctgaggtatatgattttgcatttgcagatttaacagttgtatatagagagggaaatccatttggaatatgtaaactgtgtttgcggttcttatctaaaattagtgaatatagacattataattattctgtatatggaaatacattagaacaaacagttaaaaaacctttaaatgaaatattaattaggtgtattatatgtcaaagacctttgtgtcctcaagaaaaaaaacgacatgtggatttaaacaaacgatttcataatatttcgggtcgttgggcagggcgctgtgcggcgtgttggaggtcccgacgtagagaaactgcactgtgacgtgtaaaaacgccatgagaggacacaagccaacgttaaaggaatatgttttagatttatatcctgaaccaactgacctatactgctatgagcaattaagtgacagctcagatgaggatgaaggcttggaccggccagatggacaagcacaaccagccacagctgattactacattgtaacctgttgtcacacttgtaacaccacagttcgtttatgtgtcaacagtacagcaagtgacctacgaaccatacagcaactacttatgggcacagtgaatattgtgtgccctacctgtgcacaacaataaacatcatctacaatggccgatcctgaaggtacaaatggggctgggatggggtgtactggttggtttgaggtagaagcagtcatagagagaagaacaggagataatatttcagaagatgaggatgaaacagcagatgacagtggcacggatttactagagtttatagatgattctatggaaaatagtatacaggcagacacagaggcagcccgggcattgtttaatatacaggaaggggaggatgatttaaatgctgtgtgtgcactaaaacgaaagtttgccgcatgttcacaaagtgctgcggaggacgttgttgatcgtgctgcaaacccgtgtagaacgtctattaataaaaataaagaatgcacatacagaaaacgaaaaatagatgagctagaagacagcggatatggcaatactgaagtggaaactcagcagatggtacaacaggtagaaagtcaaaatggcgacacaaacttaaatgacttagaatctagtggggtgggggatgattcagaagtaagctgtgagacaaatgtagatagctgtgaaaatgttacgttgcaggaaattagtaatgttctacatagtagtaatacaaaagcaaatatattatataaatttaaagaggcctatggaataagttttatggaattagtaagaccatttaaaagtgataaaacaagctgtacagattggtgtataacaggatatggaattagtccatcagtagcagaaagtttaaaagtattaattaaacagcatagtttgtatactcatttacaatgtttaacttgcgatagaggaataataatattattgttaattagatttaggtgtagcaaaaacaggttaacagtagcaaaactaatgagtaatttattatcaatacctgaaacatgtatggttatagagccaccaaaattacggagccaaacatgtgcattgtattggtttagaacagcaatgtcaaacattagtgatgtacaaggtacaacacctgaatggatagatagactaactgttttacaacatagctttaatgataatatatttgatttaagtgaaatggtacagtgggcatatgataacgagttaacggacgatagtgacattgcatattattatgcacaacttgcagattcaaatagtaatgctgctgcatttttaaaaagtaactcacaagcaaaaatagtaaaggactgtggaataatgtgtagacattataaaaaagcagaaaaacgtaaaatgtcaataggacaatggatacaaagtagatgtgaaaaaacaaatgatggaggaaattggagaccaatagtacagttgttaagatatcaaaacattgaatttacagcatttttaggtgcatttaaaaagtttttaaaaggtataccaaaaaaaagctgtatgctaatttgtggaccagcaaatacaggaaagtcatattttggaatgagtttaatacagtttttaaaagggtgtgttatatcatgtgtaaattctaaaagtcacttttggttgcagccattatcagatgcaaaaataggaatgatagatgatgtaacgccaataagttggacatatatagatgattacatgagaaatgcgttagatggaaatgaaatttcaatagatgtgaaacatagggcattagtgcaattaaaatgtccaccactgcttcttacctcaaatacaaatgcaggcacagactctagatggccatatttacatagtagattaacagtatttgaatttaaaaatccattcccatttgatgaaaatggtaacccagtgtatgcaataaatgatgaaaattggaaatcctttttctcaaggacgtggtgcaaattagatttaatagaggaagaggacaaggaaaaccatggaggaaatatcagcacgtttaaatgcagtgcaggagaaaatactagatctttacgaagctgataaaactgatttaccatcacaaattgaacattggaaactgatacgcatggagtgtgctttattgtatacagccaaacaaatgggattttcacatttatgccaccaggtggtgccttctttgttagcatcaaagaccaaagcatttcaagtaattgaactacaaatggcattagagacattaagtaaatcacagtatagtacaagccaatggacattgcaacaaacaagcttagaggtgtggctttgtgaaccaccaaaatgttttaaaaaacaaggagaaacagtaactgtgcaatatgacaatgacaaaaaaaatacaatggattatacaaactggggtgaaatatatattatagaggaagatacatgtactatggttacagggaaagtagattatataggtatgtattatatacataactgtgaaaaggtatattttaaatattttaaagaggatgctgcaaagtattctaaaacacaaatgtgggaagtacatgtgggtggtcaggtaattgtttgtcctacgtctatatctagcaaccaaatatccactactgaaactgctgacatacagacagacaacgataaccgaccaccacaagcagcggccaaacgacgacgacctgcagacaccacagacaccgcccagccccttacaaagctgttctgtgcagaccccgccttggacaatagaacagcacgtactgcaactaactgcacaaacaagcagcggactgtgtgtagttctaacgttgcacctatagtgcatttaaaaggtgaatcaaatagtttaaaatgtttaagatacagattaaaaccttataaagagttgtatagttctatgtcatccacctggcattggaccagtgacaacaaaaatagtaaaaatggaattgtaactgtaacatttgtaactgaacagcaacaacaaatgtttttaggtaccgtaaaaataccacctactgtgcaaataagtactggatttatgacattataagtgtacatcacaagccaatatgtgctgctaattgtatataaccatgatatttgtttttgtattatgttttatattgtttttatgcttatccttattattacgtcctttaatactttccatttctacctatgcttggttgctggtgttggtattgctgctttgggtgtttgtgggatctcctttaaaaatttttttttgctatttgttgtttttatatttaccaatgatgtgtattaattttcatgcacagcatatgacacaacaagagtaatgtatatacatgtatatattgtttgtatatatgtgcacatggtggtgttttaacattgttgttgttattttagtttttttttttttgtattactaataaatacctttatattttagcagtgtattattatgagacacaaacgatctacaaggcgcaagcgtgcatctgcaacacaactataccaaacatgcaaggccacaggcacctgcccacccgatgttattcctaaagtggaaggaagtaccatagcagatcaaattcttaaatatggcagtttaggggttttttttggtggtttaggtattggcacaggctctggttcaggtggaaggactggctatgtacctattggtactgacccacctacagctgcaatccccttgcagcctatacgtcctccggttactgtagacactgttggacctttagactcgtctatagtgtcattaatagaagaaacaagttttatagaggcaggtgcaccagccccatctattcctacaccatcaggttttgatgttactacatctgcagatactacacctgcaattattaatgtttcatctgttggggagtcatctattcaaactatttctacacatttaaatcccacatttactgaaccatctgtactacaccctccagcgcctgcagaagcctctggacattttatattttcttcccctactgttagcacacaaagttatgaaaacataccaatggatacctttgttgtttccacagacagtagtaatgtaacatcaagcacgcccattccagggtctcgccctgtggcacgccttggtttatatagtcgcaatacccaacaggttaaggttgttgaccctgcttttttaacatcgcctcataaacttataacatatgataatcctgcatttgaaagctttgaccctgaagacacattacaatttcaacatagtgatatatcacctgctcctgatcctgactttctagatattattgcattacataggcctgctattacatctcgtagacatactgtgcgttttagtagagtaggtcaaaaagccacacttaaaactcgcagtggtaaacaaattggagctagaatacattattatcaggatttaagtcctattgtgcctttagaccacaccgtgccaaatgaacaatatgaattacagcctttacatgatacttctacatcgtcttatagtattaatgatggtttgtatgatgtttatgctgacgatgtggataatgtacacaccccaatgcaacactcatacagtacgtttgcaacaacacgtaccagcaatgtgtctatacctttaaatacaggatttgatactcctgttatgtctggccctgatataccttcccctttatttcccacatctagcccatttgttcctatttcgcctttttttccttttgacaccattgttgtagacggtgctgactttgttttacatcctagttattttattttacgtcgcaggcgtaaacgttttccatatttttttacagatgtccgtgtggcggcctagtgaggccacagtgtacctgcctcctgtacctgtatctaaagttgtcagcactgatgaatatgtgtctcgcacaagcatttattattatgctggtagttccagacttcttgctgttggccatccatatttttctattaaaaatcctactaacgctaaaaaattattggtacccaaagtatcaggcttgcaatatagggtttttagggtccgtttaccagatcctaataaatttggatttcctgacacctccttttataaccctgatacacaacgattagtatgggcatgtgtaggccttgaaataggtagagggcagccattaggcgttggcataagtggtcatcctttattaaacaaatttgatgacactgaaaccggtaacaagtatcctggacaaccgggtgctgataatagggaatgtttatccatggattataaacaaacacagttatgtttacttggatgtaagcctccaacaggggaacattggggtaaaggtgttgcttgtactaatgcagcacctgccaatgattgtccacctttagaacttataaatactattattgaggatggtgatatggtggacacaggatttggttgcatggattttaaaacattgcaggctaataaaagtgatgttcctattgatatttgtggcagtacatgcaaatatccagattatttaaaaatgactagtgagccttatggtgatagtttatttttctttcttcgacgtgaacaaatgtttgtaagacacttttttaatagggctggtacattaggagaggctgttcccgatgacctgtacattaaaggttcaggaactactgcctctattcaaagcagtgctttttttcccactcctagtggatcaatggttacttccgaatctcagttatttaataagccatattggctacaacgtgcacaaggtcataataatggtatttgttggggcaatcaggtatttgttactgtggtagataccactcgcagtactaatatgactttatgcacacaagtaactagtgacagtacatataaaaatgaaaattttaaagaatatataagacatgttgaagaatatgatctacagtttgtttttcaactatgcaaagttaccttaactgcagaagttatgacatatattcatgctatgaatccagatattttagaagattggcaatttggtttaacacctcctccatctgctagtttacaggatacctataggtttgttacctctcaggctattacgtgtcaaaaaacagtacctccaaaggaaaaggaagaccccttaggtaaatatacattttgggaagtggatttaaaggaaaaattttcagcagatttagatcagtttcctttgggacgcaagtttttattacaggcaggtcttaaagcaaaacctaaacttaaacgtgcagcccccacatccacccgcacatcgtctgcaaaacgcaaaaaggttaaaaaataacactttgtgtaattgtgttatgttgttgttttgttctgtctatgtactttgtgttgttgtgttgtgttgttgtttgttttttgtgtatgtgttacaatgtatgttatgttgtatgttactgtgtttgttttatgtgtacttgtttgtgtgcatgttctatgtacttgtcagtttcctgtttgtgtatatgttaataaaacattgtgtgtatttgttaaactatttgtatgtatgttatgtatatgggtgtacctatatgagtaaggagttgtattgcttgccctaccctgcattgcaatgtacctacctttatttccctatatttgtagtacctacatgtttagtattgctttaccttttgacatactagtgtccatattgtacaatttcctccattttgtatgcctaaccgttttcggttacttggcatacataccctatgacattggcagaacagttaatccttttctttcctgcactgtgtttgtctgtacttgctgcattggcatacataccctatgacattggcagaacagttaatccttttctttcctgcactgtgtttgtctgtacttgctgcattgactcatatatacatgcagtgcaattgcaaaatacttaattgtactaatagtttacacatgcttttaggcacatatttttactttactttcaaaccttaagtgcagttttggcttacacaattgctttgtatgccaaactatgccttgtaaaagtgagtcactacctgtttattaccaggtgtggactaaccgttttaggtcatattggtcatttataatcttttatataata

SEQ ID NO: 24 - DNA, 7858 bp, Human Papillomavirus HPV45 (X74479.1)
aatacttttaacaattatactacataaaaaagggtgtaaccgaaaacggttgcaaccaaaaacggtgcatataaaagctttgtggaaaagtgcattacaggatggcgcgctttgacgatccaaagcaacgaccctacaagctaccagatttgtgcacagaattgaatacatcactacaagacgtatctattgcctgtgtatattgcaaagcaacattggaacgcacagaggtatatcaatttgcttttaaagatttatgtatagtgtatagagactgtatagcatatgctgcatgccataaatgtatagacttttattccagaattagagaattaagatattattcaaactctgtatatggagagacactggaaaaaataactaatacagagttgtataatttgttaataaggtgcctgcggtgccagaaaccattgaacccagcagaaaaacgtagacaccttaaggacaaacgaagatttcacagcatagctggacagtaccgagggcagtgtaatacatgttgtgaccaggcacggcaagaaagacttcgcagacgtagggaaacacaagtatagcaataagtatgcatggaccccgggaaacactgcaagaaattgtattgcatttggaacctcagaatgaattagatcctgttgacctgttgtgttacgagcaattaagcgagtcagaggaggaaaacgatgaagcagatggagttagtcatgcacaactaccagcccgacgagccgaaccacagcgtcacaaaattttgtgtgtatgttgtaagtgtgacggcagaattgagcttacagtagagagctcggcagaggaccttagaacactacagcagctgtttttgagcaccttgtcctttgtgtgtccgtggtgtgcaactaaccaataatctacaatggcggatccagaaggtaccgacggggagggaacggggtgtaatggctggttctttgtagaaacaattgtagagaaaaaaacaggggatgtaatatcagatgatgaggatgaaactgcaacagatacagggtcggatatggtagattttattgacacacaattatccatttgtgaacaggcagagcaagagacagcacaggcattgttccatgcgcaggaagttcagaatgatgcacaggtgttgcatcttttaaaacgaaagtttgcaggaggcagcaaggaaaacagtccattaggggagcagctaagtgtggatacggatctaagtccacggttacaagaaatttcattaaatagtgggcacaaaaaagcaaaacgacggttgtttacaatatcagatagtggctatggctgttctgaagtggaagctgcagagactcaggtaactgtaaacactaatgcggaaaatggcggcagtgtacatagtacacaaagtagtggtggggatagtagtgacaatgcagaaaatgtagatccgcattgcagtattacagaactaaaggagctattacaagcaagtaacaaaaaggctgcaatgctggcagtatttaaagacatatatgggctgtcatttacggatttggttagaaattttaaaagtgataaaacaacatgtacagattgggtaatggctatatttggagttaatccaacggtagcagaaggctttaaaacattaattaaaccagcaacgttatacgcccatatccaatgtttagattgtaaatggggagtattaatattagctttattaagatataaatgtggcaaaaatagactaactgttgcaaaaggcttaagcacattgttgcacgtacctgaaacatgtatgttaattgaaccaccaaaattgcgaagtagtgttgcagcattatactggtatagaacaggtatatccaatattagtgaagtaagtggagacacacctgagtggatacaaagactgacaattattcaacatggtattgacgatagtaattttgatttgtcagacatggtgcaatgggcatttgataatgaccttacagatgaaagtgatatggcatttcaatatgcccaattagcagactgcaacagtaatgcagctgcatttttaaaaagtaactgccaagccaaatatttaaaagattgtgctgtaatgtgtagacattataaaagagcacaaaaacgccaaatgaatatgtctcaatggattaaatatagatgttccaaaatagatgaaggtggggattggagacccatagtacaattcctaagatatcagggagtagaatttattagctttttaagggcactaaaggaatttcttaaaggaacaccaaaaaaaaattgtatactgttatatggacctgcaaatacaggaaaatcgtattttggaatgagttttatacatttcctacaaggtgcaataatatcatttgtaaattcaaacagccatttttggttagaaccgttagcagatactaaggtagccatgttggatgatgccacacacacgtgttggacatattttgataattatatgagaaatgcattagatggtaatcctataagtatagacagaaagcataaaccattattacagctaaaatgtcctccaatcctattaacatccaatattgatccagcaaaagataataaatggccatatttagaaagtagggtgacggtatttacatttccacatgcatttccatttgataaaaatggtaatccagtatatgaaataaatgataaaaattggaaatgtttttttgaaaggacatggtccagattagatttgcacgaggacgatgaagatgcagacaccgaaggaatccctttcggaacgtttaagtgcgttacaggacaaaatactagaccactatgaaaatgacagtaaagacataaacagccaaataagttattggcaacttatacgtttggaaaatgcaatactatttacagcaagggaacatggtattaccaaactaaaccaccaggtggtgcctcctattaacatttcaaaaagcaaagcacataaagctattgaactgcaaatggccttaaagggccttgcacaaagcaagtataacaatgaggaatggacactgcaagatacatgcgaggaactatggaatacagaaccgtcgcagtgttttaaaaaaggcggtaaaaccgtgcacgtatactttgatggcaacaaggacaactgtatgaactatgtagtatgggacagtatatattatataactgagacagggatatgggacaaaacagcagcatgtgttagctattggggtgtatattatataaaagatggagataccacatattatgtacaatttaaaagcgaatgtgagaaatatggaaatagtaatacgtgggaagtacaatatgggggcaatgtaattgattgtaatgactctatgtgcagtaccagtgacgacacggtatccgctactcagattgttagacagctacaacacgcctccacgtcgacccccaaaaccgcatccgtgggcaccccaaaaccccacatccagacgccggctactaagcgacctagacagtgtggactcacagagcagcaccacggacgtgtcaacacccacgtgcacaacccgctcctgtgttcaagtacaagtaacaacaaaagaaggaaagtgtgtagtggtaacactacgcctataatacacttaaaaggtgacaaaaacagtttgaaatgtttaagatataggctacgcaaatatgcagaccattactcagaaatatcctccacctggcattggacaggttgtaataaaaacactggtatattaactgtaacatataatagtgaggtacaaagaaatacctttttggatgtagttactattcctaacagtgtacaaatctcggtgggatacatgactatatgaatctgtatattgtatacagtatgtaacattactatgctatctttagtgtttttattgtgcttttctgtgtgcctttatgtgtgctgcaatgtcccgcttgtgcagtctgtctatgtgtgtgcttttgcttggttgttggtgtttctttttatagttgttattacatccccattaacagcatttgctgtatacatttgttgctatttactacctatgtttgtattacatatgcatgctttacacaccatacaataattactataatgtacagtacagtgtaacatacctgtgatgtgcatgttgttgtatttttgtatttttgtatttttgtatttttgtattttatatgtttaataaaccatggtatcccaccgtgcagcacgtcgcaagcgggcctctgcaactgacttatatagaacatgtaagcaatccggtacgtgcccccctgatgttattaacaaagtggaaggcacaaccttagctgataaaattttacagtggtctagccttgggatatttttgggtggccttggcattggtaccggcagtggttctggaggccgtacgggctatgtacccttagggggcaggtctaatactgttgtggatgttggccccactaggccacctgtggttattgaacctgtagggcctactgatccatctattgttacgttggtagaggattccagtgttgttgcctctggtgctccggttcccacatttaccggaacctctgggtttgaaattacgtcttctggtactaccacaccagctgtgttggacatcacacctaccgtggactctgtttctatttcgtcaactagttttacaaatcctgcattttctgatccctctattattgaggtgccccaaacaggggaggtatcaggtaatatatttgttggtacaccaacatcgggcagccatggatatgaggaaatacctttacaaacatttgcatcttctgggtcaggtacggaacccattagtagtacccccctccctactgtgcggcgggtacggggtccccgcctgtatagtagggctaatcaacaggtccgtgtgtccacctcacagtttttaacacatccctcatcgttggttacatttgataatccagcttatgagcccctggacaccacactatcctttgagcctaccagtaatgttcctgattccgattttatggatattattcgtttgcataggccagcattatcctctagacgtggcactgttagatttagtagattgggtcaaagggcaaccatgtttacacgtagtggtaaacaaatagggggtagggtacatttttaccatgatataagccccattgctgctacagaggaaattgaattgcagcctttaattagtgctacaaatgatagtgacctgtttgatgtatatgcagacttcccacctcctgcgtccactacacctagcactatacacaaatcatttacatatccaaagtattccttgaccatgccttctactgctgcatcctcttacagtaatgttacagtaccattaacatctgcatgggatgtacctatatatactggcccggacattatattgccatcccatactcctatgtggcctagtacatctcctaccaatgcttccaccaccacctatataggtattcatggcacacaatattatttatggccatggtattattattttcctaaaaaacgtaaacgtattccctatttttttgcagatggctttgtggcggcctagtgacagtacggtatatcttccaccaccttctgtggccagagttgtcagcactgatgattatgtgtctcgcacaagcatattttatcatgcaggcagttcccgattattaactgtaggcaatccatattttagggttgtacctaatggtgcaggtaataaacaggctgttcctaaggtatccgcatatcagtatagggtgtttagagtagctttacccgatcctaataaatttggattacctgattctactatatataatcctgaaacacaacgtttggtttgggcatgtgtaggtatggaaattggtcgtgggcagcctttaggtattggcctaagtggccatccattttataataaattggatgatacagaaagtgctcatgcagctacagctgttattacgcaggatgttagggataatgtgtcagttgattataagcaaacacagctgtgtattttaggttgtgtacctgctattggtgagcactgggccaagggcacactttgtaaacctgcacaattgcaacctggtgactgtcctcctttggaacttaaaaacaccattattgaggatggtgatatggtggatacaggttatggggcaatggattttagtacattgcaggatacaaagtgcgaggttccattagacatttgtcaatccatctgtaaatatccagattatttgcaaatgtctgctgatccctatggggattctatgtttttttgcctacgccgtgaacaactgtttgcaagacatttttggaatagggcaggtgttatgggtgacacagtacctacggacctatatattaaaggcactagcgctaatatgcgtgaaacccctggcagttgtgtgtattccccttctcccagtggctctattattacttctgattctcaattatttaataagccatattggttacataaggcccagggccataacaatggtatttgttggcataatcagttgtttgttactgtagtggacactacccgcagtactaatttaacattatgtgcctctacacaaaatcctgtgccaagtacatatgaccctactaagtttaagcagtatagtagacatgtggaggaatatgatttacagtttatttttcagttgtgcactattactttaactgcagaggttatgtcatatatccatagtatgaatagtagtatattagaaaattggaattttggtgtccctccaccacctactacaagtttggtggatacatatcgttttgtgcaatcagttgctgttacctgtcaaaaggatactacacctccagaaaagcaggatccatatgataaattaaagttttggactgttgacctaaaggaaaaattttcctccgatttggatcaatatccccttggtcgaaagtttttagttcaggctgggttacgtcgtaggcctaccataggacctcgtaagcgtcctgctgcttccacgtctactgcatctactgcatctaggcctgccaaacgtgtacgtatacgtagtaagaaataatatgttagcacatatatgtatgtttgtatgtatggttttgtatgttgtatgtatgtatgtatttgtgtgatatattactgtattttgtttgtttgcgtgcgtgtatgtatgaatgtgccttgtggcatgtatggtgttactgtacataattgtggtattaaataaagtatgctaatagtgttgtgtagggttgcacccttgtgagtaacaatactatttgtgtgtatgtgtattgctttgtaccctatattctttcctgtatttcaagttataaacttgcatactacacagcatccattttacttataatcctccattttgctgtgcaaccgatttcggttgcctgtggcttatatgtgaccttttaaacataatacctaaactggcacatttacaacccctacatagtttaacctactggcgcgccttcttggcgtacatgtggcacacctggtattagtcattttcctgtccaggtgtactaaaacaatggcttgcacaactgtatccacaccctatgtaataaaactgcttttaggcacatattttagtctgtttttacctgtgctaattgtataattggcgtgtagaaccactttcttatccaacaatctgtctacttgttacataaactataaactgactcacttatacatacatagtttatgcaaccgaaaaaggttgggccctataacacataccttttctt